(19) United States
(12) Patent Application Publication
(10) Pub. No.: US 2025/0296936 A1
(43) Pub. Date: Sep. 25, 2025

(54) SARS-COV2 MAIN PROTEASE INHIBITORS

(71) Applicant: Gilead Sciences, Inc., Foster City, CA (US)

(72) Inventors: Stephen E. Ammann, Foster City, CA (US); Eda Y. Canales, San Mateo, CA (US); Xinpei Cai, San Mateo, CA (US); Weng K. Chang, San Lorenzo, CA (US); Henok H. Kinfe, Foster City, CA (US); Devan Naduthambi, San Bruno, CA (US); Kevin X. Rodriguez, San Mateo, CA (US); Scott D. Schroeder, Union City, CA (US); Christopher J. Swank, San Mateo, CA (US)

(21) Appl. No.: 19/045,761

(22) Filed: Feb. 5, 2025

Related U.S. Application Data
(60) Provisional application No. 63/550,933, filed on Feb. 7, 2024.

(51) Int. Cl. C07D 491/048; A61K 31/519; A61P 31/14; C07D 519/00

(52) U.S. Cl. CPC C07D 491/048; A61K 31/519; A61P 31/14; C07D 519/00

(57) ABSTRACT

The present disclosure relates to compounds of Formula (I):

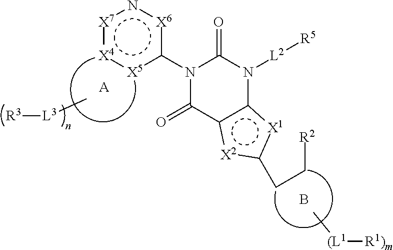

and pharmaceutically acceptable salts thereof, pharmaceutical compositions thereof, useful in the treatment of treating viral infections, for example, coronaviridae infections.

### CROSS REFERENCE TO RELATED APPLICATIONS

This application claims the benefit of U.S. Provisional Application No. 63/550,933, filed on Feb. 7, 2024, the entire contents of which is hereby incorporated by reference in its entirety.

### TECHNICAL FIELD

The present disclosure relates to compounds and methods for treating or preventing viral infections.

### BACKGROUND

Coronaviruses, named for the crown-like spikes on their surfaces, infect mostly bats, pigs and small mammals. They mutate easily and can jump from animals to humans, and from one human to another. In recent years, they have become a growing player in infectious-disease outbreaks world-wide. There is a need for compounds and methods for treating viral infections, for example coronaviridae infections. The present disclosure addresses these and other needs.

### SUMMARY

In one embodiment, the present disclosure provides a compound of Formula (I):

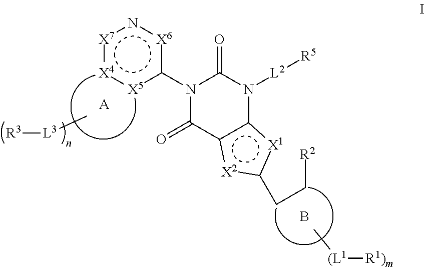

or a pharmaceutically acceptable salt thereof, wherein
X¹ is —O—, and X² is —N— or —C(R^{x2})═; or
X² is —O—, and X¹ is —N— or —C(R^{x1})═;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
ring \textcircled{A} taken together with X⁴ and X⁵ is phenyl, 5- to 6-membered heteroaryl, C₅₋₁₀ cycloalkyl, or 5- to 10-membered heterocyclyl,
   wherein each X⁴ and X⁵ is independently N or C;
alternatively, ring \textcircled{A} is absent, wherein X⁴ is N or C-L^{x4}-R^{x4}, and X⁵ is N or C-L^{x5}-R^{x5};
each L^{x4} and L^{x5} is independently a bond, —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl)-, —(C₁₋₆ alkyl)N(R^{L})S(O)₂—, —N(R^{L})S(O)₂—, —C(O)—, —(C₁₋₆ alkyl)C(O)—, or —N(R^{L})C(O)—;
each R^{x4} and R^{x5} is independently hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl;
   wherein each C₁₋₆ alkyl, C₂₋₆ alkynyl, C₁₋₆ alkoxy, C₃₋₈ cycloalkyl, and 4- to 10-membered heterocyclyl of R^{x4} and R^{x5} is optionally substituted with one to four R^{4a};
each R^{4a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂, wherein each C₁₋₆ alkyl, C₃₋₈ cycloalkyl, 4- to 10-membered heterocyclyl, phenyl, and 5- to 10-membered heteroaryl of R^{4a} is optionally substituted with one to three R^{4b};
each R^{4b} is independently C₁₋₆ alkyl, C₁₋₆ haloalkyl, C₃₋₆ cycloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂;
each L³ is independently a bond, —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl)-, —(C₁₋₆ alkyl)N(R^{L})S(O)₂—, —N(R^{L})S(O)₂—, —C(O)—, —(C₁₋₆ alkyl)C(O)—, or —N(R^{L})C(O)—;
each R³ is independently a hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, phenyl, 5- to 10-membered heteroaryl, —NH₂, —NH(C₁₋₆ alkyl), —N(C₁₋₆ alkyl)₂, or —OC₃₋₈ cycloalkyl;
   wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to four R^{3a};
each R^{3a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂,
   wherein each C₁₋₆ alkyl, C₃₋₈ cycloalkyl, 4- to 10-membered heterocyclyl, phenyl, and 5- to 10-membered heteroaryl of R³ is optionally substituted with one to three R^{3b};
each R^{3b} is independently C₁₋₆ alkyl, C₁₋₆ haloalkyl, C₃₋₆ cycloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(C₆₋₁₀aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂;
each R^{L} is independently hydrogen, C₁₋₆ alkyl, C₁₋₆ haloalkyl, or C₃₋₈ cycloalkyl;
n is an integer from 0 to 3;
each X⁶ and X⁷ is independently N, CH, or CF,
   wherein no more than two of X⁴, X⁵, X⁶, and X⁷ are N;
ring \textcircled{B} is C₆₋₁₀ aryl or 5- to 10-membered heteroaryl;
each L¹ is independently a bond, —O—, —(C₁₋₆ alkyl)O—, —O(C₁₋₆ alkyl)-, —C₁₋₆ alkyl-O(C₁₋₆ alkyl)-, —C(O)—, —N(R^{L})C(O)—, —C(O)N(R^{L})—, —(C₁₋₆ alkyl)(R^{L})NC(O)—, —(C₁₋₆ alkyl)C(O)N(R^{L})—, —N(R^{L})C(O)(C₁₋₆ alkyl)-, —C(O)N(R^{L})(C₁₋₆ alkyl)-, —(C₁₋₆ alkyl)N(R^{L})C(O)(C₁₋₆ alkyl)-, —(C₁₋₆ alkyl)C(O)N(R^{L})(C₁₋₆ alkyl)-, —S(O)₂—, —S(O)₂N(R^{L})—, —N(R^{L})—S(O)₂—, —(C₁₋₆ alkyl)S(O)₂N(R^{L})—, —(C₁₋₆ alkyl)N(R^{L})S(O)₂—, —S(O)₂N(R^{L})(C₁₋₆ alkyl)-, —N(R^{L})S(O)₂(C₁₋₆ alkyl)-, —(C₁₋₆ alkyl)S(O)₂N(R^{L})(C₁₋₆ alkyl)-, or —(C₁₋₆ alkyl)N(R^{L})S(O)₂(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
   wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
alternatively, two R¹ taken together with the atoms of ring \textcircled{B} to which they are attached form 5- to 10-membered heterocyclyl optionally substituted with one to three R^{1a};
each R^{1a} is independently C₁₋₆ alkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —N(5- to 10-membered heterocyclyl)₂, —N(phenyl)₂, —N(5- to 10-membered heteroaryl)₂, —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)O(C₁₋₆ alkyl), —C(O)O(C₃₋₈ cycloalkyl), —C(O)O(5- to 10-membered heterocyclyl), —C(O)O(phenyl), —C(O)O(5- to 10-membered heteroaryl), —C(O)NH₂, —C(O)NH(C₁₋₆ alkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₃₋₈ cycloalkyl)₂, —C(O)N(5- to 10-membered heterocyclyl)₂, —C(O)N(phenyl)₂, —C(O)N(5- to 10-membered heteroaryl)₂, —NHC(O)(C₁₋₆ alkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —NHS(O)(C₁₋₆ alkyl), —N(C₁₋₆ alkyl)(S(O)(C₁₋₆ alkyl), —S(O)₂(C₁₋₆ alkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂,
   wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
alternatively, R¹ is phenyl, and two R^{1a} taken together with the atoms of the phenyl to which they are attached form 5- to 10-membered heterocyclyl optionally substituted with one to three R^{1b};
each R^{1b} is independently C₁₋₆ alkyl, C₁₋₆ haloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(phenyl), —O(5- to 10-membered heteroaryl), —NH(C₁. 6 alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₂₋₆ alkynyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, —(C₁₋₆ alkyl)O(C₁₋₆ alkyl)-, —(C₁₋₆ alkyl)(R^{L2})NC(O)—, —(C₁₋₆ alkyl)C(O)N(R^{L2})—, —(C₁₋₆ alkyl)S(O)₂N(R^{L2})—, —(C₁₋₆ alkyl)N(R^{L2})S(O)₂—, —(C₁₋₆ alkyl)S(O)₂N(R^{L2})(C₁₋₆ alkyl)-, or —(C₁₋₆ alkyl)S(O)₂—(C₁₋₆ alkyl)-;
R^{L2} is hydrogen or C₁₋₆ alkyl;
R⁵ is hydrogen, —CN, —OR^{5a}, —C(O)NR^{5a}₂, —NR^{5a}C(O)R^{5a}, —NR^{5a}₂ C₁₋₆ alkyl, C₃₋₈ cycloalkyl, phenyl, 4- to 10-membered heterocyclyl, or 5- to 10-membered heteroaryl,
   wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{5b};
each R^{5a} is independently hydrogen or C₁₋₆ alkyl; and
each R^{5b} is independently halogen, oxo, cyclopropyl, hydroxy, —CN, C₁₋₃ alkyl, C₁₋₃ alkoxy, —OCF₃, or —OCF₂H.

In another embodiment, the present disclosure provides a pharmaceutical composition comprising a pharmaceutically effective amount of a compound of the present disclosure, or a pharmaceutically acceptable salt thereof, and a pharmaceutically acceptable carrier or excipient.

In another embodiment, the present disclosure provides a method of treating a viral infection in a subject in need thereof, the method comprising administering to the subject a therapeutically effective amount of a compound of the present disclosure, or a pharmaceutically acceptable salt thereof, or a pharmaceutical composition of the present disclosure.

### DETAILED DESCRIPTION

The present disclosure relates generally to methods and compounds for treating or preventing viral infections, for example coronaviridae infections.

### Definitions

As used in the present specification, the following words, phrases and symbols are generally intended to have the meanings as set forth below, except to the extent that the context in which they are used indicates otherwise.

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆ and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatrienyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxyalkyl” refers an alkoxy group linked to an alkyl group which is linked to the remainder of the compound such that the alkyl group is divalent. Alkoxyalkyl can have any suitable number of carbon, such as from 2 to 6 (C₂₋₆ alkoxyalkyl), 2 to 5 (C₂₋₅ alkoxyalkyl), 2 to 4 (C₂₋₄ alkoxyalkyl), or 2 to 3 (C₂₋₃ alkoxyalkyl). Alkoxy and alkyl are as defined above where the alkyl is divalent, and can include, but is not limited to, methoxymethyl (CH₃OCH₂—), methoxyethyl (CH₃OCH₂CH₂—) and others.

“Alkoxy-alkoxy” refers an alkoxy group linked to a second alkoxy group which is linked to the remainder of the compound. Alkoxy is as defined above, and can include, but is not limited to, methoxy-methoxy (CH₃OCH₂O—), methoxy-ethoxy (CH₃OCH₂CH₂O—) and others.

“Halo” or “halogen” as used herein refers to fluoro (—F), chloro (—C₁), bromo (—Br) and iodo (—I).

The term “oxo” as used herein is intended to mean an oxygen atom attached to a carbon atom as a substituent by a double bond (═O).

“Hydroxyl” and “hydroxy” are used interchangeably and refer to —OH.

“Haloalkyl” as used herein refers to an alkyl as defined herein, wherein one or more hydrogen atoms of the alkyl are independently replaced by a halo substituent, which may be the same or different. For example, C₁₋₄ haloalkyl is a C₁₋₄ alkyl wherein one or more of the hydrogen atoms of the C₁₋₄ alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having 6 to 12 annular carbon atoms such as bicyclo[3.1.0]hexane and bicyclo[2.1.1]hexane), and polycyclic carbocycles (e.g., tricyclic and tetracyclic carbocycles with up to 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocyclyl” or “heterocycle” or “heterocycloalkyl” as used herein refers to a single saturated or partially unsaturated non-aromatic ring or a multiple ring system having at least one heteroatom in the ring (i.e., at least one annular heteroatom selected from oxygen, nitrogen, and sulfur) wherein the multiple ring system includes at least non-aromatic ring containing at least one heteroatom. The multiple ring system can also include other aromatic rings and non-aromatic rings. Unless otherwise specified, a heterocyclyl group has from 3 to 20 annular atoms, for example from 3 to 12 annular atoms, for example from 3 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from 1 to 6 annular carbon atoms and from 1 to 3 annular heteroatoms selected from the group consisting of oxygen, nitrogen and sulfur in the ring. The heteroatoms can optionally be oxidized to form —N(—OH)—, ═N(—O—)—, —S(═O)— or —S(═O)₂—. The rings of the multiple condensed ring (e.g., bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above. Alkyl-heterocycloalkyl groups can be substituted or unsubstituted.

“Aryl” as used herein refers to a single all carbon aromatic ring or a multiple condensed all carbon ring system wherein at least one of the rings is aromatic. For example, in some embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having 9 to 20 carbon atoms in which at least one ring is aromatic and wherein the other rings may be aromatic or not aromatic (i.e., carbocycle) and may be saturated or partially saturated. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1,2,3,4-tetrahydronaphthyl. Non-limiting examples of aryl groups include, but are not limited to, phenyl, indenyl, naphthyl, 1,2,3,4-tetrahydronaphthyl, anthracenyl, and the like.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from 1 to 6 carbon atoms and 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. The other rings may be aromatic or not aromatic (i.e., carbocycle) and may be saturated or partially saturated. Thus, a heteroaryl (a single aromatic ring or multiple condensed ring system) has 1-20 carbon atoms and 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, and triazolyl.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

A “compound of the present disclosure” includes compounds disclosed herein, for example a compound of the present disclosure includes compounds of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), and (VII-D), including the compounds of the Examples.

“Composition” as used herein is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and deleterious to the recipient thereof.

“Pharmaceutically effective amount” refers to an amount of a compound of the present disclosure in a formulation or combination thereof, that provides the desired therapeutic or pharmaceutical result.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

The terms “treatment” or “treat” or “treating” also encompass alleviating or eliminating symptoms of a viral infection and/or reducing viral load in a patient. The term “treatment” also encompasses the administration of a compound or composition according to the embodiments disclosed herein post-exposure of the individual to the virus but before the appearance of symptoms of the disease, and/or prior to the detection of the virus in the blood, to prevent the appearance of symptoms of the disease and/or to prevent the virus from reaching detectible levels in the blood.

“Therapeutically effective amount” or “effective amount” as used herein refers to an amount that is effective to elicit the desired biological or medical response, including the amount of a compound that, when administered to a subject for treating a disease, is sufficient to effect such treatment for the disease. The effective amount can vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device, e.g., a mini-osmotic pump, to the subject. The administration can be carried out according to a schedule specifying frequency of administration, dose for administration, and other factors.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

“Subject” refers to animals such as mammals, including, but not limited to, primates (e.g., humans), cows, sheep, goats, horses, dogs, cats, rabbits, rats, mice and the like. In certain embodiments, the subject is a human.

“Disease” or “condition” refer to a state of being or health status of a patient or subject capable of being treated with a compound, pharmaceutical composition, or method provided herein.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds of described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁵N, ¹⁵O, ¹⁷O, ¹⁸O, ³¹P, ³²P ³⁵S, ¹⁸F, ³⁶Cl, ¹²³I and ¹²⁵I respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The compounds of the embodiments disclosed herein, or their pharmaceutically acceptable salts may be “atropisomers”, i.e., stereoisomers arising due to hindered rotation about a single bond, where the barrier to rotation about the bond is high enough to allow for isolation of individual stereoisomers. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

“Racemates” refers to a mixture of enantiomers. The mixture can comprise equal or unequal amounts of each enantiomer.

“Stereoisomer” and “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

Atropisomers described herein may be represented using bold bonds (“ [symbol] ”) wedged bonds (“ [symbol] ”), hashed bonds (“ [symbol] ”), and hashed wedged bonds (“ [symbol] ”) in a manner that is well understood by those skilled in the art. By way of example, the following shows an atropisomeric pair:

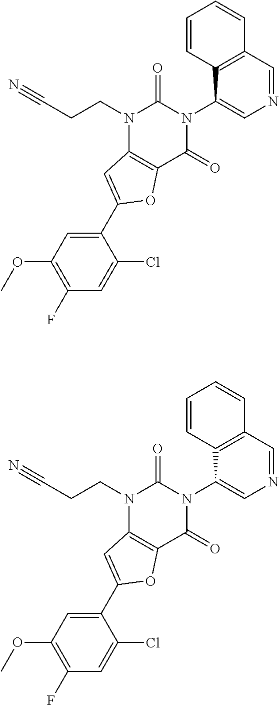

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly understood by one of ordinary skill in the art. A dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cᵤ₋ᵥ” or (Cᵤ-Cᵥ) indicates that the following group has from u to v carbon atoms. For example, “C₁₋₆alkyl” and “C₁-C₆ alkyl” both indicate that the alkyl group has from 1 to 6 carbon atoms.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal.

Whenever a compound described herein is substituted with more than one of the same designated group, e.g., “R” or “R”, then it will be understood that the groups may be the same or different, i.e., each group is independently selected, unless indicated otherwise.

Wavy lines, [symbol] , indicate the site of covalent bond attachments to the adjoining substructures, groups, moieties, or atoms.

The prefixes “Cᵤ₋ᵥ,” and “(Cᵤ₋ᵥ)” indicate that the following group has from u to v carbon atoms. For example, “C₁₋₈ alkyl” indicates that the alkyl group has from 1 to 8 carbon atoms.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively.

Unless indicated explicitly otherwise, where combinations of groups are referred to herein as one moiety, e.g., arylalkyl, the last mentioned group contains the atom by which the moiety is attached to the rest of the molecule.

The term “substituted” means that any one or more hydrogen atoms on the designated atom or group is replaced with one or more substituents other than hydrogen, provided that the designated atom's normal valence is not exceeded. The one or more substituents unless indicated otherwise include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof.

The term “optionally substituted” refers to any one or more hydrogen atoms on the designated atom or group may or may not be replaced by a moiety other than hydrogen.

### Compounds

The present disclosure provides compounds that are inhibitors of the SARS-CoV-2 main protease. In some embodiments, the disclosure provides compounds of Formula (I) as described herein, and/or pharmaceutically acceptable salt(s) thereof. In some embodiments, a compound is provided according to Formula (I):

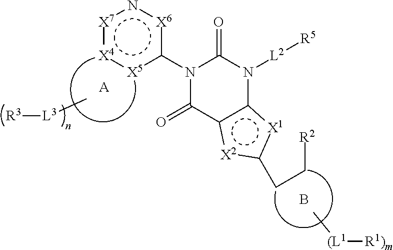

and/or pharmaceutically acceptable salt(s) thereof.

In some embodiments, the compound of Formula (I) is

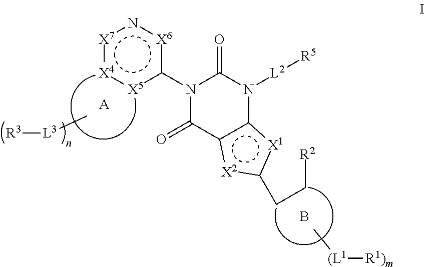

or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
ring \textcircled{A} taken together with X⁴ and X⁵ is phenyl, 5- to 6-membered heteroaryl, C₅₋₁₀ cycloalkyl, or 5- to 10-membered heterocyclyl,
   wherein each X⁴ and X⁵ is independently N or C;
alternatively, ring \textcircled{A} is absent, wherein X⁴ is N or C-L^{x4}-R^{x4}, and X⁵ is N or C-L^{x5}-R^{x5};
each L^{x4} and L^{x5} is independently a bond, —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl)-, —(C₁₋₆ alkyl)N(R^{L})S(O)₂—, —N(R^{L})S(O)₂—, —C(O)—, —(C₁₋₆ alkyl)C(O)—, or —N(R^{L})C(O)—;
each R^{x4} and R^{x5} is independently hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl;
   wherein each C₁₋₆ alkyl, C₂₋₆ alkynyl, C₁₋₆ alkoxy, C₃₋₈ cycloalkyl, and 4- to 10-membered heterocyclyl of R^{x4} and R^{x5} is optionally substituted with one to four R^{4a};
each R^{4a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂,
   wherein each C₁₋₆ alkyl, C₃₋₈ cycloalkyl, 4- to 10-membered heterocyclyl, phenyl, and 5- to 10-membered heteroaryl of R^{4a} is optionally substituted with one to three R^{4b};
each R^{4b} is independently C₁₋₆ alkyl, C₁₋₆ haloalkyl, C₃₋₆ cycloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(5- to 10-membered heteroaryl), —NHC(O)NH(C₁₆ alkyl), S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂;
each L³ is independently a bond, —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl)-, —(C₁₋₆ alkyl)N(R^{L})S(O)₂—, —N(R^{L})S(O)₂—, —C(O)—, —(C₁₋₆ alkyl)C(O)—, or —N(R^{L})C(O)—;
each R³ is independently a hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, phenyl, 5- to 10-membered heteroaryl, —NH₂, —NH(C₁₋₆ alkyl), —N(C₁₋₆ alkyl)₂, or —OC₃₋₈ cycloalkyl;
   wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to four R^{3a};
each R^{3a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂,
   wherein each C₁₋₆ alkyl, C₃₋₈ cycloalkyl, 4- to 10-membered heterocyclyl, phenyl, and 5- to 10-membered heteroaryl of R³ is optionally substituted with one to three R^{3b};
each R^{3b} is independently C₁₋₆ alkyl, C₁₋₆ haloalkyl, C₃₋₆ cycloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂;
each R^{L} is independently hydrogen, C₁₋₆ alkyl, C₁₋₆ haloalkyl, or C₃₋₈ cycloalkyl; n is an integer from 0 to 3;
each X⁶ and X⁷ is independently N, CH, or CF,
   wherein no more than two of X⁴, X⁵, X⁶, and X⁷ are N;
ring \textcircled{B} is C₆₋₁₀ aryl or 5- to 10-membered heteroaryl;
each L¹ is independently a bond, —O—, —(C₁₋₆ alkyl)O—, —O(C₁₋₆ alkyl)-, —C₁₋₆ alkyl-O(C₁₋₆ alkyl)-, —C(O)—, —N(R^{L})C(O)—, —C(O)N(R^{L})—, —(C₁₋₆ alkyl)(R^{L})NC(O)—, —(C₁₋₆ alkyl)C(O)N(R^{L})—, —N(R^{L})C(O)(C₁₋₆ alkyl)-, —C(O)N(R^{L})(C₁₋₆ alkyl)-, —(C₁₋₆ alkyl)N(R^{L})C(O)(C₁₋₆ alkyl)-, —(C₁₋₆ alkyl)C(O)N(R^{L})(C₁₋₆ alkyl)-, —S(O)₂—, —S(O)₂N(R^{L})—, —N(R^{L})—S(O)₂—, —(C₁₋₆ alkyl)S(O)₂N(R^{L})—, —(C₁₋₆ alkyl)N(R^{L})S(O)₂—, —S(O)₂N(R^{L})(C₁₋₆ alkyl)-, —N(R^{L})S(O)₂(C₁₋₆ alkyl)-, —(C₁₋₆ alkyl)S(O)₂N(R^{L})(C₁₋₆ alkyl)-, or —(C₁₋₆ alkyl)N(R^{L})S(O)₂(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
   wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
alternatively, two R¹ taken together with the atoms of ring \textcircled{B} to which they are attached form 5- to 10-membered heterocyclyl optionally substituted with one to three R^{1a};
each R^{1a} is independently C₁₋₆ alkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —N(5- to 10-membered heterocyclyl)₂, —N(phenyl)₂, —N(5- to 10-membered heteroaryl)₂, —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)O(C₁₋₆ alkyl), —C(O)O(C₃₋₈ cycloalkyl), —C(O)O(5- to 10-membered heterocyclyl), —C(O)O(phenyl), —C(O)O(5- to 10-membered heteroaryl), —C(O)NH₂, —C(O)NH(C₁₋₆ alkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₃₋₈ cycloalkyl)₂, —C(O)N(5- to 10-membered heterocyclyl)₂, —C(O)N(phenyl)₂, —C(O)N(5- to 10-membered heteroaryl)₂, —NHC(O)(C₁₋₆ alkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —NHS(O)(C₁₋₆ alkyl), —N(C₁₋₆ alkyl)(S(O)(C₁₋₆ alkyl), —S(O)₂(C₁₋₆ alkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂,
   wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
alternatively, R¹ is phenyl, and two R^{1a} taken together with the atoms of the phenyl to which they are attached form 5- to 10-membered heterocyclyl optionally substituted with one to three R^{1b};
each R^{1b} is independently C₁₋₆ alkyl, C₁₋₆ haloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₂₋₆ alkynyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, —(C₁₋₆ alkyl)O(C₁₋₆ alkyl)-, —(C₁₋₆ alkyl)(R^{L2})NC(O)—, —(C₁₋₆ alkyl)C(O)N(R^{L2})—, —(C₁₋₆ alkyl)S(O)₂N(R^{L2})—, —(C₁₋₆ alkyl)N(R^{L2})S(O)₂—, —(C₁₋₆ alkyl)S(O)₂N(R^{L2})(C₁₋₆ alkyl)-, or —(C₁₋₆ alkyl)S(O)₂—(C₁₋₆ alkyl)-;
R^{L2} is hydrogen or C₁₋₆ alkyl;
R⁵ is hydrogen, —CN, —OR^{5a}, —C(O)NR^{5a}₂, —NR^{5a}C(O)R^{5a}, —NR^{5a}₂ C₁₋₆ alkyl, C₃₋₈ cycloalkyl, phenyl, 4- to 10-membered heterocyclyl, or 5- to 10-membered heteroaryl,
   wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{5b};
each R^{5a} is independently hydrogen or C₁₋₆ alkyl; and
each R^{5b} is independently halogen, oxo, cyclopropyl, hydroxy, —CN, C₁₋₃ alkyl, C₁₋₃ alkoxy, —OCF₃, or —OCF₂H.

In some embodiments, the compound of Formula (I) is

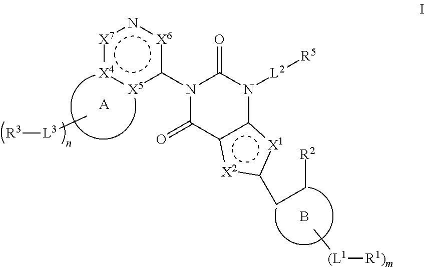

or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
ring \textcircled{A} taken together with X⁴ and X⁵ is phenyl, 5- to 6-membered heteroaryl, C₅₋₁₀ cycloalkyl, or 5- to 10-membered heterocyclyl,
   wherein each X⁴ and X⁵ is independently N or C; alternatively, ring \textcircled{A} is absent, wherein X⁴ is N or C-L^{x4}-R^{x4}, and X⁵ is N or C-L^{x5}-R^{x5};
each L^{x4} and L^{x5} is independently a bond or N(R^{L})S(O)₂—;
each R^{x4} and R^{x5} is independently hydrogen, C₁₋₆ alkyl, —NH₂, —NH(C₁₋₆ alkyl), —N(C₁₋₆ alkyl)₂, C₁₋₆ alkoxy, C₃-C₈ cycloalkyl, or 5- to 10-membered heterocyclyl;
   wherein each C₁₋₆ alkyl, C₁₋₆ alkoxy, C₃₋₈ cycloalkyl, and 5- to 10-membered heterocyclyl of R^{x4} and R^{x5} is optionally substituted with one to four R^{4a};
each R^{4a} is independently C₁₋₆ alkyl, —C(O)(5- to 10-membered heterocyclyl), or —O(C₃₋₈ cycloalkyl),
   wherein each C₁₋₆ alkyl, C₃₋₈ cycloalkyl, and 5- to 10-membered heterocyclyl of R^{4a} is optionally substituted with one to three R^{4b};
each R^{4b} is independently halogen, —O(C₁₋₆ alkyl), or —O(C₁₋₆ haloalkyl);
each L³ is independently a bond, —(C₁₋₆ alkyl)O—, —(C₁₋₆ alkyl)-, —C(O)—, or —(C₁₋₆ alkyl)C(O)—;
each R³ is independently a hydrogen, halogen, hydroxy, —CN, C₁₋₆ alkyl, C₂₋₆ alkynyl, C₁₋₆ alkoxy, C₃₋₈ cycloalkyl, or 4- to 10-membered heterocyclyl;
   wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to two R^{3a};
each R^{3a} is independently halogen, —C(O)(C₁-C₆ alkyl), —OH, —O(C₁₋₆ alkyl), 5- to 10-membered heterocyclyl, —C(O)NH₂, —C(O)N(H)(C₁-C₆ alkyl), or —C(O)N(C₁-C₆ alkyl)₂;
n is an integer from 0 to 3;
each X⁶ and X⁷ is independently N or CH,
   wherein no more than two of X⁴, X⁵, X⁶, and X⁷ are N;
ring \textcircled{B} is C₆₋₁₀ aryl;
each L¹ is independently bond, —O—, —(C₁₋₆ alkyl)O—, —O(C₁₋₆ alkyl)-, or —C₁₋₆ alkyl-O(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
   wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl,
   wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
each R^{1b} is independently C₁₋₆ alkyl, C₁₋₆ haloalkyl, halogen, oxo, —OH, or —NH₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-; R⁵ is hydrogen, —CN, C₁₋₆ alkyl, C₃₋₈ cycloalkyl, phenyl, 4- to 10-membered heterocyclyl, or 5- to 10-membered heteroaryl,
   wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{b};
each R^{5a} is independently hydrogen or C₁₋₆ alkyl; and
each R^{5b} is independently oxo, C₁₋₃ alkyl, or C₁₋₃ alkoxy.

In some embodiments, the disclosure provides a compound according to the structure of Formula (I-A):

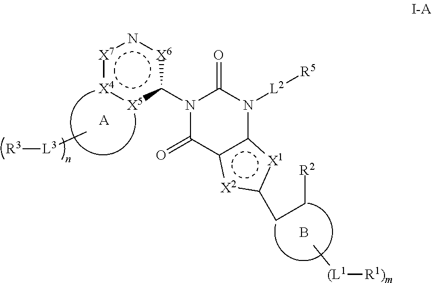

or a pharmaceutically acceptable salt thereof, wherein ring \textcircled{A}, ring \textcircled{B}, X¹, X², X⁴, X⁵, X⁶, X⁷, L¹, L², L³, R¹, R², R³, R⁵, m, and n are as described herein.

In some embodiments, the disclosure provides a compound according to the structure of Formula (II-A):

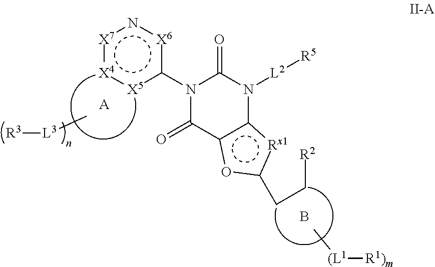

or a pharmaceutically acceptable salt thereof, wherein ring \textcircled{A}, ring \textcircled{B}, R^{x1}, X⁴, X⁵, X⁶, X⁷, L¹, L², L³, R¹, R², R³, R⁵, m, and n are as described herein.

In some embodiments, the disclosure provides a compound according to the structure of Formula (II-B):

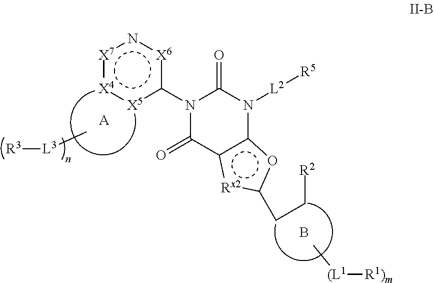

or a pharmaceutically acceptable salt thereof, wherein ring \textcircled{A}, ring \textcircled{B}, R^{x2}, X⁴, X⁵, X⁶, X⁷, L¹, L², L³, R¹, R², R³, R⁵, m, and n are as described herein.

In some embodiments, the disclosure provides a compound according to the structure of Formula (II-C):

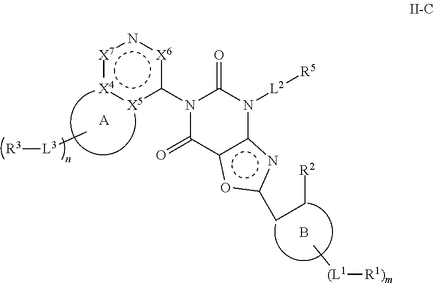

or a pharmaceutically acceptable salt thereof, wherein ring \textcircled{A}, ring \textcircled{B}, X⁴, X⁵, X⁶, X⁷, L¹, L², L³, R¹, R², R³, R⁵, m, and n are as described herein.

In some embodiments, the disclosure provides a compound according to the structure of Formula (II-D):

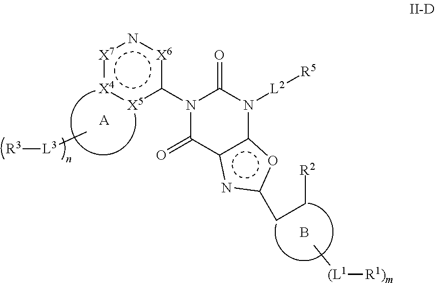

or a pharmaceutically acceptable salt thereof, wherein ring \textcircled{A}, ring \textcircled{B}, X⁴, X⁵, X⁶, X⁷, L¹, L², L³, R¹, R², R³, R⁵, m, and n are as described herein.

In some embodiments, a compound of the disclosure is a compound, or pharmaceutically acceptable salt thereof, wherein X² is —O—. In some embodiments, a compound of the disclosure is a compound, or pharmaceutically acceptable salt thereof, wherein X² is —O—, and X¹ is —C(R^{x1})=. In some embodiments, a compound of the disclosure is a compound, or pharmaceutically acceptable salt thereof, wherein X² is —O—, and X¹ is —N—.

In some embodiments, a compound of the disclosure is a compound, or pharmaceutically acceptable salt thereof, wherein X¹ is —O—. In some embodiments, a compound of the disclosure is a compound, or pharmaceutically acceptable salt thereof, wherein X¹ is —O—, and X² is —C(R^{x2})=. In some embodiments, a compound of the disclosure is a compound, or pharmaceutically acceptable salt thereof, wherein Xⁱ is —O—, and X² is —N—.

In some embodiments, a compound of the disclosure is a compound, or pharmaceutically acceptable salt thereof, wherein R^{x1} is halogen, C₁₋₃ alkyl, or C₁₋₃ alkoxy. In some embodiments, a compound of the disclosure is a compound, or pharmaceutically acceptable salt thereof, wherein R^{x1} is chloro, fluoro, methyl or methoxy.

In some embodiments, a compound of the disclosure is a compound, or pharmaceutically acceptable salt thereof, wherein R^{x1} is halogen or C₁₋₃ alkyl. In some embodiments, a compound of the disclosure is a compound, or pharmaceutically acceptable salt thereof, wherein R^{x1} is chloro, fluoro, or methyl.

In some embodiments, a compound of the disclosure is a compound, or pharmaceutically acceptable salt thereof, wherein R^{x1} is hydrogen or C₁₋₃ alkyl. In some embodiments, a compound of the disclosure is a compound, or pharmaceutically acceptable salt thereof, wherein R^{x1} is hydrogen or methyl.

In some embodiments, a compound of the disclosure is a compound, or pharmaceutically acceptable salt thereof, wherein R^{x1} is hydrogen.

In some embodiments, a compound of the disclosure is a compound, or pharmaceutically acceptable salt thereof, wherein R^{x2} is halogen, C₁₋₃ alkyl, or C₁₋₃ alkoxy. In some embodiments, a compound of the disclosure is a compound, or pharmaceutically acceptable salt thereof, wherein R^{x2} is chloro, fluoro, methyl or methoxy.

In some embodiments, a compound of the disclosure is a compound, or pharmaceutically acceptable salt thereof, wherein R^{x2} is halogen or C₁₋₃ alkyl. In some embodiments, a compound of the disclosure is a compound, or pharmaceutically acceptable salt thereof, wherein R^{x2} is chloro, fluoro, or methyl.

In some embodiments, a compound of the disclosure is a compound, or pharmaceutically acceptable salt thereof, wherein R^{x2} is hydrogen or C₁₋₃ alkyl. In some embodiments, a compound of the disclosure is a compound, or pharmaceutically acceptable salt thereof, wherein R^{x2} is hydrogen or methyl.

In some embodiments, a compound of the disclosure is a compound, or pharmaceutically acceptable salt thereof, wherein R^{x2} is hydrogen.

In some embodiments, a compound of the disclosure is a compound, or pharmaceutically acceptable salt thereof, wherein X⁶ is N or CH. In some embodiments, a compound of the disclosure is a compound, or pharmaceutically acceptable salt thereof, wherein X⁶ is N. In some embodiments, a compound of the disclosure is a compound, or pharmaceutically acceptable salt thereof, wherein X⁶ is CH.

In some embodiments, a compound of the disclosure is a compound, or pharmaceutically acceptable salt thereof, wherein X⁷ is N or CH. In some embodiments, a compound of the disclosure is a compound, or pharmaceutically acceptable salt thereof, wherein X⁷ is N. In some embodiments, a compound of the disclosure is a compound, or pharmaceutically acceptable salt thereof, wherein X⁷ is CH.

In some embodiments, a compound of the disclosure is a compound, or pharmaceutically acceptable salt thereof, wherein X⁶ and X⁷ are CH.

In some embodiments, a compound of the disclosure is a compound, or pharmaceutically acceptable salt thereof, wherein no more than two of X⁴, X⁵, X⁶, and X⁷ are N. In some embodiments, a compound of the disclosure is a compound, or pharmaceutically acceptable salt thereof, wherein two of X⁴, X⁵, X⁶, and X⁷ are N. In some embodiments, a compound of the disclosure is a compound, or pharmaceutically acceptable salt thereof, wherein one of X⁴, X⁵, X⁶, and X⁷ is N. In some embodiments, a compound of the disclosure is a compound, or pharmaceutically acceptable salt thereof, wherein none of X⁴, X⁵, X⁶, and X⁷ is N.

In some embodiments, a compound of the disclosure is a compound, or pharmaceutically acceptable salt thereof, wherein
ring \textcircled{A} taken together with X⁴ and X⁵ is phenyl, 5- to 6-membered heteroaryl, C₅₋₁₀ cycloalkyl, or 5- to 10-membered heterocyclyl, and
X⁴ and X⁵ are each C.

In some embodiments, a compound of the disclosure is a compound, or pharmaceutically acceptable salt thereof, wherein
ring \textcircled{A} taken together with X⁴ and X⁵ is phenyl, 5- to 6-membered heteroaryl, or C₅. 10 cycloalkyl, and
X⁴ and X⁵ are each C.

In some embodiments, a compound of the disclosure is a compound, or pharmaceutically acceptable salt thereof, wherein
ring \textcircled{A} taken together with X⁴ and X⁵ is 5- to 6-membered heteroaryl, and
X⁴ and X⁵ are each independently C or N.

In some embodiments, a compound of the disclosure is a compound, or pharmaceutically acceptable salt thereof, wherein \textcircled{A} ring is phenyl.

In some embodiments, a compound of the disclosure is a compound, or pharmaceutically acceptable salt thereof, wherein ring \textcircled{A} is C₅₋₇ cycloalkyl.

In some embodiments, a compound of the disclosure is a compound, or pharmaceutically acceptable salt thereof, wherein ring \textcircled{A} is 5-membered heteroaryl.

In some embodiments, a compound of the disclosure is a compound, or pharmaceutically acceptable salt thereof, wherein ring \textcircled{A} is 6-membered heteroaryl.

In some embodiments, a compound of the disclosure is a compound, or pharmaceutically acceptable salt thereof, wherein X⁶ is CH and X⁷ is N. In some embodiments, a compound of the disclosure is a compound, or pharmaceutically acceptable salt thereof, wherein X⁶ is N and X⁷ is CH. In some embodiments, a compound of the disclosure is a compound, or pharmaceutically acceptable salt thereof, wherein X⁶ and X⁷ are CH.

In some embodiments, the disclosure provides a compound according to the structure of Formula (III):

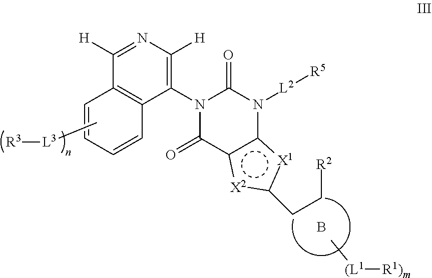

or a pharmaceutically acceptable salt thereof, wherein ring \textcircled{B}, X¹, X², L¹, L², L³, R¹, R², R³, R⁵, m, and n are as described herein.

In some embodiments, the compound of Formula (III) is:

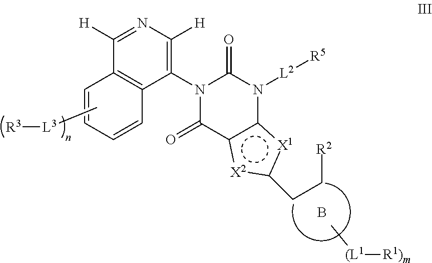

or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
each L³ is independently a bond, —(C₁₋₆ alkyl)O—, —(C₁₋₆ alkyl)-, —C(O)—, or —(C₁₋₆ alkyl)C(O)—;
each R³ is independently a hydrogen, halogen, hydroxy, —CN, C₁₋₆ alkyl, C₂₋₆ alkynyl, C₁₋₆ alkoxy, C₃₋₈ cycloalkyl, or 4- to 10-membered heterocyclyl;
   wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to two R^{3a};
each R^{3a} is independently halogen, —C(O)(C₁-C₆ alkyl), —OH, —O(C₁₋₆ alkyl), 5- to 10-membered heterocyclyl, —C(O)NH₂, —C(O)N(H)(C₁-C₆ alkyl), or —C(O)N(C₁-C₆ alkyl)₂;
n is an integer from 0 to 3;
ring \textcircled{B} is C₆₋₁₀ aryl;
each L¹ is independently bond, —O—, —(C₁₋₆ alkyl)O—, —O(C₁₋₆ alkyl)-, or —C₁₋₆ alkyl-O(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
   wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl,
   wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
each R^{1b} is independently C₁₋₆ alkyl, C₁₋₆ haloalkyl, halogen, oxo, —OH, or —NH₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-;
R⁵ is hydrogen, —CN, C₁₋₆ alkyl, C₃₋₈ cycloalkyl, phenyl, 4- to 10-membered heterocyclyl, or 5- to 10-membered heteroaryl,
   wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{b};
each R^{5a} is independently hydrogen or C₁₋₆ alkyl; and
each R^{5b} is independently oxo, C₁₋₃ alkyl, or C₁₋₃ alkoxy.

In some embodiments, the disclosure provides a compound according to the structure of Formula (IV-A):

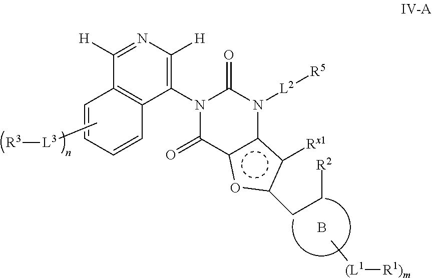

or a pharmaceutically acceptable salt thereof, wherein ring \textcircled{B}, R^{x1}, L¹, L², L³, R¹, R², R³, R⁵, m, and n are as described herein.

In some embodiments, the disclosure provides a compound according to the structure of Formula (IV-B):

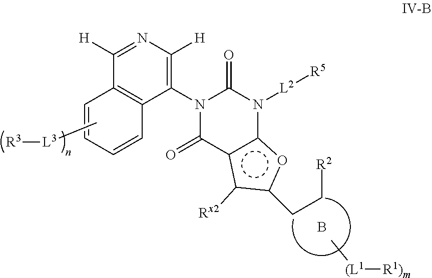

or a pharmaceutically acceptable salt thereof, wherein ring \textcircled{B}, R^{x2}, L¹, L², L³, R¹, R², R³, R⁵, m, and n are as described herein.

In some embodiments, the disclosure provides a compound according to the structure of Formula (IV-C):

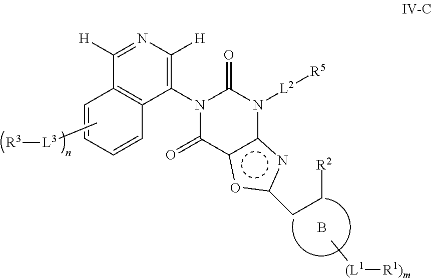

or a pharmaceutically acceptable salt thereof, wherein ring \textcircled{B}, L¹, L², L³, R¹, R², R³, R⁵, m, and n are as described herein.

In some embodiments, the disclosure provides a compound according to the structure of Formula (IV-D):

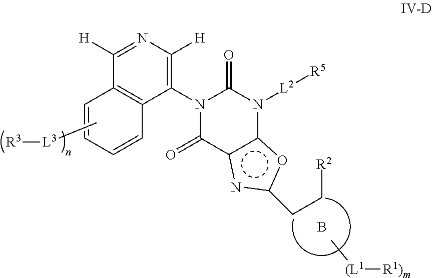

or a pharmaceutically acceptable salt thereof, wherein ring \textcircled{B}, L¹, L², L³, R¹, R², R³, R⁵, m, and n are as described herein.

In some embodiments, a compound of the disclosure is a compound, or pharmaceutically acceptable salt thereof, wherein at least one L³ is independently —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl), —(C₁₋₆ alkyl)N(R^{L})S(O)₂—, —N(R^{L})S(O)₂—, —C(O)—, —(C₁₋₆ alkyl)C(O)—, or —N(R^{L})C(O)—, wherein R^{L} is as described herein. In some embodiments, a compound of the disclosure is a compound, or pharmaceutically acceptable salt thereof, wherein at least one L³ is independently —(C₁₋₃ alkyl)O—, —(C₁₋₃ alkyl)N(R^{L})C(O)—, —(C₁₋₃ alkyl)C(O)N(R^{L})—, —(C₁₋₃ alkyl)N(R^{L})C(O)(C₁₋₃ alkyl)-, —(C₁₋₃ alkyl)C(O)N(R^{L})(C₁₋₃ alkyl)-, —(C₁₋₃ alkyl)-, —(C₁₋₃ alkyl)N(R^{L})S(O)₂—, —N(R^{L})S(O)₂—, —C(O)—, —(C₁₋₃ alkyl)C(O)—, or —N(R^{L})C(O)—, wherein R^{L} is as described herein.

In some embodiments, a compound of the disclosure is a compound, or pharmaceutically acceptable salt thereof, wherein each L³ is independently —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl), —(C₁₋₆ alkyl)N(R^{L})S(O)₂—, —N(R^{L})S(O)₂—, —C(O)—, —(C₁₋₆ alkyl)C(O)—, or —N(R^{L})C(O)—, wherein R^{L} is as described herein. In some embodiments, a compound of the disclosure is a compound, or pharmaceutically acceptable salt thereof, wherein each L³ is independently —(C₁₋₃ alkyl)O—, —(C₁₋₃ alkyl)N(R^{L})C(O)—, —(C₁₋₃ alkyl)C(O)N(R^{L})—, —(C₁₋₃ alkyl)N(R^{L})C(O)(C₁₋₃ alkyl)-, —(C₁₋₃ alkyl)C(O)N(R^{L})(C₁₋₃ alkyl)-, —(C₁₋₃ alkyl)-, —(C₁₋₃ alkyl)N(R^{L})S(O)₂—, —N(R^{L})S(O)₂—, —C(O)—, —(C₁₋₃ alkyl)C(O)—, or —N(R^{L})C(O)—, wherein R^{L} is as described herein.

In some embodiments, a compound of the disclosure is a compound, or pharmaceutically acceptable salt thereof, wherein each L³ is a bond, —(C₁₋₆ alkyl)O—, —(C₁₋₆ alkyl)-, —C(O)—, or —(C₁₋₆ alkyl)C(O)—. In some embodiments, a compound of the disclosure is a compound, or pharmaceutically acceptable salt thereof, wherein each L³ is a bond, —(C₁₋₃ alkyl)O—, —(C₁₋₃ alkyl)-, —C(O)—, or —(C₁₋₃ alkyl)C(O)—. In some embodiments, a compound of the disclosure is a compound, or pharmaceutically acceptable salt thereof, wherein each L³ is a bond.

In some embodiments, a compound of the disclosure is a compound, or pharmaceutically acceptable salt thereof, wherein each R³ is independently a hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, phenyl, 5- to 10-membered heteroaryl, —NH₂, —NH(C₁₋₆ alkyl), —N(C₁₋₆ alkyl)₂, or —OC₃₋₈ cycloalkyl, wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to three R^{3a}, wherein R^{3a} is as described herein. In some embodiments, each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to two R^{3a}, wherein R^{3a} is as described herein. In some embodiments, each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one R^{3a}, wherein R^{3a} is as described herein. In some embodiments, a compound of the disclosure is a compound, or pharmaceutically acceptable salt thereof, wherein each R³ is independently a hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, phenyl, 5- to 10-membered heteroaryl, —NH₂, —NH(C₁₋₆ alkyl), —N(C₁₋₆ alkyl)₂, or —OC₃₋₈ cycloalkyl.

In some embodiments, a compound of the disclosure is a compound, or pharmaceutically acceptable salt thereof, wherein each R³ is independently halogen, C₁₋₆ alkyl, —NH₂, —NH(C₁₋₆ alkyl), —N(C₁₋₆ alkyl)₂, C₁₋₆ alkoxy, or 5- to 10-membered heterocyclyl, wherein each C₁₋₆ alkyl, C₁₋₆ alkoxy, and 5- to 10-membered heterocyclyl of R³ is optionally substituted with one to three R^{3a}, wherein R^{3a} is as described herein. In some embodiments, each R³ is independently halogen, C₁₋₆ alkyl, —NH₂, —NH(C₁₋₆ alkyl), —N(C₁₋₆ alkyl)₂, C₁₋₆ alkoxy, or 5- to 10-membered heterocyclyl, wherein each C₁₋₆ alkyl, C₁₋₆ alkoxy, and 5- to 10-membered heterocyclyl of R³ is optionally substituted with one to two R^{3a}, wherein R^{3a} is as described herein. In some embodiments, each C₁₋₆ alkyl, C₁₋₆ alkoxy, and 5- to 10-membered heterocyclyl of R³ is optionally substituted with one R^{3a}, wherein R^{3a} is as described herein. In some embodiments, a compound of the disclosure is a compound, or pharmaceutically acceptable salt thereof, wherein each R³ is independently halogen, C₁₋₆ alkyl, —NH₂, —NH(C₁₋₆ alkyl), —N(C₁₋₆ alkyl)₂, C₁₋₆ alkoxy, or 5- to 10-membered heterocyclyl.

In some embodiments, a compound of the disclosure is a compound, or pharmaceutically acceptable salt thereof, wherein at least one R³ is —NH₂, —NH(C₁₋₃ alkyl), —N(C₁₋₃ alkyl)₂, or —O(C₁₋₃ alkyl), wherein each C₁₋₃ alkyl of R³ is independently optionally substituted one or two R^{3a}, wherein R^{3a} is as described herein. In some embodiments, each C₁₋₃ alkyl of R³ is independently optionally substituted one R^{3a}, wherein R^{3a} is as described herein. In some embodiments, a compound of the disclosure is a compound, or pharmaceutically acceptable salt thereof, wherein at least one R³ is —NH₂, —NH(C₁₋₃ alkyl), —N(C₁₋₃ alkyl)₂, or —O(C₁₋₃ alkyl).

In some embodiments, a compound of the disclosure is a compound, or pharmaceutically acceptable salt thereof, wherein at least one R³ is 5- to 7-membered heterocyclyl optionally substituted with one to three R^{3a}, wherein R^{3a} is as described herein. In some embodiments, a compound of the disclosure is a compound, or pharmaceutically acceptable salt thereof, wherein at least one R³ is 5- to 7-membered heterocyclyl optionally substituted with one to two R^{3a}, wherein R^{3a} is as described herein. In some embodiments, a compound of the disclosure is a compound, or pharmaceutically acceptable salt thereof, wherein at least one R³ is 5- to 7-membered heterocyclyl optionally substituted with one R^{3a}, wherein R^{3a} is as described herein. In some embodiments, a compound of the disclosure is a compound, or pharmaceutically acceptable salt thereof, wherein at least one R³ is 5- to 7-membered heterocyclyl.

In some embodiments, a compound of the disclosure is a compound, or pharmaceutically acceptable salt thereof, wherein each R³ is independently halogen, C₁₋₆ alkyl, or C₁₋₆ alkoxy. In some embodiments, a compound of the disclosure is a compound, or pharmaceutically acceptable salt thereof, wherein each R³ is independently C₁₋₆ alkyl, or C₁₋₆ alkoxy. In some embodiments, a compound of the disclosure is a compound, or pharmaceutically acceptable salt thereof, wherein each R³ is independently C₁₋₆ alkyl or C₁₋₆ alkoxy. In some embodiments, a compound of the disclosure is a compound, or pharmaceutically acceptable salt thereof, wherein each R³ is independently C₁₋₆ alkyl.

In some embodiments, a compound of the disclosure is a compound, or pharmaceutically acceptable salt thereof, wherein at least one R³ is C₁₋₆ alkoxy optionally substituted with one or two R^{3a}, wherein R^{3a} is as described herein. In some embodiments, a compound of the disclosure is a compound, or pharmaceutically acceptable salt thereof, wherein at least one R³ is C₁₋₆ alkoxy optionally substituted with one R^{3a}, wherein R^{3a} is as described herein. In some embodiments, a compound of the disclosure is a compound, or pharmaceutically acceptable salt thereof, wherein at least one R³ is C₁₋₆ alkoxy. In some embodiments, a compound of the disclosure is a compound, or pharmaceutically acceptable salt thereof, wherein at least one R³ is methoxy.

In some embodiments, a compound of the disclosure is a compound, or pharmaceutically acceptable salt thereof, wherein at least one R³ is C₁₋₆ alkyl. In some embodiments, a compound of the disclosure is a compound, or pharmaceutically acceptable salt thereof, wherein at least one R³ is C₁₋₃ alkyl. In some embodiments, a compound of the disclosure is a compound, or pharmaceutically acceptable salt thereof, wherein at least one R³ is methyl.

In some embodiments, a compound of the disclosure is a compound, or pharmaceutically acceptable salt thereof, wherein at least one R³ is halogen. In some embodiments, a compound of the disclosure is a compound, or pharmaceutically acceptable salt thereof, wherein at least one R³ is fluoro or chloro.

In some embodiments, a compound of the disclosure is a compound, or pharmaceutically acceptable salt thereof, wherein each R^{3a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂, wherein each C₁₋₆ alkyl, C₃₋₈ cycloalkyl, 4- to 10-membered heterocyclyl, phenyl, and 5- to 10-membered heteroaryl of R^{3a} is optionally substituted with one to two R^{3b}, wherein R^{3b} is as described herein. In some embodiments, each C₁₋₆ alkyl, C₃₋₈ cycloalkyl, 4- to 10-membered heterocyclyl, phenyl, and 5- to 10-membered heteroaryl of R^{3a} is optionally substituted with one R^{3b}, wherein R^{3b} is as described herein.

In some embodiments, a compound of the disclosure is a compound, or pharmaceutically acceptable salt thereof, wherein each R^{3a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂.

In some embodiments, a compound of the disclosure is a compound, or pharmaceutically acceptable salt thereof, wherein each R^{3a} is independently halogen, —C(O)(C₁₋₆ alkyl), —OH, —O(C₁₋₆ alkyl), 5- to 10-membered heterocyclyl, —C(O)NH₂, —C(O)N(H)(C₁₋₆ alkyl), or —C(O)N(C₁₋₆ alkyl)₂, wherein each C₁₋₆ alkyl and 5- to 10-membered heterocyclyl of R^{3a} is optionally substituted with one to two R^{3b}, wherein R^{3b} is as described herein. In some embodiments, each C₁₋₆ alkyl and 5- to 10-membered heterocyclyl of R^{3a} is optionally substituted with one R^{3b}, wherein R^{3b} is as described herein. In some embodiments, a compound of the disclosure is a compound, or pharmaceutically acceptable salt thereof, wherein each R^{3a} is independently halogen, —C(O)(C₁₋₆ alkyl), —OH, —O(C₁₋₆ alkyl), 5- to 10-membered heterocyclyl, —C(O)NH₂, —C(O)N(H)(C₁₋₆ alkyl), or —C(O)N(C₁₋₆ alkyl)₂.

In some embodiments, a compound of the disclosure is a compound, or pharmaceutically acceptable salt thereof, wherein at least one R^{3a} is —O(C₁₋₃ alkyl), 5- to 7-membered heterocyclyl, —C(O)NH₂, —C(O)N(H)(C₁-C₃ alkyl), or —C(O)N(C₁₋₃ alkyl)₂, wherein each C₁₋₃ alkyl and 5- to 7-membered heterocyclyl of R^{3a} is optionally substituted with one to two R^{3b}, wherein R^{3b} is as described herein. In some embodiments, each C₁₋₃ alkyl and 5- to 7-membered heterocyclyl of R^{3a} is optionally substituted with one R^{3b}, wherein R^{3b} is as described herein. In some embodiments, a compound of the disclosure is a compound, or pharmaceutically acceptable salt thereof, wherein at least one R^{3a} is —O(C₁₋₃ alkyl), 5- to 7-membered heterocyclyl, —C(O)NH₂, —C(O)N(H)(C₁-C₃ alkyl), or —C(O)N(C₁₋₃ alkyl)₂.

In some embodiments, a compound of the disclosure is a compound, or pharmaceutically acceptable salt thereof, wherein at least one R^{3a} is halogen or —C(O)(C₁₋₆ alkyl). In some embodiments, a compound of the disclosure is a compound, or pharmaceutically acceptable salt thereof, wherein at least one R^{3a} is halogen or —C(O)(C₁₋₃ alkyl). In some embodiments, a compound of the disclosure is a compound, or pharmaceutically acceptable salt thereof, wherein at least one R^{3a} is chloro, fluoro, or —C(O)(C₁₋₃ alkyl).

In some embodiments, a compound of the disclosure is a compound, or pharmaceutically acceptable salt thereof, wherein each R^{3a} is independently halogen or —C(O)(C₁₋₆ alkyl). In some embodiments, a compound of the disclosure is a compound, or pharmaceutically acceptable salt thereof, wherein each R^{3a} is independently halogen or —C(O)(C₁₋₃ alkyl). In some embodiments, a compound of the disclosure is a compound, or pharmaceutically acceptable salt thereof, wherein each R^{3a} is independently chloro, fluoro, or —C(O)(C₁₋₃ alkyl).

In some embodiments, a compound of the disclosure is a compound, or pharmaceutically acceptable salt thereof, wherein at least one R^{3a} is —OH or —O(C₁₋₃ alkyl). In some embodiments, a compound of the disclosure is a compound, or pharmaceutically acceptable salt thereof, wherein at least one R^{3a} is —OH or —O(methyl).

In some embodiments, a compound of the disclosure is a compound, or pharmaceutically acceptable salt thereof, wherein each R^{3a} is independently —OH or —O(C₁₋₃ alkyl). In some embodiments, a compound of the disclosure is a compound, or pharmaceutically acceptable salt thereof, wherein each R^{3a} is independently —OH or —O(methyl).

In some embodiments, a compound of the disclosure is a compound, or pharmaceutically acceptable salt thereof, wherein at least one R^{3a} is —O(C₁₋₃ alkyl). In some embodiments, a compound of the disclosure is a compound, or pharmaceutically acceptable salt thereof, wherein at least one R^{3a} is —O(methyl).

In some embodiments, a compound of the disclosure is a compound, or pharmaceutically acceptable salt thereof, wherein each R^{3a} is independently —O(C₁₋₃ alkyl). In some embodiments, a compound of the disclosure is a compound, or pharmaceutically acceptable salt thereof, wherein each R^{3a} is independently —O(methyl).

In some embodiments, a compound of the disclosure is a compound, or pharmaceutically acceptable salt thereof, wherein at least one R^{3a} is halogen or —OH. In some embodiments, a compound of the disclosure is a compound, or pharmaceutically acceptable salt thereof, wherein at least one R^{3a} is chloro, fluoro, or —OH.

In some embodiments, a compound of the disclosure is a compound, or pharmaceutically acceptable salt thereof, wherein each R^{3a} is independently halogen or —OH. In some embodiments, a compound of the disclosure is a compound, or pharmaceutically acceptable salt thereof, wherein each R^{3a} is independently chloro, fluoro, or —OH.

In some embodiments, a compound of the disclosure is a compound, or pharmaceutically acceptable salt thereof, wherein each R^{3b} is independently halogen, —O(C₁₋₆ alkyl), or —O(C₁₋₆ haloalkyl). In some embodiments, a compound of the disclosure is a compound, or pharmaceutically acceptable salt thereof, wherein each R^{3b} is independently fluoro, chloro, —O(C₁₋₃ alkyl), or —O(C₁₋₃ haloalkyl).

In some embodiments, a compound of the disclosure is a compound, or pharmaceutically acceptable salt thereof, wherein each R^{L} is independently hydrogen or C₁₋₆ alkyl. In some embodiments, a compound of the disclosure is a compound, or pharmaceutically acceptable salt thereof, wherein each R^{L} is independently hydrogen or C₁₋₃ alkyl. In some embodiments, a compound of the disclosure is a compound, or pharmaceutically acceptable salt thereof, wherein R^{L} is hydrogen. In some embodiments, a compound of the disclosure is a compound, or pharmaceutically acceptable salt thereof, wherein R^{L} is methyl.

In some embodiments, a compound of the disclosure is a compound, or pharmaceutically acceptable salt thereof, wherein n is an integer from 0 to 2. In some embodiments, a compound of the disclosure is a compound, or pharmaceutically acceptable salt thereof, wherein n is an integer from 1 to 3. In some embodiments, a compound of the disclosure is a compound, or pharmaceutically acceptable salt thereof, wherein n is 0 or 1. In some embodiments, a compound of the disclosure is a compound, or pharmaceutically acceptable salt thereof, wherein n is 1 or 2. In some embodiments, a compound of the disclosure is a compound, or pharmaceutically acceptable salt thereof, wherein n is 2 or 3. In some embodiments, a compound of the disclosure is a compound, or pharmaceutically acceptable salt thereof, wherein n is 0. In some embodiments, a compound of the disclosure is a compound, or pharmaceutically acceptable salt thereof, wherein n is 1. In some embodiments, a compound of the disclosure is a compound, or pharmaceutically acceptable salt thereof, wherein n is 2. In some embodiments, a compound of the disclosure is a compound, or pharmaceutically acceptable salt thereof, wherein

In some embodiments, a compound of the disclosure is a compound, or pharmaceutically acceptable salt thereof, wherein ring \textcircled{A} taken together with the ring comprising X⁴, X⁵, X⁶, and X⁷ is

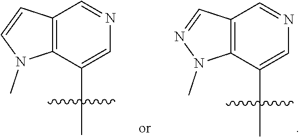 [SMILES: *c1cncc2ccn(C)c12 ; *c1cncc2cnn(C)c12]

In some embodiments, a compound of the disclosure is a compound, or pharmaceutically acceptable salt thereof, wherein ring \textcircled{A} taken together with the ring comprising X⁴, X⁵, X⁶, and X⁷ is

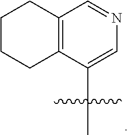 [SMILES: *c1cncc2c1CCCC2]

In some embodiments, a compound of the disclosure is a compound, or pharmaceutically acceptable salt thereof, wherein ring \textcircled{A} taken together with the ring comprising X⁴, X⁵, X⁶, and X⁷ is

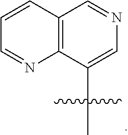 [SMILES: *c1cncc2cccnc12]

In some embodiments, a compound of the disclosure is a compound, or pharmaceutically acceptable salt thereof, wherein ring \textcircled{A} taken together with the ring comprising X⁴, X⁵, X⁶, and X⁷ is R³

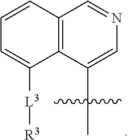

In some embodiments, a compound of the disclosure is a compound, or pharmaceutically acceptable salt thereof, wherein ring \textcircled{A} taken together with the ring comprising X⁴, X⁵, X⁶, and X⁷ is

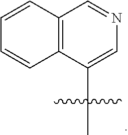 [SMILES: *c1cncc2ccccc12]

In some embodiments, the disclosure provides a compound according to the structure of Formula (V-A):

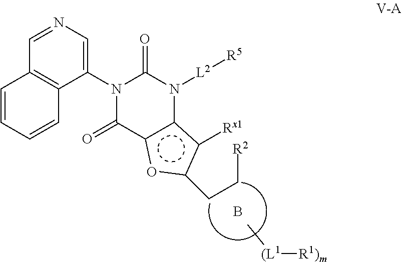

or a pharmaceutically acceptable salt thereof, wherein ring \textcircled{B}, R^{x1}, L¹, L², R¹, R², R⁵, and m are as described herein.

In some embodiments, the disclosure provides a compound according to the structure of Formula (V-B):

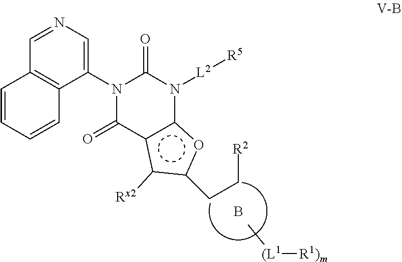

or a pharmaceutically acceptable salt thereof, wherein ring \textcircled{B}, R^{x2}, L¹, L², R¹, R², R⁵, and m are as described herein.

In some embodiments, the disclosure provides a compound according to the structure of Formula (V-C):

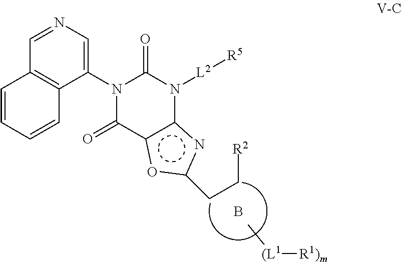

or a pharmaceutically acceptable salt thereof, wherein ring \textcircled{B}, L¹, L², R¹, R², R⁵, and m are as described herein.

In some embodiments, the disclosure provides a compound according to the structure of Formula (V-D):

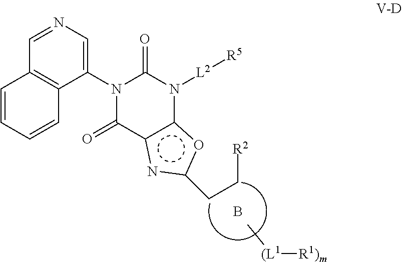

or a pharmaceutically acceptable salt thereof, wherein ring \textcircled{B}, L¹, L², R¹, R², R⁵, and m are as described herein.

In some embodiments, a compound of the disclosure is a compound, or pharmaceutically acceptable salt thereof, wherein ring \textcircled{A} is absent.

In some embodiments, a compound of the disclosure is a compound, or pharmaceutically acceptable salt thereof, wherein ring \textcircled{A} is absent, wherein X⁴ is N or C-L^{x4}-R^{x4}, and X⁵ is N or C-L^{x5}-R^{x5}. In some embodiments, a compound of the disclosure is a compound, or pharmaceutically acceptable salt thereof, wherein ring \textcircled{A} is absent, wherein X⁴ is N, and X⁵ is C-L^{x5}-R^{x5}. In some embodiments, a compound of the disclosure is a compound, or pharmaceutically acceptable salt thereof, wherein \textcircled{A} ring is absent, wherein X⁴ is C-L^{x4}-R^{x4}, and X⁵ is N. In some embodiments, a compound of the disclosure is a compound, or pharmaceutically acceptable salt thereof, wherein ring \textcircled{A} is absent, wherein X⁴ is C-L^{x4}-R^{x4}, and X⁵ is C-L^{x5}-R^{x5}.

In some embodiments, a compound of the disclosure is a compound, or pharmaceutically acceptable salt thereof, wherein L^{x4} is a bond, —(C₁₋₃ alkyl)O—, —(C₁₋₃ alkyl)N(R^{L})C(O)—, —(C₁₋₃ alkyl)C(O)N(R^{L})—, —(C₁₋₃ alkyl)N(R^{L})C(O)(C₁₋₃ alkyl)-, —(C₁₋₃ alkyl)C(O)N(R^{L})(C₁₋₃ alkyl)-, —(C₁₋₃ alkyl), —(C₁₋₃ alkyl)N(R^{L})S(O)₂—, —N(R^{L})S(O)₂—, —C(O)—, —(C₁₋₃ alkyl)C(O)—, or —N(R^{L})C(O)—, wherein R^{L} is as described herein.

In some embodiments, a compound of the disclosure is a compound, or pharmaceutically acceptable salt thereof, wherein L^{x4} is —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl-, —(C₁₋₆ alkyl), —(C₁₋₆ alkyl)N(R^{L})S(O)₂—, —N(R^{L})S(O)₂—, —C(O)—, —(C₁₋₆ alkyl)C(O)—, or —N(R^{L})C(O)—, wherein R^{L} is as described herein. In some embodiments, a compound of the disclosure is a compound, or pharmaceutically acceptable salt thereof, wherein L^{x4} is —(C₁₋₃ alkyl)O—, —(C₁₋₃ alkyl)N(R^{L})C(O)—, —(C₁₋₃ alkyl)C(O)N(R^{L})—, —(C₁₋₃ alkyl)N(R^{L})C(O)(C₁₋₃ alkyl)-, —(C₁₋₃ alkyl)C(O)N(R^{L})(C₁₋₃ alkyl)-, —(C₁₋₃ alkyl), —(C₁₋₃ alkyl)N(R^{L})S(O)₂—, —N(R^{L})S(O)₂—, —C(O)—, —(C₁₋₃ alkyl)C(O)—, or —N(R^{L})C(O)—, wherein R^{L} is as described herein.

In some embodiments, a compound of the disclosure is a compound, or pharmaceutically acceptable salt thereof, wherein L^{x4} is a bond or N(R^{L})S(O)₂—, wherein R^{L} is as defined herein. In some embodiments, a compound of the disclosure is a compound, or pharmaceutically acceptable salt thereof, wherein L^{x4} is a N(R^{L})S(O)₂—, wherein R^{L} is as defined herein. In some embodiments, a compound of the disclosure is a compound, or pharmaceutically acceptable salt thereof, wherein L^{x4} is a bond.

In some embodiments, a compound of the disclosure is a compound, or pharmaceutically acceptable salt thereof, wherein L^{x5} is a bond, —(C₁₋₃ alkyl)O—, —(C₁₋₃ alkyl)N(R^{L})C(O)—, —(C₁₋₃ alkyl)C(O)N(R^{L})—, —(C₁₋₃ alkyl)N(R^{L})C(O)(C₁₋₃ alkyl)-, —(C₁₋₃ alkyl)C(O)N(R^{L})(C₁₋₃ alkyl)-, —(C₁₋₃ alkyl), —(C₁₋₃ alkyl)N(R^{L})S(O)₂—, —N(R^{L})S(O)₂—, —C(O)—, —(C₁₋₃ alkyl)C(O)—, or —N(R^{L})C(O)—, wherein R^{L} is as described herein.

In some embodiments, a compound of the disclosure is a compound, or pharmaceutically acceptable salt thereof, wherein L^{x5} is —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl-, —(C₁₋₆ alkyl), —(C₁₋₆ alkyl)N(R^{L})S(O)₂—, —N(R^{L})S(O)₂—, —C(O)—, —(C₁₋₆ alkyl)C(O)—, or —N(R^{L})C(O)—, wherein R^{L} is as described herein. In some embodiments, a compound of the disclosure is a compound, or pharmaceutically acceptable salt thereof, wherein L^{x5} is —(C₁₋₃ alkyl)O—, —(C₁₋₃ alkyl)N(R^{L})C(O)—, —(C₁₋₃ alkyl)C(O)N(R^{L})—, —(C₁₋₃ alkyl)N(R^{L})C(O)(C₁₋₃ alkyl)-, —(C₁₋₃ alkyl)C(O)N(R^{L})(C₁₋₃ alkyl)-, —(C₁₋₃ alkyl), —(C₁₋₃ alkyl)N(R^{L})S(O)₂—, —N(R^{L})S(O)₂—, —C(O)—, —(C₁₋₃ alkyl)C(O)—, or —N(R^{L})C(O)—, wherein R^{L} is as described herein.

In some embodiments, a compound of the disclosure is a compound, or pharmaceutically acceptable salt thereof, wherein L^{x5} is a bond or N(R^{L})S(O)₂—, wherein R^{L} is as defined herein. In some embodiments, a compound of the disclosure is a compound, or pharmaceutically acceptable salt thereof, wherein L^{x5} is a N(R^{L})S(O)₂—, wherein R^{L} is as defined herein. In some embodiments, a compound of the disclosure is a compound, or pharmaceutically acceptable salt thereof, wherein L^{x5} is a bond.

In some embodiments, a compound of the disclosure is a compound, or pharmaceutically acceptable salt thereof, wherein each L^{x4} and L^{x5} is independently a bond or N(R^{L})S(O)₂—, wherein R^{L} is as defined herein. In some embodiments, a compound of the disclosure is a compound, or pharmaceutically acceptable salt thereof, wherein L^{x4} and L^{x5} are each a bond.

In some embodiments, the disclosure provides a compound according to the structure of Formula (VI):

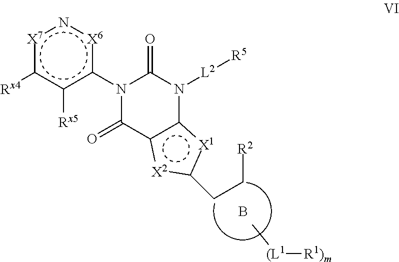

or a pharmaceutically acceptable salt thereof, wherein ring \textcircled{B}, X¹, X², X⁶, X⁷, L¹, L², R¹, R², R⁵, R^{x4}, R^{x5}, and m are as described herein.

In some embodiments, the compound of Formula (VI) is:

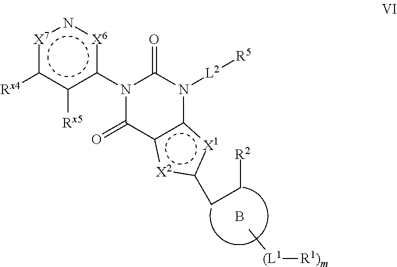

or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
each R^{x4} and R^{x5} is independently hydrogen, C₁₋₆ alkyl, —NH₂, —NH(C₁₋₆ alkyl), —N(C₁₋₆ alkyl)₂, C₁₋₆ alkoxy, C₃-C₈ cycloalkyl, or 5- to 10-membered heterocyclyl;
   wherein each C₁₋₆ alkyl, C₁₋₆ alkoxy, C₃₋₈ cycloalkyl, and 5- to 10-membered heterocyclyl of R^{x4} and R^{x5} is optionally substituted with one to four R^{4a};
each R^{4a} is independently C₁₋₆ alkyl, —C(O)(5- to 10-membered heterocyclyl), or —O(C₃₋₈ cycloalkyl),
   wherein each C₁₋₆ alkyl, C₃₋₈ cycloalkyl, and 5- to 10-membered heterocyclyl of R^{4a} is optionally substituted with one to three R^{4b};
each R^{4b} is independently halogen, —O(C₁₋₆ alkyl), or —O(C₁₋₆ haloalkyl); each X⁶ and X⁷ is independently N or CH; ring \textcircled{B} is C₆₋₁₀ aryl;
each L¹ is independently bond, —O—, —(C₁₋₆ alkyl)O—, —O(C₁₋₆ alkyl)-, or —C₁₋₆ alkyl-O(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
   wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl,
   wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
each R^{1b} is independently C₁₋₆ alkyl, C₁₋₆ haloalkyl, halogen, oxo, —OH, or —NH₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-;
R⁵ is hydrogen, —CN, C₁₋₆ alkyl, C₃₋₈ cycloalkyl, phenyl, 4- to 10-membered heterocyclyl, or 5- to 10-membered heteroaryl,
   wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{b};
each R^{5a} is independently hydrogen or C₁₋₆ alkyl; and
each R^{5b} is independently oxo, C₁₋₃ alkyl, or C₁₋₃ alkoxy.

In some embodiments, the disclosure provides a compound according to the structure of Formula (VII-A):

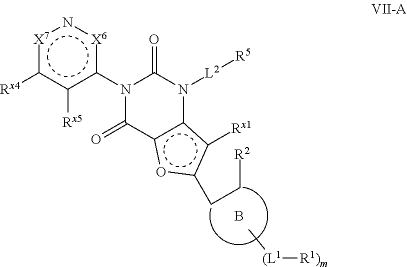

or a pharmaceutically acceptable salt thereof, wherein ring \textcircled{B}, X⁶, X⁷, L¹, L², R¹, R², R⁵, R^{x1}, R^{x4}, R^{x5}, and m are as described herein.

In some embodiments, the disclosure provides a compound according to the structure of Formula (VII-B):

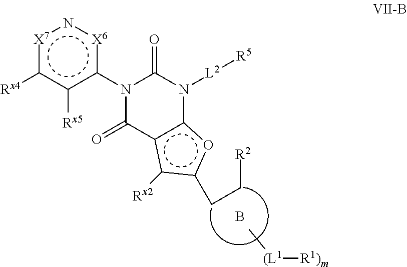

or a pharmaceutically acceptable salt thereof, wherein ring, X⁶, X⁷, L¹, L², R¹, R², R⁵, R^{x2}, R^{x4}, R^{x5}, and m are as described herein.

In some embodiments, the disclosure provides a compound according to the structure of Formula (VII-C):

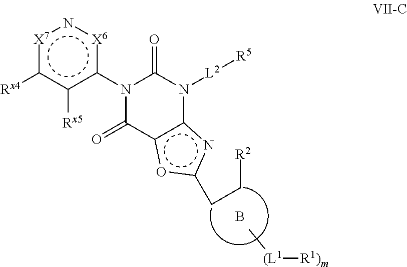

or a pharmaceutically acceptable salt thereof, wherein ring, X⁶, X⁷, L¹, L², R¹, R², R⁵, R^{x4}, R^{x5}, and m are as described herein.

In some embodiments, the disclosure provides a compound according to the structure of Formula (VII-D):

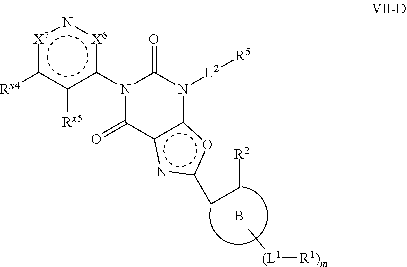

or a pharmaceutically acceptable salt thereof, wherein ring \textcircled{B}, X⁶, X⁷, L¹, L², R¹, R², R⁵, R^{x4}, R^{x5}, and m are as described herein.

In some embodiments, a compound of the disclosure is a compound, or pharmaceutically acceptable salt thereof, wherein R^{x4} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl, wherein each C₁₋₆ alkyl, C₂₋₆ alkynyl, C₁₋₆ alkoxy, C₃₋₈ cycloalkyl, and 4- to 10-membered heterocyclyl of R^{x4} is optionally substituted with one to three R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x4} is optionally substituted with one to two R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x4} is optionally substituted with one R^{4a}, wherein R^{4a} is as described herein. In some embodiments, a compound of the disclosure is a compound, or pharmaceutically acceptable salt thereof, wherein R^{x4} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl.

In some embodiments, a compound of the disclosure is a compound, or pharmaceutically acceptable salt thereof, wherein R^{x4} is hydrogen, halogen, hydroxy, —CN, C₁₋₆ alkyl, C₂₋₃ alkynyl, C₁₋₃ alkoxy, C₃₋₈ cycloalkyl, 4- to 10-membered heterocyclyl, 5- to 10-membered heteroaryl, —NH₂, —NH(C₁₋₃ alkyl), —N(C₁₋₃ alkyl)₂, —O(C₁₋₃ alkyl), or —OC₃₋₈ cycloalkyl, wherein each C₁₋₃ alkyl, C₂₋₃ alkynyl, C₁₋₃ alkoxy, C₃₋₈ cycloalkyl, and 4- to 10-membered heterocyclyl of R^{x4} is optionally substituted with one to four R^{4a}, wherein R^{4a} is as described herein. In some embodiments, each C₁₋₃ alkyl, C₂₋₃ alkynyl, C₁₋₃ alkoxy, C₃₋₈ cycloalkyl, and 4- to 10-membered heterocyclyl of R^{x4} is optionally substituted with one to three R^{4a}, wherein R^{4a} is as described herein. In some embodiments, each C₁₋₃ alkyl, C₂₋₃ alkynyl, C₁₋₃ alkoxy, C₃₋₈ cycloalkyl, and 4- to 10-membered heterocyclyl of R^{x4} is optionally substituted with one to two R^{4a}, wherein R^{4a} is as described herein. In some embodiments, each C₁₋₃ alkyl, C₂₋₃ alkynyl, C₁₋₃ alkoxy, C₃₋₈ cycloalkyl, and 4- to 10-membered heterocyclyl of R^{x4} is optionally substituted with one R^{4a}, wherein R^{4a} is as described herein. In some embodiments, a compound of the disclosure is a compound, or pharmaceutically acceptable salt thereof, wherein R^{x4} is hydrogen, halogen, hydroxy, —CN, C₁₋₃ alkyl, C₂₋₃ alkynyl, C₁₋₃ alkoxy, C₃₋₈ cycloalkyl, 4- to 10-membered heterocyclyl, 5- to 10-membered heteroaryl, —NH₂, —NH(C₁₋₃ alkyl), —N(C₁₋₃ alkyl)₂, —O(C₁₋₃ alkyl), or —OC₃₋₈ cycloalkyl.

In some embodiments, a compound of the disclosure is a compound, or pharmaceutically acceptable salt thereof, wherein R^{x4} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl, wherein each C₁₋₆ alkyl, C₂₋₆ alkynyl, C₁₋₆ alkoxy, C₃₋₈ cycloalkyl, and 4- to 10-membered heterocyclyl of R^{x4} is optionally substituted with one to three R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x4} is optionally substituted with one to two R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x4} is optionally substituted with one R^{4a}, wherein R^{4a} is as described herein. In some embodiments, a compound of the disclosure is a compound, or pharmaceutically acceptable salt thereof, wherein R^{x4} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl.

In some embodiments, a compound of the disclosure is a compound, or pharmaceutically acceptable salt thereof, wherein R^{x4} is hydrogen, C₁₋₆ alkyl, —NH₂, —NH(C₁₋₆ alkyl), —N(C₁₋₆ alkyl)₂, C₁₋₆ alkoxy, C₃₋₈ cycloalkyl, or 5- to 10-membered heterocyclyl, wherein each C₁₋₆ alkyl, C₁₋₆ alkoxy, C₃₋₈ cycloalkyl, and 5- to 10-membered heterocyclyl of R^{x4} is optionally substituted with one to three R^{4a}, wherein R^{4a} is as described herein. In some embodiments, each C₁₋₆ alkyl, C₁₋₆ alkoxy, C₃₋₈ cycloalkyl, and 5- to 10-membered heterocyclyl of R^{x4} is optionally substituted with one to two R^{4a}, wherein R^{4a} is as described herein. In some embodiments, each C₁₋₆ alkyl, C₁₋₆ alkoxy, C₃₋₈ cycloalkyl, and 5- to 10-membered heterocyclyl of R^{x4} is optionally substituted with one R^{4a}, wherein R^{4a} is as described herein. In some embodiments, a compound of the disclosure is a compound, or pharmaceutically acceptable salt thereof, wherein R^{x4} is hydrogen, C₁₋₆ alkyl, —NH₂, —NH(C₁₋₆ alkyl), —N(C₁₋₆ alkyl)₂, C₁₋₆ alkoxy, C₃₋₈ cycloalkyl, or 5- to 10-membered heterocyclyl.

In some embodiments, a compound of the disclosure is a compound, or pharmaceutically acceptable salt thereof, wherein R^{x4} is C₁₋₆ alkyl. In some embodiments, a compound of the disclosure is a compound, or pharmaceutically acceptable salt thereof, wherein R^{x4} is C₁₋₃ alkyl. In some embodiments, a compound of the disclosure is a compound, or pharmaceutically acceptable salt thereof, wherein R^{x4} is methyl.

In some embodiments, a compound of the disclosure is a compound, or pharmaceutically acceptable salt thereof, wherein R^{x4} is hydrogen.

In some embodiments, a compound of the disclosure is a compound, or pharmaceutically acceptable salt thereof, wherein R^{x5} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl, wherein each C₁₋₆ alkyl, C₂₋₆ alkynyl, C₁₋₆ alkoxy, C₃₋₈ cycloalkyl, and 4- to 10-membered heterocyclyl of R^{x5} is optionally substituted with one to three R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x5} is optionally substituted with one to two R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x5} is optionally substituted with one R^{4a}, wherein R^{4a} is as described herein. In some embodiments, a compound of the disclosure is a compound, or pharmaceutically acceptable salt thereof, wherein R^{x5} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl.

In some embodiments, a compound of the disclosure is a compound, or pharmaceutically acceptable salt thereof, wherein R^{x5} is hydrogen, halogen, hydroxy, —CN, C₁₋₆ alkyl, C₂₋₃ alkynyl, C₁₋₃ alkoxy, C₃₋₈ cycloalkyl, 4- to 10-membered heterocyclyl, 5- to 10-membered heteroaryl, —NH₂, —NH(C₁₋₃ alkyl), —N(C₁₋₃ alkyl)₂, —O(C₁₋₃ alkyl), or —OC₃₋₈ cycloalkyl, wherein each C₁₋₃ alkyl, C₂₋₃ alkynyl, C₁₋₃ alkoxy, C₃₋₈ cycloalkyl, and 4- to 10-membered heterocyclyl of R^{x5} is optionally substituted with one to four R^{4a}, wherein R^{4a} is as described herein. In some embodiments, each C₁₋₃ alkyl, C₂₋₃ alkynyl, C₁₋₃ alkoxy, C₃₋₈ cycloalkyl, and 4- to 10-membered heterocyclyl of R^{x5} is optionally substituted with one to three R^{4a}, wherein R^{4a} is as described herein. In some embodiments, each C₁₋₃ alkyl, C₂₋₃ alkynyl, C₁₋₃ alkoxy, C₃₋₈ cycloalkyl, and 4- to 10-membered heterocyclyl of R^{x5} is optionally substituted with one to two R^{4a}, wherein R^{4a} is as described herein. In some embodiments, each C₁₋₃ alkyl, C₂₋₃ alkynyl, C₁₋₃ alkoxy, C₃₋₈ cycloalkyl, and 4- to 10-membered heterocyclyl of R^{x5} is optionally substituted with one R^{4a}, wherein R^{4a} is as described herein. In some embodiments, a compound of the disclosure is a compound, or pharmaceutically acceptable salt thereof, wherein R^{x5} is hydrogen, halogen, hydroxy, —CN, C₁₋₃ alkyl, C₂₋₃ alkynyl, C₁₋₃ alkoxy, C₃₋₈ cycloalkyl, 4- to 10-membered heterocyclyl, 5- to 10-membered heteroaryl, —NH₂, —NH(C₁₋₃ alkyl), —N(C₁₋₃ alkyl)₂, —O(C₁₋₃ alkyl), or —OC₃₋₈ cycloalkyl.

In some embodiments, a compound of the disclosure is a compound, or pharmaceutically acceptable salt thereof, wherein R^{x5} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl, wherein each C₁₋₆ alkyl, C₂₋₆ alkynyl, C₁₋₆ alkoxy, C₃₋₈ cycloalkyl, and 4- to 10-membered heterocyclyl of R^{x5} is optionally substituted with one to three R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x5} is optionally substituted with one to two R^{4a}, wherein R^{4a} is as described herein. In some embodiments, each C₁₋₆ alkyl, C₂₋₆ alkynyl, C₁₋₆ alkoxy, C₃₋₈ cycloalkyl, and 4- to 10-membered heterocyclyl of R^{x5} is optionally substituted with one R^{4a}, wherein R^{4a} is as described herein. In some embodiments, a compound of the disclosure is a compound, or pharmaceutically acceptable salt thereof, wherein R^{x5} is hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl.

In some embodiments, a compound of the disclosure is a compound, or pharmaceutically acceptable salt thereof, wherein R^{x5} is hydrogen, C₁₋₆ alkyl, —NH₂, —NH(C₁₋₆ alkyl), —N(C₁₋₆ alkyl)₂, C₁₋₆ alkoxy, C₃₋₈ cycloalkyl, or 5- to 10-membered heterocyclyl, wherein each C₁₋₆ alkyl, C₁₋₆ alkoxy, C₃₋₈ cycloalkyl, and 5- to 10-membered heterocyclyl of R^{x5} is optionally substituted with one to three R^{4a}, wherein R^{4a} is as described herein. In some embodiments, each C₁₋₆ alkyl, C₁₋₆ alkoxy, C₃₋₈ cycloalkyl, and 5- to 10-membered heterocyclyl of R^{x5} is optionally substituted with one to two R^{4a}, wherein R^{4a} is as described herein. In some embodiments, each C₁₋₆ alkyl, C₁₋₆ alkoxy, C₃₋₈ cycloalkyl, and 5- to 10-membered heterocyclyl of R^{x5} is optionally substituted with one R^{4a}, wherein R^{4a} is as described herein. In some embodiments, a compound of the disclosure is a compound, or pharmaceutically acceptable salt thereof, wherein R^{x5} is independently hydrogen, C₁₋₆ alkyl, —NH₂, —NH(C₁₋₆ alkyl), —N(C₁₋₆ alkyl)₂, C₁₋₆ alkoxy, C₃₋₈ cycloalkyl, or 5- to 10-membered heterocyclyl.

In some embodiments, a compound of the disclosure is a compound, or pharmaceutically acceptable salt thereof, wherein R^{x5} is C₁₋₆ alkyl. In some embodiments, a compound of the disclosure is a compound, or pharmaceutically acceptable salt thereof, wherein R^{x5} is C₁₋₃ alkyl. In some embodiments, a compound of the disclosure is a compound, or pharmaceutically acceptable salt thereof, wherein R^{x5} is methyl.

In some embodiments, a compound of the disclosure is a compound, or pharmaceutically acceptable salt thereof, wherein R^{x5} is hydrogen.

In some embodiments, a compound of the disclosure is a compound, or pharmaceutically acceptable salt thereof, wherein one of R^{x4} and R^{x5} is hydrogen and the other is C₃₋₈ cycloalkyl optionally substituted with one or two R^{4a}, wherein R^{4a} is as described herein. In some embodiments, a compound of the disclosure is a compound, or pharmaceutically acceptable salt thereof, wherein one of R^{x4} and R^{x5} is hydrogen and the other is C₃₋₈ cycloalkyl optionally substituted with one R^{4a}, wherein R^{4a} is as described herein. In some embodiments, a compound of the disclosure is a compound, or pharmaceutically acceptable salt thereof, wherein one of R^{x4} and R^{x5} is hydrogen and the other is C₃₋₈ cycloalkyl.

In some embodiments, a compound of the disclosure is a compound, or pharmaceutically acceptable salt thereof, wherein one of R^{x4} and R^{x5} is hydrogen and the other is C₁₋₆ alkoxy optionally substituted with one or two R^{4a}, wherein R^{4a} is as described herein. In some embodiments, a compound of the disclosure is a compound, or pharmaceutically acceptable salt thereof, wherein one of R^{x4} and R^{x5} is hydrogen and the other is C₁₋₆ alkoxy optionally substituted with one R^{4a}, wherein R^{4a} is as described herein. In some embodiments, a compound of the disclosure is a compound, or pharmaceutically acceptable salt thereof, wherein one of R^{x4} and R^{x5} is hydrogen and the other is C₁₋₆ alkoxy. In some embodiments, a compound of the disclosure is a compound, or pharmaceutically acceptable salt thereof, wherein one of R^{x4} and R^{x5} is hydrogen and the other is methoxy.

In some embodiments, a compound of the disclosure is a compound, or pharmaceutically acceptable salt thereof, wherein one of R^{x4} and R^{x5} is hydrogen and the other is C₁₋₆ alkyl optionally substituted with one or two R^{4a}, wherein R^{4a} is as described herein. In some embodiments, a compound of the disclosure is a compound, or pharmaceutically acceptable salt thereof, wherein one of R^{x4} and R^{x5} is hydrogen and the other is C₁₋₆ alkyl optionally substituted with one R^{4a}, wherein R^{4a} is as described herein. In some embodiments, a compound of the disclosure is a compound, or pharmaceutically acceptable salt thereof, wherein one of R^{x4} and R^{x5} is hydrogen and the other is C₁₋₆ alkyl. In some embodiments, a compound of the disclosure is a compound, or pharmaceutically acceptable salt thereof, wherein one of R^{x4} and R^{x5} is hydrogen and the other is C₁₋₃ alkyl. In some embodiments, a compound of the disclosure is a compound, or pharmaceutically acceptable salt thereof, wherein one of R^{x4} and R^{x5} is hydrogen and the other is methyl.

In some embodiments, a compound of the disclosure is a compound, or pharmaceutically acceptable salt thereof, wherein each R^{4a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂, wherein each C₁₋₆ alkyl, C₃₋₈ cycloalkyl, 4- to 10-membered heterocyclyl, phenyl, and 5- to 10-membered heteroaryl of R^{4a} is optionally substituted with one to three R^{4b}, wherein R^{4b} is as described herein. In some embodiments, each C₁₋₆ alkyl, C₃₋₈ cycloalkyl, 4- to 10-membered heterocyclyl, phenyl, and 5- to 10-membered heteroaryl of R^{4a} is optionally substituted with one to two R^{4b}, wherein R^{4b} is as described herein. In some embodiments, each C₁₋₆ alkyl, C₃₋₈ cycloalkyl, 4- to 10-membered heterocyclyl, phenyl, and 5- to 10-membered heteroaryl of R^{4a} is optionally substituted with one R^{4b}, wherein R^{4b} is as described herein.

In some embodiments, a compound of the disclosure is a compound, or pharmaceutically acceptable salt thereof, wherein each R^{4a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂.

In some embodiments, a compound of the disclosure is a compound, or pharmaceutically acceptable salt thereof, wherein each R^{4a} is independently halogen, —C(O)(C₁₋₆ alkyl), —OH, —O(C₁₋₆ alkyl), 5- to 10-membered heterocyclyl, —C(O)NH₂, —C(O)N(H)(C₁-C₆ alkyl), or —C(O)N(C₁-C₆ alkyl)₂, wherein each C₁₋₆ alkyl and 5- to 10-membered heterocyclyl of R^{4a} is optionally substituted with one to three R^{4b}, wherein R^{4b} is as described herein. In some embodiments, a compound of the disclosure is a compound, or pharmaceutically acceptable salt thereof, wherein each R^{4a} is independently chloro, fluoro, —C(O)(C₁-C₃ alkyl), —OH, —O(C₁₋₃ alkyl), 5- to 10-membered heterocyclyl, —C(O)NH₂, —C(O)N(H)(C₁-C₃ alkyl), or —C(O)N(C₁-C₃ alkyl)₂, wherein each C₁₋₃ alkyl and 5- to 10-membered heterocyclyl of R^{4a} is optionally substituted with one to three R^{4b}, wherein R^{4b} is as described herein. In some embodiments, each C₁₋₃ alkyl and 5- to 10-membered heterocyclyl of R^{4a} is optionally substituted with one or two R^{4b}, wherein R^{4b} is as described herein. In some embodiments, each C₁₋₃ alkyl and 5- to 10-membered heterocyclyl of R^{4a} is optionally substituted with one R^{4b}, wherein R^{4b} is as described herein. In some embodiments, a compound of the disclosure is a compound, or pharmaceutically acceptable salt thereof, wherein each R^{4a} is independently chloro, fluoro, —C(O)(C₁-C₃ alkyl), —OH, —O(C₁₋₃ alkyl), 5- to 10-membered heterocyclyl, —C(O)NH₂, —C(O)N(H)(C₁-C₃ alkyl), or —C(O)N(C₁-C₃ alkyl)₂.

In some embodiments, a compound of the disclosure is a compound, or pharmaceutically acceptable salt thereof, wherein each R^{4a} is independently —O(C₁₋₃ alkyl), 5- to 7-membered heterocyclyl, —C(O)NH₂, —C(O)N(H)(C₁₋₃ alkyl), or —C(O)N(C₁-C₃ alkyl)₂, wherein each C₁₋₃ alkyl and 5- to 7-membered heterocyclyl is optionally substituted with one to three R^{4b}, wherein R^{4b} is as described herein. In some embodiments, each C₁₋₃ alkyl and 5- to 7-membered heterocyclyl is optionally substituted with one or two R^{4b}, wherein R^{4b} is as described herein. In some embodiments, each C₁₋₃ alkyl and 5- to 7-membered heterocyclyl is optionally substituted with one R^{4b}, wherein R^{4b} is as described herein. In some embodiments, a compound of the disclosure is a compound, or pharmaceutically acceptable salt thereof, wherein each R^{4a} is independently —O(C₁₋₃ alkyl), 5- to 7-membered heterocyclyl, —C(O)NH₂, —C(O)N(H)(C₁₋₃ alkyl), or —C(O)N(C₁-C₃ alkyl)₂.

In some embodiments, a compound of the disclosure is a compound, or pharmaceutically acceptable salt thereof, wherein each R^{4a} is independently halogen or —C(O)(C₁₋₆ alkyl), wherein the alkyl is optionally substituted with one to three R^{4b}. In some embodiments, a compound of the disclosure is a compound, or pharmaceutically acceptable salt thereof, wherein each R^{4a} is independently halogen or —C(O)(C₁₋₃ alkyl). In some embodiments, a compound of the disclosure is a compound, or pharmaceutically acceptable salt thereof, wherein each R^{4a} is independently chloro, fluoro, or —C(O)(methyl).

In some embodiments, a compound of the disclosure is a compound, or pharmaceutically acceptable salt thereof, wherein each R^{4a} is independently —OH or —O(C₁₋₃ alkyl). In some embodiments, a compound of the disclosure is a compound, or pharmaceutically acceptable salt thereof, wherein each R^{4a} is independently —OH or methoxy.

In some embodiments, a compound of the disclosure is a compound, or pharmaceutically acceptable salt thereof, wherein each R^{4a} is —O(C₁₋₃ alkyl). In some embodiments, a compound of the disclosure is a compound, or pharmaceutically acceptable salt thereof, wherein R^{4a} is methoxy.

In some embodiments, a compound of the disclosure is a compound, or pharmaceutically acceptable salt thereof, wherein each R^{4a} is independently halogen or —OH. In some embodiments, a compound of the disclosure is a compound, or pharmaceutically acceptable salt thereof, wherein each R^{4a} is independently chloro, fluoro, or —OH.

In some embodiments, a compound of the disclosure is a compound, or pharmaceutically acceptable salt thereof, wherein each R^{4a} is independently C₁₋₆ alkyl, —C(O)(5- to 10-membered heterocyclyl), or —O(C₃₋₈ cycloalkyl), wherein each C₁₋₆ alkyl, 5- to 10-membered heterocyclyl, and C₃₋₈ cycloalkyl of R^{4a} is optionally substituted with one to three R^{4b}, wherein R^{4b} is as described herein. In some embodiments, each C₁₋₆ alkyl, 5- to 10-membered heterocyclyl, and C₃₋₈ cycloalkyl of R^{4a} is optionally substituted with one to two R^{4b}, wherein R^{4b} is as described herein. In some embodiments, each C₁₋₆ alkyl, 5- to 10-membered heterocyclyl, and C₃₋₈ cycloalkyl of R^{4a} is optionally substituted with one R^{4b}, wherein R^{4b} is as described herein. In some embodiments, a compound of the disclosure is a compound, or pharmaceutically acceptable salt thereof, wherein each R^{4a} is independently C₁₋₆ alkyl, —C(O)(5- to 10-membered heterocyclyl), or —O(C₃₋₈ cycloalkyl).

In some embodiments, a compound of the disclosure is a compound, or pharmaceutically acceptable salt thereof, wherein each R^{4b} is independently halogen, —O(C₁₋₆ alkyl), or —O(C₁₋₆ haloalkyl). In some embodiments, a compound of the disclosure is a compound, or pharmaceutically acceptable salt thereof, wherein each R^{4b} is independently fluoro, chloro —O(C₁₋₃ alkyl), or —O(C₁₋₃ haloalkyl).

In some embodiments, a compound of the disclosure is a compound, or pharmaceutically acceptable salt thereof, wherein ring \textcircled{B} is C₆₋₁₀ aryl or 5- to 10-membered heteroaryl. In some embodiments, a compound of the disclosure is a compound, or pharmaceutically acceptable salt thereof, wherein ring \textcircled{B} is C₆₋₁₀ aryl. In some embodiments, a compound of the disclosure is a compound, or pharmaceutically acceptable salt thereof, wherein ring \textcircled{B} is phenyl. In some embodiments, a compound of the disclosure is a compound, or pharmaceutically acceptable salt thereof, wherein ring \textcircled{B} is C₁₀ aryl. In some embodiments, a compound of the disclosure is a compound, or pharmaceutically acceptable salt thereof, wherein ring \textcircled{B} is indanyl.

In some embodiments, a compound of the disclosure is a compound, or pharmaceutically acceptable salt thereof, wherein \textcircled{B} ring is 5- to 10-membered heteroaryl. In some embodiments, a compound of the disclosure is a compound, or pharmaceutically acceptable salt thereof, wherein \textcircled{B} ring is 5- to 9-membered heteroaryl. In some embodiments, a compound of the disclosure is a compound, or pharmaceutically acceptable salt thereof, wherein \textcircled{B} ring is 6- to 10-membered heteroaryl. In some embodiments, a compound of the disclosure is a compound, or pharmaceutically acceptable salt thereof, wherein ring \textcircled{B} is 6- to 9-membered heteroaryl. In some embodiments, a compound of the disclosure is a compound, or pharmaceutically acceptable salt thereof, wherein ring \textcircled{B} is 5-, 6-, 7-, 8-, 9-, or 10-membered heteroaryl. In some embodiments, a compound of the disclosure is a compound, or pharmaceutically acceptable salt thereof, wherein ring \textcircled{B} is a nitrogen-containing heteroaryl. In some embodiments, a compound of the disclosure is a compound, or pharmaceutically acceptable salt thereof, wherein ring \textcircled{B} is a sulfur-containing heteroaryl. In some embodiments, a compound of the disclosure is a compound, or pharmaceutically acceptable salt thereof, wherein ring \textcircled{B} is an oxygen-containing heteroaryl. In some embodiments, a compound of the disclosure is a compound, or pharmaceutically acceptable salt thereof, wherein ring \textcircled{B} is a pyridine, a pyrimidine, an oxazole, a pyrrole, a pyrazole, an imidazole, a triazole, or a thiophene, each of which is optionally fused to another ring forming ring \textcircled{B}. In some embodiments, the pyrimidine of \textcircled{B} ring is a pyrimidine dione.

In some embodiments, a compound of the disclosure is a compound, or pharmaceutically acceptable salt thereof, wherein at least one L¹ is independently a —O—, —(C₁₋₆ alkyl)O—, —O(C₁₋₆ alkyl)-, —C₁₋₆ alkyl-O(C₁₋₆ alkyl)-, —C(O)—, —N(R^{L})C(O)—, —C(O)N(R^{L})—, —(C₁₋₆ alkyl)(R^{L})NC(O)—, —(C₁₋₆ alkyl)C(O)N(R^{L})—, —N(R^{L})C(O)(C₁₋₆ alkyl)-, —C(O)N(R^{L})(C₁₋₆ alkyl)-, —(C₁₋₆ alkyl)N(R^{L})C(O)(C₁₋₆ alkyl)-, —(C₁₋₆ alkyl)C(O)N(R^{L})(C₁₋₆ alkyl)-, —S(O)₂—, —S(O)₂N(R^{L})—, —N(R^{L})—S(O)₂—, —(C₁₋₆ alkyl)S(O)₂N(R^{L})—, —(C₁₋₆ alkyl)N(R^{L})S(O)₂—, —S(O)₂N(R^{L})(C₁₋₆ alkyl)-, —N(R^{L})S(O)₂(C₁₋₆ alkyl)-, —(C₁₋₆ alkyl)S(O)₂N(R^{L})(C₁₋₆ alkyl)-, or —(C₁₋₆ alkyl)N(R^{L})S(O)₂(C₁₋₆ alkyl)-, wherein R^{L} is as described herein. In some embodiments, a compound of the disclosure is a compound, or pharmaceutically acceptable salt thereof, wherein at least one L¹ is independently a —O—, —(C₁₋₃ alkyl)O—, —O(C₁₋₃ alkyl)-, —C₁₋₃ alkyl-O(C₁₋₃ alkyl)-, —C(O)—, —N(R^{L})C(O)—, —C(O)N(R^{L})—, —(C₁₋₃ alkyl)(R^{L})NC(O)—, —(C₁₋₃ alkyl)C(O)N(R^{L})—, —N(R^{L})C(O)(C₁₋₃ alkyl)-, —C(O)N(R^{L})(C₁₋₃ alkyl)-, —(C₁₋₃ alkyl)N(R^{L})C(O)(C₁₋₃ alkyl)-, —(C₁₋₃ alkyl)C(O)N(R^{L})(C₁₋₃ alkyl)-, —S(O)₂—, —S(O)₂N(R^{L})—, —N(R^{L})—S(O)₂—, —(C₁₋₃ alkyl)S(O)₂N(R^{L})—, —(C₁₋₃ alkyl)N(R^{L})S(O)₂—, —S(O)₂N(R^{L})(C₁₋₃ alkyl)-, —N(R^{L})S(O)₂(C₁₋₃ alkyl)-, —(C₁₋₃ alkyl)S(O)₂N(R^{L})(C₁₋₃ alkyl)-, or —(C₁₋₃ alkyl)N(R^{L})S(O)₂(C₁₋₆ alkyl)-, wherein R^{L} is as described herein.

In some embodiments, a compound of the disclosure is a compound, or pharmaceutically acceptable salt thereof, wherein each L¹ is independently a —O—, —(C₁₋₆ alkyl)O—, —O(C₁₋₆ alkyl)-, —C₁₋₆ alkyl-O(C₁₋₆ alkyl)-, —C(O)—, —N(R^{L})C(O)—, —C(O)N(R^{L})—, —(C₁₋₆ alkyl)(R^{L})NC(O)—, —(C₁₋₆ alkyl)C(O)N(R^{L})—, —N(R^{L})C(O)(C₁₋₆ alkyl)-, —C(O)N(R^{L})(C₁₋₆ alkyl)-, —(C₁₋₆ alkyl)N(R^{L})C(O)(C₁₋₆ alkyl)-, —(C₁₋₆ alkyl)C(O)N(R^{L})(C₁₋₆ alkyl)-, —S(O)₂—, —S(O)₂N(R^{L})—, —N(R^{L})—S(O)₂—, —(C₁₋₆ alkyl)S(O)₂N(R^{L})—, —(C₁₋₆ alkyl)N(R^{L})S(O)₂—, —S(O)₂N(R^{L})(C₁₋₆ alkyl)-, —N(R^{L})S(O)₂(C₁₋₆ alkyl)-, —(C₁₋₆ alkyl)S(O)₂N(R^{L})(C₁₋₆ alkyl)-, or —(C₁₋₆ alkyl)N(R^{L})S(O)₂(C₁₋₆ alkyl)-, wherein R^{L} is as described herein. In some embodiments, a compound of the disclosure is a compound, or pharmaceutically acceptable salt thereof, wherein each L¹ is independently a —O—, —(C₁₋₃ alkyl)O—, —O(C₁₋₃ alkyl)-, —C₁₋₃ alkyl-O(C₁₋₃ alkyl)-, —C(O)—, —N(R^{L})C(O)—, —C(O)N(R^{L})—, —(C₁₋₃ alkyl)(R^{L})NC(O)—, —(C₁₋₃ alkyl)C(O)N(R^{L})—, —N(R^{L})C(O)(C₁₋₃ alkyl)-, —C(O)N(R^{L})(C₁₋₃ alkyl)-, —(C₁₋₃ alkyl)N(R^{L})C(O)(C₁₋₃ alkyl)-, —(C₁₋₃ alkyl)C(O)N(R^{L})(C₁₋₃ alkyl)-, —S(O)₂—, —S(O)₂N(R^{L})—, —N(R^{L})—S(O)₂—, —(C₁₋₃ alkyl)S(O)₂N(R^{L})—, —(C₁₋₃ alkyl)N(R^{L})S(O)₂—, —S(O)₂N(R^{L})(C₁₋₃ alkyl)-, —N(R^{L})S(O)₂(C₁₋₃ alkyl)-, —(C₁₋₃ alkyl)S(O)₂N(R^{L})(C₁₋₃ alkyl)-, or —(C₁₋₃ alkyl)N(R^{L})S(O)₂(C₁₋₆ alkyl)-, wherein R^{L} is as described herein.

In some embodiments, a compound of the disclosure is a compound, or pharmaceutically acceptable salt thereof, wherein each L¹ is independently a bond, —(C₁₋₆ alkyl)O—, —O(C₁₋₆ alkyl)-, —C₁₋₆ alkyl-O(C₁₋₆ alkyl)-, —C(O)—, —N(R^{L})C(O)—, —C(O)N(R^{L})—, —(C₁₋₆ alkyl)(R^{L})NC(O)—, —(C₁₋₆ alkyl)C(O)N(R^{L})—, —N(R^{L})C(O)(C₁₋₆ alkyl)-, —C(O)N(R^{L})(C₁₋₆ alkyl)-, —(C₁₋₆ alkyl)N(R^{L})C(O)(C₁₋₆ alkyl)-, or —(C₁₋₆ alkyl)C(O)N(R^{L})(C₁₋₆ alkyl)-, wherein R^{L} is as described herein. In some embodiments, a compound of the disclosure is a compound, or pharmaceutically acceptable salt thereof, wherein each L¹ is independently a bond, —(C₁₋₃ alkyl)O—, —O(C₁₋₃ alkyl)-, —C₁₋₃ alkyl-O(C₁₋₃ alkyl)-, —C(O)—, —N(R^{L})C(O)—, —C(O)N(R^{L})—, —(C₁₋₃ alkyl)(R^{L})NC(O)—, —(C₁₋₃ alkyl)C(O)N(R^{L})—, —N(R^{L})C(O)(C₁₋₃ alkyl)-, —C(O)N(R^{L})(C₁₋₃ alkyl)-, —(C₁₋₃ alkyl)N(R^{L})C(O)(C₁₋₃ alkyl)-, or —(C₁₋₃ alkyl)C(O)N(R^{L})(C₁₋₃ alkyl)-, wherein R^{L} is as described herein.

In some embodiments, a compound of the disclosure is a compound, or pharmaceutically acceptable salt thereof, wherein each L¹ is independently —(C₁₋₆ alkyl)O—, —O(C₁₋₆ alkyl)-, or —C₁₋₆ alkyl-O(C₁₋₆ alkyl)-. In some embodiments, a compound of the disclosure is a compound, or pharmaceutically acceptable salt thereof, wherein each L¹ is independently —(C₁₋₃ alkyl)O—, —O(C₁₋₃ alkyl)-, or —C₁₋₃ alkyl-O(C₁₋₃ alkyl)-. In some embodiments, a compound of the disclosure is a compound, or pharmaceutically acceptable salt thereof, wherein each L¹ is independently -(methyl)O—, —O(methyl)-, or -methyl-O(methyl)-.

In some embodiments, a compound of the disclosure is a compound, or pharmaceutically acceptable salt thereof, wherein each L¹ is independently —(C₁₋₆ alkyl)O— or —O(C₁₋₆ alkyl)-. In some embodiments, a compound of the disclosure is a compound, or pharmaceutically acceptable salt thereof, wherein each L¹ is independently —(C₁₋₃ alkyl)O— or —O(C₁₋₃ alkyl)-. In some embodiments, a compound of the disclosure is a compound, or pharmaceutically acceptable salt thereof, wherein each L¹ is independently -(methyl)O— or —O(methyl)-.

In some embodiments, a compound of the disclosure is a compound, or pharmaceutically acceptable salt thereof, wherein at least one L¹ is a bond. In some embodiments, a compound of the disclosure is a compound, or pharmaceutically acceptable salt thereof, wherein each L¹ is a bond.

In some embodiments, a compound of the disclosure is a compound, or pharmaceutically acceptable salt thereof, wherein each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl, wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to three R^{1a}, wherein R^{1a} is as described herein. In some embodiments, each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to two R^{1a}, wherein R^{1a} is as described herein. In some embodiments, each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one R^{1a}, wherein R^{1a} is as described herein. In some embodiments, a compound of the disclosure is a compound, or pharmaceutically acceptable salt thereof, wherein each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl.

In some embodiments, a compound of the disclosure is a compound, or pharmaceutically acceptable salt thereof, wherein each R¹ is independently halogen, —CN, —C(O)NH₂, C₁₋₆ alkyl, C₂₋₆ alkynyl, C₁₋₆ alkoxy, C₃₋₈ cycloalkyl, phenyl, or 5- to 10-membered heteroaryl, wherein each C₁₋₆ alkyl, C₂₋₆ alkynyl, C₁₋₆ alkoxy, C₃₋₈ cycloalkyl, phenyl, and 5- to 10-membered heteroaryl of R¹ is optionally substituted with one to three R^{1a}, wherein R^{1a} is as described herein. In some embodiments, each C₁₋₆ alkyl, C₂₋₆ alkynyl, C₁₋₆ alkoxy, C₃₋₈ cycloalkyl, phenyl, and 5- to 10-membered heteroaryl of R¹ is optionally substituted with one to two R^{1a}, wherein R^{1a} is as described herein. In some embodiments, each C₁₋₆ alkyl, C₂₋₆ alkynyl, C₁₋₆ alkoxy, C₃₋₈ cycloalkyl, phenyl, and 5- to 10-membered heteroaryl of R¹ is optionally substituted with one R^{1a}, wherein R^{1a} is as described herein. In some embodiments, a compound of the disclosure is a compound, or pharmaceutically acceptable salt thereof, wherein each R¹ is independently halogen, —CN, —C(O)NH₂, C₁₋₆ alkyl, C₂₋₆ alkynyl, C₁₋₆ alkoxy, C₃₋₈ cycloalkyl, phenyl, or 5- to 10-membered heteroaryl.

In some embodiments, a compound of the disclosure is a compound, or pharmaceutically acceptable salt thereof, wherein each R¹ is independently halogen, —CN, —C(O)NH₂, C₁₋₆ alkyl, C₂₋₆ alkynyl, C₁₋₆ alkoxy, C₃₋₈ cycloalkyl, or 5- to 10-membered heteroaryl, wherein each C₁₋₆ alkyl, C₂₋₆ alkynyl, C₁₋₆ alkoxy, C₃₋₈ cycloalkyl, and 5- to 10-membered heteroaryl of R¹ is optionally substituted with one or two R^{1a}, wherein R^{1a} is as described herein. In some embodiments, each C₁₋₆ alkyl, C₂₋₆ alkynyl, C₁₋₆ alkoxy, C₃₋₈ cycloalkyl, and 5- to 10-membered heteroaryl of R¹ is optionally substituted with one R^{1a}, wherein R^{1a} is as described herein. In some embodiments, a compound of the disclosure is a compound, or pharmaceutically acceptable salt thereof, wherein each R¹ is independently halogen, —CN, —C(O)NH₂, C₁₋₆ alkyl, C₂₋₆ alkynyl, C₁₋₆ alkoxy, C₃₋₈ cycloalkyl, or 5- to 10-membered heteroaryl.

In some embodiments, a compound of the disclosure is a compound, or pharmaceutically acceptable salt thereof, wherein each R¹ is independently halogen, —CN, or C₁₋₆ alkoxy. In some embodiments, a compound of the disclosure is a compound, or pharmaceutically acceptable salt thereof, wherein each R¹ is independently halogen, —CN, or C₁₋₃ alkoxy. In some embodiments, a compound of the disclosure is a compound, or pharmaceutically acceptable salt thereof, wherein each R¹ is independently chloro, fluoro, —CN, or methoxy. In some embodiments, a compound of the disclosure is a compound, or pharmaceutically acceptable salt thereof, wherein each R¹ is independently fluoro, —CN, or methoxy.

In some embodiments, a compound of the disclosure is a compound, or pharmaceutically acceptable salt thereof, wherein each R¹ is independently halogen or C₁₋₃ alkoxy. In some embodiments, a compound of the disclosure is a compound, or pharmaceutically acceptable salt thereof, wherein each R¹ is independently chloro, fluoro, or methoxy. In some embodiments, a compound of the disclosure is a compound, or pharmaceutically acceptable salt thereof, wherein each R¹ is independently fluoro or methoxy.

In some embodiments, a compound of the disclosure is a compound, or pharmaceutically acceptable salt thereof, wherein each R^{1a} is independently C₁₋₆ alkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —N(5- to 10-membered heterocyclyl)₂, —N(phenyl)₂, —N(5- to 10-membered heteroaryl)₂, —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)O(C₁₋₆ alkyl), —C(O)O(C₃₋₈ cycloalkyl), —C(O)O(5- to 10-membered heterocyclyl), —C(O)O(phenyl), —C(O)O(5- to 10-membered heteroaryl), —C(O)NH₂, —C(O)NH(C₁₋₆ alkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₃₋₈ cycloalkyl)₂, —C(O)N(5- to 10-membered heterocyclyl)₂, —C(O)N(phenyl)₂, —C(O)N(5- to 10-membered heteroaryl)₂, —NHC(O)(C₁₋₆ alkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —NHS(O)(C₁₋₆ alkyl), —N(C₁₋₆ alkyl)(S(O)(C₁₋₆ alkyl), —S(O)₂(C₁₋₆ alkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂ NH(C₁₋₆ alkyl), or —S(O)₂N(C₁₋₆ alkyl)₂, wherein each C₁₋₆ alkyl, C₃₋₈ cycloalkyl, 4- to 10-membered heterocyclyl, phenyl, and 5- to 10-membered heteroaryl of R^{1a} is optionally substituted with one to two R^{1b}, wherein R^{1b} is as described herein. In some embodiments, each C₁₋₆ alkyl, C₃₋₈ cycloalkyl, 4- to 10-membered heterocyclyl, phenyl, and 5- to 10-membered heteroaryl of R^{1a} is optionally substituted with one R^{1b}, wherein R^{1b} is as described herein.

In some embodiments, a compound of the disclosure is a compound, or pharmaceutically acceptable salt thereof, wherein each R^{1a} is independently C₁₋₆ alkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —N(5- to 10-membered heterocyclyl)₂, —N(phenyl)₂, —N(5- to 10-membered heteroaryl)₂, —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)O(C₁₋₆ alkyl), —C(O)O(C₃₋₈ cycloalkyl), —C(O)O(5- to 10-membered heterocyclyl), —C(O)O(phenyl), —C(O)O(5- to 10-membered heteroaryl), —C(O)NH₂, —C(O)NH(C₁₋₆ alkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₃₋₈ cycloalkyl)₂, —C(O)N(5- to 10-membered heterocyclyl)₂, —C(O)N(phenyl)₂, —C(O)N(5- to 10-membered heteroaryl)₂, —NHC(O)(C₁₋₆ alkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —NHS(O)(C₁₋₆ alkyl), —N(C₁₋₆ alkyl)(S(O)(C₁₋₆ alkyl), —S(O)₂(C₁₋₆ alkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂.

In some embodiments, a compound of the disclosure is a compound, or pharmaceutically acceptable salt thereof, wherein each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl, wherein each C₁₋₆ alkyl, C₃₋₈ cycloalkyl, phenyl, and 5- to 10-membered heteroaryl of R^{1a} is optionally substituted with one to two R^{1b}, wherein R^{1b} is as described herein. In some embodiments, a compound of the disclosure is a compound, or pharmaceutically acceptable salt thereof, wherein each R^{1a} is independently fluoro, chloro, —OH, —CN, —C(O)NH₂, C₁₋₃ alkyl, C₁₋₃ alkoxy, C₃₋₈ cycloalkyl, phenyl, or 5- to 10-membered heteroaryl, wherein each C₁₋₃ alkyl, C₃₋₈ cycloalkyl, phenyl, and 5- to 10-membered heteroaryl of R^{1a} is optionally substituted with one to two R^{1b}, wherein R^{1b} is as described herein. In some embodiments, each C₁₋₆ alkyl, C₃₋₈ cycloalkyl, phenyl, and 5- to 10-membered heteroaryl of R^{1a} is optionally substituted with one R^{1b}, wherein R^{1b} is as described herein.

In some embodiments, a compound of the disclosure is a compound, or pharmaceutically acceptable salt thereof, wherein each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl. In some embodiments, a compound of the disclosure is a compound, or pharmaceutically acceptable salt thereof, wherein each R^{1a} is independently fluoro, chloro, —OH, —CN, —C(O)NH₂, C₁₋₃ alkyl, C₁₋₃ alkoxy, C₃₋₈ cycloalkyl, phenyl, or 5- to 10-membered heteroaryl.

In some embodiments, a compound of the disclosure is a compound, or pharmaceutically acceptable salt thereof, wherein each R^{1a} is independently halogen, —OH, —C(O)NH₂, C₁₋₆ alkyl, C₁₋₆ alkoxy, or phenyl, wherein each C₁₋₆ alkyl and phenyl of R^{1a} is optionally substituted with one to two R^{1b}, wherein R^{1b} is as described herein. In some embodiments, a compound of the disclosure is a compound, or pharmaceutically acceptable salt thereof, wherein each R^{1a} is independently chloro, fluoro, —OH, —C(O)NH₂, C₁₋₃ alkyl, C₁₋₃ alkoxy, or phenyl, wherein each C₁₋₃ alkyl and phenyl of R^{1a} is optionally substituted with one to two R^{1b}, wherein R^{1b} is as described herein. In some embodiments, each C₁₋₆ alkyl and phenyl of R^{1a} is optionally substituted with one R^{1b}, wherein R^{1b} is as described herein.

In some embodiments, a compound of the disclosure is a compound, or pharmaceutically acceptable salt thereof, wherein each R^{1a} is independently halogen, —OH, —C(O)NH₂, C₁₋₆ alkyl, C₁₋₆ alkoxy, or phenyl. In some embodiments, a compound of the disclosure is a compound, or pharmaceutically acceptable salt thereof, wherein each R^{1a} is independently chloro, fluoro, —OH, —C(O)NH₂, C₁₋₃ alkyl, C₁₋₃ alkoxy, or phenyl.

In some embodiments, a compound of the disclosure is a compound, or pharmaceutically acceptable salt thereof, wherein at least one R^{1a} is halogen. In some embodiments, a compound of the disclosure is a compound, or pharmaceutically acceptable salt thereof, wherein at least one R^{1a} is chloro or fluoro. In some embodiments, a compound of the disclosure is a compound, or pharmaceutically acceptable salt thereof, wherein at least one R^{1a} is chloro. In some embodiments, a compound of the disclosure is a compound, or pharmaceutically acceptable salt thereof, wherein at least one R^{1a} is fluoro.

In some embodiments, a compound of the disclosure is a compound, or pharmaceutically acceptable salt thereof, wherein R¹ is phenyl, and two R^{1a} taken together with the atoms of the phenyl to which they are attached form 5- to 10-membered heterocyclyl optionally substituted with one to three R^{1b}, wherein R^{1b} is as described herein. In some embodiments, a compound of the disclosure is a compound, or pharmaceutically acceptable salt thereof, wherein R¹ is phenyl, and two R^{1a} taken together with the atoms of the phenyl to which they are attached form 5- to 7-membered heterocyclyl optionally substituted with one to three R^{1b}, wherein R^{1b} is as described herein. In some embodiments, a compound of the disclosure is a compound, or pharmaceutically acceptable salt thereof, wherein R¹ is phenyl, and two R^{1a} taken together with the atoms of the phenyl to which they are attached form 5- to 7-membered heterocyclyl optionally substituted with one or two R^{1b}, wherein R^{1b} is as described herein. In some embodiments, a compound of the disclosure is a compound, or pharmaceutically acceptable salt thereof, wherein R¹ is phenyl, and two R^{1a} taken together with the atoms of the phenyl to which they are attached form a 5-membered heterocyclyl optionally substituted with one or two R^{1b}, wherein R^{1b} is as described herein.

In some embodiments, a compound of the disclosure is a compound, or pharmaceutically acceptable salt thereof, wherein each R^{1b} is independently C₁₋₃ alkyl, C₁₋₃ haloalkyl, halogen, oxo, —OH, —NH₂, CO₂H, —O(C₁₋₃ alkyl), —O(C₁₋₃ haloalkyl), —O(C₃₋₈ cycloalkyl), —O(5- to 10-membered heterocyclyl), —O(phenyl), —O(5- to 10-membered heteroaryl), —NH(C₁₋₃ alkyl), —NH(C₁₋₃ haloalkyl), —NH(C₃₋₈ cycloalkyl), —NH(5- to 10-membered heterocyclyl), —NH(phenyl), —NH(5- to 10-membered heteroaryl), —N(C₁₋₃ alkyl)₂, —N(C₃₋₈ cycloalkyl)₂, —NHC(O)(C₁₋₃ alkyl), —NHC(O)(C₁₋₃ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₃ alkyl), —NHC(O)O(C₁₋₃ haloalkyl), —NHC(O)O(C₂₋₆ alkynyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₃ alkyl), S(O)₂(C₁₋₃ alkyl), —S(O)₂(C₁₋₃ haloalkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₃ alkyl), —S(O)₂NH(C₁₋₃ alkyl), or —S(O)₂N(C₁₋₃ alkyl)₂.

In some embodiments, a compound of the disclosure is a compound, or pharmaceutically acceptable salt thereof, wherein each R^{1b} is independently C₁₋₆ alkyl, C₁₋₆ haloalkyl, halogen, oxo, —OH, or —NH₂. In some embodiments, a compound of the disclosure is a compound, or pharmaceutically acceptable salt thereof, wherein each R^{1b} is independently C₁₋₃ alkyl, C₁₋₃ haloalkyl, halogen, oxo, —OH, or —NH₂. In some embodiments, a compound of the disclosure is a compound, or pharmaceutically acceptable salt thereof, wherein each R^{1b} is independently halogen. In some embodiments, a compound of the disclosure is a compound, or pharmaceutically acceptable salt thereof, wherein each R^{1b} is independently chloro or fluoro.

In some embodiments, a compound of the disclosure is a compound, or pharmaceutically acceptable salt thereof, wherein m is an integer from 0 to 2. In some embodiments, a compound of the disclosure is a compound, or pharmaceutically acceptable salt thereof, wherein m is an integer from 1 to 3. In some embodiments, a compound of the disclosure is a compound, or pharmaceutically acceptable salt thereof, wherein m is 0 or 1. In some embodiments, a compound of the disclosure is a compound, or pharmaceutically acceptable salt thereof, wherein m is 1 or 2. In some embodiments, a compound of the disclosure is a compound, or pharmaceutically acceptable salt thereof, wherein m is 2 or 3. In some embodiments, a compound of the disclosure is a compound, or pharmaceutically acceptable salt thereof, wherein m is 0. In some embodiments, a compound of the disclosure is a compound, or pharmaceutically acceptable salt thereof, wherein m is 1. In some embodiments, a compound of the disclosure is a compound, or pharmaceutically acceptable salt thereof, wherein m is 2. In some embodiments, a compound of the disclosure is a compound, or pharmaceutically acceptable salt thereof, wherein m is 3.

In some embodiments, a compound of the disclosure is a compound, or pharmaceutically acceptable salt thereof, wherein ring \textcircled{B} is phenyl and the substituent meta to the point of attachment is hydrogen. In some embodiments, a compound of the disclosure is a compound, or pharmaceutically acceptable salt thereof, wherein ring \textcircled{B} is

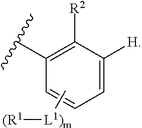

In some embodiments, a compound of the disclosure is a compound, or pharmaceutically acceptable salt thereof, wherein ring \textcircled{B} is

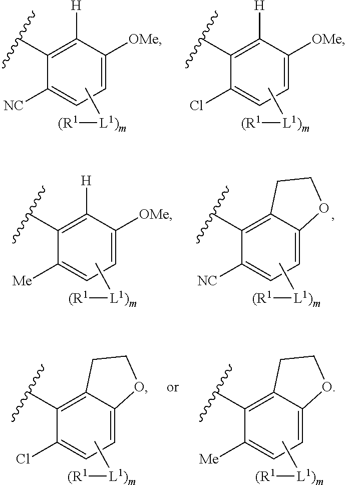

In some embodiments, a compound of the disclosure is a compound, or pharmaceutically acceptable salt thereof, wherein ring \textcircled{B} is

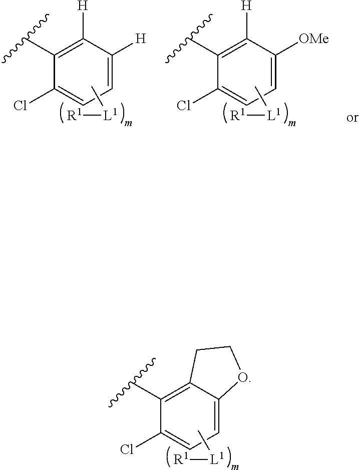

In some embodiments, a compound of the disclosure is a compound, or pharmaceutically acceptable salt thereof, wherein ring \textcircled{B} is

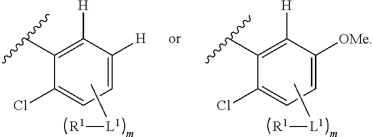

In some embodiments, a compound of the disclosure is a compound, or pharmaceutically acceptable salt thereof, wherein ring \textcircled{B} is

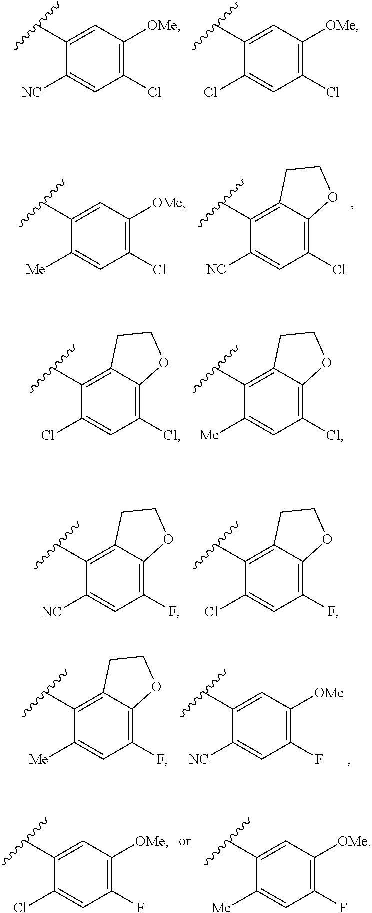

In some embodiments, a compound of the disclosure is a compound, or pharmaceutically acceptable salt thereof, wherein ring \textcircled{B} is

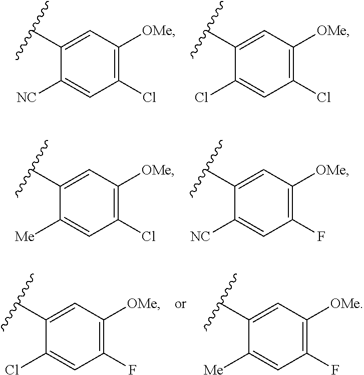 [SMILES: *c1cc(OC)c(Cl)cc1C#N ; *c1cc(OC)c(Cl)cc1Cl ; *c1cc(OC)c(Cl)cc1C ; *c1cc(OC)c(F)cc1C#N ; *c1cc(OC)c(F)cc1Cl ; *c1cc(OC)c(F)cc1C]

In some embodiments, a compound of the disclosure is a compound, or pharmaceutically acceptable salt thereof, wherein ring \textcircled{B} is

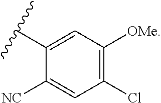 [SMILES: *c1cc(OC)c(Cl)cc1C#N]

In some embodiments, a compound of the disclosure is a compound, or pharmaceutically acceptable salt thereof, wherein ring \textcircled{B} is

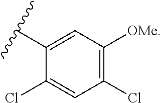 [SMILES: *c1cc(OC)c(Cl)cc1Cl]

In some embodiments, a compound of the disclosure is a compound, or pharmaceutically acceptable salt thereof, wherein \textcircled{B} ring is

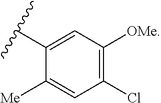 [SMILES: *c1cc(OC)c(Cl)cc1C]

In some embodiments, a compound of the disclosure is a compound, or pharmaceutically acceptable salt thereof, wherein ring \textcircled{B} is

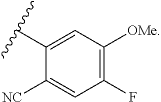 [SMILES: *c1cc(OC)c(F)cc1C#N]

In some embodiments, a compound of the disclosure is a compound, or pharmaceutically acceptable salt thereof, wherein \textcircled{B} ring is

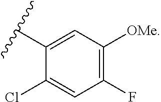 [SMILES: *c1cc(OC)c(F)cc1Cl]

In some embodiments, a compound of the disclosure is a compound, or pharmaceutically acceptable salt thereof, wherein \textcircled{B} ring is

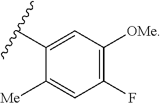 [SMILES: *c1cc(OC)c(F)cc1C]

In some embodiments, a compound of the disclosure is a compound, or pharmaceutically acceptable salt thereof, wherein \textcircled{B} ring is

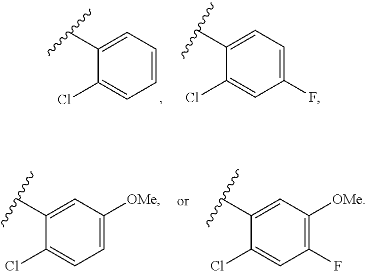

In some embodiments, a compound of the disclosure is a compound, or pharmaceutically acceptable salt thereof, wherein \textcircled{B} ring is

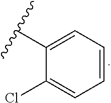 [SMILES: *c1ccccc1Cl]

In some embodiments, a compound of the disclosure is a compound, or pharmaceutically acceptable salt thereof, wherein \textcircled{B} ring is

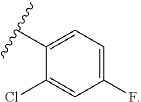 [SMILES: *c1ccc(F)cc1Cl]

In some embodiments, a compound of the disclosure is a compound, or pharmaceutically acceptable salt thereof, wherein \textcircled{B} ring is

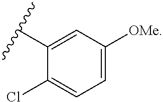 [SMILES: *c1cc(OC)ccc1Cl]

In some embodiments, a compound of the disclosure is a compound, or pharmaceutically acceptable salt thereof, wherein ring \textcircled{B} is

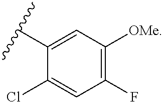 [SMILES: *c1cc(OC)c(F)cc1Cl]

In some embodiments, a compound of the disclosure is a compound, or pharmaceutically acceptable salt thereof, wherein R² is hydrogen, halogen, —CN, C₁₋₃ alkyl, or C₁₋₃ alkoxy. In some embodiments, a compound of the disclosure is a compound, or pharmaceutically acceptable salt thereof, wherein R² is hydrogen, fluoro, chloro, —CN, methyl, or methoxy.

In some embodiments, a compound of the disclosure is a compound, or pharmaceutically acceptable salt thereof, wherein R² is hydrogen. In some embodiments, a compound of the disclosure is a compound, or pharmaceutically acceptable salt thereof, wherein R² is halogen. In some embodiments, a compound of the disclosure is a compound, or pharmaceutically acceptable salt thereof, wherein R² is chloro or fluoro. In some embodiments, a compound of the disclosure is a compound, or pharmaceutically acceptable salt thereof, wherein R² is chloro. In some embodiments, a compound of the disclosure is a compound, or pharmaceutically acceptable salt thereof, wherein R² is fluoro.

In some embodiments, a compound of the disclosure is a compound, or pharmaceutically acceptable salt thereof, wherein L² is a bond, C₁₋₃ alkyl, —(C₀₋₃ alkyl)-cyclopropyl-(C₀₋₃ alkyl)-, —(C₁₋₃ alkyl)O—, —(C₁₋₃ alkyl)O(C₁₋₃ alkyl)-, —(C₁₋₃ alkyl)(R^{L2})NC(O)—, —(C₁₋₃ alkyl)C(O)N(R^{L2})—, —(C₁₋₃ alkyl)S(O)₂N(R^{L2})—, —(C₁₋₃ alkyl)N(R^{L2})S(O)₂—, —(C₁₋₃ alkyl)S(O)₂N(R^{L2})(C₁₋₃ alkyl)-, or —(C₁₋₃ alkyl)S(O)₂—(C₁₋₃ alkyl)-, wherein R^{L2} is as described herein.

In some embodiments, a compound of the disclosure is a compound, or pharmaceutically acceptable salt thereof, wherein L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-, wherein R^{L2} is as described herein. In some embodiments, a compound of the disclosure is a compound, or pharmaceutically acceptable salt thereof, wherein L² is a bond, C₁₋₃ alkyl, —(C₀₋₃ alkyl)-cyclopropyl-(C₀₋₃ alkyl)-, —(C₁₋₃ alkyl)O—, or —(C₁₋₃ alkyl)O(C₁₋₃ alkyl)-, wherein R^{L2} is as described herein.

In some embodiments, a compound of the disclosure is a compound, or pharmaceutically acceptable salt thereof, wherein L² is a bond.

In some embodiments, a compound of the disclosure is a compound, or pharmaceutically acceptable salt thereof, wherein L² is C₁₋₃ alkyl. In some embodiments, a compound of the disclosure is a compound, or pharmaceutically acceptable salt thereof, wherein L² is methyl. In some embodiments, a compound of the disclosure is a compound, or pharmaceutically acceptable salt thereof, wherein L² is ethyl. In some embodiments, a compound of the disclosure is a compound, or pharmaceutically acceptable salt thereof, wherein L² is propyl.

In some embodiments, a compound of the disclosure is a compound, or pharmaceutically acceptable salt thereof, wherein R^{L2} is hydrogen or C₁₋₃ alkyl. In some embodiments, a compound of the disclosure is a compound, or pharmaceutically acceptable salt thereof, wherein R^{L2} is hydrogen. In some embodiments, a compound of the disclosure is a compound, or pharmaceutically acceptable salt thereof, wherein R^{L2} is methyl.

In some embodiments, a compound of the disclosure is a compound, or pharmaceutically acceptable salt thereof, wherein R⁵ is hydrogen, —CN, —OR^{5a}, —C(O)NR^{5a}₂, —NR^{5a}C(O)R^{5a}, —NR^{5a}₂, C₁₋₆ alkyl, C₃₋₈ cycloalkyl, phenyl, 4- to 10-membered heterocyclyl, or 5- to 10-membered heteroaryl, wherein each C₁₋₆ alkyl, C₃₋₈ cycloalkyl, phenyl, 4- to 10-membered heterocyclyl, and 5- to 10-membered heteroaryl of R⁵ is optionally substituted with one R^{5b}, wherein R^{5b} is as described herein. In some embodiments, a compound of the disclosure is a compound, or pharmaceutically acceptable salt thereof, wherein R⁵ is hydrogen, —CN, —OR^{5a}, —C(O)NR^{5a}₂, —NR^{5a}C(O)R^{5a}, —NR^{5a}₂ C₁₋₆ alkyl, C₃₋₈ cycloalkyl, phenyl, 4- to 10-membered heterocyclyl, or 5- to 10-membered heteroaryl.

In some embodiments, a compound of the disclosure is a compound, or pharmaceutically acceptable salt thereof, wherein R⁵ is hydrogen, —CN, C₁₋₆ alkyl, C₁₋₆ haloalkyl, C₃₋₈ cycloalkyl, phenyl, 4- to 10-membered heterocyclyl, or 5- to 10-membered heteroaryl, wherein each C₃₋₈ cycloalkyl, phenyl, 4- to 10-membered heterocyclyl, and 5- to 10-membered heteroaryl of R⁵ is optionally substituted with one or two R^{5b}, wherein R^{5b} is as described herein. In some embodiments, each C₃₋₈ cycloalkyl, phenyl, 4- to 10-membered heterocyclyl, and 5- to 10-membered heteroaryl of R⁵ is optionally substituted with one R^{5b}, wherein R^{5b} is as described herein. In some embodiments, a compound of the disclosure is a compound, or pharmaceutically acceptable salt thereof, wherein R⁵ is hydrogen, —CN, C₁₋₆ alkyl, C₁₋₆ haloalkyl, C₃₋₈ cycloalkyl, phenyl, 4- to 10-membered heterocyclyl, or 5- to 10-membered heteroaryl.

In some embodiments, a compound of the disclosure is a compound, or pharmaceutically acceptable salt thereof, wherein R⁵ is C₁₋₃ alkyl, C₅₋₆ cycloalkyl, phenyl, 5- to 7-membered heterocyclyl, or 5- to 9-membered heteroaryl, wherein each C₅₋₆ cycloalkyl, phenyl, 5- to 7-membered heterocyclyl, and 5- to 9-membered heteroaryl of R⁵ is optionally substituted with one or two R^{5b}, wherein R^{5b} is as described herein. In some embodiments, each C₅₋₆ cycloalkyl, phenyl, 5- to 7-membered heterocyclyl, and 5- to 9-membered heteroaryl of R⁵ is optionally substituted with one R^{5b}, wherein R^{5b} is as described herein. In some embodiments, a compound of the disclosure is a compound, or pharmaceutically acceptable salt thereof, wherein R⁵ is C₁₋₃ alkyl, C₅₋₆ cycloalkyl, phenyl, 5- to 7-membered heterocyclyl, or 5- to 9-membered heteroaryl.

In some embodiments, a compound of the disclosure is a compound, or pharmaceutically acceptable salt thereof, wherein R⁵ is hydrogen. In some embodiments, a compound of the disclosure is a compound, or pharmaceutically acceptable salt thereof, wherein R⁵ is —CN.

In some embodiments, a compound of the disclosure is a compound, or pharmaceutically acceptable salt thereof, wherein each R^{5a} is independently hydrogen or C₁₋₃ alkyl. In some embodiments, a compound of the disclosure is a compound, or pharmaceutically acceptable salt thereof, wherein each R^{5a} is independently hydrogen. In some embodiments, a compound of the disclosure is a compound, or pharmaceutically acceptable salt thereof, wherein each R^{5a} is independently C₁₋₃ alkyl. In some embodiments, a compound of the disclosure is a compound, or pharmaceutically acceptable salt thereof, wherein each R^{5a} is independently methyl.

In some embodiments, a compound of the disclosure is a compound, or pharmaceutically acceptable salt thereof, wherein each R^{5b} is independently floruo, chloro, oxo, cyclopropyl, hydroxy, —CN, methyl, methoxy, —OCF₃, or —OCF₂H.

In some embodiments, a compound of the disclosure is a compound, or pharmaceutically acceptable salt thereof, wherein each R^{5b} is independently oxo, C₁₋₃ alkyl, or C₁₋₃ alkoxy. In some embodiments, a compound of the disclosure is a compound, or pharmaceutically acceptable salt thereof, wherein each R^{5b} is independently oxo, methyl, or methoxy. In some embodiments, a compound of the disclosure is a compound, or pharmaceutically acceptable salt thereof, wherein each R^{5b} is methyl. In some embodiments, a compound of the disclosure is a compound, or pharmaceutically acceptable salt thereof, wherein each R^{5b} is oxo.

In some embodiments, a compound of the disclosure is a compound, or pharmaceutically acceptable salt thereof, having a structure as shown in Scheme A:

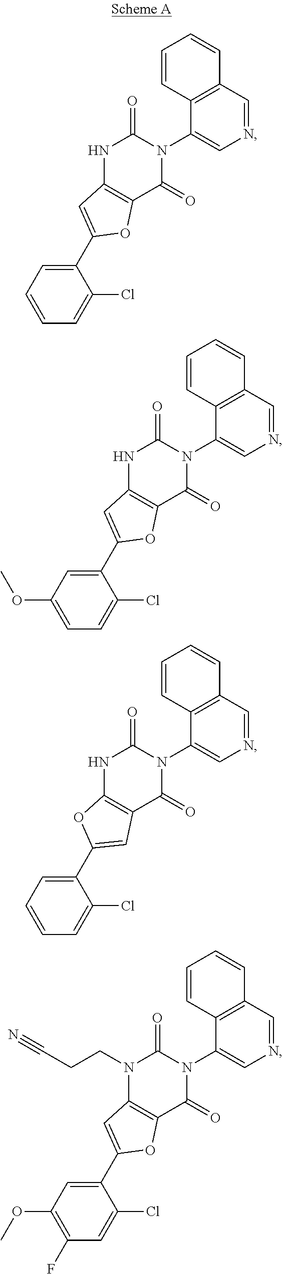 [SMILES: O=c1[nH]c2cc(-c3ccccc3Cl)oc2c(=O)n1-c1cncc2ccccc12 ; COc1ccc(Cl)c(-c2cc3[nH]c(=O)n(-c4cncc5ccccc45)c(=O)c3o2)c1 ; O=c1[nH]c2oc(-c3ccccc3Cl)cc2c(=O)n1-c1cncc2ccccc12 ; COc1cc(-c2cc3c(o2)c(=O)n(-c2cncc4ccccc24)c(=O)n3CCC#N)c(Cl)cc1F]

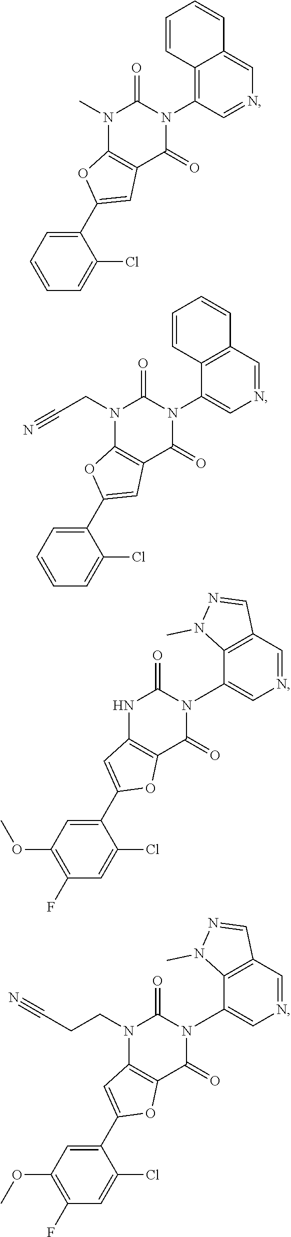 [SMILES: Cn1c(=O)n(-c2cncc3ccccc23)c(=O)c2cc(-c3ccccc3Cl)oc21 ; N#CCn1c(=O)n(-c2cncc3ccccc23)c(=O)c2cc(-c3ccccc3Cl)oc21 ; COc1cc(-c2cc3[nH]c(=O)n(-c4cncc5cnn(C)c45)c(=O)c3o2)c(Cl)cc1F ; COc1cc(-c2cc3c(o2)c(=O)n(-c2cncc4cnn(C)c24)c(=O)n3CCC#N)c(Cl)cc1F]

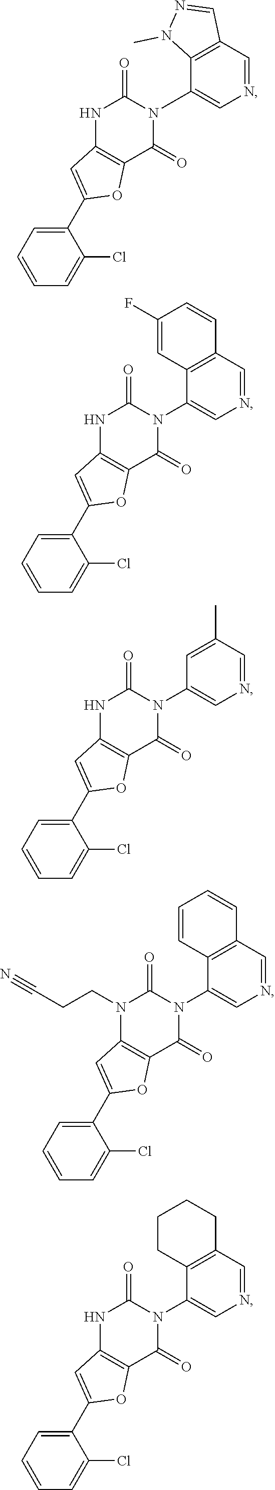 [SMILES: Cn1ncc2cncc(-n3c(=O)[nH]c4cc(-c5ccccc5Cl)oc4c3=O)c21 ; O=c1[nH]c2cc(-c3ccccc3Cl)oc2c(=O)n1-c1cncc2ccc(F)cc12 ; Cc1cncc(-n2c(=O)[nH]c3cc(-c4ccccc4Cl)oc3c2=O)c1 ; N#CCCn1c(=O)n(-c2cncc3ccccc23)c(=O)c2oc(-c3ccccc3Cl)cc21 ; O=c1[nH]c2cc(-c3ccccc3Cl)oc2c(=O)n1-c1cncc2c1CCCC2]

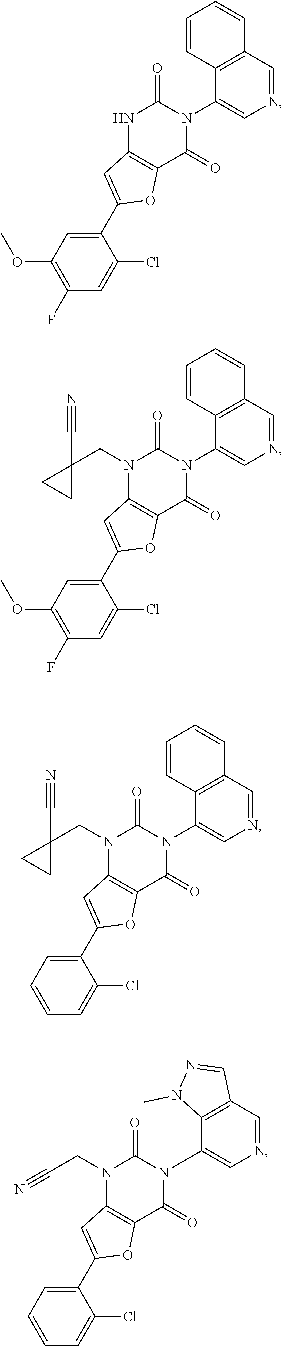 [SMILES: COc1cc(-c2cc3[nH]c(=O)n(-c4cncc5ccccc45)c(=O)c3o2)c(Cl)cc1F ; COc1cc(-c2cc3c(o2)c(=O)n(-c2cncc4ccccc24)c(=O)n3CC2(C#N)CC2)c(Cl)cc1F ; N#CC1(Cn2c(=O)n(-c3cncc4ccccc34)c(=O)c3oc(-c4ccccc4Cl)cc32)CC1 ; Cn1ncc2cncc(-n3c(=O)c4oc(-c5ccccc5Cl)cc4n(CC#N)c3=O)c21]

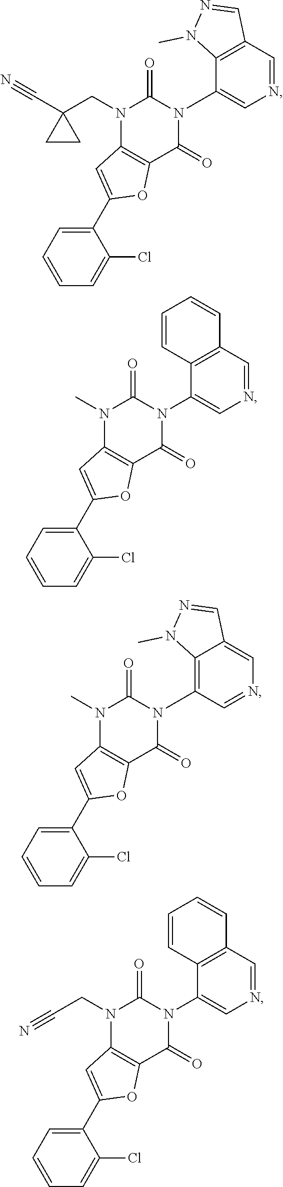 [SMILES: Cn1ncc2cncc(-n3c(=O)c4oc(-c5ccccc5Cl)cc4n(CC4(C#N)CC4)c3=O)c21 ; Cn1c(=O)n(-c2cncc3ccccc23)c(=O)c2oc(-c3ccccc3Cl)cc21 ; Cn1ncc2cncc(-n3c(=O)c4oc(-c5ccccc5Cl)cc4n(C)c3=O)c21 ; N#CCn1c(=O)n(-c2cncc3ccccc23)c(=O)c2oc(-c3ccccc3Cl)cc21]

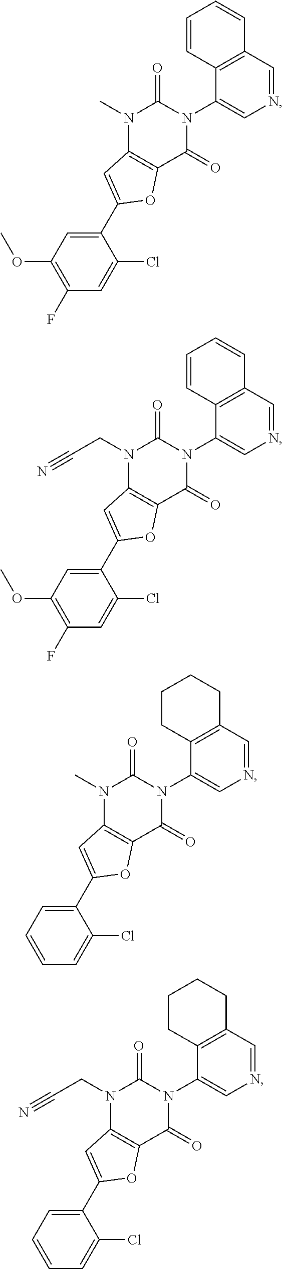 [SMILES: COc1cc(-c2cc3c(o2)c(=O)n(-c2cncc4ccccc24)c(=O)n3C)c(Cl)cc1F ; COc1cc(-c2cc3c(o2)c(=O)n(-c2cncc4ccccc24)c(=O)n3CC#N)c(Cl)cc1F ; Cn1c(=O)n(-c2cncc3c2CCCC3)c(=O)c2oc(-c3ccccc3Cl)cc21 ; N#CCn1c(=O)n(-c2cncc3c2CCCC3)c(=O)c2oc(-c3ccccc3Cl)cc21]

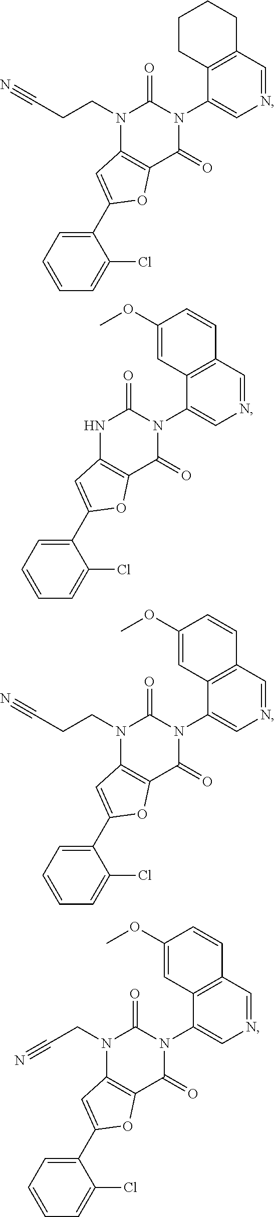

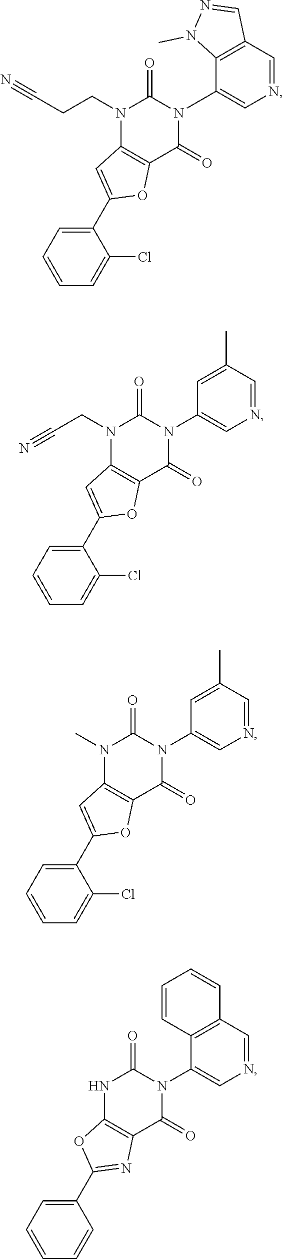 [SMILES: Cn1ncc2cncc(-n3c(=O)c4oc(-c5ccccc5Cl)cc4n(CCC#N)c3=O)c21 ; Cc1cncc(-n2c(=O)c3oc(-c4ccccc4Cl)cc3n(CC#N)c2=O)c1 ; Cc1cncc(-n2c(=O)c3oc(-c4ccccc4Cl)cc3n(C)c2=O)c1 ; O=c1[nH]c2oc(-c3ccccc3)nc2c(=O)n1-c1cncc2ccccc12]

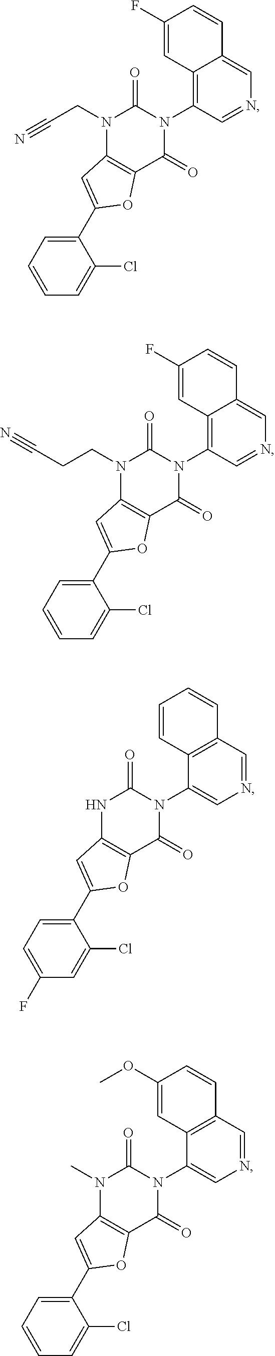 [SMILES: N#CCn1c(=O)n(-c2cncc3ccc(F)cc23)c(=O)c2oc(-c3ccccc3Cl)cc21 ; N#CCCn1c(=O)n(-c2cncc3ccc(F)cc23)c(=O)c2oc(-c3ccccc3Cl)cc21 ; O=c1[nH]c2cc(-c3ccc(F)cc3Cl)oc2c(=O)n1-c1cncc2ccccc12 ; COc1ccc2cncc(-n3c(=O)c4oc(-c5ccccc5Cl)cc4n(C)c3=O)c2c1]

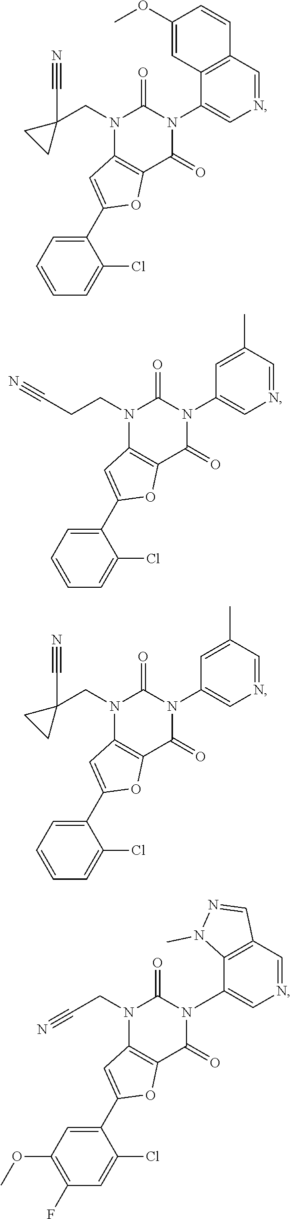 [SMILES: COc1ccc2cncc(-n3c(=O)c4oc(-c5ccccc5Cl)cc4n(CC4(C#N)CC4)c3=O)c2c1 ; Cc1cncc(-n2c(=O)c3oc(-c4ccccc4Cl)cc3n(CCC#N)c2=O)c1 ; Cc1cncc(-n2c(=O)c3oc(-c4ccccc4Cl)cc3n(CC3(C#N)CC3)c2=O)c1 ; COc1cc(-c2cc3c(o2)c(=O)n(-c2cncc4cnn(C)c24)c(=O)n3CC#N)c(Cl)cc1F]

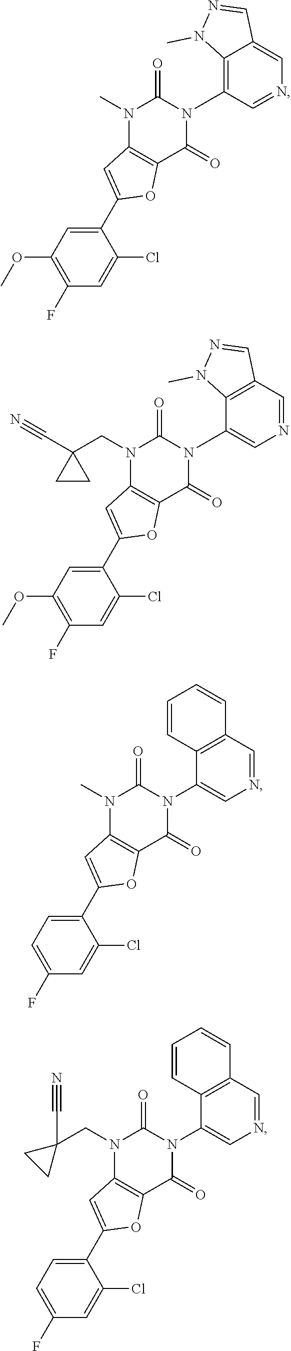 [SMILES: COc1cc(-c2cc3c(o2)c(=O)n(-c2cncc4cnn(C)c24)c(=O)n3C)c(Cl)cc1F ; COc1cc(-c2cc3c(o2)c(=O)n(-c2cncc4cnn(C)c24)c(=O)n3CC2(C#N)CC2)c(Cl)cc1F ; Cn1c(=O)n(-c2cncc3ccccc23)c(=O)c2oc(-c3ccc(F)cc3Cl)cc21 ; N#CC1(Cn2c(=O)n(-c3cncc4ccccc34)c(=O)c3oc(-c4ccc(F)cc4Cl)cc32)CC1]

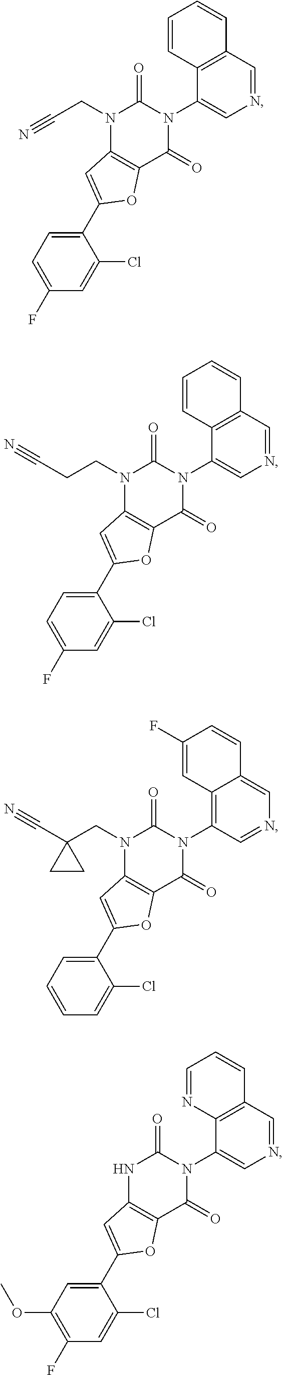 [SMILES: N#CCn1c(=O)n(-c2cncc3ccccc23)c(=O)c2oc(-c3ccc(F)cc3Cl)cc21 ; N#CCCn1c(=O)n(-c2cncc3ccccc23)c(=O)c2oc(-c3ccc(F)cc3Cl)cc21 ; N#CC1(Cn2c(=O)n(-c3cncc4ccc(F)cc34)c(=O)c3oc(-c4ccccc4Cl)cc32)CC1 ; COc1cc(-c2cc3[nH]c(=O)n(-c4cncc5cccnc45)c(=O)c3o2)c(Cl)cc1F]

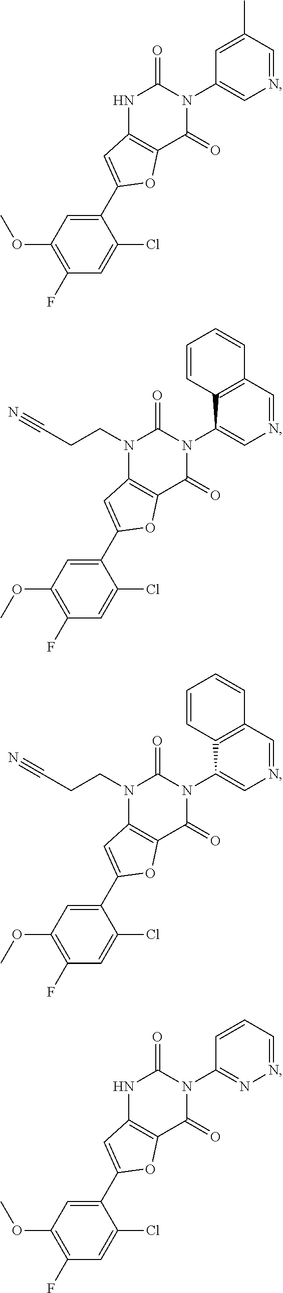

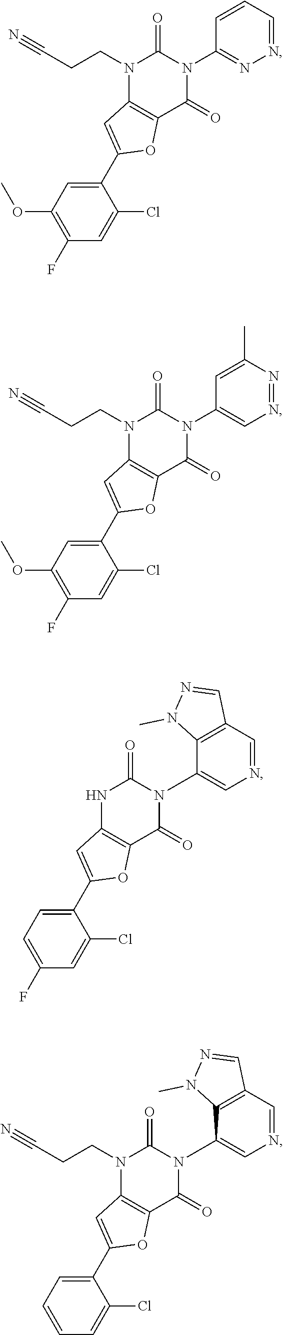

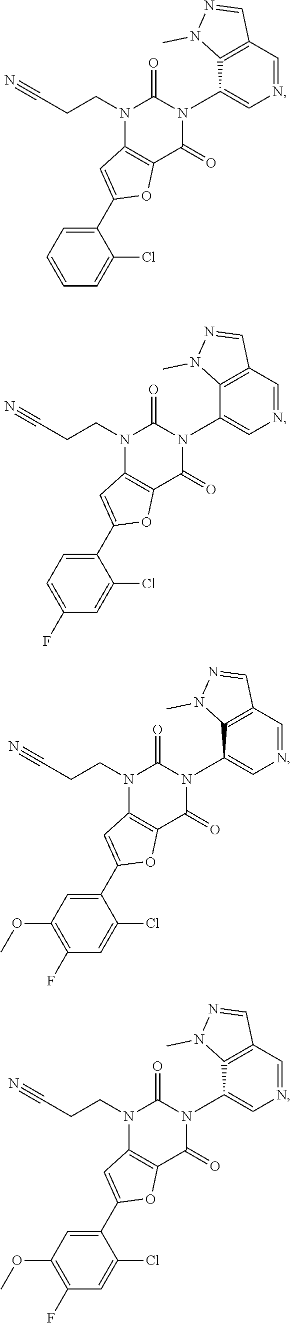

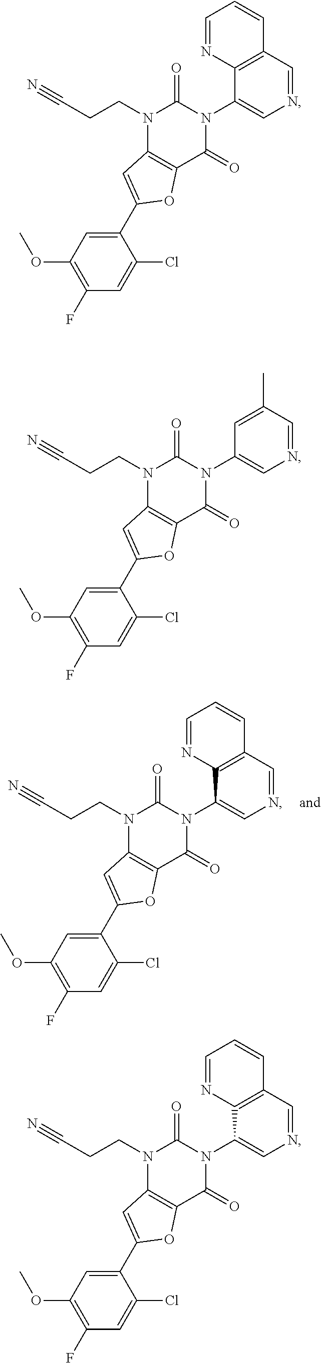

or a pharmaceutically acceptable salt thereof.

In some embodiments, a compound of the disclosure is

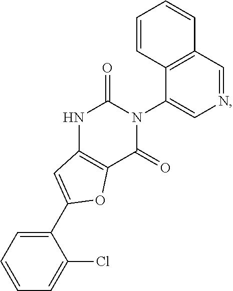 [SMILES: O=c1[nH]c2cc(-c3ccccc3Cl)oc2c(=O)n1-c1cncc2ccccc12]

or a pharmaceutically acceptable salt thereof.

In some embodiments, a compound of the disclosure is

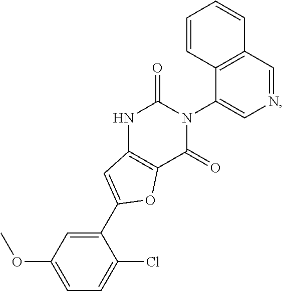 [SMILES: COc1ccc(Cl)c(-c2cc3[nH]c(=O)n(-c4cncc5ccccc45)c(=O)c3o2)c1]

or a pharmaceutically acceptable salt thereof.

In some embodiments, a compound of the disclosure is

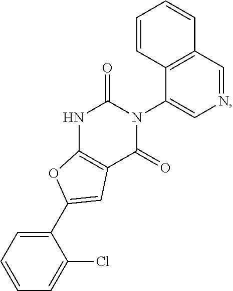 [SMILES: O=c1[nH]c2oc(-c3ccccc3Cl)cc2c(=O)n1-c1cncc2ccccc12]

or a pharmaceutically acceptable salt thereof.

In some embodiments, a compound of the disclosure is

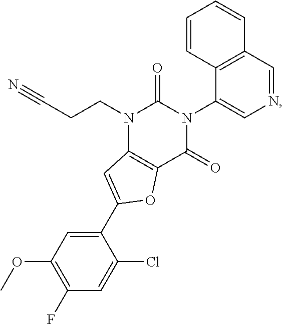 [SMILES: COc1cc(-c2cc3c(o2)c(=O)n(-c2cncc4ccccc24)c(=O)n3CCC#N)c(Cl)cc1F]

or a pharmaceutically acceptable salt thereof.

In some embodiments, a compound of the disclosure is

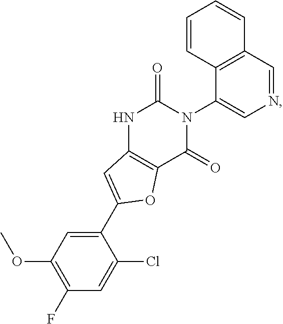 [SMILES: COc1cc(-c2cc3[nH]c(=O)n(-c4cncc5ccccc45)c(=O)c3o2)c(Cl)cc1F]

or a pharmaceutically acceptable salt thereof.

In some embodiments, a compound of the disclosure is a compound selected from any one of Examples 1 to 66, or a pharmaceutically acceptable salt thereof.

Also falling within the scope herein are the in vivo metabolic products of the compounds described herein, to the extent such products are novel and unobvious over the prior art. Such products may result for example from the oxidation, reduction, hydrolysis, amidation, esterification and the like of the administered compound, primarily due to enzymatic processes. Accordingly, included are novel and unobvious compounds produced by a process comprising contacting a compound with a mammal for a period of time sufficient to yield a metabolic product thereof. Such products typically are identified by preparing a radiolabelled (e.g. ¹⁴C or ³H) compound, administering it parenterally in a detectable dose (e.g. greater than about 0.5 mg/kg) to an animal such as rat, mouse, guinea pig, monkey, or to man, allowing sufficient time for metabolism to occur (typically about 30 seconds to about 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies well-known to those skilled in the art.

The compounds of the present disclosure can be prepared by a variety of methods. For example, Schemes 1-5 show representative syntheses of the compounds of the present disclosure. Variables shown in the following schemes are for illustrative purposes only and are independent from those described elsewhere herein.

### General Reaction Scheme 1:

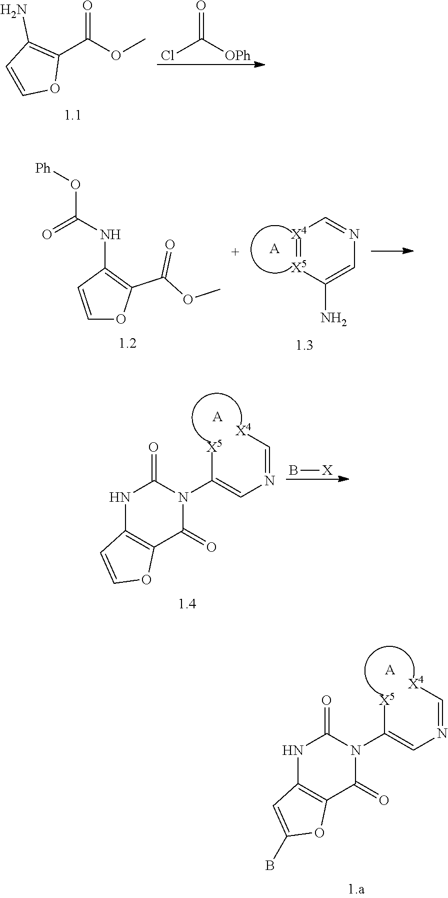

Intermediate 1.1 can be reacted with phenyl chloroformate in the presence of a suitable base (e.g., DIPEA) and with heating to give intermediate 1.2. Condensation between intermediate 1.2 and intermediate 1.3 in the presence of base and with heating gives intermediate 1.4. Metal mediated C—H arylation with a suitable coupling partner B—X (where B is aryl or heteroaryl and X is —Cl, —Br, —I, or -OTf) in the presence of a suitable catalyst (e.g., Pd) can be used to provide a compound of formula (1.a).

### General Reaction Scheme 2:

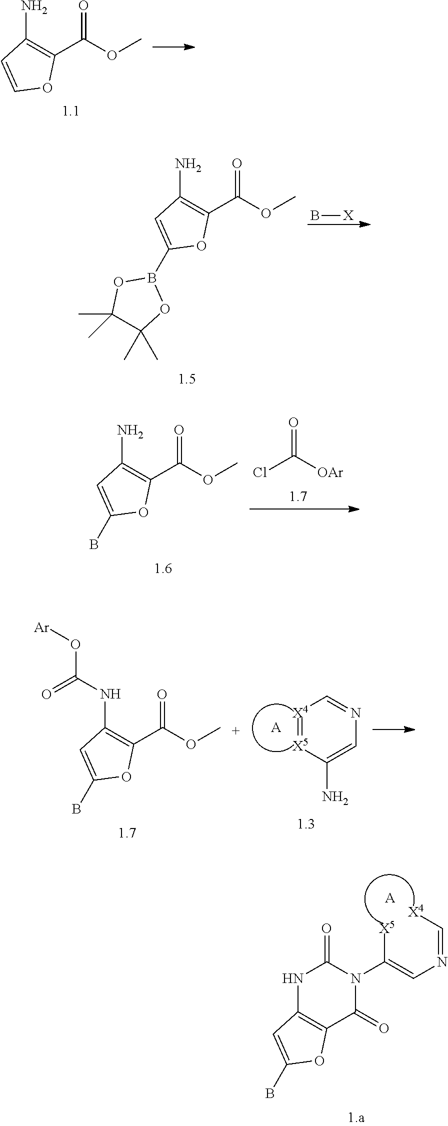

Iridium mediated borylation in the presence of intermediate 1.1, bis(pinacolato)diboron, and a suitable Ir catalyst can used to provide intermediate 1.5. Metal mediated cross-coupling with a suitable coupling partner B—X (where B is aryl or heteroaryl and X is —Cl, —Br, —I, or -OTf) in the presence of a suitable catalyst (e.g., Pd or Ni) can be used to provide intermediate 1.6. Intermediate 1.6 can be reacted with intermediate 1.7 in the presence of a suitable base (e.g., DIPEA) and with heating to give intermediate 1.7. Condensation between intermediate 1.7 and intermediate 1.3 in the presence of base and with heating can be used to provide a compound of formula (1.a).

### General Reaction Scheme 3:

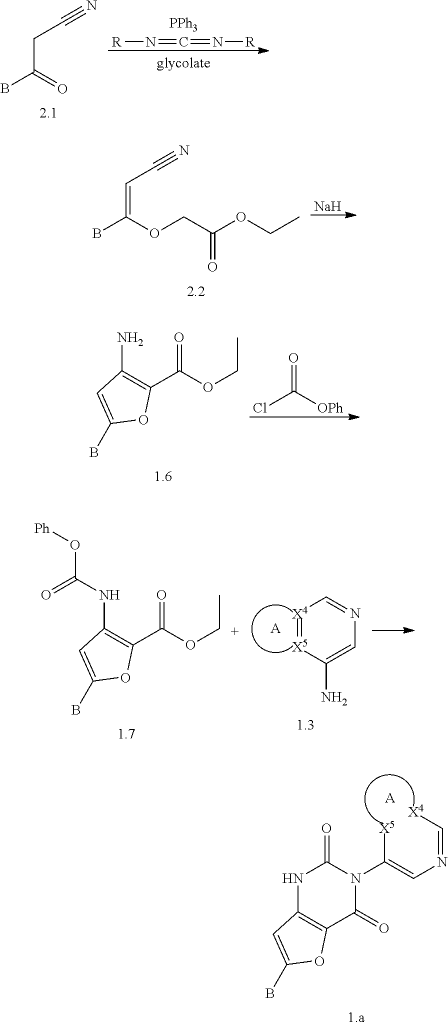

Phosphine mediated O-alkylation in the presence of intermediate 2.1, triarylphosphine, ethyl glycolate, and a suitable carbodiimide can used to provide intermediate 2.2. Base mediated cyclization with a suitable base (e.g., NaH) can be used to provide intermediate 1.6. Intermediate 1.6 can be reacted with phenyl chloroformate in the presence of a suitable base (e.g., DIPEA) and with heating to give intermediate 1.7. Condensation between intermediate 1.7 and intermediate 1.3 in the presence of base and with heating can be used to provide a compound of formula (1.a).

### General Reaction Scheme 4:

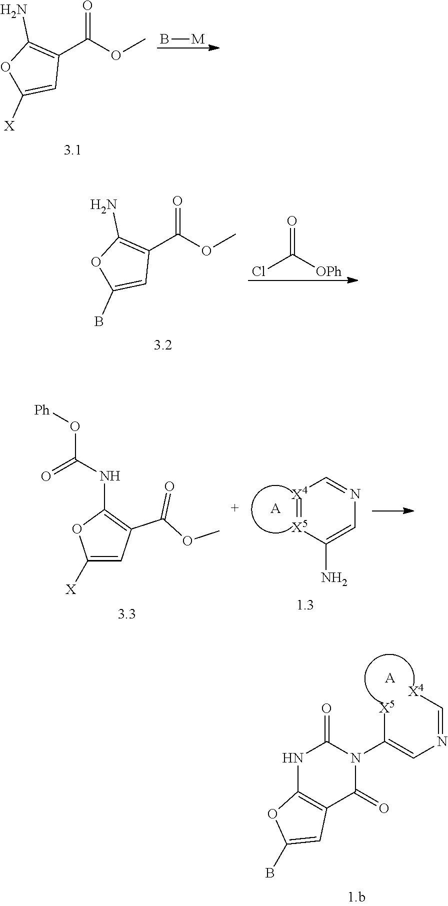

Intermediate 3.1 (where X is —Cl, —Br, —I, or -OTf), can undergo a metal mediated cross-coupling with a suitable coupling partner B-M (where B is aryl or heteroaryl and M is —B, —Sn, —Zn, —Si, or —Mg) in the presence of a suitable catalyst (e.g., Pd or Ni) to provide intermediate 3.2. Intermediate 3.2 can be reacted with phenyl chloroformate in the presence of a suitable base (e.g., DIPEA) and with heating to give intermediate 3.3. Condensation between intermediate 3.3 and intermediate 1.3 in the presence of base and with heating provides a compound of formula (1.b).

### General Reaction Scheme 5:

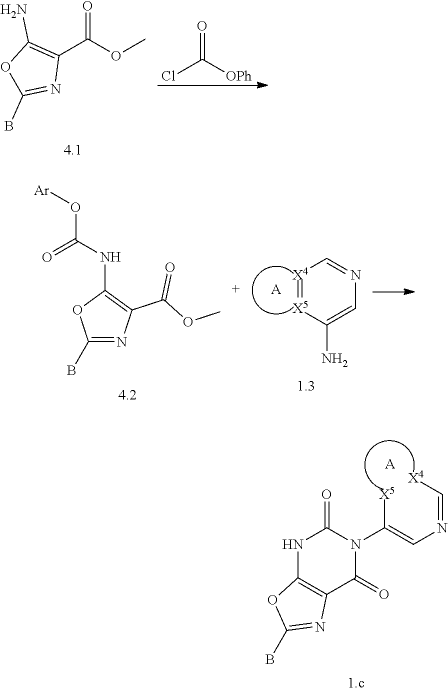

Intermediate 4.1 (where B is aryl) can be reacted with phenyl chloroformate in the presence of a suitable base (e.g., DIPEA) and with heating to give intermediate 4.2. Condensation between intermediate 4.2 and intermediate 1.3 in the presence of base and with heating can be used to provide a compound of formula (1.c).

### Pharmaceutical Formulations

In some embodiments, the present disclosure provides a pharmaceutical formulation comprising a pharmaceutically effective amount of a compound of the present disclosure, or a pharmaceutically acceptable salt thereof, and a pharmaceutically acceptable carrier or excipient. Also provided herein is a pharmaceutical formulation comprising a pharmaceutically effective amount of a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D), or a pharmaceutically acceptable salt thereof, and a pharmaceutically acceptable carrier or excipient.

In some embodiments, the pharmaceutical composition is for use in treating a virus.

In some embodiments, the pharmaceutical composition further comprises one or more additional therapeutic agents. Any suitable additional therapeutic agent or combination therapy can be used with the compounds of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), and (VII-D), or a pharmaceutically acceptable salt thereof, such as the agents and therapies described within.

In some embodiments, the pharmaceutical composition comprises a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D), and an additional therapeutic agent, wherein the additional therapeutic agent is as described herein under the heading “Combination Therapy.”

In some embodiments, compounds disclosed herein are formulated with conventional carriers and excipients, which can be selected in accord with ordinary practice. Tablets can contain excipients, glidants, fillers, binders and the like. Aqueous formulations can be prepared in sterile form, and can be isotonic, for instance when intended for delivery by other than oral administration. In some embodiments, formulations can optionally contain excipients such as those set forth in the “Handbook of Pharmaceutical Excipients” (1986). Excipients can include, for example, ascorbic acid and other antioxidants, chelating agents such as EDTA, carbohydrates such as dextran, hydroxyalkylcellulose, hydroxyalkylmethylcellulose, stearic acid and the like. The pH of the formulations ranges from about 3 to about 11, for example from about 7 to about 10.

In some embodiments, the compounds disclosed herein are administered alone. In some embodiments, compounds disclosed herein are administered in pharmaceutical formulations. In some embodiments a formulation, for veterinary and/or for human use, comprises at least one compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), and (VII-D), or a pharmaceutically acceptable salt thereof, together with one or more acceptable carriers and optionally other therapeutic ingredients, such as those additional therapeutic ingredients discussed herein. In some embodiments, carrier(s) are “acceptable” in the sense of being compatible with the other ingredients of the formulation and physiologically innocuous to the recipient thereof.

In some embodiments, formulations of the disclosure include those suitable for the foregoing administration routes. In some embodiments, formulations are presented in unit dosage form. Formulations may be prepared by methods known in the art of pharmacy. Techniques and formulations can be found, for example, in Remington's Pharmaceutical Sciences (Mack Publishing Co., Easton, PA). Such methods include, for instance, a step of bringing into association the active ingredient with a carrier comprising one or more accessory ingredients. In some embodiments, formulations are prepared by bringing into association the active ingredient with liquid carriers or finely divided solid carriers or both, and then, in some embodiments, shaping the product.

In some embodiments, the pharmaceutical formulation is for subcutaneous, intramuscular, intravenous, oral, or inhalation administration.

Formulations suitable for oral administration may be presented as discrete units such as capsules, cachets or tablets each containing a predetermined amount of active ingredient, such as a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D), or a pharmaceutically acceptable salt thereof; as a powder or granules; as a solution or a suspension in an aqueous or non-aqueous liquid; or as an oil-in-water liquid emulsion or a water-in-oil liquid emulsion. In some embodiments, an active ingredient is administered as a bolus, electuary or paste.

A tablet can be made, for example, by compression or molding, optionally with one or more accessory ingredients. Compressed tablets may be prepared, for example, by compressing in a suitable machine the active ingredient in a free-flowing form such as a powder or granules, optionally mixed with a binder, lubricant, inert diluent, preservative, surface active or dispersing agent. Molded tablets may be made, for instance, by molding in a suitable machine a mixture of the powdered active ingredient moistened with an inert liquid diluent. The tablets may optionally be coated or scored. In some embodiments, tablets are formulated so as to provide slow or controlled release of the active ingredient therefrom.

For infections of the eye or other external tissues e.g. mouth and skin, the formulations can be applied as a topical ointment or cream containing a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D), in an amount of, for example, about 0.075 to about 20% w/w (including active ingredient(s) in a range between about 0.1% and about 20% in increments of about 0.1% w/w such as about 0.6% w/w, about 0.7% w/w, etc.), such as about 0.2 to about 15% w/w and such as about 0.5 to about 10% w/w. When formulated in an ointment, a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D) may be employed with either a paraffinic or a water-miscible ointment base. Alternatively, a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D) may be formulated in a cream with an oil-in-water cream base.

If desired, the aqueous phase of the cream base may include, for example, at least about 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may in some embodiments include a compound which enhances absorption or penetration of the active ingredient through the skin or other affected areas. Examples of such dermal penetration enhancers include dimethyl sulfoxide and related analogs.

The oily phase of the emulsions may be constituted from known ingredients in a known manner. While the phase may comprise merely an emulsifier (otherwise known as an emulgent), it can comprise, for example, a mixture of at least one emulsifier with a fat or an oil or with both a fat and an oil. In some embodiments, a hydrophilic emulsifier is included together with a lipophilic emulsifier which acts as a stabilizer. In some embodiments, an emulsion includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation include, for instance, Tween® 60, Span® 80, cetostearyl alcohol, benzyl alcohol, myristyl alcohol, glyceryl mono-stearate and sodium lauryl sulfate. Further emulgents and emulsion stabilizers suitable for use in the formulation include Tween® 80.

The choice of suitable oils or fats for the formulation is based on achieving the desired properties. The cream can be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Pharmaceutical formulations according to the present disclosure comprise a compound according to the disclosure together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the disclosure contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin. In some examples, the suspending agent is Sulfobutyl ether beta-cyclodextrin (SEB-beta-CD), for example Captisol®.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the disclosure may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 mg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

In some embodiments, the compounds disclosed herein are administered by inhalation. In some embodiments, formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents. In some embodiments, the compounds used herein are formulated and dosed as dry powder. In some embodiments, the compounds used herein are formulated and dosed as a nebulized formulation. In some embodiments, the compounds used herein are formulated for delivery by a face mask. In some embodiments, the compounds used herein are formulated for delivery by a face tent.

Another embodiment provides an inhalable composition comprising a compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D), or a pharmaceutically acceptable salt thereof. In In some embodiments, pharmaceutically acceptable salts are inorganic acid salts including hydrochloride, hydrobromide, sulfate or phosphate salts. For example, such salts may cause less pulmonary irritation relative to other salts. In some embodiments, an inhalable composition is delivered to the endobronchial space in an aerosol comprising particles with a mass median aerodynamic diameter (MMAD) between about 1 and about 5 μm. In some embodiments, the compound of Formula (I), (I-A), (II-A), (II-B), (II-C), (II-D), (III), (IV-A), (IV-B), (IV-C), (IV-D), (V-A), (V-B), (V-C), (V-D), (VI), (VII-A), (VII-B), (VII-C), or (VII-D), or a pharmaceutically acceptable salt thereof, is formulated for aerosol delivery using a nebulizer, pressurized metered dose inhaler (pMDI), or dry powder inhaler (DPI).

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this disclosure may include other agents conventional in the art having regard to the type of formulation in question, for example those suitable for oral administration may include flavoring agents.

The disclosure further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the disclosure are used to provide controlled release pharmaceutical formulations containing as active ingredient one or more compounds of the disclosure (“controlled release formulations”) in which the release of the active ingredient are controlled and regulated to allow less frequency dosing or to improve the pharmacokinetic or toxicity profile of a given active ingredient.

Effective dose of active ingredient depends at least on the nature of the condition being treated, toxicity, the method of delivery, and the pharmaceutical formulation, and can be determined by the clinician using conventional dose escalation studies. It can be expected to be from about 0.0001 to about 100 mg/kg body weight per day; typically, from about 0.01 to about 10 mg/kg body weight per day; more typically, from about 0.01 to about 5 mg/kg body weight per day; most typically, from about 0.05 to about 0.5 mg/kg body weight per day. For example, the daily candidate dose for an adult human of about 70 kg body weight can range from about 1 mg to about 1000 mg, such as between about 5 mg and about 500 mg, and may take the form of single or multiple doses.

### Methods of Use

The present disclosure also provides a method of treating or preventing a viral infection in a subject (e.g., human) in need thereof, the method comprising administering to the subject a compound described herein.

In some embodiments, the present disclosure provides a method of treating a viral infection in a subject (e.g., human) in need thereof, the method comprising administering to a subject in need thereof a compound described herein.

In some embodiments, the compound described herein is administered to the human via oral, intramuscular, intravenous, subcutaneous, or inhalation administration.

In some embodiments, the present disclosure provides for methods of treating or preventing a viral infection in a subject (e.g., human) in need thereof, the method comprising administering to the subject a compound disclosed herein and at least one additional active therapeutic or prophylactic agent.

In some embodiments, the present disclosure provides for methods of treating a viral infection in a subject (e.g., human) in need thereof, the method comprising administering to the subject a compound disclosed herein, and at least one additional active therapeutic or prophylactic agent.

In one embodiment, the present disclosure provides for methods of inhibiting a viral polymerase in a cell, the methods comprising contacting the cell infected a virus with a compound disclosed herein, whereby the viral polymerase is inhibited.

In one embodiment, the present disclosure provides for methods of inhibiting a viral protease in a cell, the methods comprising contacting the cell infected a virus with a compound disclosed herein, and at least one additional active therapeutic agent, whereby the viral protease is inhibited.

Also provided here are uses of a compound disclosed herein, or a pharmaceutically acceptable salt thereof, or a pharmaceutical composition thereof, for treating or preventing a viral infection in a human in need thereof. For example, provided herein are uses of a compound disclosed herein, or a pharmaceutically acceptable salt thereof, or a pharmaceutical composition thereof, for treating a viral infection in a human in need thereof.

Also provided here are uses of a compound disclosed herein, or a pharmaceutically acceptable salt thereof, or a pharmaceutical composition thereof, for the manufacture of a medicament for the treatment or prevention of a viral infection in a human in need thereof.

Also provided here are methods for manufacturing a medicament for treating or preventing a viral infection in a human in need thereof, characterized in that a compound disclosed herein, or a pharmaceutically acceptable salt thereof, or a pharmaceutical composition thereof, is used.

Also provided here are compositions comprising a compound disclosed herein, or a pharmaceutically acceptable salt thereof, or a pharmaceutical composition thereof, for use in treatment or prevention of a viral infection in a human in need thereof.

In some embodiments, the viral infection is a coronavirus infection in a human in need thereof, wherein the method comprises administering to the human a compound provided herein. In some embodiments, the coronavirus infection is a Severe Acute Respiratory Syndrome (SARS-CoV) infection, Middle Eastern Respiratory Syndrome (MERS) infection, SARS-CoV-2 infection, other human coronavirus (229E, NL63, OC43, HKU1, or WIV1) infections, zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection. In some embodiments, the viral infection is a zoonotic coronavirus infection. In some embodiments, the viral infection is caused by a virus having at least 70% sequence homology to a viral polymerase selected from the group consisting of SARS-CoV polymerase, MERS-CoV polymerase and SARS-CoV-2. In some embodiments, the viral infection is caused by a virus having at least 80% sequence homology to a viral polymerase selected from the group consisting of SARS-CoV polymerase, MERS-CoV polymerase and SARS-CoV-2. In some embodiments, the viral infection is caused by a virus having at least 90% sequence homology to a viral polymerase selected from the group consisting of SARS-CoV polymerase, MERS-CoV polymerase and SARS-CoV-2. In some embodiments, the viral infection is caused by a virus having at least 95% sequence homology to a viral polymerase selected from the group consisting of SARS-CoV polymerase, MERS-CoV polymerase and SARS-CoV-2.

In some embodiments, the viral infection is caused by a variant of SARS-CoV-2, for example by the B.1.1.7 variant (the UK variant), B.1.351 variant (the South African variant), P.1 variant (the Brazil variant), B.1.1.7 with E484K variant, B.1.1.207 variant, B.1.1.317 variant, B.1.1.318 variant, B.1.429 variant, B.1.525 variant, or P.3 variant. In some embodiments, the viral infection is caused by the B.1.1.7 variant of SARS-CoV-2. In some embodiments, the viral infection is caused by the B.1.351 variant of SARS-CoV-2. In some embodiments, the viral infection is caused by the P.1 variant of SARS-CoV-2.

In some embodiments, the present disclosure provides a compound for use in the treatment of a coronavirus virus infection in a human in need thereof. In some embodiments, the coronavirus infection is a Severe Acute Respiratory Syndrome (SARS) infection, Middle Eastern Respiratory Syndrome (MERS) infection, SARS-CoV-2 infection, other human coronavirus (229E, NL63, OC43, HKU1, or WIV1) infections, and zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection (COVID19).

As described more fully herein, the compounds described herein can be administered with one or more additional therapeutic agent(s) to an individual (e.g., a human) infected with a viral infection. The additional therapeutic agent(s) can be administered to the infected individual at the same time as the compound of the present disclosure or before or after administration of the compound of the present disclosure.

### Administration

One or more compounds of the disclosure may be administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, the compounds disclosed herein are administered by inhalation or intravenously. In some embodiments, the compounds disclosed herein are administered orally. It will be appreciated that the preferred route may vary with for example the condition of the recipient.

In the methods of the present disclosure for the treatment of a viral infection, the compounds of the present disclosure can be administered at any time to a human who may come into contact with the virus or is already suffering from the viral infection. In some embodiments, the compounds of the present disclosure can be administered prophylactically to humans coming into contact with humans suffering from the viral infection or at risk of coming into contact with humans suffering from the viral infection, e.g., healthcare providers. In some embodiments, administration of the compounds of the present disclosure can be to humans testing positive for the viral infection but not yet showing symptoms of the viral infection. In some embodiments, administration of the compounds of the present disclosure can be to humans upon commencement of symptoms of the viral infection.

In some embodiments, the methods disclosed herein comprise event driven administration of the compound of the present disclosure, or a pharmaceutically acceptable salt thereof, to the subject.

As used herein, the terms “event driven” or “event driven administration” refer to administration of the compound described herein, e.g., the compound of the present disclosure, or a pharmaceutically acceptable salt thereof, (1) prior to an event (e.g., 2 hours, 1 day, 2 days, 5 day, or 7 or more days prior to the event) that would expose the individual to the virus (or that would otherwise increase the individual's risk of acquiring the viral infection); and/or (2) during an event (or more than one recurring event) that would expose the individual to the virus (or that would otherwise increase the individual's risk of acquiring the viral infection); and/or (3) after an event (or after the final event in a series of recurring events) that would expose the individual to the virus (or that would otherwise increase the individual's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods disclosed herein involve administration prior to and/or after an event that would expose the individual to the virus or that would otherwise increase the individual's risk of acquiring the viral infection, e.g., as pre-exposure prophylaxis (PrEP) and/or as post-exposure prophylaxis (PEP). In some embodiments, the methods disclosed herein comprise pre-exposure prophylaxis (PrEP). In some embodiments, methods disclosed herein comprise post-exposure prophylaxis (PEP).

In some embodiments, the compound of the present disclosure, or a pharmaceutically acceptable salt thereof, is administered before exposure of the subject to the virus.

In some embodiments, the compound of the present disclosure, or a pharmaceutically acceptable salt thereof, is administered before and after exposure of the subject to the virus.

In some embodiments, the compound of the present disclosure, or a pharmaceutically acceptable salt thereof, is administered after exposure of the subject to the virus.

An example of event driven dosing regimen includes administration of the compound of the present disclosure, or a pharmaceutically acceptable salt thereof, within 24 to 2 hours prior to the virus, followed by administration of the compound of the present disclosure, or a pharmaceutically acceptable salt, every 24 hours during the period of exposure, followed by a further administration of the compound of the present disclosure, or a pharmaceutically acceptable salt thereof, after the last exposure, and one last administration of the compound of Formula A or Formula B, or a pharmaceutically acceptable salt thereof, 24 hours later.

A further example of an event driven dosing regimen includes administration of the compound of the present disclosure, or a pharmaceutically acceptable salt thereof, within 24 hours before the viral exposure, then daily administration during the period of exposure, followed by a last administration approximately 24 hours later after the last exposure (which may be an increased dose, such as a double dose).

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of a compound of the present disclosure, or a pharmaceutically acceptable salt thereof, may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount. In some embodiments, the compound is administered once daily.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per day, or from about 30 mg to about 300 mg per day, or from about 0.3 mg to about 30 mg per day, or from about 30 mg to about 300 mg per day.

A compound of the present disclosure may be combined with one or more additional therapeutic agents in any dosage amount of the compound of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amount of the compound of the present disclosure is about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amount of the compound of the present disclosure is about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a compound provided herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1-4,000 mg/day, between about 1-3,000 mg/day, between 1-2,000 mg/day, about 1-1,000 mg/day, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg/day. In some embodiments, the total daily dosage for a human subject may be about 100-200, 100-300, 100-400, 100-500, 100-600, 100-700, 100-800, 100-900, 100-1000, 500-1100, 500-1200, 500-1300, 500-1400, 500-1500, 500-1600, 500-1700, 500-1800, 500-1900, 500-2000, 1500-2100, 1500-2200, 1500-2300, 1500-2400, 1500-2500, 2000-2600, 2000-2700, 2000-2800, 2000-2900, 2000-3000, 2500-3100, 2500-3200, 2500-3300, 2500-3400, 2500-3500, 3000-3600, 3000-3700, 3000-3800, 3000-3900, or 3000-4000 mg/day.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose. In some embodiments, the total daily dosage for a human subject may be about 1500 mg/day administered in a single dose. In some embodiments, the total daily dosage for a human subject may be about 2000 mg/day administered in a single dose. In some embodiments, the total daily dosage for a human subject may be about 2500 mg/day administered in a single dose. In some embodiments, the total daily dosage for a human subject may be about 3000 mg/day administered in a single dose. In some embodiments, the total daily dosage for a human subject may be about 4000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein. In some embodiments, a compound disclosed herein is administered three times daily in a method disclosed herein.

In some embodiments, a compound disclosed herein is administered once daily in the total daily dose of 100-4000 mg/day. In some embodiments, a compound disclosed herein is administered twice daily in the total daily dose of 100-4000 mg/day. In some embodiments, a compound disclosed herein is administered three times daily in the total daily dose of 100-4000 mg/day.

The frequency of dosage of the compound of the present disclosure will be determined by the needs of the individual patient and can be, for example, once per day or twice, or more times, per day. Administration of the compound continues for as long as necessary to treat the viral infection. For example, a compound can be administered to a human being infected with the virus for a period of from 20 days to 180 days or, for example, for a period of from 20 days to 90 days or, for example, for a period of from 30 days to 60 days.

Administration can be intermittent, with a period of several or more days during which a patient receives a daily dose of the compound of the present disclosure followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

The compounds of the present disclosure or the pharmaceutical compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles may be alternated with resting periods of about 1 to 28 days, commonly about 7 days or about 14 days, between cycles. The treatment cycles, in other embodiments, may also be continuous.

### Combination Therapy

The compounds described herein can also be used in combination with one or more additional therapeutic agents. As such, also provided herein are methods of treatment of a viral infection in a subject in need thereof, wherein the methods comprise administering to the subject a compound disclosed therein and a therapeutically effective amount of one or more additional therapeutic or prophylactic agents.

In some embodiments, the additional therapeutic agent is an antiviral agent. Any suitable antiviral agent can be used in the methods described herein.

In some embodiments, the additional therapeutic agent a 2,5-Oligoadenylate synthetase stimulator, 5-HT 2a receptor antagonist, 5-Lipoxygenase inhibitor, ABL family tyrosine kinase inhibitor, Abl tyrosine kinase inhibitor, Acetaldehyde dehydrogenase inhibitor, Acetyl CoA carboxylase inhibitor, Actin antagonist, Actin modulator, Activity-dependent neuroprotector modulator, Adenosine A3 receptor agonist, Adrenergic receptor antagonist, Adrenomedullin ligand, Adrenomedullin ligand inhibitor, Advanced glycosylation product receptor antagonist, Advanced glycosylation product receptor modulator, AKT protein kinase inhibitor, Alanine proline rich secreted protein stimulator, Aldose reductase inhibitor, Alkaline phosphatase stimulator, Alpha 2 adrenoceptor antagonist, Alpha 2B adrenoceptor agonist, AMP activated protein kinase stimulator, AMPA receptor modulator, Amyloid protein deposition inhibitor, Androgen receptor antagonist, Angiotensin II AT-1 receptor antagonist, Angiotensin II AT-2 receptor agonist, Angiotensin II receptor modulator, Angiotensin converting enzyme 2 inhibitor, Angiotensin converting enzyme 2 modulator, Angiotensin converting enzyme 2 stimulator, Angiotensin receptor modulator, Annexin A5 stimulator, Anoctamin 1 inhibitor, Anti-coagulant, Anti-histamine, Anti-hypoxic, Anti-thrombotic, AP1 transcription factor modulator, Apelin receptor agonist, APOA1 gene stimulator, Apolipoprotein A1 agonist, Apolipoprotein B antagonist, Apolipoprotein B modulator, Apolipoprotein C3 antagonist, Aryl hydrocarbon receptor agonist, Aryl hydrocarbon receptor antagonist, ATP binding cassette transporter B5 modulator, Axl tyrosine kinase receptor inhibitor, Bactericidal permeability protein inhibitor, Basigin inhibitor, Basigin modulator, BCL2 gene inhibitor, BCL2L11 gene stimulator, Bcr protein inhibitor, Beta 1 adrenoceptor modulator, Beta 2 adrenoceptor agonist, Beta adrenoceptor agonist, Beta-arrestin stimulator, Blood clotting modulator, BMP10 gene inhibitor, BMP15 gene inhibitor, Bone morphogenetic protein-10 ligand inhibitor, Bone morphogenetic protein-15 ligand inhibitor, Bradykinin B2 receptor antagonist, Brain derived neurotrophic factor ligand, Bromodomain containing protein 2 inhibitor, Bromodomain containing protein 4 inhibitor, Btk tyrosine kinase inhibitor, C-reactive protein modulator, Ca2+ release activated Ca2+ channel 1 inhibitor, Cadherin-5 modulator, Calcium activated chloride channel inhibitor, Calcium channel modulator, Calpain-I inhibitor, Calpain-II inhibitor, Calpain-IX inhibitor, Cannabinoid CB2 receptor agonist, Cannabinoid receptor modulator, Casein kinase II inhibitor, CASP8-FADD-like regulator inhibitor, Caspase inhibitor, Catalase stimulator, CCL26 gene inhibitor, CCR2 chemokine antagonist, CCR5 chemokine antagonist, CD11a agonist, CD122 agonist, CD3 antagonist, CD4 agonist, CD40 ligand, CD40 ligand modulator, CD40 ligand receptor agonist, CD40 ligand receptor modulator, CD49d agonist, CD70 antigen modulator, CD73 agonist, CD73 antagonist, CD95 antagonist, CFTR inhibitor, CGRP receptor antagonist, Chemokine receptor-like 1 agonist, Chloride channel inhibitor, Chloride channel modulator, Cholera enterotoxin subunit B inhibitor, Cholesterol ester transfer protein inhibitor, Collagen modulator, Complement C1s subcomponent inhibitor, Complement C₃ inhibitor, Complement C₅ factor inhibitor, Complement C₅a factor inhibitor, Complement Factor H stimulator, Complement cascade inhibitor, Complement factor C₂ inhibitor, Complement factor D inhibitor, Connective tissue growth factor ligand inhibitor, Coronavirus nucleoprotein modulator, Coronavirus small envelope protein modulator, Coronavirus spike glycoprotein inhibitor, Coronavirus spike glycoprotein modulator, COVID19 envelope small membrane protein modulator, COVID19 non structural protein 8 modulator, COVID19 nucleoprotein modulator, COVID19 Protein 3a inhibitor, COVID19 replicase polyprotein 1a inhibitor, COVID19 replicase polyprotein 1a modulator, COVID19 replicase polyprotein 1ab inhibitor, COVID19 replicase polyprotein 1ab modulator, COVID19 Spike glycoprotein inhibitor, COVID19 Spike glycoprotein modulator, COVID19 structural glycoprotein modulator, CRF-2 receptor agonist, CSF-1 agonist, CSF-1 antagonist, CX3CR1 chemokine antagonist, CXC10 chemokine ligand inhibitor, CXC5 chemokine ligand inhibitor, CXCL1 gene modulator, CXCL2 gene modulator, CXCL3 gene modulator, CXCR1 chemokine antagonist, CXCR2 chemokine antagonist, CXCR4 chemokine antagonist, Cyclin D1 inhibitor, Cyclin E inhibitor, Cyclin-dependent kinase-1 inhibitor, Cyclin-dependent kinase-2 inhibitor, Cyclin-dependent kinase-5 inhibitor, Cyclin-dependent kinase-7 inhibitor, Cyclin-dependent kinase-9 inhibitor, Cyclooxygenase 2 inhibitor, Cyclooxygenase inhibitor, Cysteine protease inhibitor, Cytochrome P450 3A4 inhibitor, Cytokine receptor antagonist, Cytotoxic T lymphocyte protein gene modulator, Cytotoxic T-lymphocyte protein-4 inhibitor, Cytotoxic T-lymphocyte protein-4 stimulator, Dehydrogenase inhibitor, Dehydropeptidase-1 modulator, Deoxyribonuclease I stimulator, Deoxyribonuclease gamma stimulator, Deoxyribonuclease stimulator, Dihydroceramide delta 4 desaturase inhibitor, Dihydroorotate dehydrogenase inhibitor, Dipeptidyl peptidase I inhibitor, Dipeptidyl peptidase III inhibitor, Diuretic, DNA binding protein inhibitor, DNA methyltransferase inhibitor, Dopamine transporter inhibitor, E selectin antagonist, Ecto NOX disulfide thiol exchanger 2 inhibitor, EGFR gene inhibitor, Elongation factor 1 alpha 2 modulator, Endoplasmin modulator, Endoribonuclease DICER modulator, Endothelin ET-A receptor antagonist, Epidermal growth factor receptor antagonist, Estrogen receptor beta agonist, Estrogen receptor modulator, Eukaryotic initiation factor 4A-I inhibitor, Exo-alpha sialidase modulator, Exportin 1 inhibitor, Factor Ia modulator, Factor IIa modulator, Factor VII antagonist, Factor Xa antagonist, Factor XIa antagonist, FGF receptor antagonist, FGF-1 ligand, FGF-1 ligand inhibitor, FGF-2 ligand inhibitor, FGF1 receptor antagonist, FGF2 receptor antagonist, FGF3 receptor antagonist, Flt3 tyrosine kinase inhibitor, Fractalkine ligand inhibitor, Free fatty acid receptor 2 agonist, Free fatty acid receptor 3 agonist, furin inhibitors, Fyn tyrosine kinase inhibitor, FYVE finger phosphoinositide kinase inhibitor, G-protein coupled bile acid receptor 1 agonist, GABA A receptor modulator, Galectin-3 inhibitor, Gamma-secretase inhibitor, GDF agonist, Gelsolin stimulator, Glial cell neurotrophic factor ligand, Glucocorticoid receptor agonist, Glutathione peroxidase stimulator, GM-CSF ligand inhibitor, GM-CSF receptor agonist, GM-CSF receptor modulator, Griffithsin modulator, Growth regulated protein alpha ligand inhibitor, Grp78 calcium binding protein inhibitor, Heat shock protein HSP90 alpha inhibitor, Heat shock protein HSP90 beta inhibitor, Heat shock protein inhibitor, Heat shock protein stimulator, Hemagglutinin modulator, Hemoglobin modulator, Hemolysin alpha inhibitor, Heparanase inhibitor, Heparin agonist, Hepatitis B structural protein inhibitor, Hepatitis C virus NS5B polymerase inhibitor, HIF prolyl hydroxylase inhibitor, HIF prolyl hydroxylase-2 inhibitor, High mobility group protein B1 inhibitor, Histamine H1 receptor antagonist, Histamine H2 receptor antagonist, Histone deacetylase-6 inhibitor, Histone inhibitor, HIV protease inhibitor, HIV-1 gp120 protein inhibitor, HIV-1 protease inhibitor, HIV-1 reverse transcriptase inhibitor, HLA class I antigen modulator, HLA class II antigen modulator, Host cell factor modulator, Hsp 90 inhibitor, Human papillomavirus E6 protein modulator, Human papillomavirus E7 protein modulator, Hypoxia inducible factor inhibitor gene inhibitor, Hypoxia inducible factor-2 alpha modulator, I-kappa B kinase inhibitor, I-kappa B kinase modulator, ICAM-1 stimulator, IgG receptor FcRn large subunit p51 modulator, IL-12 receptor antagonist, IL-15 receptor agonist, IL-15 receptor modulator, IL-17 antagonist, IL-18 receptor accessory protein antagonist, IL-2 receptor agonist, IL-22 agonist, IL-23 antagonist, IL-6 receptor agonist, IL-6 receptor antagonist, IL-6 receptor modulator, IL-7 receptor agonist, IL-8 receptor antagonist, IL12 gene stimulator, IL8 gene modulator, Immunoglobulin G modulator, Immunoglobulin G1 agonist, Immunoglobulin G1 modulator, Immunoglobulin agonist, Immunoglobulin gamma Fc receptor I modulator, Immunoglobulin kappa modulator, Inosine monophosphate dehydrogenase inhibitor, Insulin sensitizer, Integrin agonist, Integrin alpha-4/beta-7 antagonist, Integrin alpha-V/beta-1 antagonist, Integrin alpha-V/beta-6 antagonist, Interferon agonist, Interferon alpha 14 ligand, Interferon alpha 2 ligand, Interferon alpha 2 ligand modulator, Interferon alpha ligand, Interferon alpha ligand inhibitor, Interferon alpha ligand modulator, Interferon beta ligand, Interferon gamma ligand inhibitor, Interferon gamma receptor agonist, Interferon gamma receptor antagonist, Interferon receptor modulator, Interferon type I receptor agonist, Interleukin 17A ligand inhibitor, Interleukin 17F ligand inhibitor, Interleukin 18 ligand inhibitor, Interleukin 22 ligand, Interleukin-1 beta ligand inhibitor, Interleukin-1 beta ligand modulator, Interleukin-1 ligand inhibitor, Interleukin-2 ligand, Interleukin-29 ligand, Interleukin-6 ligand inhibitor, Interleukin-7 ligand, Interleukin-8 ligand inhibitor, IRAK-4 protein kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Jun N terminal kinase modulator, Kallikrein modulator, Kelch like ECH associated protein 1 modulator, Kit tyrosine kinase inhibitor, KLKB1 gene inhibitor, Lactoferrin stimulator, Lanosterol-14 demethylase inhibitor, Lck tyrosine kinase inhibitor, Leukocyte Ig like receptor A4 modulator, Leukocyte elastase inhibitor, Leukotriene BLT receptor antagonist, Leukotriene D4 antagonist, Leukotriene receptor antagonist, Listeriolysin stimulator, Liver X receptor antagonist, Low molecular weight heparin, Lung surfactant associated protein B stimulator, Lung surfactant associated protein D modulator, Lyn tyrosine kinase inhibitor, Lyn tyrosine kinase stimulator, Lysine specific histone demethylase 1 inhibitor, Macrophage migration inhibitory factor inhibitor, Mannan-binding lectin serine protease inhibitor, Mannan-binding lectin serine protease-2 inhibitor, MAO B inhibitor, MAP kinase inhibitor, MAPK gene modulator, Matrix metalloprotease modulator, Maxi K potassium channel inhibitor, MCL1 gene inhibitor, MEK protein kinase inhibitor, MEK-1 protein kinase inhibitor, Melanocortin MC1 receptor agonist, Melanocortin MC3 receptor agonist, Metalloprotease-12 inhibitor, METTL3 gene inhibitor, Moesin inhibitor, Moesin modulator, Monocyte chemotactic protein 1 ligand inhibitor, Monocyte differentiation antigen CD14 inhibitor, mRNA cap guanine N7 methyltransferase modulator, mTOR complex 1 inhibitor, mTOR complex 2 inhibitor, mTOR inhibitor, Mucolipin modulator, Muscarinic receptor antagonist, Myeloperoxidase inhibitor, NACHT LRR PYD domain protein 3 inhibitor, NAD synthase modulator, NADPH oxidase inhibitor, Neuropilin 2 modulator, Neuroplastin inhibitor, NFE2L2 gene stimulator, NK cell receptor agonist, NK1 receptor antagonist, NMDA receptor antagonist, NMDA receptor epsilon 2 subunit inhibitor, Non receptor tyrosine kinase TYK2 antagonist, Non-nucleoside reverse transcriptase inhibitor, Nuclear erythroid 2-related factor 2 stimulator, Nuclear factor kappa B inhibitor, Nuclear factor kappa B modulator, Nuclease stimulator, Nucleolin inhibitor, Nucleoprotein inhibitor, Nucleoprotein modulator, Nucleoside reverse transcriptase inhibitor, Opioid receptor agonist, Opioid receptor antagonist, Opioid receptor mu modulator, Opioid receptor sigma antagonist 1, Ornithine decarboxylase inhibitor, Outer membrane protein inhibitor, OX40 ligand, p38 MAP kinase alpha inhibitor, p38 MAP kinase inhibitor, p38 MAP kinase modulator, p53 tumor suppressor protein stimulator, Palmitoyl protein thioesterase 1 inhibitor, Papain inhibitor, PARP inhibitor, PARP modulator, PDE 10 inhibitor, PDE 3 inhibitor, PDE 4 inhibitor, PDGF receptor alpha antagonist, PDGF receptor antagonist, PDGF receptor beta antagonist, Peptidyl-prolyl cis-trans isomerase A inhibitor, Peroxiredoxin 6 modulator, PGD2 antagonist, PGI2 agonist, P-glycoprotein inhibitor, Phosphoinositide 3-kinase inhibitor, Phosphoinositide-3 kinase delta inhibitor, Phosphoinositide-3 kinase gamma inhibitor, Phospholipase A2 inhibitor, Plasma kallikrein inhibitor, Plasminogen activator inhibitor 1 inhibitor, Platelet inhibitor, Platelet glycoprotein VI inhibitor, Polo-like kinase 1 inhibitor, Poly ADP ribose polymerase 1 inhibitor, Poly ADP ribose polymerase 2 inhibitor, Polymerase cofactor VP35 inhibitor, PPAR alpha agonist, Progesterone receptor agonist, Programmed cell death protein 1 modulator, Prolyl hydroxylase inhibitor, Prostaglandin E synthase-1 inhibitor, Protease inhibitor, Proteasome inhibitor, Protein arginine deiminase IV inhibitor, Protein tyrosine kinase inhibitor, Protein tyrosine phosphatase beta inhibitor, Protein tyrosine phosphatase-2C inhibitor, Proto-oncogene Mas agonist, Purinoceptor antagonist, Raf protein kinase inhibitor, RANTES ligand, Ras gene inhibitor, Retinoate receptor responder protein 2 stimulator, Rev protein modulator, Ribonuclease stimulator, RIP-1 kinase inhibitor, RNA helicase inhibitor, RNA polymerase inhibitor, RNA polymerase modulator, S phase kinase associated protein 2 inhibitor, SARS coronavirus 3C protease like inhibitor, Serine protease inhibitor, Serine threonine protein kinase ATR inhibitor, Serine threonine protein kinase TBK1 inhibitor, Serum amyloid A protein modulator, Signal transducer CD24 stimulator, Sodium channel stimulator, Sodium glucose transporter-2 inhibitor, Sphingosine kinase 1 inhibitor, Sphingosine kinase 2 inhibitor, Sphingosine kinase inhibitor, Sphingosine-1-phosphate receptor-1 agonist, Sphingosine-1-phosphate receptor-1 antagonist, Sphingosine-1-phosphate receptor-1 modulator, Sphingosine-1-phosphate receptor-5 agonist, Sphingosine-1-phosphate receptor-5 modulator, Spike glycoprotein inhibitor, Src tyrosine kinase inhibitor, STAT-1 modulator, STAT-3 inhibitor, STAT-5 inhibitor, STAT3 gene inhibitor, Stem cell antigen-1 inhibitor, Stimulator of interferon genes protein stimulator, Sulfatase inhibitor, Superoxide dismutase modulator, Superoxide dismutase stimulator, Syk tyrosine kinase inhibitor, T cell immunoreceptor Ig ITIM protein inhibitor, T cell receptor agonist, T cell surface glycoprotein CD28 inhibitor, T-cell differentiation antigen CD6 inhibitor, T-cell surface glycoprotein CD8 stimulator, T-cell transcription factor NFAT modulator, Tankyrase-1 inhibitor, Tankyrase-2 inhibitor, Tek tyrosine kinase receptor stimulator, Telomerase modulator, Tetanus toxin modulator, TGF beta receptor antagonist, TGFB2 gene inhibitor, Thymosin beta 4 ligand, Thyroid hormone receptor beta agonist, Tissue factor inhibitor, Tissue plasminogen activator modulator, Tissue plasminogen activator stimulator, TLR agonist, TLR modulator, TLR-2 agonist, TLR-2 antagonist, TLR-3 agonist, TLR-4 agonist, TLR-4 antagonist, TLR-6 agonist, TLR-7 agonist, TLR-7 antagonist, TLR-8 antagonist, TLR-9 agonist, TMPRSS2 gene inhibitor, TNF alpha ligand inhibitor, TNF alpha ligand modulator, TNF binding agent, TNF gene inhibitor, Topoisomerase inhibitor, Transcription factor EB stimulator, Transferrin modulator, Transketolase inhibitor, Translocation associated protein inhibitor, Transmembrane serine protease 2 inhibitor, Transthyretin modulator, TREM receptor 1 antagonist, TRP cation channel C₁ modulator, TRP cation channel C₆ inhibitor, TRP cation channel V6 inhibitor, Trypsin 1 inhibitor, Trypsin 2 inhibitor, Trypsin 3 inhibitor, Trypsin inhibitor, Tubulin alpha inhibitor, Tubulin beta inhibitor, Tumor necrosis factor 14 ligand inhibitor, TYK2 gene inhibitor, Type I IL-1 receptor antagonist, Tyrosine protein kinase ABL1 inhibitor, Ubiquinol cytochrome C reductase 14 kDa inhibitor, Ubiquitin ligase modulator, Unspecified GPCR agonist, Unspecified cytokine receptor modulator, Unspecified enzyme stimulator, Unspecified gene inhibitor, Unspecified receptor modulator, Urokinase plasminogen activator inhibitor, Vascular cell adhesion protein 1 agonist, Vasodilator, VEGF ligand inhibitor, VEGF receptor antagonist, VEGF-1 receptor antagonist, VEGF-1 receptor modulator, VEGF-2 receptor antagonist, VEGF-3 receptor antagonist, Vimentin inhibitor, Vimentin modulator, VIP receptor agonist, Viral envelope protein inhibitor, Viral protease inhibitor, Viral protease modulator, Viral protein target modulator, Viral ribonuclease inhibitor, Viral structural protein modulator, Vitamin D3 receptor agonist, X-linked inhibitor of apoptosis protein inhibitor, Xanthine oxidase inhibitor, or Zonulin inhibitor.

In some embodiments, the compounds and compositions of the present disclosure may be administered in combination with a Sars-Cov-2 treatment, such as parenteral fluids (including dextrose saline and Ringer's lactate), nutrition, antibiotics (including azithromycin, metronidazole, amphotericin B, amoxicillin/clavulanate, trimethoprim/sulfamethoxazole, R-327 and cephalosporin antibiotics, such as ceftriaxone and cefuroxime), antifungal prophylaxis, fever and pain medication, antiemetic (such as metoclopramide) and/or antidiarrheal agents, vitamin and mineral supplements (including Vitamin K, vitamin D, cholecalciferol, vitamin C and zinc sulfate), anti-inflammatory agents (such as ibuprofen or steroids), corticosteroids such as dexamethasone, methylprednisolone, prednisone, mometasone, immunomodulatory medications (eg interferon), vaccines, and pain medications.

In some embodiments, the additional therapeutic agent is an Abl tyrosine kinase inhibitor, such as radotinib or imatinib.

In some embodiments, the additional therapeutic agent is an acetaldehyde dehydrogenase inhibitor, such as ADX-629.

In some embodiments, the additional therapeutic agent is an adenosine A3 receptor agonist, such as piclidenoson.

In some embodiments, the additional therapeutic agent is an adrenomedullin ligand such as adrenomedullin.

In some embodiments, the additional therapeutic agent is a p38 MAPK+PPAR gamma agonist/insulin sensitizer such as KIN-001.

In some embodiments, the additional therapeutic agent is an aldose reductase inhibitor, such as caficrestat.

In some embodiments, the additional therapeutic agent is an AMPA receptor modulator, such as traneurocin.

In some embodiments, the additional therapeutic agent is an annexin A5 stimulator, such as AP-01 or SY-005.

In some embodiments, the additional therapeutic agent is an anti-coagulant, such as heparins (heparin and low molecular weight heparin), aspirin, apixaban, dabigatran, edoxaban, argatroban, enoxaparin, or fondaparinux.

In some embodiments, the additional therapeutic agent is an androgen receptor antagonist such as bicalutamide, enzalutamide, or pruxelutamide (proxalutamide).

In some embodiments, the additional therapeutic agent is anti-hypoxic, such as trans-sodium crocetinate.

In some embodiments, the additional therapeutic agent is an anti-thrombotic, such as defibrotide, rivaroxaban, alteplase, tirofiban, clopidogrel, prasugrel, bemiparin, bivalirudin, sulodexide, or tenecteplase.

In some embodiments, the additional therapeutic agent is an antihistamine, such as cloroperastine or clemastine.

In some embodiments, the additional therapeutic agent is an apolipoprotein A1 agonist, such as CER-001.

In some embodiments, the additional therapeutic agent is a phospholipase A2 inhibitor, such as icosapent ethyl.

In some embodiments, the additional therapeutic agent is an axl tyrosine kinase receptor inhibitor, such as bemcentinib.

In some embodiments, the additional therapeutic agent is a corticosteroid/beta 2 adrenoceptor agonist, such as budesonide+formoterol fumarate.

In some embodiments, the additional therapeutic agent is a BET bromodomain inhibitor/APOA1 gene stimulator such as apabetalone.

In some embodiments, the additional therapeutic agent is a blood clotting modulator, such as lanadelumab.

In some embodiments, the additional therapeutic agent is a bradykinin B2 receptor antagonist, such as icatibant.

In some embodiments, the additional therapeutic agent is an EGFR gene inhibitor/Btk tyrosine kinase inhibitor, such as abivertinib.

In some embodiments, the additional therapeutic agent is a Btk tyrosine kinase inhibitor, such as ibrutinib or zanubrutinib.

In some embodiments, the additional therapeutic agent is a calpain-III/IX inhibitor, such as BLD-2660.

In some embodiments, the additional therapeutic agent is a Ca2+ release activated Ca2+ channel 1 inhibitor, such as zegocractin (CM-4620).

In some embodiments, the additional therapeutic agent is a cadherin-5 modulator, such as FX-06.

In some embodiments, the additional therapeutic agent is a casein kinase II inhibitor, such as silmitasertib.

In some embodiments, the additional therapeutic agent is a caspase inhibitor, such as emricasan.

In some embodiments, the additional therapeutic agent is a catalase stimulator/superoxide dismutase stimulator, such as MP-1032.

In some embodiments, the additional therapeutic agent is a CCR2 chemokine antagonist/CCR5 chemokine antagonist such as cenicriviroc.

In some embodiments, the additional therapeutic agent is a CCR5 chemokine antagonist, such as maraviroc.

In some embodiments, the additional therapeutic agent is a CD122 agonist/IL-2 receptor agonist, such as bempegaldesleukin.

In some embodiments, the additional therapeutic agent is a CD73 agonist/interferon beta ligand, such as FP-1201.

In some embodiments, the additional therapeutic agent is a cholesterol ester transfer protein inhibitor, such as dalcetrapib.

In some embodiments, the additional therapeutic agent is a Mannan-binding lectin serine protease/complement C₁s subcomponent inhibitor/myeloperoxidase inhibitor, such as RLS-0071.

In some embodiments, the additional therapeutic agent is a complement C₅ factor inhibitor/leukotriene BLT receptor antagonist, such as nomacopan.

In some embodiments, the additional therapeutic agent is a complement C₅ factor inhibitor, such as zilucoplan.

In some embodiments, the additional therapeutic agent is a CXCR4 chemokine antagonist, such as motixafortide.

In some embodiments, the additional therapeutic agent is a cytochrome P450 3A4 inhibitor/peptidyl-prolyl cis-trans isomerase A inhibitor, such as alisporivir.

In some embodiments, the additional therapeutic agent is a cysteine protease inhibitor, such as SLV-213.

In some embodiments, the additional therapeutic agent is a dihydroorotate dehydrogenase inhibitor, such as brequinar, RP-7214, or emvododstat.

In some embodiments, the additional therapeutic agent is a dehydropeptidase-1 modulator, such as Metablok.

In some embodiments, the additional therapeutic agent is a dihydroorotate dehydrogenase inhibitor/IL-17 antagonist, such as vidofludimus.

In some embodiments, the additional therapeutic agent is a diuretic, such as an aldosterone antagonist, such as spironolactone.

In some embodiments, the additional therapeutic agent is a deoxyribonuclease I stimulator, such as GNR-039 or dornase alfa.

In some embodiments, the additional therapeutic agent is a NET inhibitor, such as NTR-441.

In some embodiments, the additional therapeutic agent is a dihydroceramide delta 4 desaturase inhibitor/sphingosine kinase 2 inhibitor, such as opaganib.

In some embodiments, the additional therapeutic agent is a DNA methyltransferase inhibitor, such as azacytidine.

In some embodiments, the additional therapeutic agent is an LXR antagonist, such as larsucosterol.

In some embodiments, the additional therapeutic agent is a dipeptidyl peptidase I inhibitor, such as brensocatib.

In some embodiments, the additional therapeutic agent is an elongation factor 1 alpha 2 modulator, such as plitidepsin.

In some embodiments, the additional therapeutic agent is a eukaryotic initiation factor 4A-I inhibitor, such as zotatifin.

In some embodiments, the additional therapeutic agent is an exo-alpha sialidase modulator, such as DAS-181.

In some embodiments, the additional therapeutic agent is an exportin 1 inhibitor, such as selinexor.

In some embodiments, the additional therapeutic agent is a fractalkine ligand inhibitor, such as KAND-567.

In some embodiments, the additional therapeutic agent is a FYVE finger phosphoinositide kinase inhibitor/IL-12 receptor antagonist/IL-23 antagonist, such as apilimod dimesylate.

In some embodiments, the additional therapeutic agent is a GABA A receptor modulator, such as brexanolone.

In some embodiments, the additional therapeutic agent is a glucocorticoid receptor agonist, such as ciclesonide, hydrocortisone, dexamethasone, dexamethasone phosphate, or 101-PGC-005.

In some embodiments, the additional therapeutic agent is a GM-CSF receptor agonist, such as sargramostim.

In some embodiments, the additional therapeutic agent is a GPCR agonist, such as esuberaprost sodium.

In some embodiments, the additional therapeutic agent is a Griffithsin modulator, such as Q-Griffithsin.

In some embodiments, the additional therapeutic agent is a leukotriene D4 antagonist, such as montelukast.

In some embodiments, the additional therapeutic agent is a histamine H1 receptor antagonist, such as ebastine, tranilast, levocetirizine dihydrochloride.

In some embodiments, the additional therapeutic agent is a histamine H2 receptor antagonist, such as famotidine.

In some embodiments, the additional therapeutic agent is a heat shock protein stimulator/insulin sensitizer/PARP inhibitor, such as BGP-15.

In some embodiments, the additional therapeutic agent is a histone inhibitor, such as STC-3141.

In some embodiments, the additional therapeutic agent is a histone deacetylase-6 inhibitor, such as CKD-506.

In some embodiments, the additional therapeutic agent is a HIF prolyl hydroxylase-2 inhibitor, such as desidustat.

In some embodiments, the additional therapeutic agent is an HIF prolyl hydroxylase inhibitor, such as vadadustat.

In some embodiments, the additional therapeutic agent is an IL-8 receptor antagonist, such as reparixin.

In some embodiments, the additional therapeutic agent is an IL-7 receptor agonist, such as CYT-107.

In some embodiments, the additional therapeutic agent is an IL-7 receptor agonist/interleukin-7 ligand, such as efineptakin alfa.

In some embodiments, the additional therapeutic agent is an IL-22 agonist, such as efmarodocokin alfa.

In some embodiments, the additional therapeutic agent is an IL-22 agonist/interleukin 22 ligand, such as F-652.

In some embodiments, the additional therapeutic agent is an integrin alpha-V/beta-1 antagonist/integrin alpha-V/beta-6 antagonist, such as bexotegrast.

In some embodiments, the additional therapeutic agent is an interferon alpha 2 ligand, such as interferon alfa-2b or Virafin.

In some embodiments, the additional therapeutic agent is an interferon beta ligand, such as interferon beta-1a follow-on biologic, interferon beta-1b, or SNG-001.

In some embodiments, the additional therapeutic agent is an interferon receptor modulator, such as peginterferon lambda-1a.

In some embodiments, the additional therapeutic agent is an interleukin-2 ligand, such as aldesleukin.

In some embodiments, the additional therapeutic agent is an IRAK-4 protein kinase inhibitor, such as zimlovisertib.

In some embodiments, the additional therapeutic agent is a JAK inhibitor, for example the additional therapeutic agent is baricitinib, filgotinib, jaktinib, tofacitinib, or nezulcitinib (TD-0903).

In some embodiments, the additional therapeutic agent is a neutrophil elastase inhibitor, such as alvelestat.

In some embodiments, the additional therapeutic agent is a lung surfactant associated protein D modulator, such as AT-100.

In some embodiments, the additional therapeutic agent is a plasma kallikrein inhibitor, such as donidalorsen.

In some embodiments, the additional therapeutic agent is a lysine specific histone demethylase 1/MAO B inhibitor, such as vafidemstat.

In some embodiments, the additional therapeutic agent is a Mannan-binding lectin serine protease inhibitor, such as conestat alfa.

In some embodiments, the additional therapeutic agent is a maxi K potassium channel inhibitor, such as ENA-001.

In some embodiments, the additional therapeutic agent is a MEK protein kinase inhibitor, such as zapnometinib.

In some embodiments, the additional therapeutic agent is a MEK-1 protein kinase inhibitor/Ras gene inhibitor, such as antroquinonol.

In some embodiments, the additional therapeutic agent is a melanocortin MC1 receptor agonist, such as PL-8177.

In some embodiments, the additional therapeutic agent is a matrix metalloprotease-12 inhibitor, such as FP-025.

In some embodiments, the additional therapeutic agent is a NACHT LRR PYD domain protein 3 inhibitor, such as dapansutrile, DFV-890, or ZYIL-1.

In some embodiments, the additional therapeutic agent is a NADPH oxidase inhibitor, such as isuzinaxib.

In some embodiments, the additional therapeutic agent is a neuropilin 2 modulator, such as efzofitimod.

In some embodiments, the additional therapeutic agent is an NK1 receptor antagonist, such as aprepitant or tradipitant.

In some embodiments, the additional therapeutic agent is an NMDA receptor antagonist, such as transcrocetin or ifenprodil.

In some embodiments, the additional therapeutic agent is a nuclear factor kappa B inhibitor/p38 MAP kinase inhibitor, such as zenuzolac.

In some embodiments, the additional therapeutic agent is an ornithine decarboxylase inhibitor, such as eflornithine.

In some embodiments, the additional therapeutic agent is an opioid receptor sigma antagonist 1, such as MR-309.

In some embodiments, the additional therapeutic agent is a PGD2 antagonist, such as asapiprant.

In some embodiments, the additional therapeutic agent is a PDGF receptor antagonist/TGF beta receptor antagonist/p38 MAP kinase inhibitor, such as deupirfenidone.

In some embodiments, the additional therapeutic agent is a phospholipase A2 inhibitor, such as varespladib methyl.

In some embodiments, the additional therapeutic agent is a phosphoinositide 3-kinase inhibitor/mTOR complex inhibitor, such as dactolisib.

In some embodiments, the additional therapeutic agent is a phosphoinositide-3 kinase delta/gamma inhibitor, such as duvelisib.

In some embodiments, the additional therapeutic agent is a plasminogen activator inhibitor 1 inhibitor, such as TM-5614.

In some embodiments, the additional therapeutic agent is a protein tyrosine phosphatase beta inhibitor, such as razuprotafib.

In some embodiments, the additional therapeutic agent is a RIP-1 kinase inhibitor, such as DNL-758 or SIR-0365.

In some embodiments, the additional therapeutic agent is a Rev protein modulator, such as obefazimod.

In some embodiments, the additional therapeutic agent is an S phase kinase associated protein 2 inhibitor, such as niclosamide or DWRX-2003.

In some embodiments, the additional therapeutic agent is a signal transducer CD24 stimulator, such as EXO-CD24.

In some embodiments, the additional therapeutic agent is a sodium glucose transporter-2 inhibitor, such as dapagliflozin propanediol.

In some embodiments, the additional therapeutic agent is a sodium channel stimulator, such as solnatide.

In some embodiments, the additional therapeutic agent is a sphingosine-1-phosphate receptor-1 agonist/sphingosine-1-phosphate receptor-5 agonist, such as ozanimod.

In some embodiments, the additional therapeutic agent is a non-steroidal anti-inflammatory drug, such as Ampion.

In some embodiments, the additional therapeutic agent is a superoxide dismutase stimulator, such as avasopasem manganese.

In some embodiments, the additional therapeutic agent is a Syk tyrosine kinase inhibitor, such as fostamatinib disodium.

In some embodiments, the additional therapeutic agent is a Tie2 tyrosine kinase receptor agonist, such as AV-001.

In some embodiments, the additional therapeutic agent is a TGFB2 gene inhibitor, such as trabedersen.

In some embodiments, the additional therapeutic agent is a tissue factor inhibitor, such as AB-201.

In some embodiments, the additional therapeutic agent is a TLR-3 agonist, such as rintatolimod.

In some embodiments, the additional therapeutic agent is a TLR-4 antagonist, such as ApTLR-4FT, EB-05, or eritoran.

In some embodiments, the additional therapeutic agent is a TLR-7/8 antagonist, such as enpatoran.

In some embodiments, the additional therapeutic agent is a TLR-2/6 agonist, such as INNA-051.

In some embodiments, the additional therapeutic agent is a TLR-7 agonist, such as PRTX-007.

In some embodiments, the additional therapeutic agent is a TLR agonist, such as PUL-042.

In some embodiments, the additional therapeutic agent is a TLR-4 agonist, such as REVTx-99.

In some embodiments, the additional therapeutic agent is a TLR-2/4 antagonist, such as VB-201.

In some embodiments, the additional therapeutic agent is a TNF alpha ligand inhibitor, such as pegipanermin.

In some embodiments, the additional therapeutic agent is a type I IL-1 receptor antagonist, such as anakinra.

In some embodiments, the additional therapeutic agent is a TREM receptor 1 antagonist, such as nangibotide.

In some embodiments, the additional therapeutic agent is a trypsin inhibitor, such as ulinastatin.

In some embodiments, the additional therapeutic agent is a tubulin inhibitor such as sabizabulin, CCI-001, PCNT-13, CR-42-24, albendazole, entasobulin, SAR-132885, or ON-24160.

In some embodiments, the additional therapeutic agent is a VIP receptor agonist, such as aviptadil.

In some embodiments, the additional therapeutic agent is a xanthine oxidase inhibitor, such as oxypurinol.

In some embodiments, the additional therapeutic agent is a vasodilator, such as iloprost, epoprostenol (VentaProst), zavegepant, TXA-127, USB-002, ambrisentan, nitric oxide nasal spray (NORS), pentoxifylline, propranolol, RESP301, sodium nitrite, or dipyridamole.

In some embodiments, the additional therapeutic agent is a vitamin D3 receptor agonist, such as cholecalciferol.

In some embodiments, the additional therapeutic agent is a zonulin inhibitor, such as larazotide acetate.

In some embodiments, the additional therapeutic agent is a synthetic retinoid derivative, such as fenretinide.

In some embodiments, the additional therapeutic agent is a glucose metabolism inhibitor such as WP-1122.

In some embodiments, the additional therapeutic agent is AT-H201, 2-deoxy-D-glucose, AD-17002, AIC₁₋₆₄₉, astodrimer, AZD-1656, bitespiramycin, bucillamine, budesonide, CNM-AgZn-17, Codivir, didodecyl methotrexate, DW-2008S (DW-2008), EDP-1815, EG-009A, Fabencov, Gamunex, genistein, GLS-1200, hzVSF-v13, imidazolyl ethanamide pentandioic acid, IMM-101, MAS-825, MRG-001, Nasitrol, Nylexa, OP-101, OPN-019, Orynotide rhesus theta defensin-1, pyronaridine+ artesunate, dapsone, RPH-104, sodium pyruvate, Sulforadex, tafenoquine, TB-006, telacebec, Tempol, TL-895, thimesoral, trimodulin, XC-221, XC-7, zunsemetinib, metformin glycinate, lucinactant, EOM-613, mosedipimod, ivermectin, leflunomide, ibudilast, RBT-9, raloxifene, prothione, gemcabene, or idronoxil.

In some embodiments, the additional therapeutic agent is a CD73 antagonist, such as AK-119.

In some embodiments, the additional therapeutic agent is a CD95 protein fusion, such as asunercept.

In some embodiments, the additional therapeutic agent is a complement factor C₂ modulator, such as ARGX-117.

In some embodiments, the additional therapeutic agent is a complement C₃ inhibitor, such as NGM-621.

In some embodiments, the additional therapeutic agent is a CXC10 chemokine ligand inhibitor, such as EB-06.

In some embodiments, the additional therapeutic agent is a cytotoxic T-lymphocyte protein-4 fusion protein, such as abatacept.

In some embodiments, the additional therapeutic agent is an anti-S. Aureus antibody, such as tosatoxumab.

In some embodiments, the additional therapeutic agent is an anti-LPS antibody, such as IM-124-E.

In some embodiments, the additional therapeutic agent is an adrenomedullin ligand inhibitor, such as enibarcimab.

In some embodiments, the additional therapeutic agent is a basigin inhibitor, such as meplazumab.

In some embodiments, the additional therapeutic agent is a CD3 antagonist, such as foralumab.

In some embodiments, the additional therapeutic agent is a connective tissue growth factor ligand inhibitor, such as pamrevlumab.

In some embodiments, the additional therapeutic agent is a complement C₅a factor inhibitor, such as BDB-1 or vilobelimab.

In some embodiments, the additional therapeutic agent is a complement C₅ factor inhibitor, such as ravulizumab.

In some embodiments, the additional therapeutic agent is a mannan-binding lectin serine protease-2 inhibitor, such as narsoplimab.

In some embodiments, the additional therapeutic agent is a GM-CSF modulator, such as gimsilumab, namilumab, plonmarlimab, otolimab, or lenzilumab.

In some embodiments, the additional therapeutic agent is a heat shock protein inhibitor/IL-6 receptor antagonist, such as siltuximab.

In some embodiments, the additional therapeutic agent is an IL-6 receptor antagonist, such as clazakizumab, levilimab, olokizumab, tocilizumab, or sirukumab.

In some embodiments, the additional therapeutic agent is an IL-8 receptor antagonist, such as BMS-986253.

In some embodiments, the additional therapeutic agent is an interleukin-1 beta ligand inhibitor, such as canakinumab.

In some embodiments, the additional therapeutic agent is an interferon gamma ligand inhibitor, such as emapalumab.

In some embodiments, the additional therapeutic agent is an anti-ILT7 antibody, such as daxdilimab.

In some embodiments, the additional therapeutic agent is a monocyte differentiation antigen CD14 inhibitor, such as atibuclimab.

In some embodiments, the additional therapeutic agent is a plasma kallikrein inhibitor, such as lanadelumab.

In some embodiments, the additional therapeutic agent is a platelet glycoprotein VI inhibitor, such as glenzocimab.

In some embodiments, the additional therapeutic agent is a T-cell differentiation antigen CD6 inhibitor, such as itolizumab.

In some embodiments, the additional therapeutic agent is a TNF alpha ligand inhibitor/TNF binding agent, such as infliximab.

In some embodiments, the additional therapeutic agent is an anti-LIGHT antibody, such as AVTX-002.

In some embodiments, the additional therapeutic agent is COVID-HIG.

In some embodiments, a compound of the disclosure, or a pharmaceutically acceptable salt thereof, is co-administered with one or more agents useful for the treatment and/or prophylaxis of COVID-19.

Non-limiting examples of such agents include corticosteroids, such as dexamethasone, hydrocortisone, methylprednisolone, or prednisone; interleukin-6 (IL-6) receptor blockers, such as tocilizumab or sarilumab; Janus kinase (JAK) inhibitors, such as baricitinib, ruxolitinib, or tofacitinib; and antiviral agents, such as molnupiravir, sotrovimab, or remdesivir.

In further embodiments, a compound of the disclosure, or a pharmaceutically acceptable salt thereof, is co-administered with two or more agents useful for the treatment of COVID-19. Agents useful for the treatment and/or prophylaxis of COVID-19 include but are not limited to a compound of the disclosure and two additional therapeutic agents, such as nirmatrelvir and ritonavir, casirivimab and imdevimab, or ruxolitinib and tofacitinib.

In some embodiments, the additional therapeutic agent is an antiviral agent. In some embodiments, the antiviral agent is an entry inhibitor. In some embodiments, the antiviral agent is a protease inhibitor. In some embodiments, the antiviral agent is an RNA polymerase inhibitor. In some embodiments, the additional therapeutic agent is a RNA-dependent RNA polymerase (RdRp) inhibitor.

In some embodiments, the antiviral agent is selected from angiotensin converting enzyme 2 inhibitors, angiotensin converting enzyme 2 modulators, angiotensin converting enzyme 2 stimulators, angiotensin II AT-2 receptor agonists, angiotensin II AT-2 receptor antagonists, angiotensin II receptor modulators, coronavirus nucleoprotein modulators, coronavirus small envelope protein modulators, coronavirus spike glycoprotein inhibitors, coronavirus spike glycoprotein modulators, COVID19 envelope small membrane protein inhibitors, COVID19 envelope small membrane protein modulators, COVID19 MPro inhibitors, COVID19 non structural protein 8 modulators, COVID19 nucleoprotein inhibitors, COVID19 nucleoprotein modulators, COVID19 protein 3a inhibitors, COVID19 replicase polyprotein 1a inhibitors, COVID19 replicase polyprotein 1a modulators, COVID19 replicase polyprotein 1ab inhibitors, COVID19 replicase polyprotein 1ab modulators, COVID19 spike glycoprotein inhibitors, COVID19 spike glycoprotein modulators, COVID19 structural glycoprotein modulators, papain inhibitors, protease inhibitors, protease modulators, RNA polymerase inhibitors, RNA polymerase modulators, RNA-dependent RNA polymerase (RdRp) inhibitors, SARS coronavirus 3C protease like inhibitors, 3 CLpro/Mpro inhibitors, serine protease inhibitors, transmembrane serine protease 2 inhibitors, transmembrane serine protease 2 modulators, viral envelope protein inhibitors, viral protease inhibitors, viral protease modulators, viral protein target modulators, viral ribonuclease inhibitors, and viral structural protein modulators.

In some embodiments, the additional therapeutic agent is an entry inhibitor. For example, in some embodiments the additional therapeutic agent is an ACE2 inhibitor, a fusion inhibitor, or a protease inhibitor.

In some embodiments, the additional therapeutic agent is an angiotensin converting enzyme 2 inhibitor, such as SBK-001.

In some embodiments, the additional therapeutic agent is an angiotensin converting enzyme 2 modulator, such as neumifil or JN-2019.

In some embodiments, the additional therapeutic agent is an entry inhibitor such as MU-UNMC-1.

In some embodiments, the additional therapeutic agent is an angiotensin converting enzyme 2 stimulator, such as alunacedase alfa.

In some embodiments, the additional therapeutic agent is an angiotensin II AT-2 receptor agonist, such as VP-01.

In some embodiments, the additional therapeutic agent is an ACE II receptor antagonist, such as DX-600.

In some embodiments, the additional therapeutic agent is an angiotensin II receptor modulator, such as TXA-127.

In some embodiments, the additional therapeutic agent is a transmembrane serine protease 2 modulator, such as BC-201.

In some embodiments, the additional therapeutic agent is a viral envelope protein inhibitor, such as MXB-9 or MXB-004.

In some embodiments, the additional therapeutic agent is a vaccine. For example, in some embodiments, the additional therapeutic agent is a DNA vaccine, RNA vaccine, live-attenuated vaccine, inactivated vaccine (i.e., inactivated SARS-CoV-2 vaccine), therapeutic vaccine, prophylactic vaccine, protein-based vaccine, viral vector vaccine, cellular vaccine, or dendritic cell vaccine.

In some embodiments, the additional therapeutic agent is a vaccine such as tozinameran, NVX—CoV2373, elasomeran, KD-414, Janssen COVID-19 Vaccine, Vaxzevria, SCB-2019, AKS-452, VLA-2001, 5-268019, MVC-COV1901, mRNA-1273.214, NVX—CoV2515, Covaxin, BBIBP-CorV, GBP-510, mRNA-1273.351+mRNA-1273.617 (SARS-CoV-2 multivalent mRNA vaccine, COVID-19), Ad5-nCoV, Omicron-based COVID-19 vaccine (mRNA vaccine, COVID-19), SARS-CoV-2 Protein Subunit Recombinant Vaccine, Sputnik M, ZyCoV-D, COVID-19 XWG-03, mRNA-1273.529, mRNA-1010, CoronaVac, AZD-2816, Sputnik V, inactivated SARS-CoV-2 vaccine (Vero cell, COVID-19), DS-5670, PHH-1V, INO-4800, UB-612, coronavirus vaccine (whole-virion, inactivated/purified), ReCOV, MT-2766, ARCT-154, SP-0253, CORBEVAX, mRNA-1273.211, ZF-2001, Sputnik Light, recombinant protein vaccine (COVID-19/SARS-CoV-2 infection), VSV vector-based vaccine targeting spike glycoprotein (COVID-19), VLA-2101, GRAd-COV2, VPM-1002, COViran Barekat, Ad5-nCoV-IH, ARCoV, Covax-19, recombinant SARS-CoV-2 vaccine (protein subunit/CHO cell, COVID-19), BBV-154, RAZI Cov Pars, COVID-19 vaccine (inactivated/Vero cells/intramuscular, SARS-CoV-2 infection), COVID-19 vaccine (inactivated, Vero cells/intramuscular), BNT-162b2s01, CIGB-66, mRNA-1273.617, Mycobacterium w, ERUCOV-VAC, AG-0301-COVID19, fakhravac, AV-COVID-19, peptide vaccine (COVID-19), Nanocovax, SARS-CoV-2 vaccine (inactivated/Vero cells/intramuscular, COVID-19), QAZCOVID-IN, 5-875670 nasal vaccine, or BNT162b5.

In some embodiments, the additional therapeutic agent is a protease inhibitor. For example, in some embodiments the additional therapeutic agent is a 3C-like cysteine protease inhibitor (3 CLpro, also called Main protease, Mpro), a papain-like protease inhibitor (PLpro), serine protease inhibitor, or transmembrane serine protease 2 inhibitor (TMPRSS2).

In some embodiments, the additional therapeutic agent is a 3 CLpro/Mpro inhibitor, such as CDI-873, GC-373, GC-376, PBI-0451, UCI-1, DC-402234, DC-402267, RAY-1216, MPI-8, SH-879, SH-580, EDP-235, VV-993, CDI-988, MI-30, nirmatrelvir, ensitrelvir, ASC-11, EDDC-2214, SIM-0417, CDI-45205, COR-803, ALG-097111, TJC₁₋₆₄₂, CVD-0013943, eravacycline, cynarine, or prexasertib.

In some embodiments, the additional therapeutic agent is a papain-like protease inhibitor (PLpro), such as SBFM-PL4 or GRL-0617.

In some embodiments, the additional therapeutic agent is a SARS-CoV-2 helicase Nsp13 inhibitor, such as EIS-4363.

In some embodiments, the additional therapeutic agent is a SARS-CoV-2 spike (S) and protease modulator, such as ENU-200.

In some embodiments, the additional therapeutic agent is a protease inhibitor, such as ALG-097558 or MRX-18.

In some embodiments, the additional therapeutic agent is a serine protease inhibitor, such as upamostat, nafamostat, camostat mesylate, nafamostat mesylate, or camostat.

In some embodiments, the additional therapeutic agent is a 3 CLpro/transmembrane serine protease 2 inhibitor, such as SNB-01 or SNB-02.

In some embodiments, the additional therapeutic agent is a viral protease inhibitor, such as Pan-Corona, Cov-X, or bepridil.

In some embodiments, the additional therapeutic agent is an RNA polymerase inhibitor. For example, in some embodiments, the additional therapeutic agent is an RNA polymerase inhibitor, or a RNA-dependent RNA polymerase (RdRp) inhibitor.

In some embodiments, the additional therapeutic agent is an RNA-dependent RNA polymerase (RdRp) inhibitor, such as remdesivir, NV—CoV-2-R, NV—CoV-1 encapsulated remdesivir, GS-621763, GS-5245, GS-441524, DEP remdesivir, ATV-006, VV-116, LGN-20, CMX-521 and compounds disclosed in WO2022142477, WO2021213288, WO2022047065.

In some embodiments, the additional therapeutic agent is an RNA polymerase inhibitor, such as molnupiravir (EIDD-2801), favipiravir, bemnifosbuvir, sofosbuvir, ASC-10, or galidesivir.

In some embodiments, the additional therapeutic agent is viral entry inhibitor, such as brilacidin.

In some embodiments, the additional therapeutic agent is an antibody that binds to a coronavirus, for example an antibody that binds to SARS or MERS.

In some embodiments, the additional therapeutic agent is an antibody, for example a monoclonal antibody. For example, the additional therapeutic agent is an antibody against SARS-CoV-2, neutralizing nanobodies, antibodies that target the SARS-CoV-2 spike protein, fusion proteins, multispecific antibodies, and antibodies that can neutralize SARS-CoV-2 (SARS-CoV-2 neutralizing antibodies).

In some embodiments, the additional therapeutic agent is an antibody that targets specific sites on ACE2. In some embodiments, the additional therapeutic agent is a polypeptide targeting SARS-CoV-2 spike protein (S-protein).

In some embodiments, the additional therapeutic agent is a SARS-CoV-2 virus antibody.

In some embodiments, the antibody is ABBV-47D11, COVI-GUARD (STI-1499), C144-LS+C135-LS, DXP-604, JMB-2002, LY-CovMab, bamlanivimab (LY-CoV555), S309, SAB-185, etesevimab (CB6), COR-101, JS016, VNAR, VIR-7832 and/or sotrovimab (VIR-7831), casirivimab+imdevimab (REGN-COV2 or REGN10933+RGN10987), BAT2020, BAT2019, 47D11, YBSW-015, or PA-001.

In some embodiments, the additional therapeutic agent is STI-9199 (COVI-SHIELD) or AR-701 (AR-703 and AR-720).

In some embodiments, the additional therapeutic agent is BRII-196, BRII-198, ADG-10, ADG-20, ABP-300, BI-767551, CT-P63, JS-026, sotrovimab (GSK-4182136), tixagevimab+cilgavimab (AZD-7442), regdanvimab, SAB-301, AOD-01, plutavimab (COVI-AMG), 9MW-3311 (MW-33), DXP-593, BSVEQAb, anti-SARS-CoV-2 IgY, COVID-EIG, CSL-760, REGN-3048-3051, SARS-CoV-2 monoclonal antibodies (COVID-19, ADM-03820), enuzovimab (HFB-30132A), INM-005, SCTA01, TY-027, XAV-19, amubarvimab+romlusevimab, SCTA-01, bebtelovimab, beludavimab, IBI-0123, IGM-6268. FYB-207, REGN-14256, XVR-011, TB202-3, TB181-36, LQ-050, COVAB-36, MAD-0004J08, STI-2099, or ACV-200-17.

In some embodiments, the additional therapeutic agent is an engineered ACE-2-IgG1-Fc-fusion protein targeting SARS-Cov-2 RBD, such as EU-129, bivalent ACE2-IgG Fc null fusion protein (SI—F019).

In some embodiments, the additional therapeutic agent is an ACE2-Fc receptor fusion protein, such as HLX-71.

In some embodiments, the additional therapeutic agent is ensovibep.

In some embodiments, the additional therapeutic agent is SYZJ-001.

In some embodiments, the additional therapeutic agent is an HIV-1 protease inhibitor, such as ASC-09F (ASC-09+ritonavir) or lopinavir+ritonavir.

In some embodiments, the additional therapeutic agent is a non-nucleoside reverse transcriptase inhibitor, such as elsulfavirine.

In some embodiments, the additional therapeutic agent is a nucleoside reverse transcriptase inhibitor, such as azvudine.

In some embodiments, the additional therapeutic agent is Abbv-990, NED-260, ALG-097431, ENOB-CV-01, EIS-10700, beta-521, SIM-0417, molnupiravir, Pan-Corona, Tollovir, nirmatrelvir+ritonavir (Paxlovid®), favipiravir, GC-376, upamostat, LeSoleil-01, LeSoleil-02+, benfovir, VV-116, VV-993, SNB-01, EDP-235, Cov-X, ensitrelvir, MPI-8, masitinib, ALG-097558, ASC-11, PBI-0451, nafamostat, nafamostat mesylate, CDI-45205, COR-803, ALG-097111, BC-201, SH-879, CDI-873, CDI-988, remdesivir, NV—CoV-2-R, NV—CoV-1 encapsulated remdesivir, NA-831+remdesivir, DEP remdesivir, GS-621763, GS-5245, GLS-5310, bemnifosbuvir, QLS-1128, ASC-10, SBFM-PL4, camostat mesylate, UCI-1, DC-402234, ebselen, SH-580, LeSoleil-01, LeSoleil-02+, MRX-18, MXB-9, MI-09, MI-30, SNB-02, TJC₁₋₆₄₂, ENU-200, CVD-0013943, GS-441524, bepridil, MXB-004, eravacycline, GRL-0617, camostat, GC-373, nitazoxanide, cynarine, prexasertib, RAY-1216, SACT-COVID-19, MP-18, EIDD-1931, EDDC-2214, nitric oxide, apabetalone, AnQlar, SBK-001, LQ-050, CG-SpikeDown, bamlanivimab, HLX-71, FYB-207, ensovibep, SYZJ-001, EU-129, neumifil, JN-2019, AR-701, vostesyl, PLM-402, PJS-539, CTB-ACE2, TB181-36, TB202-3, ABP-300, XVR-011, MU-UNMC-1, MU-UNMC-2, alunacedase alfa, VP-01, TRV-027, DX-600, TXA-127, mRNA-1273.214, Omicron-based COVID-19 vaccine, NVX—CoV2515, tozinameran, elasomeran, Ad5-nCoV, BBIBP-CorV, CoronaVac, MVC-COV1901, NVX—CoV2373, sotrovimab, Sputnik V, Vaxzevria, ZF-2001, or ZyCoV-D.

It is also possible to combine any compound of the disclosure with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the disclosure with one or more other active therapeutic agents generally refers to simultaneous or sequential administration of a compound of the disclosure and one or more other active therapeutic agents, such that therapeutically effective amounts of the compound of the disclosure and one or more other active therapeutic agents are both present in the body of the patient.

Co-administration includes administration of unit dosages of the compounds of the disclosure before or after administration of unit dosages of one or more other active therapeutic agents, for example, administration of the compounds of the disclosure within seconds, minutes, or hours of the administration of one or more other active therapeutic agents. For example, a unit dose of a compound of the disclosure can be administered first, followed within seconds or minutes by administration of a unit dose of one or more other active therapeutic agents. Alternatively, a unit dose of one or more other therapeutic agents can be administered first, followed by administration of a unit dose of a compound of the disclosure within seconds or minutes. In some cases, it may be desirable to administer a unit dose of a compound of the disclosure first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the disclosure.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect, which is greater than the predicted purely additive effects of the individual compounds of the combination.

### Kits

Also provided herein are kits that includes a compound disclosed herein, a pharmaceutically acceptable salt, stereoisomer, mixture of stereoisomers or tautomer thereof. In some embodiments the kits described herein may comprise a label and/or instructions for use of the compound in the treatment of a disease or condition in a subject (e.g., human) in need thereof. In some embodiments, the disease or condition is viral infection.

In some embodiments, the kit may also comprise one or more additional therapeutic agents and/or instructions for use of additional therapeutic agents in combination with the compound described herein in the treatment of the disease or condition in a subject (e.g., human) in need thereof.

In some embodiments, the kits provided herein comprises individual dose units of a compound as described herein, or a pharmaceutically acceptable salt, racemate, enantiomer, diastereomer, tautomer, polymorph, pseudopolymorph, amorphous form, hydrate or solvate thereof. Examples of individual dosage units may include pills, tablets, capsules, prefilled syringes or syringe cartridges, IV bags, inhalers, nebulizers etc., each comprising a therapeutically effective amount of the compound in question, or a pharmaceutically acceptable salt, racemate, enantiomer, diastereomer, tautomer, polymorph, pseudopolymorph, amorphous form, hydrate or solvate thereof. In some embodiments, the kit may contain a single dosage unit and in others, multiple dosage units are present, such as the number of dosage units required for a specified regimen or period.

Also provided are articles of manufacture that include a compound described herein, or a pharmaceutically acceptable salt, stereoisomer, mixture of stereoisomers or tautomer thereof, and a container. In some embodiments, the container of the article of manufacture is a vial, jar, ampoule, preloaded syringe, blister package, tin, can, bottle, box, an intravenous bag, an inhaler, or a nebulizer.

The invention will be described in greater detail by way of specific examples. The following examples are offered for illustrative purposes and are not intended to limit the invention in any manner. Those of skill in the art will readily recognize a variety of non-critical parameters, which can be changed or modified to yield essentially the same results.

### EXAMPLES

Certain abbreviations and acronyms are used in describing the experimental details. Although most of these would be understood by one skilled in the art, Table 1 contains a list of many of these abbreviations and acronyms.

TABLE 1
List of Abbreviations and Acronyms

| Abbreviation | Meaning |
|---|---|
| Ac | acetate |
| AcOH | acetic acid |
| ACN | acetonitrile |
| AmPhos | di-tert-butyl(4-dimethylaminophenyl)phosphine |
| AQ | aqueous |
| Bn | benzyl |
| Bpin | (pinacolato)boron |
| B2Pin2 | bis(pinacolato)diboron |
| Boc | tert-butoxycarbonyl |
| Boc₂O | di-tert-butyl dicabonate |
| Bu | Butyl |
| Bz | benzoyl |
| BzCl | benzoyl chloride |
| cataCXium ® | Mesylate[(di(1-adamantyl)-n- |
| A Pd G3 | butylphosphine)-2-(2′-amino-1,1′- |
| | biphenyl)]palladium(II) |
| CDI | 1,1′-carbonyldiimidazole |
| DBAD | di-tert-butyl azodicarboxylate |
| DBU | 1,8-Diazabicyclo[5.4.0]undec-7-ene |
| DCE | 1,2-dichloroethane |
| DCM | dichloromethane |
| DEA | diethylamine |
| Deoxofluor | Bis(2-methoxyethyl)aminosulfur trifluoride |
| DIPEA | N,N-diisopropylethylamine |
| DIAD | diisopropyl azodicarboxylate |
| DMA | dimethylacetamide |
| 4-DMAP, DMAP | 4-dimethylaminopyridine |
| DME | dimethoxyethane |
| DMF | dimethylformamide |
| DMSO | dimethylsulfoxide |
| dppf | 1,1′-Ferrocenediy1-bis(diphenylphosphine) |
| dtbbpy | 4,4′-Di-tert-butyl-2,2′-dipyridyl |
| EDC | N-(3-dimethylaminopropyl)-N′- |
| | ethylcarbodiimide hydrochloride |
| EDCI | N-(3-dimethylaminopropyl)-N′- |
| | ethylcarbodiimide hydrochloride |
| ES/MS | electron spray mass spectrometry |
| Et | ethyl |
| HATU | 1-[Bis(dimethylamino)methylene]-1H-1,2,3- |
| | triazolo[4,5-b]pyridinium 3- oxide |
| | hexafluorophosphate |
| HEX | hexanes |
| HNMR | hydrogen nuclear magnetic resonance |
| IBX | 2-Iodoxybenzoic acid |
| IPA | isopropanol |
| JohnPhos | (2-Biphenyl)di-tert-butylphosphine |
| KOtBu | potassium tert-butoxide |
| LC | liquid chromatography |
| LCMS | liquid chromatography/mass spectrometry |
| MCPBA | meta-chloroperbenzoic acid |
| Me | methyl |
| Ms | methanesulfonyl |
| m/z | mass to charge ratio |
| MS or ms | mass spectrum |
| MW | microwave |
| NBS | N-bromosuccinimide |
| NCS | N-chlorosuccinimide |
| NIS | N-iodosuccinimide |
| NMP | N-methyl-2-pyrrolidone |
| NMR | nuclear magnetic resonance |
| Pd(AmPhos)₂Cl₂ | Bis(di-tert-butyl(4- |
| | dimethylaminophenyl)pho- |
| | sphine)dichloropalladium(II) |
| Pd(dppf)Cl₂ | [1,1′-Bis(diphenylphosphino)ferrocene]di- |
| | chloropalladium(II) |
| Ph | phenyl |
| Ph3P | triphenylphosphine |
| Pg | Protecting group |
| pin | pinacol |
| Piv | pivaloyl |
| PMB | para-methoxybenzyl |
| PPT | precipitate |
| Pyr | pyridine |
| RBF | round bottom flask |
| RP-HPLC | reverse phase high performance |
| | liquid chromatography |
| RT | room temperature |
| SAT | saturated |
| SEM | [2-(trimethylsilyl)ethoxy]methyl |
| SFC | supercritical fluid chromatography |
| STAB | Sodium triacetoxyborohydride |
| TLC | thin layer chromatography |
| tBuXPhos Pd G3 | [(2-Di-tert-butylphosphino-2′,4′,6′- |
| | triisopropyl-1,1′-biphenyl)-2- |
| | (2′- amino-1,1′-biphenyl)] |
| | palladium(II) methanesulfonate |
| XantPhos | 4,5-Bis(diphenylphosphino)-9,9-dimethylxanthene |
| XPhos Pd G2 | Chloro(2-dicyclohexylphosphino- |
| | 2′,4′,6′-triisopropyl-1,1′-biphenyl)[2- |
| | (2′-amino-1,1′-biphenyl)]palladium(II) |
| XPhos Pd G3 | (2-Dicyclohexylphosphino-2′,4′,6′- |
| | triisopropyl-1,1′-biphenyl)[2-(2′- |
| | amino-1,1′-biphenyl)]palladium(II) methanesulfonate |
| T3P | Propanephosphonic acid anhydride |
| TBAF | Tetrabutylammonium fluoride |
| TBAI | Tetrabutylammonium iodide |
| TCFH | Chloro-N,N,N′,N′-tetramethylformamidinium |
| | hexafluorophosphate |
| TEA | triethylamine |
| TFA | trifluoroacetic acid |
| THF | tetrahydrofuran |
| Tf | trifluoromethanesulfonyl |
| Tf₂O | trifluoromethanesulfonic anhydride |
| TMEDA | tetramethylethylenediamine |
| TMS | tetramethylsilyl |
| Ts | 4-toluenesulfonyl |
| δ | parts per million referenced to residual solvent peak |

### I. Intermediates

### Intermediate I-1

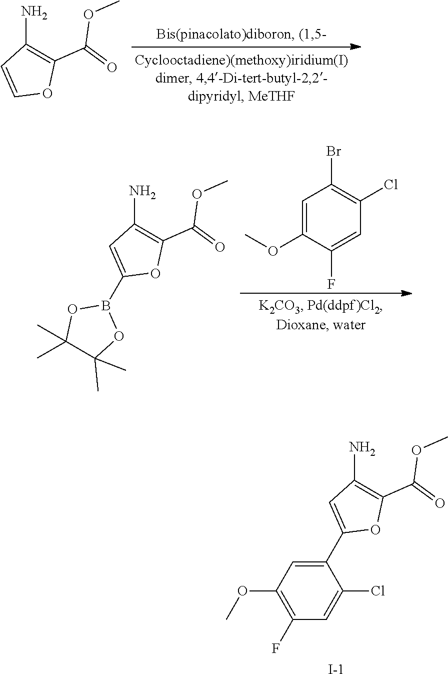

Methyl 3-amino-5-(4,4,5,5-tetramethyl-1,3,2-dioxaborolan-2-yl)furan-2-carboxylate: To a microwave vial was added methyl 3-amino-5-bromofuran-2-carboxylate (500 mg, 3.5 mmol), bis(pinacolato)diboron (630 mg, 2.5 mmol) 4,4′-di-tert-butyl-2,2′-dipyridyl (9.5 mg, 0.04 mmol), and (1,5-cyclooctadiene)(methoxy)iridium(I) dimer (12 mg, 0.02 mmol). The solids were suspended in Me-THf (10 mL). The mixture was sparged with argon, sealed and heated to 80° C. overnight. The mixture was cooled to rt, filtered over celite, rinsing with Me-THf, and concentrated under reduced pressure to provide a residue. The residue was carried on to the next step without further purification.

ES/MS: 268.2 (M⁺).

Methyl 3-amino-5-(2-chloro-4-fluoro-5-methoxyphenyl)furan-2-carboxylate (I-1): To a stirring solution of methyl 3-amino-5-(4,4,5,5-tetramethyl-1,3,2-dioxaborolan-2-yl)furan-2-carboxylate (1.6 g, 6 mmol) in 1,4-dioxane (10 mL) and water (3 mL) was added 1-bromo-2-chloro-4-fluoro-5-methoxy-benzene (1.4 g, 6 mmol), K₂CO₃ (2.5 g, 18 mmol), and 1,1′-bis(diphenylphosphino)ferrocene-palladium(II)dichloride dichloromethane complex (248 mg, 0.30 mmol). The mixture was sparged with argon, fitted with a reflux condenser, and heated to 100° C. under N₂ overnight. The mixture was cooled to rt, filtered over celite, and extracted with EtOAc (2×). The mixture was concentrated and purified by flash column chromatograph to obtain methyl 3-amino-5-(2-chloro-4-fluoro-5-methoxyphenyl)furan-2-carboxylate I-1.

ES/MS: 300.2 (M⁺)

### Intermediate I-2

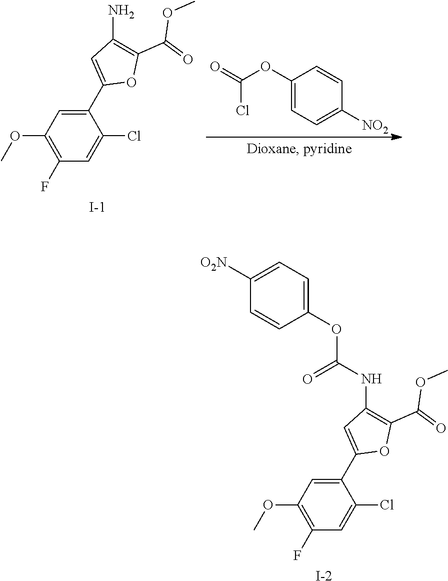

Methyl 5-(2-chloro-4-fluoro-5-methoxyphenyl)-3-(((4-nitrophenoxy)carbonyl)amino)furan-2-carboxylate (I-2): To a solution of methyl 3-amino-5-(2-chloro-4-fluoro-5-methoxyphenyl)furan-2-carboxylate I-1 (200 mg, 0.67 mmol) in 1,4-dioxane (6.3 mL) placed in a RT water bath was added 4-nitrophenyl chloroformate (221 mg, 1.1 mmol, dissolved in 1 mL acetonitrile). The mixture was stirred for 10 min., then pyridine (0.1 ml, 1.2 mmol) was added slowly. The mixture was stirred for 15 min., diluted with 6 mL acetonitrile, and then stirred for 10 minutes. Solids including the product I-2 were filtered from the mixture, rinsed with acetonitrile, and then dried under vacuum. The solid was carried on to the next step without further purification.

¹H NMR (400 MHz, DMSO) δ 12.02 (s, 1H), 9.55 (s, 1H), 9.12 (dd, 1H), 8.81 (s, 1H), 8.74 (dd, 1H), 7.80 (dd, 1H), 7.74 (d, 1H), 7.62 (d, 1H), 7.19 (s, 1H), 3.96 (s, 3H)

### Intermediate I-3

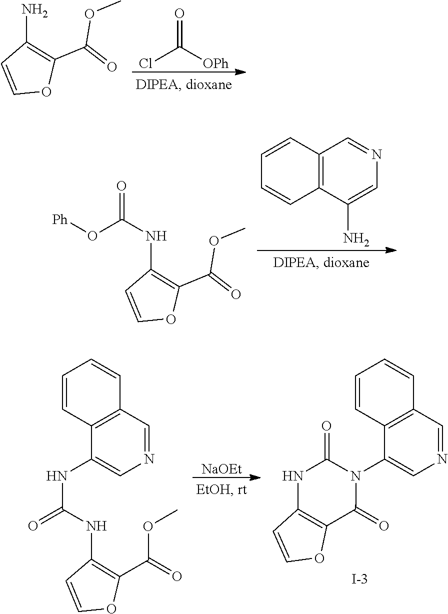

Methyl 3-((phenoxycarbonyl)amino)furan-2-carboxylate: To a stirring solution of methyl 3-aminofuran-2-carboxylate (2.0 g, 14.2 mmol, 1.0 equiv.) in 1,4-dioxane (70 mL, 0.25 mL) was added phenyl chloroformate (2.77 mL, 21.3 mmol, 1.5 equiv.) and DIPEA (4.94 mL, 28.3 mmol, 2.0 equiv.). The mixture was heated to 95° C. and stirred for 6 hr., after which LCMS analysis showed full consumption of amine. The mixture was cooled to rt and concentrated under reduced pressure to provide a residue including the product. The residue was carried on to the next step without further purification.

ES/MS: 261.9 (M⁺)

Methyl 3-(4-isoquinolylcarbamoylamino)furan-2-carboxylate: To a stirring solution of methyl 3-((phenoxycarbonyl)amino)furan-2-carboxylate (3.70 g, 14.2 mmol, 1.0 equiv.) in 1,4-dioxane (55 mL, 0.25 mL) was added isoquinolin-4-amine (2.45 g, 17.0 mmol, 1.1 equiv.) and DIPEA (4.94 mL, 28.3 mmol, 2.0 equiv.). The mixture was heated to 90° C. and stirred for 12 hr., after which full consumption of amine was observed. The mixture was cooled to rt and concentrated under reduced pressure. To the mixture was added MeCN (50 mL). The mixture was sonicated for 5 min and filtered to deliver methyl 3-(4-isoquinolylcarbamoylamino)furan-2-carboxylate, which was used without any further purification.

ES/MS: 311.8 (M⁺)

3-(4-isoquinolyl)-1H-furo[3,2-d]pyrimidine-2,4-dione (I-3): To a suspension of methyl 3-(4-isoquinolylcarbamoylamino)furan-2-carboxylate (2.0 g, 6.42 mmol, 1.0 equiv.) in ethanol (20 mL, 0.32 M) was added a solution of sodium ethoxide (3.6 mL, 2M in ethanol, 1.5 equiv.) at room temperature. The mixture stirred at room temperature for 2 hr. Upon addition of 1M HCl (5 mL), a precipitate formed in the mixture. The precipitate was filtered and discarded, and the filtrate was concentrated under reduced pressure to provide a solid. To the solid was added H₂O (20 mL). The mixture was sonicated for 5 min., and the solid was collected via filtration to deliver the target compound I-3 as an HCl salt.

ES/MS: 275.9 (M⁺)

¹H NMR (400 MHz, DMSO-d₆) δ 11.99 (s, 1H), 9.55 (s, 1H), 8.61 (s, 1H), 8.39-8.30 (m, 1H), 8.21 (d, J=2.0 Hz, 1H), 7.98-7.77 (m, 3H), 6.75 (d, J=2.0 Hz, 1H)

### Intermediate I-4

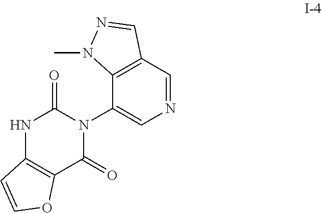 [SMILES: Cn1ncc2cncc(-n3c(=O)[nH]c4ccoc4c3=O)c21]

3-(1-methylpyrazolo[4,3-c]pyridin-7-yl)-1H-furo[3,2-d]pyrimidine-2,4-dione (I-4): Prepared analogously to I-3, substituting isoquinolin-4-amine with 1-methylpyrazolo[4,3-c]pyridin-7-amine.

ES/MS: 283.9 (M⁺)

### Intermediate I-5

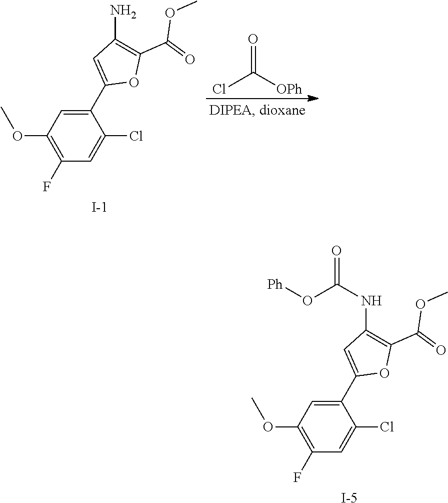

Methyl 5-(2-chloro-4-fluoro-5-methoxy-phenyl)-3-(phenoxycarbonylamino)furan-2-carboxylate (I-5): To a stirring solution of methyl 3-amino-5-(2-chloro-4-fluoro-5-methoxy-phenyl)furan-2-carboxylate I-1 (400 mg, 1.33 mmol, 1.0 equiv.) in dioxane (5.8 mL, 0.22 M) was added DIPEA (0.47 mL, 2.7 mmol, 2.0 equiv.) followed by phenyl chloroformate (0.26 mL, 2.0 mmol, 1.5 equiv.). The mixture was stirred at 90° C. overnight, after which full consumption of the starting material was observed. The mixture was cooled to rt and concentrated under reduced pressure. Upon addition of hexanes (10 mL), a precipitate formed in the mixture. The mixture was sonicated (15 min) and cooled in an ice bath. The mixture was filtered and rinsed with additional hexanes (10 mL) to afford the target compound I-5 as an DIPEA-HCl salt. The DIPEA-HCl salt was stirred for 15 minutes in water (10 mL), after which the suspension was filtered. The precipitate was dried to furnish the product methyl 5-(2-chloro-4-fluoro-5-methoxy-phenyl)-3-(phenoxycarbonylamino)furan-2-carboxylate I-5.

ES/MS: 419.8 (M⁺)

¹H NMR (400 MHz, DMSO-d₆) δ 9.44 (s, 1H), 7.72-7.61 (m, 2H), 7.55-7.42 (m, 3H), 7.35-7.25 (m, 3H), 3.94 (s, 3H), 3.91 (s, 3H)

### Intermediate I-6

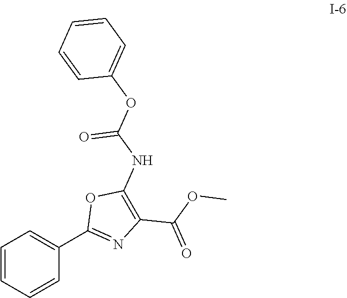 [SMILES: COC(=O)c1nc(-c2ccccc2)oc1NC(=O)Oc1ccccc1]

Methyl 5-((phenoxycarbonyl)amino)-2-phenyloxazole-4-carboxylate (I-6): Prepared analogously to I-5, substituting 3-amino-5-(2-chloro-4-fluoro-5-methoxy-phenyl)furan-2-carboxylate with methyl 5-amino-2-phenyloxazole-4-carboxylate.

ES/MS: 338.9 (M+H⁺)

### Intermediate I-7

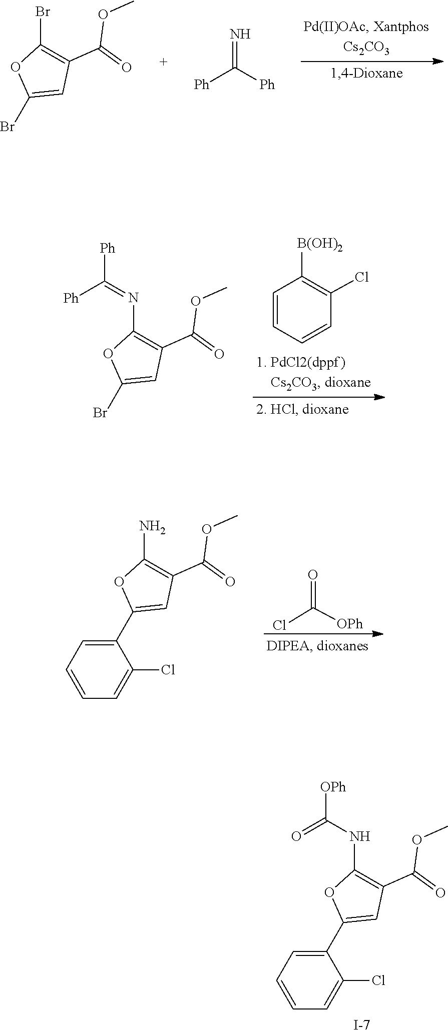

Methyl 2-(benzhydrylideneamino)-5-bromo-furan-3-carboxylate: To a flame-dried vial was added methyl 2,5-dibromofuran-3-carboxylate (100 mg, 0.35 mmol, 1.0 equiv.), palladium(II) acetate (8.0 mg, 0.04 mmol, 10 mol %), Xantphos (40.8 mg, 0.07 mmol, 20 mol %), cesium carbonate (115 mg, 0.35 mmol, 1.0 equiv.), and benzophenone imine (76.6 mg, 0.42 mmol, 0.071 mL, 1.2 equiv.), followed by 1,4-dioxane (1.5 mL, 0.2 M). The mixture was degassed with Ar over 10 min., and subsequently heated at 90° C. for 6 hours. The mixture was then cooled to room temperature, concentrated under reduced pressure, and was purified by silica chromatography (eluent: EtOAc in hexanes (1:5)) to provide the product.

ES/MS: 455.9 (M⁺)

Methyl 5-bromo-2-((diphenylmethylene)amino)furan-3-carboxylate: To a microwave vial containing a stir bar was added methyl 2-(benzhydrylideneamino)-5-bromo-furan-3-carboxylate (100 mg, 0.25 mmol, 1.0 equiv.), (2-chlorophenyl)boronic acid (48 mg, 0.31 mmol, 1.3 equiv.), PdCl₂(dppf) (19 mg, 0.025 mmol, 10 mol %), and cesium carbonate (252 mg, 0.74 mmol, 3.0 equiv.), followed by 1,4-dioxane (2.2 mL, 0.11 M). The vial was sealed with Teflon, and the mixture was degassed under Ar (5 min.), and heated to 95° C. under microwave irradiation for 25 min. The crude product was filtered through celite and used without further purification to the next step.

ES/MS: 251.9 (M⁺)

Methyl 2-amino-5-(2-chlorophenyl)furan-3-carboxylate: To a stirring solution of methyl 5-bromo-2-((diphenylmethylene)amino)furan-3-carboxylate (107 mg, 0.27 mmol) in 1,4-dioxane (0.75 mL, 0.35 M), was added 4 M HCl (0.35 mL). The mixture was diluted in acetonitrile/water/trifluoroacetic acid (1 mL; 5:1:0.2), filtered through an acrodisc, and purified directly by RP-HPLC (0.1% TFA-ACN in 0.1% TFA water, Column: Gemini 5 uM, NX—C18 110 Angstrom, 250×21.2 mm) to give the title compound as a trifluoroacetate salt.

ES/MS: 183.0 (M⁺)

¹H NMR (400 MHz, DMSO-d₆) δ 7.65 (dd, J=8.0, 1.6 Hz, 1H), 7.50 (dd, J=8.0, 1.2 Hz, 1H), 7.40 (ddd, J=8.0, 7.4, 1.7 Hz, 1H), 7.23 (ddd, J=8.0, 7.4, 1.7 Hz, 1H), 7.14 (s, 1H), 6.54 (s, 2H), 3.71 (s, 3H)

Methyl 5-(2-chlorophenyl)-2-((phenoxycarbonyl)amino)furan-3-carboxylate (I-7): To a stirring solution of methyl 2-amino-5-(2-chlorophenyl)furan-3-carboxylate (200 mg, 0.80 mmol, 1.0 equiv.) in 1,4-dioxane (3.4 mL, 0.22 M) was added DIPEA (0.28 mL, 1.59 mmol, 2.0 equiv.), followed by phenyl chloroformate (0.16 mL, 1.19 mmol, 1.5 equiv.). The mixture was stirred at 90° C. for 6 hr., after which full consumption of the starting material was observed. The mixture was cooled to rt and concentrated under reduced pressure. Upon addition of hexanes (10 mL), a precipitate began to form. The mixture was sonicated (15 min) and cooled in an ice bath. The mixture was filtered and rinsed with additional hexane (10 mL) to afford the target compound I-7 as an DIPEA-HCl salt. The DIPEA-HCl salt was stirred for 15 minutes in water (10 mL), after which the suspension was filtered. The precipitate was dried to furnish the product methyl 5-(2-chlorophenyl)-2-((phenoxycarbonyl)amino)furan-3-carboxylate I-7.

ES/MS: 371.8 (M⁺)

### Intermediate I-8

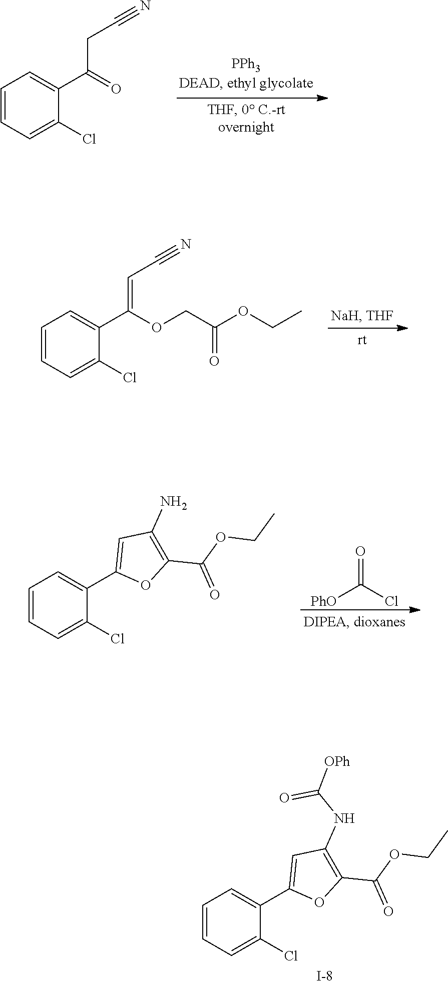

Ethyl (Z)-2-((1-(2-chlorophenyl)-2-cyanovinyl)oxy)acetate): To a solution of triphenyl phosphine (10.2 g, 39.0 mmol) in anhydrous THF (140 mL) at 0° C. was added diethyl azodicarboxy-late (6.79 g, 39.0 mmol), ethyl glycolate (4.06 g, 39.0 mmol), and 3-(2-chlorophenyl)-3-oxo-propanenitrile (5.00 g, 27.8 mmol). The mixture was allowed to warm to room temperature and stirred overnight. The mixture was then concentrated under reduced pressure to provide a residue. The residue was purified by flash chromatography using a mixture of ethyl acetate and hexane as eluent to provide the title compound.

ES/MS: 265.9 (M⁺)

Ethyl 3-amino-5-(2-chlorophenyl)furan-2-carboxylate: To a slurry of sodium hydride (1.28 g, 33.8 mmol, 60% dispersion in oil) in anhydrous THF (100 mL) was added ethyl (Z)-2-((1-(2-chlorophenyl)-2-cyanovinyl)oxy)acetate (5.0 g, 20.7 mmol). The mixture was stirred at room temperature for 3 hr. Upon completion of reaction based on TLC, the mixture was treated with saturated ammonium chloride solution (10 mL), and then concentrated under reduced pressure to provide a residue. The residue was purified by flash chromatography using a mixture of ethyl acetate and hexane as eluent to yield the title compound.

ES/MS: 265.9 (M⁺)

¹H NMR (400 MHz, DMSO) δ 7.84 (dd, J=7.7, 1.9 Hz, 1H), 7.63-7.55 (m, 1H), 7.53-7.36 (m, 2H), 6.88 (s, 1H), 5.77 (s, 2H), 4.26 (q, J=7.1 Hz, 2H), 1.30 (t, J=7.1 Hz, 3H)

Ethyl 5-(2-chlorophenyl)-3-(phenoxycarbonylamino)furan-2-carboxylate (I-8): To a stirring solution of ethyl 3-amino-5-(2-chlorophenyl)furan-2-carboxylate (1.2 g, 4.35 mmol, 1.0 equiv.) in 1,4-dioxane (22 mL, 0.20 M) was added DIPEA (1.52 mL, 8.71 mmol, 2.0 equiv.) followed by phenyl chloroformate (1.16 mL, 8.92 mmol, 2.05 equiv.). The mixture was stirred at 95° C. for 30 min., after which full consumption of the starting material was observed. The mixture was cooled to rt and concentrated under reduced pressure. Upon addition of hexane (50 mL), a precipitate began to form. The mixture was sonicated (15 min) and cooled in an ice bath. The mixture was filtered and rinsed with additional MeCN (20 mL) to afford the target compound ethyl 5-(2-chlorophenyl)-3-(phenoxycarbonylamino)furan-2-carboxylate I-8.

ES/MS: 385.8 (M⁺)

¹H NMR (400 MHz, DMSO-d₆) δ 9.34 (s, 1H), 7.90 (dd, J=7.7, 1.9 Hz, 1H), 7.73 (s, 1H), 7.64 (dd, J=7.5, 1.7 Hz, 1H), 7.58-7.42 (m, 4H), 7.35-7.25 (m, 3H), 4.39 (q, J=7.1 Hz, 2H), 1.36 (t, J=7.1 Hz, 3H).

### Intermediate I-9

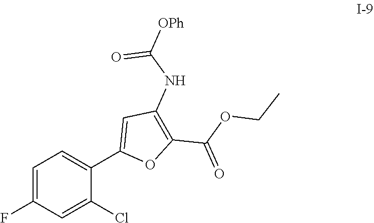 [SMILES: CCOC(=O)c1oc(-c2ccc(F)cc2Cl)cc1NC(=O)Oc1ccccc1]

Ethyl 3-amino-5-(2-chloro-4-fluorophenyl)furan-2-carboxylate (I-9): Prepared analogously to I-8, substituting 3-(2-chlorophenyl)-3-oxo-propanenitrile with 3-(2-chloro-4-fluoro-phenyl)-3-oxo-propanenitrile.

ES/MS: 403.9 (M⁺)

### II. Compounds

### Procedure 1: Example 1

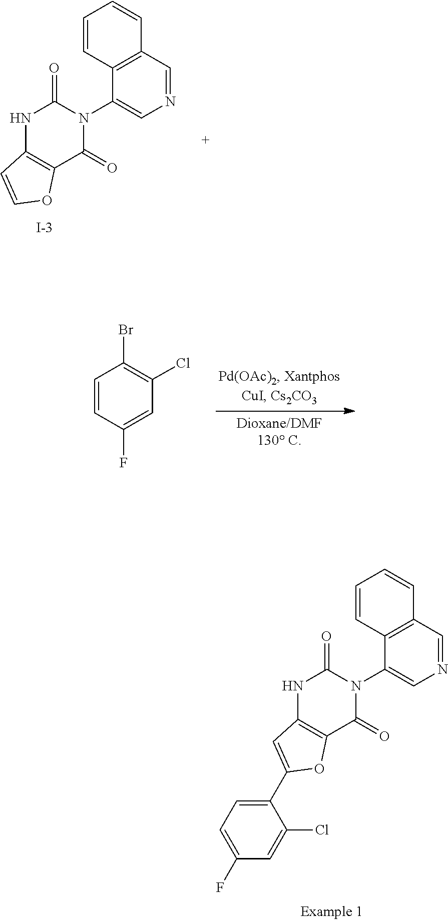

6-(2-chlorophenyl)-3-(isoquinolin-4-yl)thieno[3,2-d]pyrimidine-2,4(1H,3H)-dione (Example 1): To a 20 mL microwave vial containing a stir bar was added 3-(4-isoquinolyl)-1H-furo[3,2-d]pyrimidine-2,4-dione I-3 (HCl salt) (300 mg, 0.85 mmol, 1.0 equiv), 1-bromo-2-chloro-4-fluoro-benzene (358 mg, 1.71 mmol, 2.0 equiv.), Pd(OAc)₂ (76.8 mg, 40 mol %), XantPhos (400 mg, 0.68 mmol, 0.8 equiv.), Cs₂CO₃ (1.67 g, 5.13 mmol, 6.0 equiv.), and CuI (81.4 mg, 0.43 mmol, 0.5 equiv.). Dioxane (12.3 mL, 0.07 M) was added. The mixture was degassed under Ar (5 min), and heated to 130° C. under conventional heating overnight. The mixture was diluted in acetonitrile/water/trifluoroacetic acid (10 mL; 5:1:0.2), filtered through an acrodisc, and purified directly by RP-HPLC (0.1% TFA-ACN in 0.1% TFA water, Column: Gemini 5 uM, NX—C18 110 Angstrom, 250×21.2 mm) to give the title compound Example 1 as a trifluoroacetate salt.

ES/MS: 407.8 (M⁺)

¹H NMR (400 MHz, DMSO-d₆) δ 12.02 (s, 1H), 9.49 (s, 1H), 8.58 (s, 1H), 8.34-8.27 (m, 1H), 8.04 (dd, J=8.9, 6.1 Hz, 1H), 7.90 (d, J=8.0 Hz, 1H), 7.88-7.71 (m, 3H), 7.51-7.41 (m, 1H), 7.18 (s, 1H)

The following Examples were made in an analogous fashion according to Procedure 1 and are shown below in Table 2. To prepare the below Examples, different reagents/starting materials were used as compared to some of those described in Procedure 1, and are noted in the last column of Table 2—“Changes to Procedure 1: Different Reagents/Starting Materials”. A person of ordinary skill in the art will readily recognize which reagents/starting materials of Procedure 1 were replaced with the different reagents/starting materials noted below.

TABLE 2

| | | | | Changes to |
|---|---|---|---|---|
| | | | | Procedure 1: |
| | | ES/MS | | Different Reagents/ |
| Ex. | Structure | m/z | ¹H-NMR | Starting Materials |
| 2 | 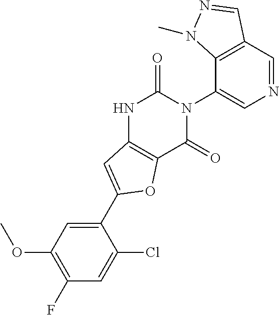 [SMILES: COc1cc(-c2cc3[nH]c(=O)n(-c4cncc5cnn(C)c45)c(=O)c3o2)c(Cl)cc1F] | 367.0 | ¹H NMR (400 MHz, DMSO- d₆) δ 12.24 (s, 1H), 8.53 (s, 1H), 7.74 (d, J = 11.0 Hz, 1H), 7.60 (d, J = 8.9 Hz, 1H), 7.21 (s, 1H), 3.96 (s, 3H), 3.91 (s, 3H). | I-4, 1-bromo-2- chloro-4-fluoro-5- methoxy-benzene |
| 3 | 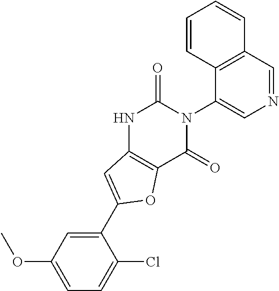 [SMILES: COc1ccc(Cl)c(-c2cc3[nH]c(=O)n(-c4cncc5ccccc45)c(=O)c3o2)c1] | 420.2 | ¹H NMR (400 MHz, DMSO- d₆) δ 12.01 (s, 1H), 9.45 (s, 1H), 8.55 (s, 1H), 8.29 (dd, J = 7.4, 1.5 Hz, 1H), 7.88 (d, J = 8.1 Hz, 1H), 7.82 (dd, J = 6.8, 1.5 Hz, 1H), 7.80-7.75 (m, 1H), 7.60 (d, J = 8.9 Hz, 1H), 7.43 (d, J = 3.1 Hz, 1H), 7.23 (s, 1H), 7.15 (dd, J = 8.9, 3.1 Hz, 1H), 3.86 (s, 3H). | 2-bromo-1-chloro- 4-methoxy-benzene |

### Procedure 2: Example 4

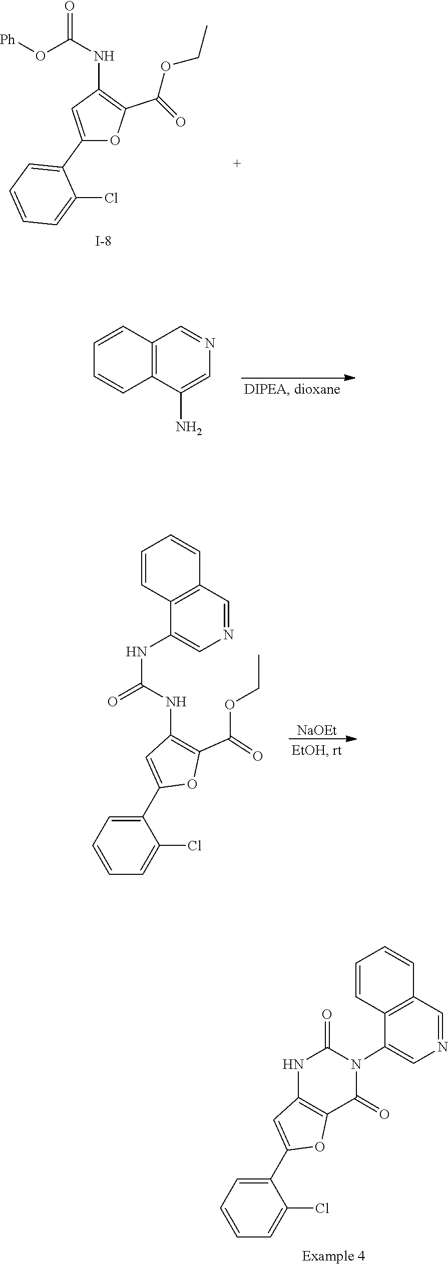

Ethyl 5-(2-chlorophenyl)-3-(4-isoquinolylcarbamoylamino)furan-2-carboxylate: To a suspension of ethyl 5-(2-chlorophenyl)-3-(phenoxycarbonylamino)furan-2-carboxylate I-8 (460 mg, 1.12 mmol, 1.0 equiv.) in dioxane (6.0 mL, 0.2 M) was added isoquinolin-4-amine (206 mg, 1.43 mmol, 1.2 equiv.) followed by DIPEA (0.42 mL, 2.38 mmol, 2.0 equiv.). The mixture was stirred at 95° C. overnight, cooled to rt, and concentrated under reduced pressure. Upon addition of MeCN (15 mL), a precipitate began to form. The mixture was stirred for 30 min., after which the solid was isolated by filtration. The precipitate was washed with additional MeCN (15 mL) and dried overnight to deliver the target product ethyl 5-(2-chlorophenyl)-3-(4-isoquinolylcarbamoylamino)furan-2-carboxylate.

ES/MS: 435.8 (M⁺)

6-(2-chlorophenyl)-3-(4-isoquinolyl)-1H-furo[3,2-d]pyrimidine-2,4-dione (Example 4): To a suspension of ethyl 5-(2-chlorophenyl)-3-(4-isoquinolylcarbamoylamino)furan-2-carboxylate (165 mg, 0.38 mmol, 1.0 equiv.) in EtOH (4 mL, 0.32 M) was added sodium ethoxide (21% wt. in EtOH; 0.67 mL, 1.79 mmol, 1.5 equiv.) at room temperature. The mixture was stirred for 30 min after which 1 M HCl (2 mL) was added. The mixture was diluted in acetonitrile/water/trifluoroacetic acid (5 mL; 5:1:0.2), filtered through an acrodisc, and purified directly by RP-HPLC (0.1% TFA-ACN in 0.1% TFA water, Column: Gemini 5 μM, NX—C18 110 Angstrom, 250×21.2 mm) to give the title compound 6-(2-chlorophenyl)-3-(4-isoquinolyl)-1H-furo[3,2-d]pyrimidine-2,4-dione Example 4 as a trifluoroacetate salt.

ES/MS: 389.9 (M⁺)

¹H NMR (400 MHz, DMSO-d₆) δ 12.00 (s, 1H), 9.47 (s, 1H), 8.57 (s, 1H), 8.32-8.26 (m, 1H), 8.02-7.96 (m, 1H), 7.88 (d, J=7.6 Hz, 1H), 7.84-7.74 (m, 2H), 7.73-7.68 (m, 1H), 7.60-7.53 (m, 2H), 7.22 (s, 1H)

The following Examples were made in an analogous fashion according to Procedure 2 and are shown below in Table 3. To prepare the below Examples, different reagents/starting materials were used as compared to some of those described in Procedure 2, and are noted in the last column of Table 3—“Changes to Procedure 2: Different Reagents/Starting Materials”. A person of ordinary skill in the art will readily recognize which reagents/starting materials of Procedure 2 were replaced with the different reagents/starting materials noted below.

TABLE 3

| | | | | Changes to |
|---|---|---|---|---|
| | | | | Procedure 2: |
| | | ES/MS | | Different Reagents/ |
| Ex. | Structure | m/z | ¹H-NMR | Starting Materials |
| 5 | 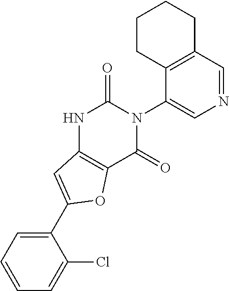 [SMILES: O=c1[nH]c2cc(-c3ccccc3Cl)oc2c(=O)n1-c1cncc2c1CCCC2] | 393.9 | ¹H NMR (400 MHz, DMSO- d₆) δ 12.04 (s, 1H), 8.52 (s, 1H), 8.46 (s, 1H), 8.01- 7.92 (m, 1H), 7.74-7.65 (m, 1H), 7.60-7.51 (m, 2H), 7.18 (s, 1H), 2.88 (t, J = 6.0 Hz, 2H), 2.55 (t, J = 6.1 Hz, 2H), 1.81-1.68 (m, 4H). | 5,6,7,8- tetrahydroisoquinolin- 4-amine |
| 6 | 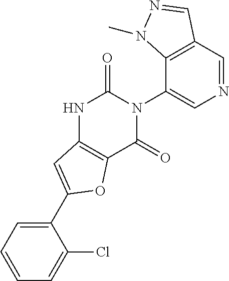 [SMILES: Cn1ncc2cncc(-n3c(=O)[nH]c4cc(-c5ccccc5Cl)oc4c3=O)c21] | 393.8 | ¹H NMR (400 MHz, DMSO- d₆) δ 12.23 (s, 1H), 9.35 (s, 1H), 8.58 (s, 1H), 8.51 (s, 1H), 7.99 (dd, J = 6.0, 3.5 Hz, 1H), 7.76-7.65 (m, 1H), 7.60-7.52 (m, 2H), 7.23 (s, 1H), 3.94 (s, 3H) | 1- methylpyrazolo[4,3- c]pyridin-7-amine |
| 7 | 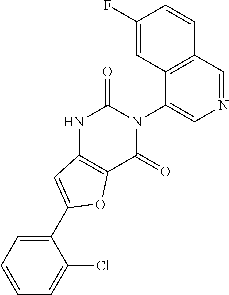 [SMILES: O=c1[nH]c2cc(-c3ccccc3Cl)oc2c(=O)n1-c1cncc2ccc(F)cc12] | 407.8 | ¹H NMR (400 MHz, DMSO- d₆) δ 11.95 (s, 1H), 9.45 (s, 1H), 8.57 (s, 1H), 8.41 (dd, J = 9.1, 5.6 Hz, 1H), 8.04- 7.93 (m, 1H), 7.85 (dd, J = 10.3, 2.4 Hz, 1H), 7.80- 7.59 (m, 2H), 7.59-7.50 (m, 2H), 7.21 (s, 1H). | 6-fluoroisoquinolin- 4-amine |
| 8 | 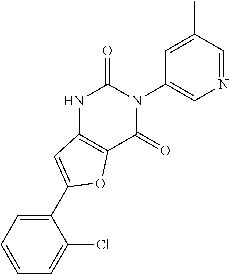 [SMILES: Cc1cncc(-n2c(=O)[nH]c3cc(-c4ccccc4Cl)oc3c2=O)c1] | 353.8 | ¹H NMR (400 MHz, DMSO) δ 11.93 (s, 1H), 8.53 (s, 1H), 8.46-8.41 (m, 1H), 8.01- 7.93 (m, 1H), 7.77 (t, J = 2.3 Hz, 1H), 7.74-7.65 (m, 1H), 7.60-7.50 (m, 2H), 7.16 (s, 1H), 2.40 (s, 3H). | 5-methylpyridin-3- amine |
| 9 | 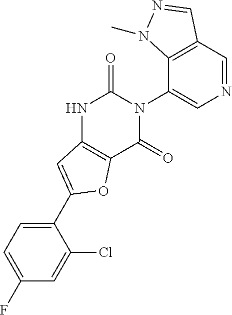 [SMILES: Cn1ncc2cncc(-n3c(=O)[nH]c4cc(-c5ccc(F)cc5Cl)oc4c3=O)c21] | 411.8 | ¹H NMR (400 MHz, DMSO) δ 12.25 (s, 1H), 9.36 (s, 1H), 8.60 (s, 1H), 8.53 (s, 1H), 8.05 (dd, J = 8.9, 6.1 Hz, 1H), 7.75 (dd, J = 8.8, 2.6 Hz, 1H), 7.46 (ddd, J = 8.9, 8.0, 2.6 Hz, 1H), 7.20 (s, 1H), 3.94 (s, 3H). | I-9; 1- methylpyrazolo[4,3- c]pyridin-7-amine |
| 10 | 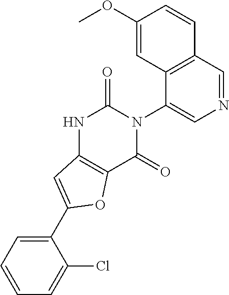 [SMILES: COc1ccc2cncc(-n3c(=O)[nH]c4cc(-c5ccccc5Cl)oc4c3=O)c2c1] | 419.9 | ¹H NMR (400 MHz, DMSO- d₆) δ 11.98 (s, 1H), 9.44 (s, 1H), 8.58 (s, 1H), 8.32 (d, J = 9.1 Hz, 1H), 8.06-7.93 (m, 1H), 7.77-7.66 (m, 1H), 7.61-7.46 (m, 3H), 7.25 (d, J = 13.6 Hz, 2H), 3.92 (s, 3H). | 6- methoxyisoquinolin- 4-amine |

### Procedure 3: Example 11

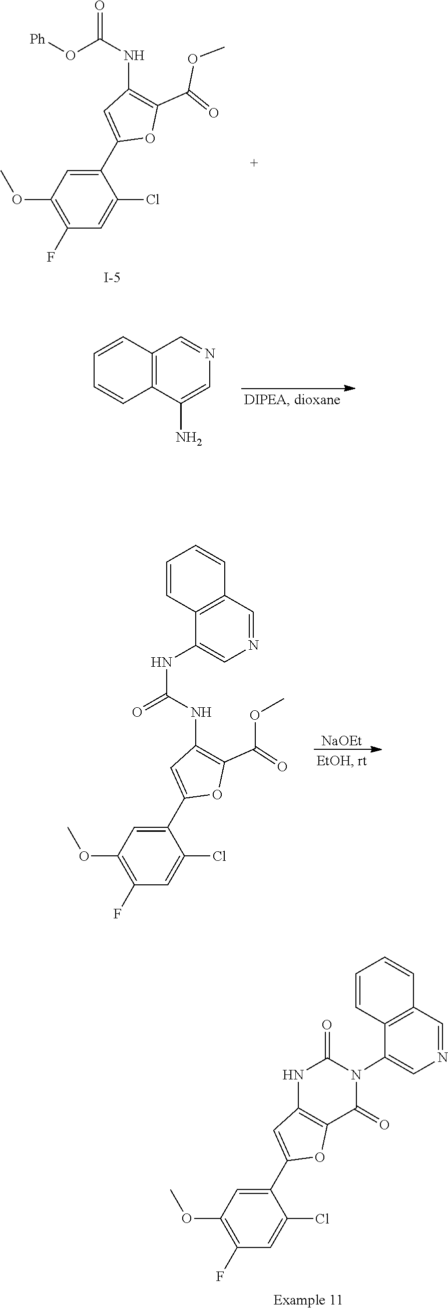

Methyl 5-(2-chloro-4-fluoro-5-methoxyphenyl)-3-(3-(isoquinolin-4-yl)ureido)furan-2-carboxylate: To a suspension of methyl 5-(2-chloro-4-fluoro-5-methoxy-phenyl)-3-(phenoxycarbonylamino)furan-2-carboxylate I-5 (300 mg, 0.72 mmol) in dioxane (2.8 mL) was added isoquinolin-4-amine (124 mg, 0.86 mmol, 1.2 equiv.) followed by DIPEA (0.25 mL, 1.43 mmol, 2.0 equiv.). The mixture was stirred at 95° C. overnight, cooled to rt, and concentrated under reduced pressure. Upon addition of MeCN (15 mL), a precipitate began to form. The mixture was stirred for 30 min., after which the solid was isolated by filtration. The precipitate was washed with additional MeCN (15 mL) and dried overnight to deliver the target product methyl 5-(2-chloro-4-fluoro-5-methoxyphenyl)-3-(3-(isoquinolin-4-yl)ureido)furan-2-carboxylate.

ES/MS: 469.8 (M⁺)

6-(2-chloro-4-fluoro-5-methoxy-phenyl)-3-(4-isoquinolyl)-1H-furo[3,2-d]pyrimidine-2,4-dione (Example 11): To a suspension of methyl 5-(2-chloro-4-fluoro-5-methoxy-phenyl)-3-(4-isoquinolylcarbamoylamino)furan-2-carboxylate (200 mg, 0.43 mmol, 1.0 equiv.) in EtOH (1.34 mL, 0.32 M) was added sodium ethoxide (21% wt. in EtOH; 0.24 mL, 0.64 mmol, 1.5 equiv.) at room temperature. The mixture was stirred for 30 min after which 1 M HCl (2 mL) was added. The mixture was diluted in acetonitrile/water/trifluoroacetic acid (5 mL; 5:1:0.2), filtered through an acrodisc, and purified directly by RP-HPLC (0.1% TFA-ACN in 0.1% TFA water, Column: Gemini 5 μM, NX—C18 110 Angstrom, 250×21.2 mm) to give the title compound 6-(2-chloro-4-fluoro-5-methoxy-phenyl)-3-(4-isoquinolyl)-1H-furo[3,2-d]pyrimidine-2,4-dione Example 11 as a trifluoroacetate salt.

ES/MS: 437.8 (M⁺)

¹H NMR (400 MHz, DMSO-d₆) δ 12.04 (s, 1H), 9.46 (s, 1H), 8.56 (s, 1H), 8.30 (dd, J=7.6, 1.5 Hz, 1H), 7.91-7.70 (m, 4H), 7.61 (d, J=8.9 Hz, 1H), 7.20 (s, 1H), 3.96 (s, 3H)

The following Examples were made in an analogous fashion according to Procedure 3 and are shown below in Table 4. To prepare the below Examples, different reagents/starting materials were used as compared to some of those described in Procedure 3, and are noted in the last column of Table 4—“Changes to Procedure 3: Different Reagents/Starting Materials”. A person of ordinary skill in the art will readily recognize which reagents/starting materials of Procedure 3 were replaced with the different reagents/starting materials noted below.

TABLE 4

| | | | | Changes to |
|---|---|---|---|---|
| | | | | Procedure 3: |
| | | ES/MS | | Different Reagents/ |
| Ex. | Structure | m/z | ¹H-NMR | Starting Materials |
| 12 | 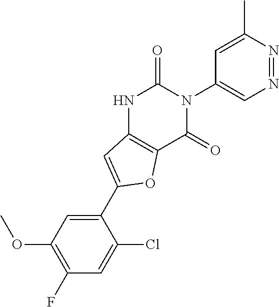 [SMILES: COc1cc(-c2cc3[nH]c(=O)n(-c4cnnc(C)c4)c(=O)c3o2)c(Cl)cc1F] | 402.8 | ¹H NMR (400 MHz, DMSO-d₆) δ 12.10 (s, 1H), 9.14 (d, J = 2.3 Hz, 1H), 7.77-7.69 (m, 2H), 7.59 (d, J = 8.9 Hz, 1H), 7.16 (s, 1H), 3.96 (s, 3H), 2.72 (s, 3H). | 6-methylpyridazin-4- amine |
| 13 | 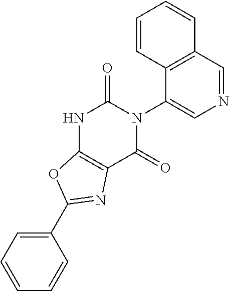 [SMILES: O=c1[nH]c2oc(-c3ccccc3)nc2c(=O)n1-c1cncc2ccccc12] | 356.9 | ¹H NMR (400 MHz, DMSO-d₆) δ 9.46 (s, 1H), 8.53 (s, 1H), 8.30 (d, J = 8.0 Hz, 1H), 8.02 (dd, J = 6.6, 3.0 Hz, 2H), 7.92 (d, J = 8.3 Hz, 1H), 7.88-7.73 (m, 2H), 7.67-7.60 (m, 3H). | I-6 |
| 14 | 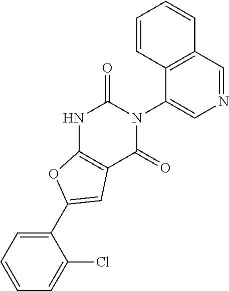 [SMILES: O=c1[nH]c2oc(-c3ccccc3Cl)cc2c(=O)n1-c1cncc2ccccc12] | 389.8 | ¹H NMR (400 MHz, DMSO-d₆) δ 9.46 (s, 1H), 8.55 (s, 1H), 8.29 (dd, J = 7.5, 1.5 Hz, 1H), 7.95- 7.88 (m, 1H), 7.87-7.73 (m, 3H), 7.64 (dd, J = 8.0, 1.3 Hz, 1H), 7.54 (td, J = 7.6, 1.3 Hz, 1H), 7.48- 7.39 (m, 2H). | I-7 |
| 15 | 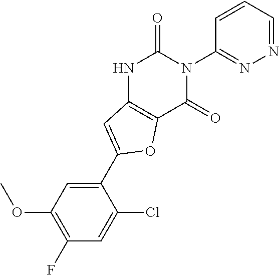 [SMILES: COc1cc(-c2cc3[nH]c(=O)n(-c4cccnn4)c(=O)c3o2)c(Cl)cc1F] | 388.80 | ¹H NMR (400 MHz, DMSO-d₆) δ 12.10 (s, 1H), 9.36 (dd, J = 4.7, 1.7 Hz, 1H), 8.02-7.89 (m, 2H), 7.73 (d, J = 11.0 Hz, 1H), 7.61 (d, J = 8.9 Hz, 1H), 7.18 (s, 1H), 3.97 (s, 3H). | pyridazin-3-amine |

### Procedure 4: Example 16

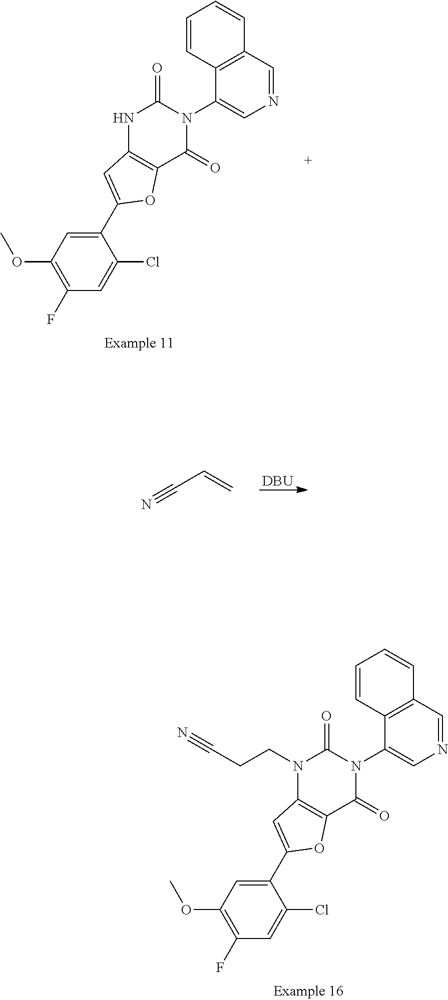

3-(6-(2-chloro-4-fluoro-5-methoxyphenyl)-3-(isoquinolin-4-yl)-2,4-dioxo-3,4-dihydrofuro[3,2-d]pyrimidin-1(2H1)-yl)propanenitrile (Example 16): To a stirring solution of 6-(2-chloro-4-fluoro-5-methoxy-phenyl)-3-(4-isoquinolyl)-1H-furo[3,2-d]pyrimidine-2,4-dione Example 11 (HCl salt) (30 mg, 0.07 mmol, 1.0 equiv.) in acrylonitrile (0.37 mL, 0.2 M) was added DBU (41 μL, 0.27 mmol, 4 equiv.). The mixture stirred for 20 hours at 80° C. after which the mixture was diluted with water (0.2 mL) and TFA (0.5 mL). The mixture was then filtered through an acrodisc, and purified directly by RP-HPLC (0.100 TFA-ACN in 0.1% TFA water, Column: Gemini 5 μM, NX—C18 110 Angstrom, 250×21.2 mm) to give the title compound Example 16 as a trifluoroacetate salt.

ES/MS: 490.8 (M⁺)

¹H NMNR (400 MHz, DMSO-d₆) δ 9.48 (d, J=0.8 Hz, 1H), 8.56 (s, 1H), 8.30 (dd, J=7.1, 1.8 Hz, 1H), 7.93-7.72 (m, 5H), 7.60 (d, J=8.8 Hz, 1H), 4.37 (hept, J=7.2 Hz, 2H), 3.97 (s, 3H), 3.01 (t, J=6.5 Hz, 2H)

The following Examples were made in an analogous fashion according to Procedure 4 and are shown below in Table 5. To prepare the below Examples, different reagents/starting materials were used as compared to some of those described in Procedure 4, and are noted in the last column of Table 5—“Changes to Procedure 4: Different Reagents/Starting Materials”. A person of ordinary skill in the art will readily recognize which reagents/starting materials of Procedure 4 were replaced with the different reagents/starting materials noted below.

TABLE 5

| | | | | Changes to |
|---|---|---|---|---|
| | | | | Procedure 4: |
| | | ES/MS | | Different Reagents/ |
| Ex. | Structure | m/z | ¹H-NMR | Starting Materials |
| 17 | 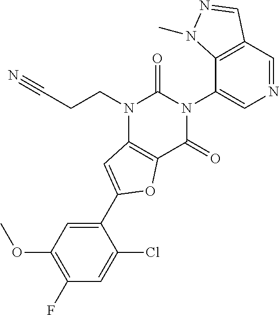 [SMILES: COc1cc(-c2cc3c(o2)c(=O)n(-c2cncc4cnn(C)c24)c(=O)n3CCC#N)c(Cl)cc1F] | 494.8 | ¹H NMR (400 MHz, DMSO- d₆) δ 9.30 (s, 1H), 8.54 (s, 1H), 8.45 (s, 1H), 7.87 (s, 1H), 7.76 (d, J = 11.0 Hz, 1H), 7.60 (d, J = 8.9 Hz, 1H), 4.55-4.25 (m, 2H), 3.97 (s, 3H), 3.92 (s, 3H), 3.05 (td, J = 6.5, 2.7 Hz, 2H) | Example 2 |
| 18 | 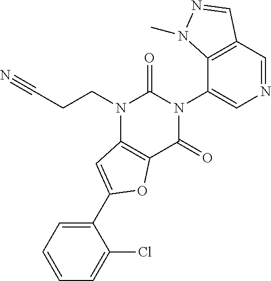 [SMILES: Cn1ncc2cncc(-n3c(=O)c4oc(-c5ccccc5Cl)cc4n(CCC#N)c3=O)c21] | 446.8 | ¹H NMR (400 MHz, DMSO- d₆) δ 9.35 (s, 1H), 8.58 (s, 1H), 8.50 (s, 1H), 8.02-7.94 (m, 1H), 7.90 (s, 1H), 7.71 (dt, J = 7.4, 3.7 Hz, 1H), 7.58 (dd, J = 6.0, 3.5 Hz, 2H), 4.49- 4.30 (m, 2H), 3.93 (s, 3H), 3.04 (td, J = 6.6, 2.3 Hz, 2H). | Example 6 |
| 19 | 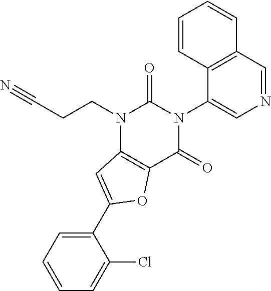 [SMILES: N#CCCn1c(=O)n(-c2cncc3ccccc23)c(=O)c2oc(-c3ccccc3Cl)cc21] | 442.9 | ¹H NMR (400 MHz, DMSO) δ 9.50 (d, J = 0.9 Hz, 1H), 8.58 (s, 1H), 8.36-8.29 (m, 1H), 8.02-7.91 (m, 2H), 7.89 (s, 1H), 7.82 (dddd, J = 14.7, 8.2, 6.9, 1.4 Hz, 2H), 7.76-7.68 (m, 1H), 7.62- 7.53 (m, 2H), 4.36 (hept, J = 6.9 Hz, 2H), 3.01 (t, J = 6.5 Hz, 2H). | Example 4 |
| 20 | 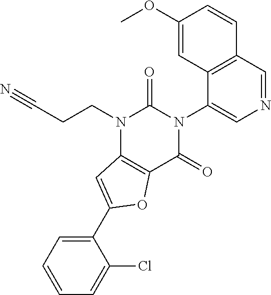 [SMILES: COc1ccc2cncc(-n3c(=O)c4oc(-c5ccccc5Cl)cc4n(CCC#N)c3=O)c2c1] | 472.8 | ¹H NMR (400 MHz, DMSO- d₆) δ 9.44 (s, 1H), 8.56 (s, 1H), 8.31 (d, J = 9.1 Hz, 1H), 8.05-7.95 (m, 1H), 7.90 (s, 1H), 7.79-7.69 (m, 1H), 7.62-7.54 (m, 2H), 7.49 (dd, J = 9.1, 2.4 Hz, 1H), 7.21 (d, J = 2.4 Hz, 1H), 4.51-4.31 (m, 2H), 3.90 (s, 3H), 3.02 (t, J = 6.6 Hz, 2H). | Example 10 |
| 21 | 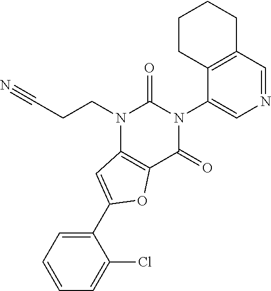 [SMILES: N#CCCn1c(=O)n(-c2cncc3c2CCCC3)c(=O)c2oc(-c3ccccc3Cl)cc21] | 446.9 | ¹H NMR (400 MHz, DMSO- d₆) δ 8.50 (s, 1H), 8.40 (s, 1H), 8.00-7.92 (m, 1H), 7.84 (s, 1H), 7.75-7.66 (m, 1H), 7.62-7.53 (m, 2H), 4.45-4.25 (m, 2H), 3.00 (d, J = 13.1 Hz, 2H), 2.87 (t, J = 6.1 Hz, 2H), 2.50 (m, 1H), 2.49 (m, 1H), 1.74 (s, 3H), 1.80-1.66 (m, 1H). | Example 5 |
| 22 | 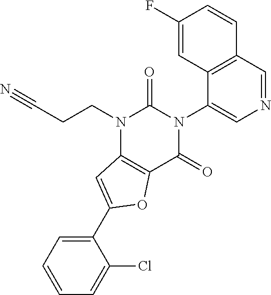 [SMILES: N#CCCn1c(=O)n(-c2cncc3ccc(F)cc23)c(=O)c2oc(-c3ccccc3Cl)cc21] | 460.8 | ¹H NMR (400 MHz, DMSO- d₆) δ 9.47 (d, J = 0.8 Hz, 1H), 8.58 (s, 1H), 8.42 (dd, J = 9.1, 5.6 Hz, 1H), 8.01-7.92 (m, 1H), 7.92-7.83 (m, 2H), 7.77-7.65 (m, 2H), 7.64- 7.51 (m, 2H), 4.36 (td, J = 6.7, 4.1 Hz, 2H), 2.99 (t, J = 6.6 Hz, 2H). | Example 7 |
| 23 | 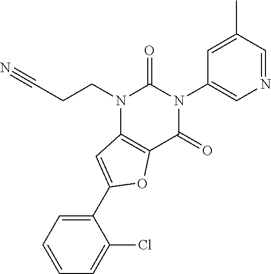 [SMILES: Cc1cncc(-n2c(=O)c3oc(-c4ccccc4Cl)cc3n(CCC#N)c2=O)c1] | 406.8 | ¹H NMR (400 MHz, DMSO) δ 8.53 (d, J = 2.0 Hz, 1H), 8.41 (d, J = 2.2 Hz, 1H), 7.99- 7.92 (m, 1H), 7.83 (s, 1H), 7.74 (d, J = 2.3 Hz, 1H), 7.73- 7.66 (m, 1H), 7.62-7.52 (m, 2H), 4.33 (t, J = 6.5 Hz, 2H), 2.97 (t, J = 6.5 Hz, 2H), 2.40 (s, 3H). | Example 8 |
| 24 | 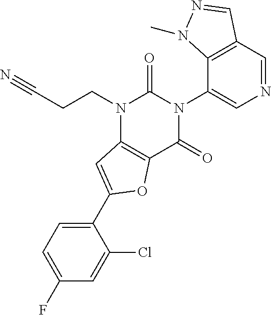 [SMILES: Cn1ncc2cncc(-n3c(=O)c4oc(-c5ccc(F)cc5Cl)cc4n(CCC#N)c3=O)c21] | 464.8 | ¹H NMR (400 MHz, DMSO) δ 9.37 (s, 1H), 8.60 (s, 1H), 8.52 (s, 1H), 8.05 (dd, J = 8.9, 6.1 Hz, 1H), 7.89 (s, 1H), 7.77 (dd, J = 8.8, 2.6 Hz, 1H), 7.49 (ddd, J = 8.9, 8.0, 2.6 Hz, 1H), 4.47-4.34 (m, 2H), 3.94 (s, 3H), 3.05 (td, J = 7.0, 6.5, 2.3 Hz, 2H). | Example 9 |
| 25 | 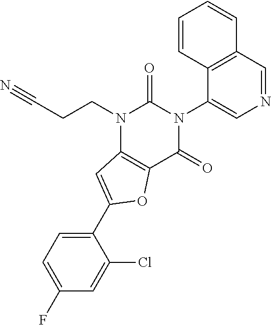 [SMILES: N#CCCn1c(=O)n(-c2cncc3ccccc23)c(=O)c2oc(-c3ccc(F)cc3Cl)cc21] | 460.80 | ¹H NMR (400 MHz, DMSO- d₆) δ 9.49 (s, 1H), 8.57 (s, 1H), 8.35-8.27 (m, 1H), 8.04 (dd, J = 8.9, 6.1 Hz, 1H), 7.92 (dd, J = 8.1, 1.4 Hz, 1H), 7.86 (s, 1H), 7.85-7.74 (m, 3H), 7.48 (td, J = 8.5, 2.6 Hz, 1H), 4.37 (hept, J = 7.7, 7.2 Hz, 2H), 3.00 (t, J = 6.6 Hz, 2H). | Example 1 |
| 26 | 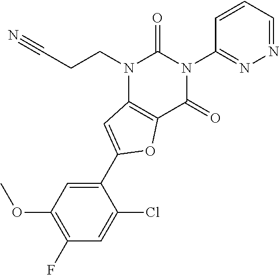 [SMILES: COc1cc(-c2cc3c(o2)c(=O)n(-c2cccnn2)c(=O)n3CCC#N)c(Cl)cc1F] | 441.80 | ¹H NMR (400 MHz, DMSO- d₆) δ 9.38 (dd, J = 4.8, 1.7 Hz, 1H), 8.00 (dd, J = 8.6, 4.8 Hz, 1H), 7.92 (dd, J = 8.5, 1.6 Hz, 1H), 7.83 (s, 1H), 7.75 (d, J = 11.0 Hz, 1H), 7.60 (d, J = 8.9 Hz, 1H), 4.34 (s, 2H), 3.97 (s, 3H), 3.00 (t, J = 6.6 Hz, 2H). | Example 15 |
| 27 | 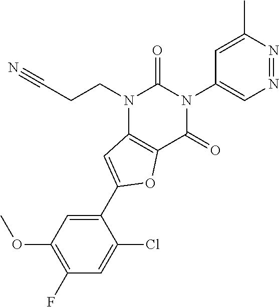 [SMILES: COc1cc(-c2cc3c(o2)c(=O)n(-c2cnnc(C)c2)c(=O)n3CCC#N)c(Cl)cc1F] | 455.8 | ¹H NMR (400 MHz, DMSO- d₆) d 9.14 (d, J = 2.2 Hz, 1H), 7.82 (s, 1H), 7.79-7.69 (m, 2H), 7.57 (d, J = 8.9 Hz, 1H), 4.33 (t, J = 6.5 Hz, 2H), 3.97 (s, 3H), 2.98 (t, J = 6.5 Hz, 2H), 2.73 (s, 3H). | Example 12 |

### Procedure 5: Example 28

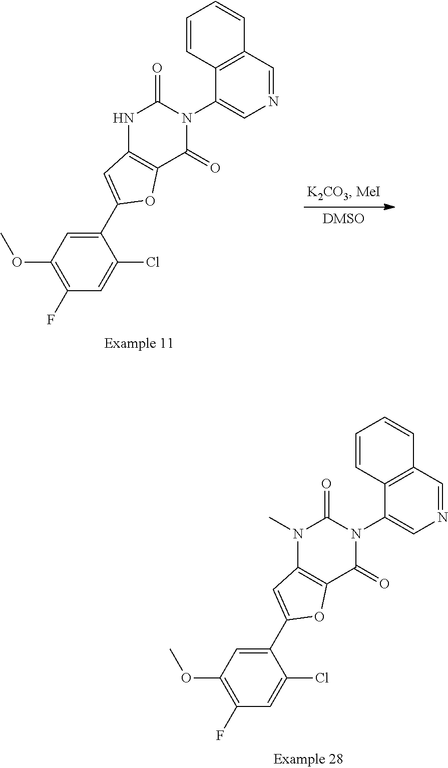 [SMILES: COc1cc(-c2cc3[nH]c(=O)n(-c4cncc5ccccc45)c(=O)c3o2)c(Cl)cc1F ; COc1cc(-c2cc3c(o2)c(=O)n(-c2cncc4ccccc24)c(=O)n3C)c(Cl)cc1F]

6-(2-chloro-4-fluoro-5-methoxy-phenyl)-3-(4-isoquinolyl)-1-methyl-furo[3,2-d]pyrimidine-2,4-dione (Example 28): To a solution of 6-(2-chloro-4-fluoro-5-methoxy-phenyl)-3-(4-isoquinolyl)-1H-furo[3,2-d]pyrimidine-2,4-dione Example 11 (HCl salt) (25 mg, 0.05 mmol, 1.0 equiv.) in dry DMSO (0.35 mL, 0.15 M) was added K₂CO₃ (22 mg, 0.16 mmol, 3.0 equiv.) followed by Mel (4 μL, 0.06 mmol, 1.2 equiv.). The mixture was stirred at 55° C. for 1 hr. The mixture was diluted in acetonitrile/water/trifluoroacetic acid (3 mL; 5:1:0.2), filtered through an acrodisc, and purified directly by RP-HPLC (0.1% TFA-ACN in 0.1% TFA water, Column: Gemini 5 μM, NX—C18 110 Angstrom, 250×21.2 mm) to give the title compound Example 28 as a trifluoroacetate salt.

ES/MS: 451.8 (M⁺)

¹H NMR (400 MHz, DMSO-d₆) δ 9.47 (d, J=0.8 Hz, 1H), 8.54 (s, 1H), 8.30 (dd, J=7.3, 1.6 Hz, 1H), 7.89 (dd, J=8.1, 1.4 Hz, 1H), 7.87-7.71 (m, 3H), 7.69 (s, 1H), 7.61 (d, J=8.9 Hz, 1H), 3.97 (s, 3H), 3.55 (s, 3H)

The following Examples were made in an analogous fashion according to Procedure 5 and are shown below in Table 6. To prepare the below Examples, different reagents/starting materials were used as compared to some of those described in Procedure 5, and are noted in the last column of Table 6—“Changes to Procedure 5: Different Reagents/Starting Materials”. A person of ordinary skill in the art will readily recognize which reagents/starting materials of Procedure 5 were replaced with the different reagents/starting materials noted below.

TABLE 6

| | | | | Changes to |
|---|---|---|---|---|
| | | | | Procedure 5: |
| | | ES/MS | | Different Reagents/ |
| Ex. | Structure | m/z | ¹H-NMR | Starting Materials |
| 29 | 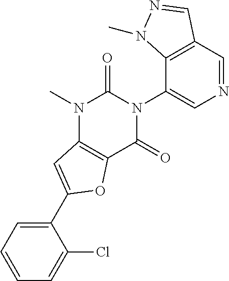 [SMILES: Cn1ncc2cncc(-n3c(=O)c4oc(-c5ccccc5Cl)cc4n(C)c3=O)c21] | 407.8 | ¹H NMR (400 MHz, DMSO- d₆) δ 9.36 (s, 1H), 8.59 (s, 1H), 8.50 (s, 1H), 7.98 (dt, J = 7.1, 3.6 Hz, 1H), 7.75-7.66 (m, 2H), 7.58 (td, J = 5.7, 2.6 Hz, 2H), 3.92 (s, 3H), 3.58 (s, 3H). | Example 6 |
| 30 | 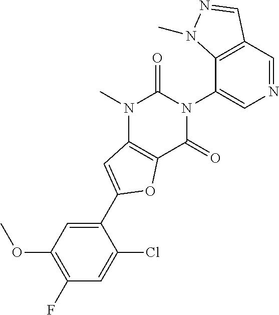 [SMILES: COc1cc(-c2cc3c(o2)c(=O)n(-c2cncc4cnn(C)c24)c(=O)n3C)c(Cl)cc1F] | 455.9 | ¹H NMR (400 MHz, DMSO- d₆) δ 9.31 (s, 1H), 8.55 (s, 1H), 8.44 (s, 1H), 7.75 (d, J = 11.1 Hz, 1H), 7.71 (s, 1H), 7.61 (d, J = 8.9 Hz, 1H), 3.96 (s, 3H), 3.90 (s, 3H), 3.58 (s, 3H). | Example 2 |
| 31 | 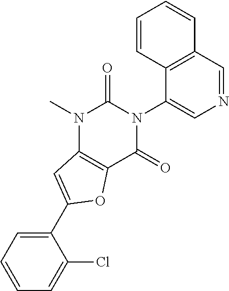 [SMILES: Cn1c(=O)n(-c2cncc3ccccc23)c(=O)c2oc(-c3ccccc3Cl)cc21] | 403.9 | ¹H NMR (400 MHz, DMSO) δ 9.49 (d, J = 0.9 Hz, 1H), 8.56 (s, 1H), 8.35-8.28 (m, 1H), 8.03-7.95 (m, 1H), 7.95-7.88 (m, 1H), 7.82 (dddd, J = 16.0, 8.1, 6.8, 1.4 Hz, 2H), 7.73-7.69 (m, 2H), 7.62-7.53 (m, 2H), 3.56 (s, 3H). | Example 4 |
| 32 | 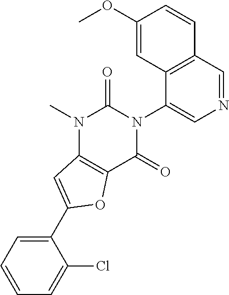 [SMILES: COc1ccc2cncc(-n3c(=O)c4oc(-c5ccccc5Cl)cc4n(C)c3=O)c2c1] | 433.9 | ¹H NMR (400 MHz, DMSO- d₆) δ 9.44 (s, 1H), 8.55 (s, 1H), 8.31 (d, J = 9.1 Hz, 1H), 8.08-7.84 (m, 1H), 7.78- 7.67 (m, 2H), 7.63-7.54 (m, 2H), 7.50 (dd, J = 9.1, 2.4 Hz, 1H), 7.26 (d, J = 2.3 Hz, 1H), 3.91 (s, 3H), 3.57 (s, 3H). | Example 10 |
| 33 | 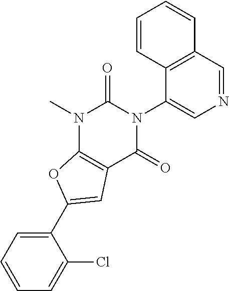 [SMILES: Cn1c(=O)n(-c2cncc3ccccc23)c(=O)c2cc(-c3ccccc3Cl)oc21] | 403.8 | ¹H NMR (400 MHz, DMSO- d₆) δ 9.47 (d, J = 0.9 Hz, 1H), 8.52 (s, 1H), 8.30 (dd, J = 7.5, 1.5 Hz, 1H), 8.01 (dd, J = 7.9, 1.7 Hz, 1H), 7.93 (dd, J = 8.3, 1.3 Hz, 1H), 7.81 (dddd, J = 16.5, 8.1, 6.8, 1.4 Hz, 2H), 7.65 (dd, J = 8.0, 1.3 Hz, 1H), 7.59-7.50 (m, 2H), 7.45 (td, J = 7.7, 1.7 Hz, 1H), 3.61 (s, 3H). | Example 14 |
| 34 | 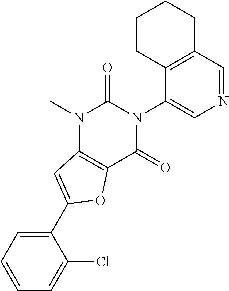 [SMILES: Cn1c(=O)n(-c2cncc3c2CCCC3)c(=O)c2oc(-c3ccccc3Cl)cc21] | 407.9 | ¹H NMR (400 MHz, DMSO- d₆) δ 8.49 (s, 1H), 8.38 (s, 1H), 8.01-7.92 (m, 1H), 7.75-7.64 (m, 2H), 7.61- 7.52 (m, 2H), 3.53 (s, 3H), 2.86 (t, J = 6.1 Hz, 2H), 2.52 (s, 2H), 1.80-1.67 (m, 4H). | Example 5 |
| 35 | 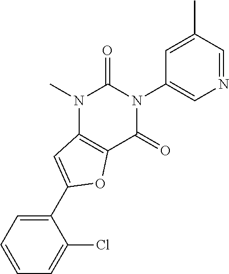 [SMILES: Cc1cncc(-n2c(=O)c3oc(-c4ccccc4Cl)cc3n(C)c2=O)c1] | 367.9 | ¹H NMR (400 MHz, DMSO) δ 8.58-8.53 (m, 1H), 8.46- 8.41 (m, 1H), 8.00-7.92 (m, 1H), 7.77 (td, J = 2.0, 0.8 Hz, 1H), 7.74-7.67 (m, 1H), 7.65 (s, 1H), 7.61-7.52 (m, 2H), 3.52 (s, 3H), 2.41 (s, 3H). | Example 8; DMF |
| 36 | 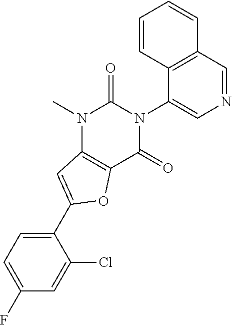 [SMILES: Cn1c(=O)n(-c2cncc3ccccc23)c(=O)c2oc(-c3ccc(F)cc3Cl)cc21] | 421.80 | ¹H NMR (400 MHz, DMSO- d₆) δ 9.48 (s, 1H), 8.55 (s, 1H), 8.31 (dd, J = 7.4, 1.7 Hz, 1H), 8.04 (dd, J = 8.9, 6.1 Hz, 1H), 7.91 (dd, J = 8.2, 1.4 Hz, 1H), 7.89-7.74 (m, 3H), 7.69 (s, 1H), 7.52-7.43 (m, 1H), 3.55 (s, 3H). | Example 1 |

### Procedure 6: Example 37

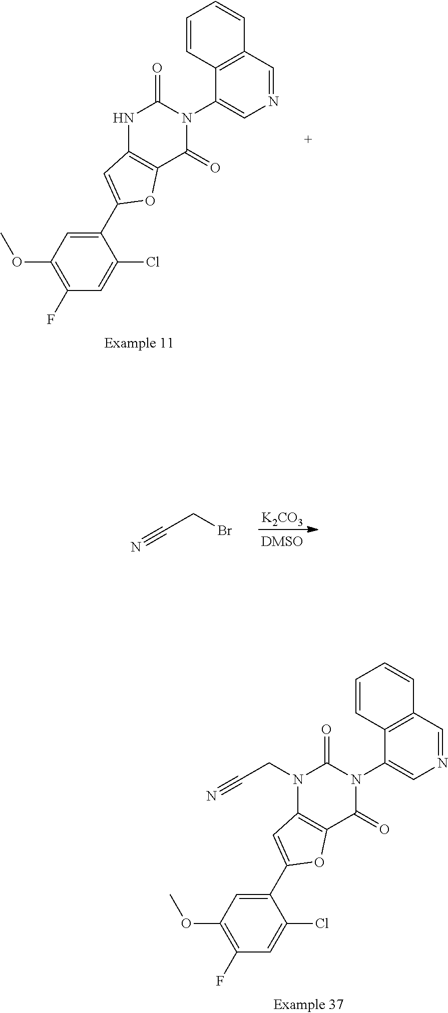

2-(6-(2-chloro-4-fluoro-5-methoxyphenyl)-3-(isoquinolin-4-yl)-2,4-dioxo-3,4-dihydrofuro[3,2-d]pyrimidin-1(2H)-yl)acetonitrile (Example 37): To a solution of 6-(2-chloro-4-fluoro-5-methoxy-phenyl)-3-(4-isoquinolyl)-1H-furo[3,2-d]pyrimidine-2,4-dione (Example 11, HCl salt) (25 mg, 0.05 mmol, 1.0 equiv.) in dry DMSO (0.35 mL, 0.15 M) was added 2-bromoacetonitrile (7.6 mg, 0.063 mmol, 1.2 equiv.) followed by (K₂CO₃ (22 mg, 0.16 mmol, 3.0 equiv.). The mixture stirred for 2 hours at rt. The mixture was diluted in acetonitrile/water/trifluoroacetic acid (5 mL; 5:1:0.2), filtered through an acrodisc, and purified directly by RP-HPLC (0.1% TFA-ACN in 0.1% TFA water, Column: Gemini 5 μM, NX—C18 110 Angstrom, 250×21.2 mm) to give the title compound Example 37 as a trifluoroacetate salt.

ES/MS: 476.8 (M⁺)

¹H NMR (400 MHz, DMSO-d₆) δ 9.51 (d, J=0.8 Hz, 1H), 8.62 (s, 1H), 8.36-8.29 (m, 1H), 7.98 (dd, J=8.3, 1.2 Hz, 1H), 7.91 (s, 1H), 7.90-7.73 (m, 3H), 7.62 (d, J=8.9 Hz, 1H), 5.29 (d, J=1.5 Hz, 2H), 3.97 (s, 3H)

The following Examples were made in an analogous fashion according to Procedure 6 and are shown below in Table 7. To prepare the below Examples, different reagents/starting materials were used as compared to some of those described in Procedure 6, and are noted in the last column of Table 7—“Changes to Procedure 6: Different Reagents/Starting Materials”. A person of ordinary skill in the art will readily recognize which reagents/starting materials of Procedure 6 were replaced with the different reagents/starting materials noted below.

TABLE 7

| | | | | Changes to |
|---|---|---|---|---|
| | | | | Procedure 6: |
| | | ES/MS | | Different Reagents/ |
| Ex. | Structure | m/z | ¹H-NMR | Starting Materials |
| 38 | 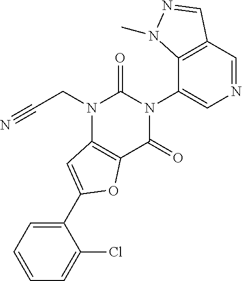 [SMILES: Cn1ncc2cncc(-n3c(=O)c4oc(-c5ccccc5Cl)cc4n(CC#N)c3=O)c21] | 432.8 | ¹H NMR (400 MHz, DMSO- d₆) δ 9.35 (s, 1H), 8.58 (s, 1H), 8.53 (s, 1H), 8.00 (dd, J = 6.0, 3.5 Hz, 1H), 7.97 (s, 1H), 7.73 (dd, J = 6.0, 3.4 Hz, 1H), 7.63-7.52 (m, 2H), 5.42-5.22 (m, 2H), 3.93 (s, 3H) | Example 6 |
| 39 | 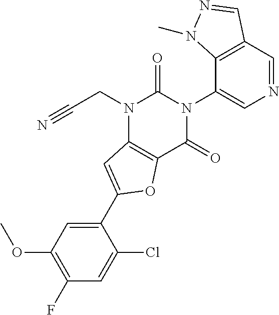 [SMILES: COc1cc(-c2cc3c(o2)c(=O)n(-c2cncc4cnn(C)c24)c(=O)n3CC#N)c(Cl)cc1F] | 480.8 | ¹H NMR (400 MHz, DMSO- d₆) δ 9.33 (s, 1H), 8.56 (s, 1H), 8.51 (s, 1H), 7.93 (s, 1H), 7.77 (d, J = 11.0 Hz, 1H), 7.61 (d, J = 8.9 Hz, 1H), 5.46-5.19 (m, 2H), 3.97 (s, 3H), 3.92 (s, 3H) | Example 2 |
| 40 | 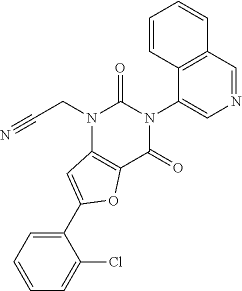 [SMILES: N#CCn1c(=O)n(-c2cncc3ccccc23)c(=O)c2oc(-c3ccccc3Cl)cc21] | 428.8 | ¹H NMR (400 MHz, DMSO) δ 9.50 (d, J = 0.9 Hz, 1H), 8.62 (s, 1H), 8.35-8.28 (m, 1H), 8.05-7.96 (m, 2H), 7.94 (s, 1H), 7.82 (dddd, J = 17.3, 8.1, 6.9, 1.4 Hz, 2H), 7.77-7.69 (m, 1H), 7.63- 7.54 (m, 2H), 5.30 (s, 2H). | Example 4 |
| 41 | 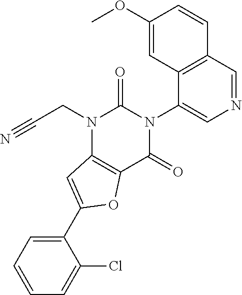 [SMILES: COc1ccc2cncc(-n3c(=O)c4oc(-c5ccccc5Cl)cc4n(CC#N)c3=O)c2c1] | 458.8 | ¹H NMR (400 MHz, DMSO- d₆) δ 9.43 (s, 1H), 8.59 (s, 1H), 8.31 (d, J = 9.0 Hz, 1H), 8.12-7.90 (m, 2H), 7.79- 7.69 (m, 1H), 7.65-7.54 (m, 2H), 7.49 (dd, J = 9.1, 2.4 Hz, 1H), 7.31 (d, J = 2.4 Hz, 1H), 5.48-5.16 (m, 2H), 3.90 (s, 3H). | Example 10 |
| 42 | 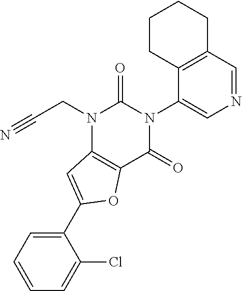 [SMILES: N#CCn1c(=O)n(-c2cncc3c2CCCC3)c(=O)c2oc(-c3ccccc3Cl)cc21] | 432.8 | ¹H NMR (400 MHz, DMSO- d₆) δ 8.47 (s, 1H), 8.38 (s, 1H), 8.03-7.93 (m, 1H), 7.89 (s, 1H), 7.72 (dd, J = 6.0, 3.3 Hz, 1H), 7.63-7.53 (m, 2H), 5.27 (d, J = 2.1 Hz, 2H), 2.86 (t, J = 6.1 Hz, 2H), 2.54 (d, J = 6.7 Hz, 2H), 1.79- 1.68 (m, 4H). | Example 5 |
| 43 | 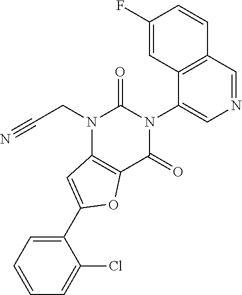 [SMILES: N#CCn1c(=O)n(-c2cncc3ccc(F)cc23)c(=O)c2oc(-c3ccccc3Cl)cc21] | 446.8 | ¹H NMR (400 MHz, DMSO- d₆) δ 9.47 (s, 1H), 8.62 (s, 1H), 8.42 (dd, J = 9.1, 5.6 Hz, 1H), 8.05-7.94 (m, 2H), 7.93 (s, 1H), 7.77-7.66 (m, 2H), 7.63-7.54 (m, 2H), 5.29 (s, 2H). | Example 7 |
| 44 | 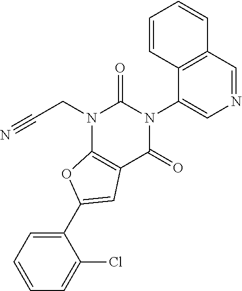 [SMILES: N#CCn1c(=O)n(-c2cncc3ccccc23)c(=O)c2cc(-c3ccccc3Cl)oc21] | 428.8 | ¹H NMR (400 MHz, DMSO) δ 9.46 (s, 1H), 8.56 (s, 1H), 8.30-8.28 (m, 1H), 8.11 (dd, J = 7.9, 1.7 Hz, 1H), 8.00 (d, J = 8.4 Hz, 1H), 7.87-7.74 (m, 3H), 7.67-7.64 (m, 1H), 7.58-7.54 (m, 2H), 7.49- 7.44 (m, 1H), 5.38 (s, 2H). | Example 14 |
| 45 | 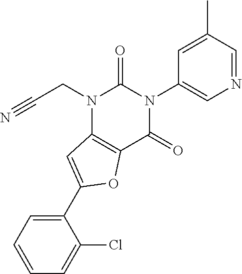 [SMILES: Cc1cncc(-n2c(=O)c3oc(-c4ccccc4Cl)cc3n(CC#N)c2=O)c1] | 392.8 | ¹H NMR (400 MHz, DMSO) δ 8.54 (d, J = 1.9 Hz, 1H), 8.42 (d, J = 2.2 Hz, 1H), 8.02- 7.93 (m, 1H), 7.88 (s, 1H), 7.77-7.67 (m, 2H), 7.62- 7.53 (m, 2H), 5.27 (s, 2H), 2.40 (s, 3H). | Example 8; DMF |
| 46 | 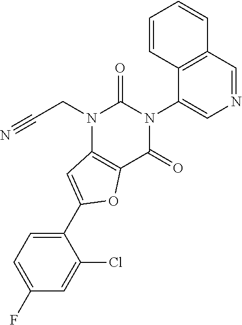 [SMILES: N#CCn1c(=O)n(-c2cncc3ccccc23)c(=O)c2oc(-c3ccc(F)cc3Cl)cc21] | 446.80 | ¹H NMR (400 MHz, DMSO- d₆) δ 9.48 (s, 1H), 8.60 (s, 1H), 8.34-8.27 (m, 1H), 8.06 (dd, J = 8.9, 6.0 Hz, 1H), 8.01-7.94 (m, 1H), 7.91 (s, 1H), 7.88-7.73 (m, 3H), 7.48 (td, J = 8.4, 2.7 Hz, 1H), 5.29 (d, J = 1.1 Hz, 2H). | Example 1 |

### Procedure 7: Example 47

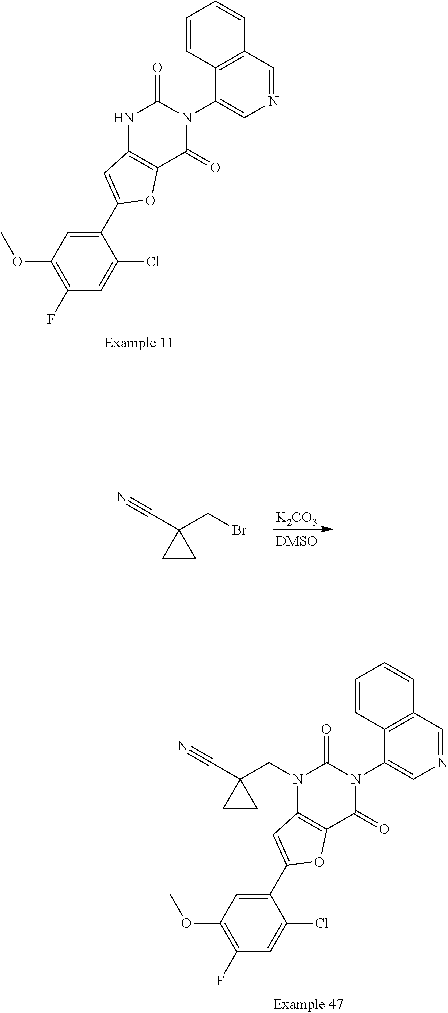

1-((6-(2-chloro-4-fluoro-5-methoxyphenyl)-3-(isoquinolin-4-yl)-2,4-dioxo-3,4-dihydrofuro[3,2-d]pyrimidin-1(2H)-yl)methyl)cyclopropane-1-carbonitrile (Example 47): To a solution of 6-(2-chloro-4-fluoro-5-methoxy-phenyl)-3-(4-isoquinolyl)-1H-furo[3,2-d]pyrimidine-2,4-dione Example 11 (HCl salt) (25 mg, 0.05 mmol, 1.0 equiv.) in dry DMSO (0.35 mL, 0.15 M) was added 1-(bromomethyl)cyclopropanecarbonitrile (10.1 mg, 0.063 mmol, 1.2 equiv.) followed by K₂CO₃ (22 mg, 0.16 mmol, 3.0 equiv.). The mixture stirred for 5 hours at 50° C. The mixture was diluted in acetonitrile/water/trifluoroacetic acid (5 mL; 5:1:0.2), filtered through an acrodisc, and purified directly by RP-HPLC (0.1% TFA-ACN in 0.1% TFA water, Column: Gemini 5 μM, NX—C18 110 Angstrom, 250×21.2 mm) to give the title compound Example 47 as a trifluoroacetate salt.

ES/MS: 516.8 (M⁺)

¹H NMR (400 MHz, DMSO-d₆) δ 9.48 (s, 1H), 8.58 (s, 1H), 8.31 (dd, J=7.4, 1.9 Hz, 1H), 7.92-7.65 (m, 5H), 7.61 (d, J=8.9 Hz, 1H), 4.39 (d, J=15.0 Hz, 1H), 4.23 (d, J=15.1 Hz, 1H), 3.97 (s, 3H), 1.43-1.25 (m, 4H)

The following Examples were made in an analogous fashion according to Procedure 7 and are shown below in Table 8. To prepare the below Examples, different reagents/starting materials were used as compared to some of those described in Procedure 7, and are noted in the last column of Table 8—“Changes to Procedure 7: Different Reagents/Starting Materials”. A person of ordinary skill in the art will readily recognize which reagents/starting materials of Procedure 7 were replaced with the different reagents/starting materials noted below.

TABLE 8

| | | | | Changes to |
|---|---|---|---|---|
| | | | | Procedure 7: |
| | | ES/MS | | Different Reagents/ |
| Ex. | Structure | m/z | ¹H-NMR | Starting Materials |
| 48 | 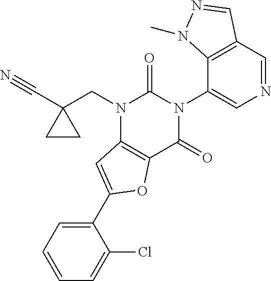 [SMILES: Cn1ncc2cncc(-n3c(=O)c4oc(-c5ccccc5Cl)cc4n(CC4(C#N)CC4)c3=O)c21] | 472.8 | ¹H NMR (400 MHz, DMSO- d₆) δ 9.38 (s, 1H), 8.61 (s, 1H), 8.55 (s, 1H), 8.02-7.96 (m, 1H), 7.93 (s, 1H), 7.76- 7.69 (m, 1H), 7.63-7.54 (m, 2H), 4.34 (dd, J = 15.1 Hz, 2H), 3.96 (s, 3H), 1.36 (ddt, J = 14.9, 8.9, 4.6 Hz, 4H). | Example 6 |
| 49 | 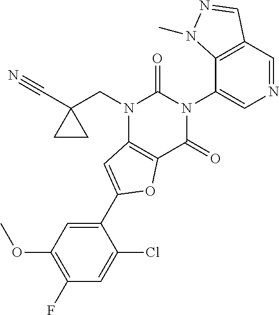 [SMILES: COc1cc(-c2cc3c(o2)c(=O)n(-c2cncc4cnn(C)c24)c(=O)n3CC2(C#N)CC2)c(Cl)cc1F] | 520.9 | ¹H NMR (400 MHz, DMSO- d₆) δ 9.31 (s, 1H), 8.55 (s, 1H), 8.48 (s, 1H), 7.89 (s, 1H), 7.76 (d, J = 11.0 Hz, 1H), 7.61 (d, J = 8.9 Hz, 1H), 4.50-4.13 (m, 2H), 3.97 (s, 3H), 3.94 (s, 3H), 1.36 (tdd, J = 17.2, 8.9, 4.9 Hz, 4H). | Example 2 |
| 50 | 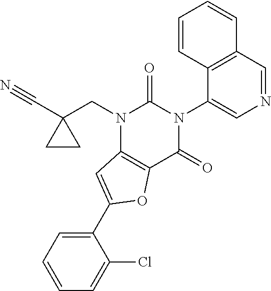 [SMILES: N#CC1(Cn2c(=O)n(-c3cncc4ccccc34)c(=O)c3oc(-c4ccccc4Cl)cc32)CC1] | 468.8 | ¹H NMR (400 MHz, DMSO) δ 9.52 (d, J = 0.9 Hz, 1H), 8.62 (s, 1H), 8.37-8.30 (m, 1H), 8.02-7.96 (m, 1H), 7.94 (dd, J = 8.2, 1.4 Hz, 1H), 7.91 (s, 1H), 7.82 (dddd, J = 16.0, 8.1, 6.8, 1.4 Hz, 2H), 7.77-7.68 (m, 1H), 7.63- 7.53 (m, 2H), 4.41 (d, J = 15.1 Hz, 1H), 4.24 (d, J = 15.1 Hz, 1H), 1.38-1.26 (m, 4H). | Example 4 |
| 51 | 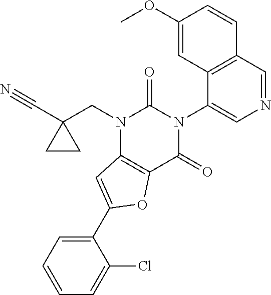 [SMILES: COc1ccc2cncc(-n3c(=O)c4oc(-c5ccccc5Cl)cc4n(CC4(C#N)CC4)c3=O)c2c1] | 498.9 | ¹H NMR (400 MHz, DMSO- d₆) δ 9.44 (s, 1H), 8.59 (s, 1H), 8.31 (d, J = 9.1 Hz, 1H), 8.05-7.96 (m, 1H), 7.91 (s, 1H), 7.78-7.69 (m, 1H), 7.64-7.55 (m, 2H), 7.49 (dd, J = 9.1, 2.4 Hz, 1H), 7.22 (d, J = 2.3 Hz, 1H), 4.46 (d, J = 15.0 Hz, 1H), 4.20 (d, J = 15.0 Hz, 1H), 3.90 (s, 3H), 1.53-1.21 (m, 4H). | Example 10 |
| 52 | 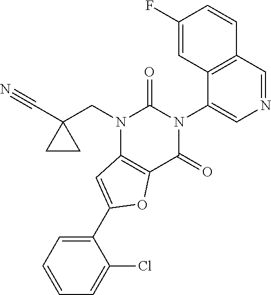 [SMILES: N#CC1(Cn2c(=O)n(-c3cncc4ccc(F)cc34)c(=O)c3oc(-c4ccccc4Cl)cc32)CC1] | 486.8 | ¹H NMR (400 MHz, DMSO- d₆) δ 9.48 (d, J = 0.8 Hz, 1H), 8.62 (s, 1H), 8.42 (dd, J = 9.1, 5.6 Hz, 1H), 8.01-7.95 (m, 1H), 7.93-7.83 (m, 2H), 7.79-7.63 (m, 2H), 7.63- 7.53 (m, 2H), 4.41 (d, J = 15.1 Hz, 1H), 4.21 (d, J = 15.1 Hz, 1H), 1.34 (tq, J = 4.8, 2.5 Hz, 4H). | Example 7 |
| 53 | 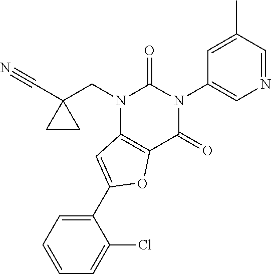 [SMILES: Cc1cncc(-n2c(=O)c3oc(-c4ccccc4Cl)cc3n(CC3(C#N)CC3)c2=O)c1] | 432.8 | ¹H NMR (400 MHz, DMSO) δ 8.54 (d, J = 2.0 Hz, 1H), 8.43 (d, J = 2.3 Hz, 1H), 7.98- 7.93 (m, 1H), 7.86 (s, 1H), 7.77 (t, J = 2.2 Hz, 1H), 7.73- 7.68 (m, 1H), 7.59-7.53 (m, 2H), 4.44 (bs, 1H), 4.21 (bs, 1H), 2.40 (s, 3H), 1.34 (s, 4H). | Example 8 |
| 54 | 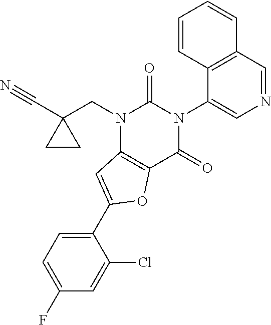 [SMILES: N#CC1(Cn2c(=O)n(-c3cncc4ccccc34)c(=O)c3oc(-c4ccc(F)cc4Cl)cc32)CC1] | 486.80 | ¹H NMR (400 MHz, DMSO- d₆) δ 9.51 (s, 1H), 8.61 (s, 1H), 8.32 (dd, J = 7.3, 1.7 Hz, 1H), 8.04 (dd, J = 8.9, 6.1 Hz, 1H), 7.92 (dd, J = 8.2, 1.4 Hz, 1H), 7.88 (s, 1H), 7.86-7.77 (m, 2H), 7.81-7.72 (m, 1H), 7.49 (td, J = 8.5, 2.6 Hz, 1H), 4.40 (d, J = 15.1 Hz, 1H), 4.23 (d, J = 15.1 Hz, 1H), 1.42-1.27 (m, 4H). | Example 1 |

### Procedure 8: Example 55

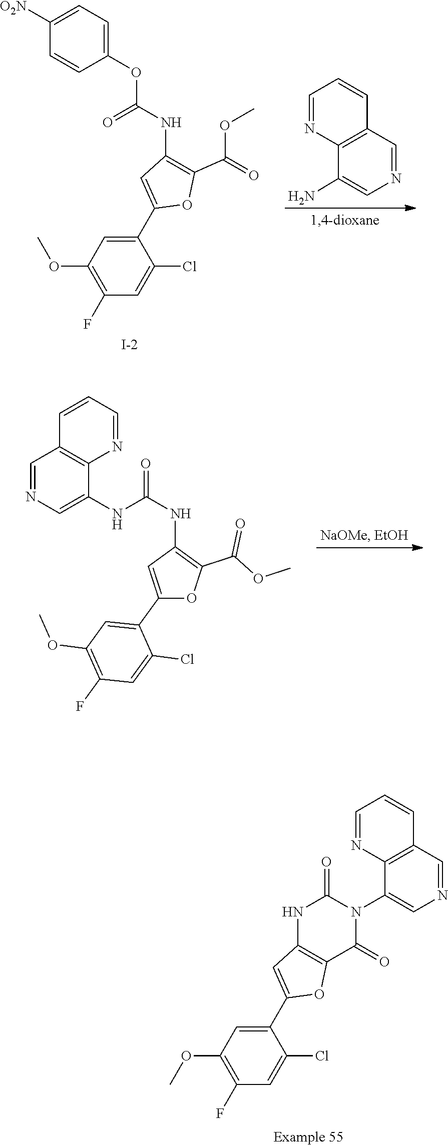

Methyl 3-(3-(1,6-naphthyridin-8-yl)ureido)-5-(2-chloro-4-fluoro-5-methoxyphenyl)furan-2-carboxylate: To a solution of methyl 5-(2-chloro-4-fluoro-5-methoxyphenyl)-3-(((4-nitrophenoxy)carbonyl)amino)furan-2-carboxylate 1-2 (110 mg, 0.24 mmol) in 1,4-dioxane (2 mL), was added 1,6-naphthyridin-8-amine (103 mg, 0.71 mmol). The mixture was heated at 80° C. for 2 hours, until LC/MS showed completion of reaction. The mixture was cooled, diluted with 3 ml acetonitrile, and filtered to provide a solid including the product. The solid was dried under vacuum and carried on to the next step without further purification.

ES/MS: 471.1 (M+H⁺)

6-(2-chloro-4-fluoro-5-methoxyphenyl)-3-(1,6-naphthyridin-8-yl)furo[3,2-d]pyrimidine-2,4(1H,3H)-dione (Example 55): To a solution of methyl 3-(3-(1,6-naphthyridin-8-yl)ureido)-5-(2-chloro-4-fluoro-5-methoxyphenyl)furan-2-carboxylate (110 mg, 0.23 mmol) in ethanol (2 mL) was added sodium methoxide (0.5 ml, 25% in methanol). The mixture was stirred for 2 hours, until LC/MS showed completion of reaction, and then concentrated to provide a residue. The residue was dissolved in DMF, filtered, and purified by HPLC to obtain the product Example 55.

ES/MS: 439.2 (M+H⁺)

¹H NMR (400 MHz, DMSO) δ 12.02 (s, 1H), 9.55 (s, 1H), 9.12 (dd, 1H), 8.81 (s, 1H), 8.74 (dd, 1H), 7.80 (dd, 1H), 7.74 (d, 1H), 7.62 (d, 1H), 7.19 (s, 1H), 3.96 (s, 3H)

The following Examples were made in an analogous fashion according to Procedure 8 and are shown below in Table 9. To prepare the below Examples, different reagents/starting materials were used as compared to some of those described in Procedure 8, and are noted in the last column of Table 9—“Changes to Procedure 8: Different Reagents/Starting Materials”. A person of ordinary skill in the art will readily recognize which reagents/starting materials of Procedure 8 were replaced with the different reagents/starting materials noted below.

TABLE 9

| | | | | Changes to |
|---|---|---|---|---|
| | | | | Procedure 8: |
| | | ES/MS | | Different Reagents/ |
| Ex. | Structure | m/z | ¹H-NMR | Starting Materials |
| 56 | 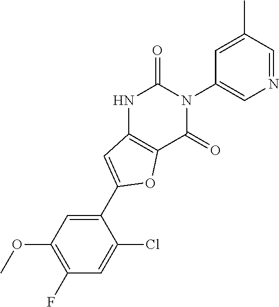 [SMILES: COc1cc(-c2cc3[nH]c(=O)n(-c4cncc(C)c4)c(=O)c3o2)c(Cl)cc1F] | 402.2 | ¹H NMR (400 MHz, DMSO) δ 11.94 (s, 1H), 8.50 (d, 1H), 8.38 (d, 1H), 7.76-7.68 (m, 2H), 7.58 (d, 1H), 7.14 (s, 1H), 3.96 (s, 3H), 2.39 (s, 3H). | 5-methylpyridin-3- amine |

### Procedure 9: Example 57

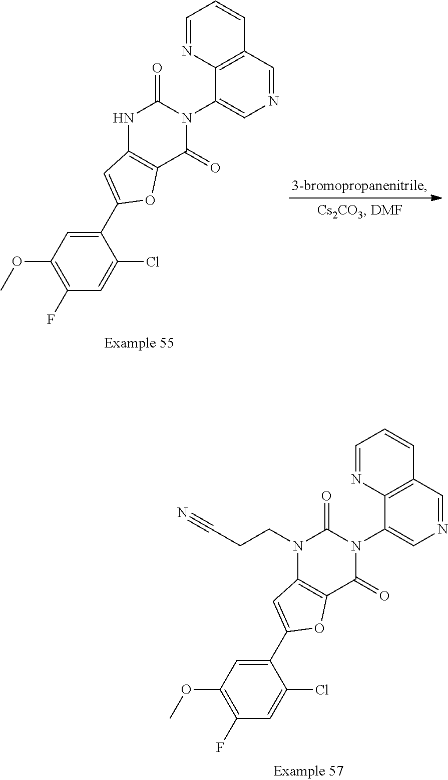 [SMILES: COc1cc(-c2cc3[nH]c(=O)n(-c4cncc5cccnc45)c(=O)c3o2)c(Cl)cc1F ; COc1cc(-c2cc3c(o2)c(=O)n(-c2cncc4cccnc24)c(=O)n3CCC#N)c(Cl)cc1F]

3-(6-(2-chloro-4-fluoro-5-methoxyphenyl)-3-(1,6-naphthyridin-8-yl)-2,4-dioxo-3,4-dihydrofuro[3,2-d]pyrimidin-1(2H)-yl)propanenitrile (Example 57): To a solution of 6-(2-chloro-4-fluoro-5-methoxyphenyl)-3-(1,6-naphthyridin-8-yl)furo[3,2-d]pyrimidine-2,4(1H,3H)-dione (Example 55) (200 mg, 0.46 mmol) in DMF (2 mL) was added cesium carbonate (743 mg, 2.3 mmol) and 3-bromopropanenitrile (0.17 mL, 2.1 mmol). The mixture was stirred at 80° C., filtered, and purified by HPLC to obtain the product.

ES/MS: 492.1 (M+H⁺)

¹H NMR (400 MHz, DMSO) δ 9.57 (s, 1H), 9.11 (dd, 1H), 8.82 (s, 1H), 8.75 (dd, 1H), 7.86-7.76 (m, 2H), 7.76 (d, 1H), 7.62 (d, 1H), 4.36 (t, 2H), 3.97 (s, 3H), 3.00 (td, 2H)

The following Examples were made in an analogous fashion according to Procedure 9 and are shown below in Table 10. To prepare the below Examples, different reagents/starting materials were used as compared to some of those described in Procedure 9, and are noted in the last column of Table 10—“Changes to Procedure 9: Different Reagents/Starting Materials”. A person of ordinary skill in the art will readily recognize which reagents/starting materials of Procedure 9 were replaced with the different reagents/starting materials noted below.

TABLE 10

| | | | | Changes to |
|---|---|---|---|---|
| | | | | Procedure 9: |
| | | ES/MS | | Different Reagents/ |
| Ex. | Structure | m/z | ¹H-NMR | Starting Materials |
| 58 | 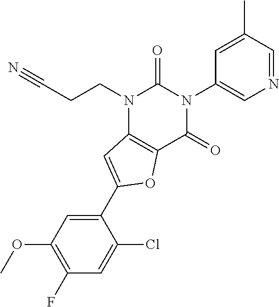 [SMILES: COc1cc(-c2cc3c(o2)c(=O)n(-c2cncc(C)c2)c(=O)n3CCC#N)c(Cl)cc1F] | 455.2 | ¹H NMR (400 MHz, DMSO) δ 8.51 (d, 1H), 8.38 (d, 1H), 7.79 (s, 1H), 7.75 (d, 1H), 7.70-7.67 (m, 1H), 7.57 (d, 1H), 4.32 (t, 2H), 3.97 (s, 3H), 2.98 (t, 2H), 2.39 (s, 3H). | Example 56 |

### Procedure 10: Example 59 and Example 60

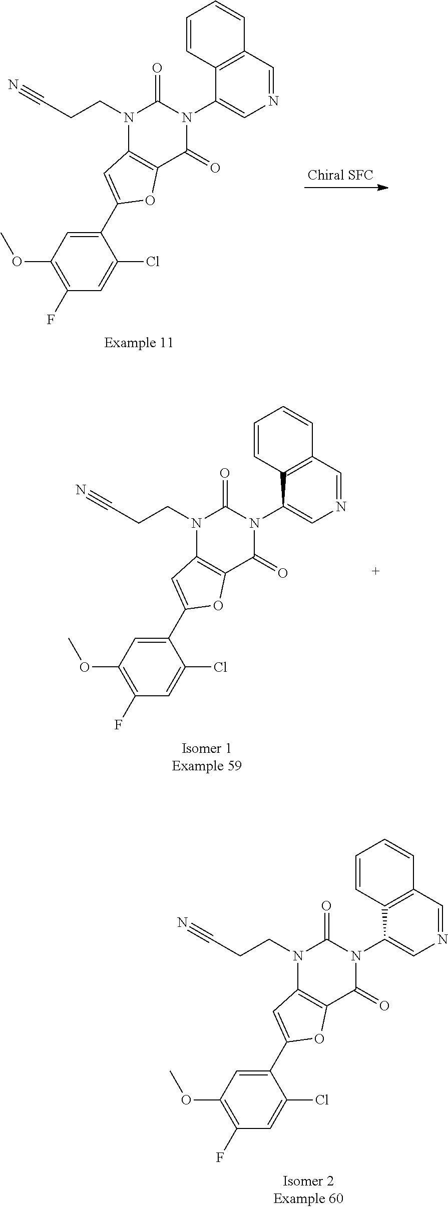

3-[6-(2-chloro-4-fluoro-5-methoxy-phenyl)-3-(4-isoquinolyl)-2,4-dioxo-furo[3,2-d]pyrimidin-1-yl]propanenitrile (Example 59 and Example 60): 3-[6-(2-chloro-4-fluoro-5-methoxy-phenyl)-3-(4-isoquinolyl)-2,4-dioxo-furo[3,2-d]pyrimidin-1-yl]propanenitrile Example 11 as a mixture of 2 atropisomers was separated by chiral SFC (ID Sum 21×250 mm column with 45% MeOH cosolvent) to give two enantiomers, Isomer 1 and Isomer 2. The stereochemistry was assigned for the first-eluting Example 59 (Isomer 1), and the second-eluting Example 60 (Isomer 2).

### Isomer 1

3-[6-(2-chloro-4-fluoro-5-methoxy-phenyl)-3-(4-isoquinolyl)-2,4-dioxo-furo[3,2-d]pyrimidin-1-yl]propanenitrile (Example 59)

ES/MS: 490.8 (M⁺)

¹H NMR (400 MHz, DMSO-d₆) δ 9.47 (d, J=0.8 Hz, 1H), 8.55 (s, 1H), 8.30 (dd, J=7.4, 2.0 Hz, 1H), 7.93-7.72 (m, 6H), 7.60 (d, J=8.9 Hz, 1H), 4.36 (hept, J=6.7 Hz, 2H), 3.97 (s, 3H), 3.01 (t, J=6.6 Hz, 2H)

### Isomer 2

### 3-[6-(2-chloro-4-fluoro-5-methoxy-phenyl)-3-(4-isoquinolyl)-2,4-dioxo-furo[3,2-d]pyrimidin-1-yl]propanenitrile (Example 60)

ES/MS: 490.8 (M+)

¹H NMR (400 MHz, DMSO-d₆) δ 9.47 (d, J=0.8 Hz, 1H), 8.56 (s, 1H), 8.30 (dd, J=7.3, 1.9 Hz, 1H), 7.93-7.72 (m, 5H), 7.60 (d, J=8.9 Hz, 1H), 4.37 (hept, J=7.3 Hz, 2H), 3.97 (s, 3H), 3.01 (t, J=6.5 Hz, 2H)

### Procedure 11: Example 61 and Example 62

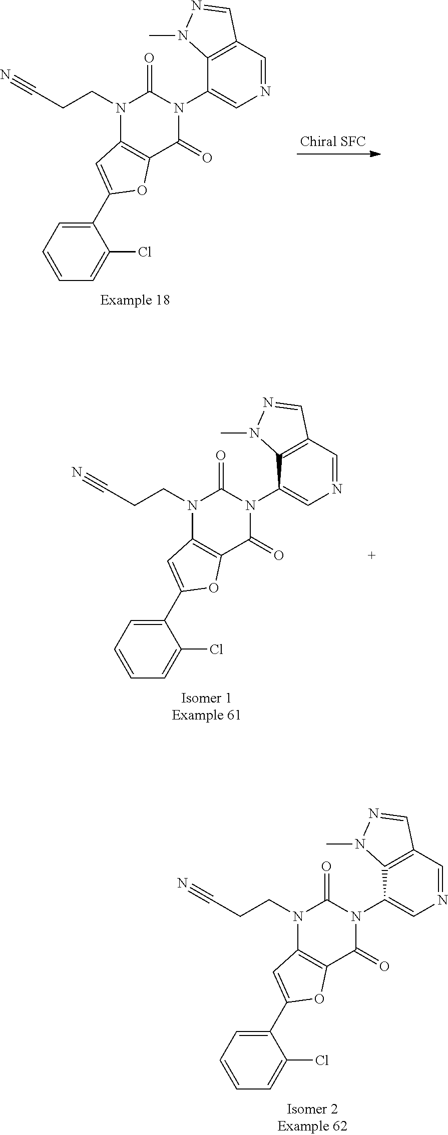

3-[6-(2-chlorophenyl)-3-(1-methylpyrazolo[4,3-c]pyridin-7-yl)-2,4-dioxo-furo[3,2-d]pyrimidin-1-yl]propanenitrile (Example 61 and Example 62): 3-[6-(2-Chlorophenyl)-3-(1-methylpyrazolo[4,3-c]pyridin-7-yl)-2,4-dioxo-furo[3,2-d]pyrimidin-1-yl]propanenitrile Example 18 as a mixture of 2 enantiomers was separated by chiral SFC (AD-H 5 um-4.6×100 mm column with 45% MeOH cosolvent) to give two enantiomers, Isomer 1 and Isomer 2. The stereochemistry was assigned for the first-eluting Example 61 (Isomer 1), and the second-eluting Example 62 (Isomer 2).

### Isomer 1

### 3-[6-(2-chlorophenyl)-3-(1-methylpyrazolo[4,3-c]pyridin-7-yl)-2,4-dioxo-furo[3,2-d]pyrimidin-1-yl]propanenitrile (Example 61)

ES/MS: 446.8 (M⁺)

¹H NMR (400 MHz, DMSO-d₆) δ 9.35 (s, 1H), 8.58 (s, 1H), 8.50 (s, 1H), 7.99 (dd, J=6.1, 3.5 Hz, 1H), 7.90 (s, 1H), 7.71 (dt, J=7.4, 3.7 Hz, 1H), 7.63-7.53 (m, 2H), 4.51-4.28 (m, 2H), 3.93 (s, 3H), 3.04 (td, J=6.5, 2.3 Hz, 2H)

### Isomer 2

### 3-[6-(2-chlorophenyl)-3-(1-methylpyrazolo[4,3-c]pyridin-7-yl)-2,4-dioxo-furo[3,2-d]pyrimidin-1-yl]propanenitrile (Example 62)

ES/MS: 446.8 (M⁺)

¹H NMR (400 MHz, DMSO-d₆) δ 9.35 (s, 1H), 8.58 (s, 1H), 8.50 (s, 1H), 8.03-7.94 (m, 1H), 7.90 (s, 1H), 7.72 (dd, J=5.9, 3.4 Hz, 1H), 7.58 (dd, J=6.0, 3.5 Hz, 2H), 4.50-4.28 (m, 2H), 3.93 (s, 3H), 3.04 (td, J=6.5, 2.3 Hz, 2H)

### Procedure 12: Example 63 and Example 64

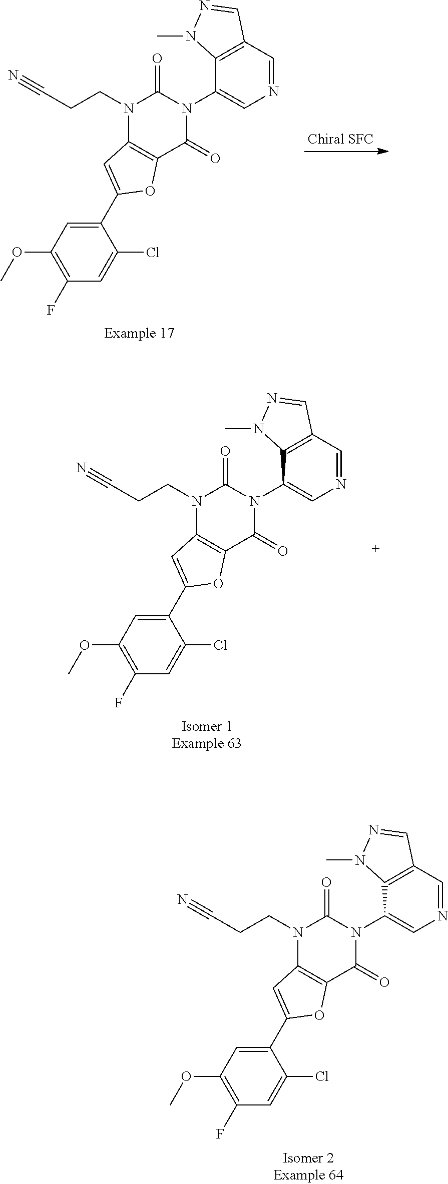

3-[6-(2-chloro-4-fluoro-5-methoxy-phenyl)-3-(1-methylpyrazolo[4,3-c]pyridin-7-yl)-2,4-dioxo-furo[3,2-d]pyrimidin-1-yl]propanenitrile (Example 63 and Example 64): 3-[6-(2-Chloro-4-fluoro-5-methoxy-phenyl)-3-(1-methylpyrazolo[4,3-c]pyridin-7-yl)-2,4-dioxo-furo[3,2-d]pyrimidin-1-yl]propanenitrile Example 17 as a mixture of 2 enantiomers was separated by chiral SFC (IB 5 um-4.6×100 mm column with 35% MeOH cosolvent) to give two enantiomers, Isomer 1 and Isomer 2. The stereochemistry was assigned for the first-eluting Example 63 (Isomer 1), and the second eluting Example 64 (Isomer 2).

### Isomer 1

### 3-[6-(2-chloro-4-fluoro-5-methoxy-phenyl)-3-(1-methylpyrazolo[4,3-c]pyridin-7-yl)-2,4-dioxo-furo[3,2-d]pyrimidin-1-yl]propanenitrile (Example 63)

ES/MS: 494.8 (M⁺)

¹H NMR (400 MHz, DMSO-d₆) δ 9.32 (s, 1H), 8.55 (s, 1H), 8.46 (s, 1H), 7.87 (s, 1H), 7.76 (d, J=11.0 Hz, 1H), 7.60 (d, J=8.9 Hz, 1H), 4.48-4.28 (m, 2H), 3.97 (s, 3H), 3.92 (s, 3H), 3.05 (td, J=6.5, 2.8 Hz, 2H)

### Isomer 2

### 3-[6-(2-chloro-4-fluoro-5-methoxy-phenyl)-3-(1-methylpyrazolo[4,3-c]pyridin-7-yl)-2,4-dioxo-furo[3,2-d]pyrimidin-1-yl]propanenitrile (Example 64)

ES/MS: 494.8 (M⁺)

¹H NMR (400 MHz, DMSO-d₆) δ 9.33 (s, 1H), 8.56 (s, 1H), 8.47 (s, 1H), 7.87 (s, 1H), 7.76 (d, J=11.0 Hz, 1H), 7.60 (d, J=8.9 Hz, 1H), 4.52-4.25 (m, 2H), 3.97 (s, 3H), 3.92 (s, 3H), 3.05 (td, J=7.0, 6.6, 2.7 Hz, 2H)

Procedure 13: Example 65 and Example 66

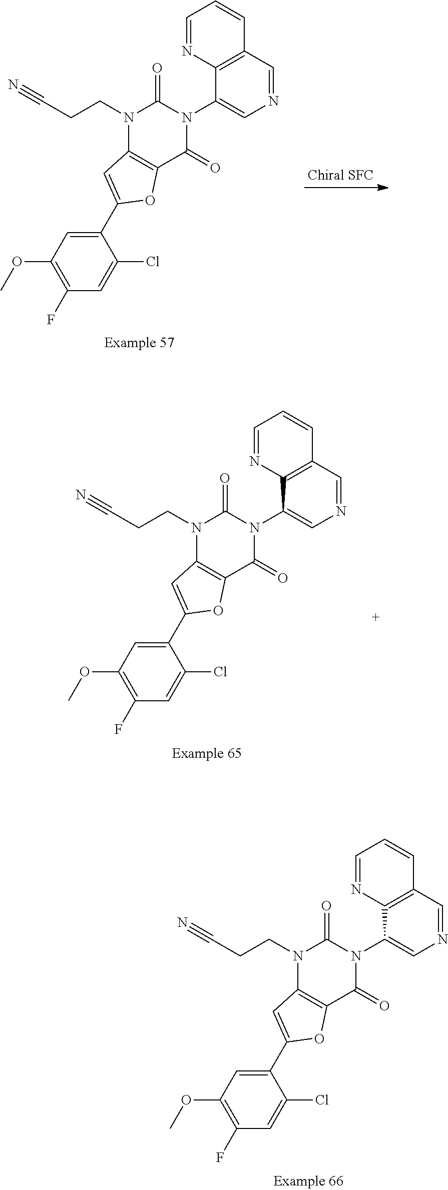

3-(6-(2-chloro-4-fluoro-5-methoxyphenyl)-3-(1,6-naphthyridin-8-yl)-2,4-dioxo-3,4-dihydrofuro[3,2-d]pyrimidin-1(2H)-yl)propanenitrile (Example 64 and Example 65): 3-(6-(2-chloro-4-fluoro-5-methoxyphenyl)-3-(1,6-naphthyridin-8-yl)-2,4-dioxo-3,4-dihydrofuro[3,2-d]pyrimidin-1(2H)-yl)propanenitrile Example 57 as a mixture of 2 atropisomers was separated by chiral SFC (AD-H 5 μm 4.6×100 mm column with 45% MeOH cosolvent) to give two enantiomers, Isomer 1 and Isomer 2. The stereochemistry was assigned for the first-eluting Example 65 (Isomer 1), and the second-eluting Example 66 (Isomer 2).

### Isomer 1

### 3-(6-(2-chloro-4-fluoro-5-methoxyphenyl)-3-(1,6-naphthyridin-8-yl)-2,4-dioxo-3,4-dihydrofuro[3,2-d]pyrimidin-1(2H)-yl)propanenitrile (Example 65)

ES/MS: 492.1 (M⁺)

¹H NMR (400 MHz, DMSO) δ 9.57 (s, 1H), 9.10 (dd, 1H), 8.82 (s, 1H), 8.75 (dd, 1H), 7.84 (s, 1H), 7.80 (dd, 1H), 7.76 (d, 1H), 7.62 (d, 1H), 4.36 (t, 2H), 3.97 (s, 3H), 3.00 (td, 2H)

### Isomer 2

### 3-(6-(2-chloro-4-fluoro-5-methoxyphenyl)-3-(1,6-naphthyridin-8-yl)-2,4-dioxo-3,4-dihydrofuro[3,2-d]pyrimidin-1(2H)-yl)propanenitrile (Example 66)

ES/MS: 492.1 (M⁺)

¹H NMR (400 MHz, DMSO) δ 9.57 (s, 1H), 9.10 (dd, 1H), 8.82 (s, 1H), 8.75 (dd, 1H), 7.84 (s, 1H), 7.80 (dd, 1H), 7.76 (d, 1H), 7.62 (d, 1H), 4.36 (t, 2H), 3.97 (s, 3H), 3.00 (td, 2H)

### III. Biological Examples

### Example A. Cov Mpro Biochemical IC50 Assay

This biochemical assay measured small molecule inhibitor IC₅₀ values against recombinant SARS-CoV-2 Main protease (Mpro). Compound dilutions were prepared and pre-incubated for 20 minutes while mixing at room temperature with 2×Mpro. The final Mpro concentration was 7.5 nM with the fluorogenic peptide substrate (DABCYL-KTSAVLQ/SGFRKME-EDANS) at its Km value of 40 μM in a reaction volume of 40 μL. Product formation was quantitated in kinetic mode every 2.5 minutes for 8 cycles by monitoring fluorescence at EX/EM wavelength of 340/460. Rates of reaction (slope with background subtracted) from inhibitor wells were normalized to DMSO wells and then curve fitted for IC₅₀ using the PRISM algorithm: log(inhibitor) vs. normalized response —Variable slope. The IC₅₀ value for each compound was defined as the concentration reducing enzyme activity by 50%.

### Example B. A549-hACE2 SARS-CoV2-NLuc 384-Well Assay

A549-hACE2 cell line was maintained in Dulbecco's Minimum Essential Medium (DMEM) (Corning, New York, NY, Cat #15-018CM) supplemented with 10% fetal bovine serum (FBS) (Hyclone, Logan, UT, Cat #SH30071-03), 1× Penicillin-Streptomycin-L-Glutamine (Corning, New York, NY, Cat #30-009-CI) and 10 pg/mL blasticidin (Life Technologies Corporation, Carlsbad, CA, Cat #A11139-03). Cells were passaged 2 times per week to maintain sub-confluent densities and were used for experiments at passage 5-20. SARS Coronavirus 2 recombinant with NanoLuc (SARS-CoV-2-NLuc) was obtained from University of Texas Medical Branch (Galveston, TX). Viral replication was determined in A549-hACE2 cells in the following manner.

Compounds were prepared in 100% DMSO in 384-well polypropylene plates (Greiner, Monroe, NC, Cat #784201) with 8 compounds per plate in grouped replicates of 4 at 10 serially diluted concentrations (1:3). The serially diluted compounds were transferred to low dead volume Echo plates (Labcyte, Sunnyvale, CA, Cat #LP-0200).

The test compounds were spotted to 384-well assay plates (Greiner, Monroe, NC, Cat #781091) at 200 nL per well using an Echo acoustic dispenser (Labcyte, Sunnyvale, CA). A549-hACE2 cells were harvested and suspended in DMEM (supplemented with 2% FBS and 1× Penicillin-Streptomycin-L-Glutamine) and seeded to the pre-spotted assay plates at 10,000 cells per well in 30 μL. SARS-CoV2-NLuc virus was diluted in DMEM (supplemented with 2% FBS and 1× Penicillin-Streptomycin-L-Glutamine) at 350,000 Infectious Units (IU) per mL and 10 μL per well was added to the assay plates containing cells and compounds, for MOI 0.35. The assay plates were incubated for 2 days at 37° C. and 5% CO₂. At the end of incubation, Nano-Glo reagent (Promega, Madison, WI, Cat #N1150) was prepared. The assay plates and Nano-Glo reagent were equilibrated to room temperature for at least 30 minutes. 40 μL per well of Nano-Glo reagent was added and the plates were incubated at room temperature for 30 minutes before reading the luminescence signal on an EnVision multimode plate reader (Perkin Elmer, Waltham, MA). Remdesivir was used as positive control and DMSO was used as negative control. Values were normalized to the positive and negative controls (as 0% and 100% replication, respectively) and data was fitted using non-linear regression analysis by Gilead's dose response tool. The EC₅₀ value for each compound was defined as the concentration reducing viral replication by 5000.

### Biological Data

Provided below in Table 11 is data related to compounds disclosed herein.

TABLE 11
Biological Data for Compounds Disclosed Herein

| Example | MPro IC50 (nM) | MPro EC50 (nM) |
|---|---|---|
| 1 | 171.78 | 448.43 |
| 2 | 50.97 | 244.54 |
| 3 | 12.83 | 23.33 |
| 4 | 92.12 | 273.32 |
| 5 | 67.6 | 211.99 |
| 6 | 52.67 | 173.67 |
| 7 | 42.23 | 79.64 |
| 8 | 35.86 | 136.67 |
| 9 | 303.82 | 1010.16 |
| 10 | 33.48 | 95.63 |
| 11 | 45.07 | 81.54 |
| 12 | >50000 | >10000 |
| 13 | 991.04 | 4235.75 |
| 14 | 30.38 | 3327.71 |
| 15 | 537.62 | >10000 |
| 16 | 9.59 | 4.36 |
| 17 | 10.6 | 20.73 |
| 18 | 13.27 | 27.83 |
| 19 | 9.39 | 12.99 |
| 20 | 13.96 | 17.1 |
| 21 | 17.11 | 29.65 |
| 22 | 18 | 18.98 |
| 23 | 11.61 | 24.15 |
| 24 | 30.98 | 71.41 |
| 25 | 24.63 | 41.72 |
| 26 | 68.27 | 407.8 |
| 27 | 934.04 | 5248.08 |
| 28 | 116.45 | 287.98 |
| 29 | 386.95 | 1167.1 |
| 30 | 272.63 | 682.95 |
| 31 | 147.46 | 564.82 |
| 32 | 147.13 | 381.13 |
| 33 | 43 | 145.37 |
| 34 | 350.3 | 960.48 |
| 35 | 310.04 | 725.97 |
| 36 | 1184.73 | 3266.29 |
| 37 | 5.63 | 15.85 |
| 38 | 5.22 | 87.99 |
| 39 | 6.3 | 50.73 |
| 40 | 6.6 | 20.14 |
| 41 | 5.94 | 16.58 |
| 42 | 7.66 | 36.66 |
| 43 | 6.98 | 25.27 |
| 45 | 4.71 | 52.31 |
| 46 | 7.42 | 58.41 |
| 47 | 27.85 | 29.41 |
| 48 | 8.69 | 8.36 |
| 49 | 8.87 | 12.19 |
| 50 | 6.82 | 6.99 |
| 51 | 11.29 | 12.13 |
| 52 | 7.67 | 11.76 |
| 53 | 5.98 | 12.81 |
| 54 | 13.8 | 17.98 |
| 55 | 48.99 | 249.73 |
| 56 | 27.76 | 195.45 |
| 57 | 9.25 | 19.68 |
| 58 | 5.48 | 19.85 |
| 59 | <3.75 | 4.65 |
| 60 | 59.57 | 82.76 |
| 61 | 3.79 | 7.16 |
| 62 | 95.57 | 233.02 |
| 63 | 4.23 | 6.24 |
| 64 | 60.41 | 111.88 |
| 65 | <3.75 | 5.78 |
| 66 | 79.3 | 111.52 |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula (I): 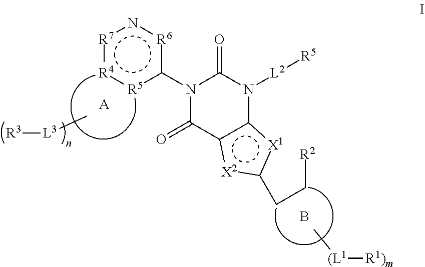
or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
ring \textcircled{A} taken together with X⁴ and X⁵ is phenyl, 5- to 6-membered heteroaryl, C₅₋₁₀ cycloalkyl, or 5- to 10-membered heterocyclyl,
wherein each X⁴ and X⁵ is independently N or C;
alternatively, ring \textcircled{A} is absent, wherein X⁴ is N or C-L^{x4}-R^{x4}, and X⁵ is N or C-L^{x5}-R^{x5};
each L^{x4} and L^{x5} is independently a bond, —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl)-, —(C₁₋₆ alkyl)N(R^{L})S(O)₂—, —N(R^{L})S(O)₂—, —C(O)—, —(C₁₋₆ alkyl)C(O)—, or —N(R^{L})C(O)—;
each R^{x4} and R^{x5} is independently hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, 5- to 10-membered heteroaryl, —NH₂, —NH(C₁₋₆ alkyl), —N(C₁₋₆ alkyl)₂, —O(C₁₋₆ alkyl), or —OC₃₋₈ cycloalkyl;
wherein each C₁₋₆ alkyl, C₂₋₆ alkynyl, C₁₋₆ alkoxy, C₃₋₈ cycloalkyl, and 4- to 10-membered heterocyclyl of R^{x4} and R^{x5} is optionally substituted with one to four R^{4a};
each R^{4a} is independently C₁₋₆ alkyl, C₁₋₆ haloalkyl, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂,
wherein each C₁₋₆ alkyl, C₃₋₈ cycloalkyl, 4- to 10-membered heterocyclyl, phenyl, and 5- to 10-membered heteroaryl of R^{4a} is optionally substituted with one to three R^{4b};
each R^{4b} is independently C₁₋₆ alkyl, C₁₋₆ haloalkyl, C₃₋₆ cycloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂;
each L³ is independently a bond, —(C₁₋₆ alkyl)O—, —(C₁₋₆ alkyl)N(R^{L})C(O)—, —(C₁₋₆ alkyl)C(O)N(R^{L})—, —(C₁₋₆ alkyl)N(R^{L})C(O)(C₁₋₆ alkyl)-, —(C₁₋₆ alkyl)C(O)N(R^{L})(C₁₋₆ alkyl)-, —(C₁₋₆ alkyl)-, —(C₁₋₆ alkyl)N(R^{L})S(O)₂—, —N(R^{L})S(O)₂—, —C(O)—, —(C₁₋₆ alkyl)C(O)—, or —N(R^{L})C(O)—;
each R³ is independently a hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, phenyl, 5- to 10-membered heteroaryl, —NH₂, —NH(C₁₋₆ alkyl), —N(C₁₋₆ alkyl)₂, or —OC₃₋₈ cycloalkyl;
wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to four R^{3a};
each R^{3a} is independently C₁₋₆ alkyl, C₁₋₆ haloalkyl, halogen, —C(O)(C₁-C₆ alkyl), C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₁₋₆ haloalkyl)₂, —N(C₃₋₈ cycloalkyl)₂, —N(C₁₋₆ alkyl)(C₁₋₆ haloalkyl), —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)NH₂, —C(O)NH(C₁₋₆ alkyl), —C(O)NH(C₁₋₆ haloalkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₁₋₆ haloalkyl)₂, —C(O)N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₁₋₆ haloalkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂,
wherein each C₁₋₆ alkyl, C₃₋₈ cycloalkyl, 4- to 10-membered heterocyclyl, phenyl, and 5- to 10-membered heteroaryl of R^{3a} is optionally substituted with one to three R^{3b};
each R^{3b} is independently C₁₋₆ alkyl, C₁₋₆ haloalkyl, C₃₋₆ cycloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(C₆₋₁₀ aryl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(5- to 10-membered heteroaryl), —NHC(O)O(C₁₋₆ alkyl), —NHC(O)O(C₁₋₆ haloalkyl), —NHC(O)O(C₃₋₈ cycloalkyl), —NHC(O)O(5- to 10-membered heterocyclyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)(NH)(C₁₋₆ alkyl), —S(O)₂NH(C₁₋₆ alkyl), or —S(O)₂N(C₁₋₆ alkyl)₂;
each R^{L} is independently hydrogen, C₁₋₆ alkyl, C₁₋₆ haloalkyl, or C₃₋₈ cycloalkyl;
n is an integer from 0 to 3;
each X⁶ and X⁷ is independently N, CH, or CF,
wherein no more than two of X⁴, X⁵, X⁶, and X⁷ are N;
ring \textcircled{B} is C₆₋₁₀ aryl or 5- to 10-membered heteroaryl;
each L¹ is independently a bond, —O—, —(C₁₋₆ alkyl)O—, —O(C₁₋₆ alkyl)-, —C₁₋₆ alkyl-O(C₁₋₆ alkyl)-, —C(O)—, —N(R^{L})C(O)—, —C(O)N(R^{L})—, —(C₁₋₆ alkyl)(R^{L})NC(O)—, —(C₁₋₆ alkyl)C(O)N(R^{L})—, —N(R^{L})C(O)(C₁₋₆ alkyl)-, —C(O)N(R^{L})(C₁₋₆ alkyl)-, —(C₁₋₆ alkyl)N(R^{L})C(O)(C₁₋₆ alkyl)-, —(C₁₋₆ alkyl)C(O)N(R^{L})(C₁₋₆ alkyl)-, —S(O)₂—, —S(O)₂N(R^{L})—, —N(R^{L})—S(O)₂—, —(C₁₋₆ alkyl)S(O)₂N(R^{L})—, —(C₁₋₆ alkyl)N(R^{L})S(O)₂—, —S(O)₂N(R^{L})(C₁₋₆ alkyl)-, —N(R^{L})S(O)₂(C₁₋₆ alkyl)-, —(C₁₋₆ alkyl)S(O)₂N(R^{L})(C₁₋₆ alkyl)-, or —(C₁₋₆ alkyl)N(R^{L})S(O)₂(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
alternatively, two R¹ taken together with the atoms of ring \textcircled{B} to which they are attached form 5- to 10-membered heterocyclyl optionally substituted with one to three R^{1a};
each R^{1a} is independently C₁₋₆ alkyl, C₁₋₆ alkoxy, halogen, C₃₋₈ cycloalkyl, 4- to 10-membered heterocyclyl, phenyl, 5- to 10-membered heteroaryl, oxo, —OH, —CN, —NH₂, —O(C₁₋₆ alkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —N(5- to 10-membered heterocyclyl)₂, —N(phenyl)₂, —N(5- to 10-membered heteroaryl)₂, —N(C₁₋₆ alkyl)(C₃₋₈ cycloalkyl), —N(C₁₋₆ alkyl)(5- to 10-membered heterocyclyl), —N(C₁₋₆ alkyl)(phenyl), —N(C₁₋₆ alkyl)(5- to 10-membered heteroaryl), —C(O)(5- to 10-membered heterocyclyl), —C(O)(5- to 10-membered heteroaryl), —C(O)O(C₁₋₆ alkyl), —C(O)O(C₃₋₈ cycloalkyl), —C(O)O(5- to 10-membered heterocyclyl), —C(O)O(phenyl), —C(O)O(5- to 10-membered heteroaryl), —C(O)NH₂, —C(O)NH(C₁₋₆ alkyl), —C(O)NH(C₃₋₈ cycloalkyl), —C(O)NH(5- to 10-membered heterocyclyl), —C(O)NH(phenyl), —C(O)NH(5- to 10-membered heteroaryl), —C(O)N(C₁₋₆ alkyl)₂, —C(O)N(C₃₋₈ cycloalkyl)₂, —C(O)N(5- to 10-membered heterocyclyl)₂, —C(O)N(phenyl)₂, —C(O)N(5- to 10-membered heteroaryl)₂, —NHC(O)(C₁₋₆ alkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), —NHC(O)NH(C₃₋₈ cycloalkyl), —NHC(O)NH(5- to 10-membered heterocyclyl), —NHC(O)NH(phenyl), —NHC(O)NH(5- to 10-membered heteroaryl), —NHS(O)(C₁₋₆ alkyl), —N(C₁₋₆ alkyl)(S(O)(C₁₋₆ alkyl), —S(O)₂(C₁₋₆ alkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂,
wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
alternatively, R¹ is phenyl, and two R^{1a} taken together with the atoms of the phenyl to which they are attached form 5- to 10-membered heterocyclyl optionally substituted with one to three R^{1b};
each R^{1b} is independently C₁₋₆ alkyl, C₁₋₆ haloalkyl, halogen, oxo, —OH, —NH₂, CO₂H, —O(C₁₋₆ alkyl), —O(C₁₋₆ haloalkyl), —O(C₃₋₈ cycloalkyl), —O(5- to 10-membered heterocyclyl), —O(phenyl), —O(5- to 10-membered heteroaryl), —NH(C₁₋₆ alkyl), —NH(C₁₋₆ haloalkyl), —NH(C₃₋₈ cycloalkyl), —NH(5- to 10-membered heterocyclyl), —NH(phenyl), —NH(5- to 10-membered heteroaryl), —N(C₁₋₆ alkyl)₂, —N(C₃₋₈ cycloalkyl)₂, —NHC(O)(C₁₋₆ alkyl), —NHC(O)(C₁₋₆ haloalkyl), —NHC(O)(C₃₋₈ cycloalkyl), —NHC(O)(5- to 10-membered heterocyclyl), —NHC(O)(phenyl), —NHC(O)(5- to 10-membered heteroaryl), —NHC(O)O(C₁₋₆ alkyl), —NHC(O)O(C₁₋₆ haloalkyl), —NHC(O)O(C₂₋₆ alkynyl), —NHC(O)O(C₃₋₈ cycloalkyl), —NHC(O)O(5- to 10-membered heterocyclyl), —NHC(O)O(phenyl), —NHC(O)O(5- to 10-membered heteroaryl), —NHC(O)NH(C₁₋₆ alkyl), S(O)₂(C₁₋₆ alkyl), —S(O)₂(C₁₋₆ haloalkyl), —S(O)₂(C₃₋₈ cycloalkyl), —S(O)₂(5- to 10-membered heterocyclyl), —S(O)₂(phenyl), —S(O)₂(5- to 10-membered heteroaryl), —S(O)(NH)(C₁₋₆ alkyl), —S(O)₂NH(C₁₋₆ alkyl), or —S(O)₂N(C₁₋₆ alkyl)₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, —(C₁₋₆ alkyl)O(C₁₋₆ alkyl)-, —(C₁₋₆ alkyl)(R^{L2})NC(O)—, —(C₁₋₆ alkyl)C(O)N(R^{L2})—, —(C₁₋₆ alkyl)S(O)₂N(R^{L2})—, —(C₁₋₆ alkyl)N(R^{L2})S(O)₂—, —(C₁₋₆ alkyl)S(O)₂N(R^{L2})(C₁₋₆ alkyl)-, or —(C₁₋₆ alkyl)S(O)₂—(C₁₋₆ alkyl)-;
R^{L2} is hydrogen or C₁₋₆ alkyl;
R⁵ is hydrogen, —CN, —OR^{5a}, —C(O)NR^{5a}₂, —NR^{5a}C(O)R^{5a}, —NR^{5a}₂ C₁₋₆ alkyl, C₃₋₈ cycloalkyl, phenyl, 4- to 10-membered heterocyclyl, or 5- to 10-membered heteroaryl,
wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{b};
each R^{5a} is independently hydrogen or C₁₋₆ alkyl; and
each R^{5b} is independently halogen, oxo, cyclopropyl, hydroxy, —CN, C₁₋₃ alkyl, C₁₋₃ alkoxy, —OCF₃, or —OCF₂H.

2-17. (canceled)

18. The compound of claim 1, or a pharmaceutically acceptable salt thereof, wherein
ring \textcircled{A} taken together with X⁴ and X⁵ is phenyl, 5- to 6-membered heteroaryl, or C₅-10 cycloalkyl, and
X⁴ and X⁵ are each C.

19-28. (canceled)

29. The compound of claim 1, or a pharmaceutically acceptable salt thereof, wherein L³ is a bond.

30. The compound of claim 1, wherein each R³ is independently a hydrogen, halogen, hydroxy, —CN, C₁₋₆ alkyl, C₂₋₆ alkynyl, C₁₋₆ alkoxy, C₃₋₈ cycloalkyl, 4- to 10-membered heterocyclyl, phenyl, 5- to 10-membered heteroaryl, —NH₂, —NH(C₁₋₆ alkyl), —N(C₁₋₆ alkyl)₂, —O(C₁₋₆ alkyl), or —O(C₃₋₈ cycloalkyl);
wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to three R^{3a}.

31-54. (canceled)

55. The compound of claim 1, or a pharmaceutically acceptable salt thereof, wherein ring \textcircled{A} is absent.

56. The compound of claim 55, or a pharmaceutically acceptable salt thereof, wherein each L^{x4} and L^{x5} is independently a bond or N(R^{L})S(O)₂—.

57-67. (canceled)

68. The compound of claim 55, or a pharmaceutically acceptable salt thereof, wherein each R^{x4} and R^{x5} is independently hydrogen, C₁₋₆ alkyl, —NH₂, —NH(C₁₋₆ alkyl), —N(C₁₋₆ alkyl)₂, C₁₋₆ alkoxy, C₃₋₈ cycloalkyl, or 5- to 10-membered heterocyclyl,
wherein each C₁₋₆ alkyl, C₁₋₆ alkoxy, C₃₋₈ cycloalkyl, and 5- to 10-membered heterocyclyl of R^{x4} and R^{x5} is optionally substituted with one to three R^{4a}.

69-74. (canceled)

75. The compound of claim 1, or a pharmaceutically acceptable salt thereof, wherein ring \textcircled{B} is phenyl.

76-77. (canceled)

78. The compound of claim 1, or a pharmaceutically acceptable salt thereof, wherein each L¹ is a bond.

79-81. (canceled)

82. The compound of claim 1, or a pharmaceutically acceptable salt thereof, wherein each R¹ is independently halogen, —CN, —C(O)NH₂, C₁₋₆ alkyl, C₂₋₆ alkynyl, C₁₋₆ alkoxy, C₃₋₈ cycloalkyl, phenyl, or 5- to 10-membered heteroaryl,
wherein each C₁₋₆ alkyl, C₂₋₆ alkynyl, C₁₋₆ alkoxy, C₃₋₈ cycloalkyl, phenyl, and 5- to 10-membered heteroaryl of R¹ is optionally substituted with one or two R^{1a}.

83-92. (canceled)

93. The compound of claim 1, or a pharmaceutically acceptable salt thereof, wherein ring \textcircled{B} is

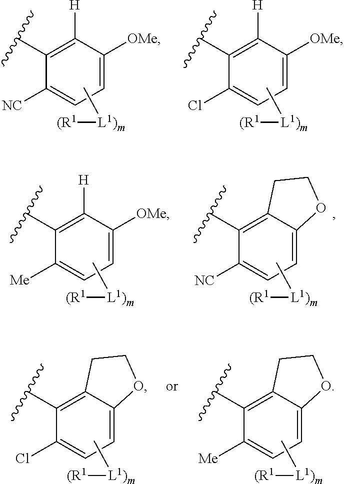

94-98. (canceled)

99. The compound of claim 1, or a pharmaceutically acceptable salt thereof, wherein R² is hydrogen, halogen, —CN, C₁₋₃ alkyl, or C₁₋₃ alkoxy.

100-101. (canceled)

102. The compound of claim 1, or a pharmaceutically acceptable salt thereof, wherein L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-.

103-106. (canceled)

107. The compound of claim 1, or a pharmaceutically acceptable salt thereof, wherein R⁵ is hydrogen, —CN, C₁₋₆ alkyl, C₃₋₈ cycloalkyl, phenyl, 4- to 10-membered heterocyclyl, or 5- to 10-membered heteroaryl, wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{5b}.

108-115. (canceled)

116. The compound of claim 1, having the structure of Formula (III): 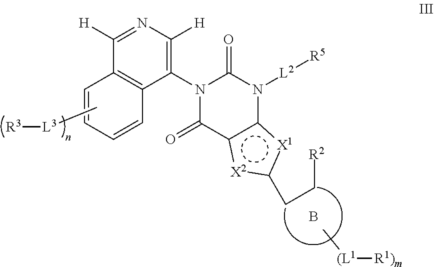
or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
each L³ is independently a bond, —(C₁₋₆ alkyl)O—, —(C₁₋₆ alkyl)-, —C(O)—, or —(C₁₋₆ alkyl)C(O)—;
each R³ is independently a hydrogen, halogen, hydroxy, —CN, C₁₋₆ alkyl, C₂₋₆ alkynyl, C₁₋₆ alkoxy, C₃₋₈ cycloalkyl, or 4- to 10-membered heterocyclyl;
wherein each C₁₋₆ alkyl, C₂₋₆ alkynyl, C₁₋₆ alkoxy, C₃₋₈ cycloalkyl, 4- to 10-membered heterocyclyl, phenyl, and 5- to 10-membered heteroaryl of R³ is optionally substituted with one to two R^{3a};
each R^{3a} is independently halogen, —C(O)(C₁-C₆ alkyl), —OH, —O(C₁₋₆ alkyl), 5- to 10-membered heterocyclyl, —C(O)NH₂, —C(O)N(H)(C₁-C₆ alkyl), or —C(O)N(C₁-C₆ alkyl)₂;
n is an integer from 0 to 3;
ring \textcircled{B} is C₆₋₁₀ aryl;
each L¹ is independently bond, —O—, —(C₁₋₆ alkyl)O—, —O(C₁₋₆ alkyl)-, or —C₁₋₆ alkyl-O(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl,
wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
each R^{1b} is independently C₁₋₆ alkyl, C₁₋₆ haloalkyl, halogen, oxo, —OH, or —NH₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-;
R⁵ is hydrogen, —CN, C₁₋₆ alkyl, C₃₋₈ cycloalkyl, phenyl, 4- to 10-membered heterocyclyl, or 5- to 10-membered heteroaryl,
wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{b};
each R^{5a} is independently hydrogen or C₁₋₆ alkyl; and
each R^{5b} is independently oxo, C₁₋₃ alkyl, or C₁₋₃ alkoxy.

117. The compound of claim 1, having the structure of Formula (VI): 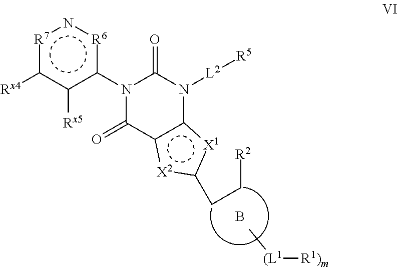
or a pharmaceutically acceptable salt thereof, wherein
X¹ is —O—, and X² is —N— or —C(R^{x2})=; or
X² is —O—, and X¹ is —N— or —C(R^{x1})=;
each R^{x1} and R^{x2} is independently hydrogen, halogen, C₁₋₃ alkyl, C₁₋₃ haloalkyl, —O(C₁₋₃ haloalkyl), C₁₋₃ alkoxy, cyclopropyl, or O-cyclopropyl;
each R^{x4} and R^{x5} is independently hydrogen, C₁₋₆ alkyl, —NH₂, —NH(C₁₋₆ alkyl), —N(C₁₋₆ alkyl)₂, C₁₋₆ alkoxy, C₃-C₈ cycloalkyl, or 5- to 10-membered heterocyclyl;
wherein each C₁₋₆ alkyl, C₁₋₆ alkoxy, C₃₋₈ cycloalkyl, and 5- to 10-membered heterocyclyl of R^{x4} and R^{x5} is optionally substituted with one to four R^{4a};
each R^{4a} is independently C₁₋₆ alkyl, —C(O)(5- to 10-membered heterocyclyl), or —O(C₃₋₈ cycloalkyl),
wherein each C₁₋₆ alkyl, C₃₋₈ cycloalkyl, and 5- to 10-membered heterocyclyl of R^{4a} is optionally substituted with one to three R^{4b};
each R^{4b} is independently halogen, —O(C₁₋₆ alkyl), or —O(C₁₋₆ haloalkyl);
each X⁶ and X⁷ is independently N or CH;
ring \textcircled{B} is C₆₋₁₀ aryl;
each L¹ is independently bond, —O—, —(C₁₋₆ alkyl)O—, —O(C₁₋₆ alkyl)-, or —C₁₋₆ alkyl-O(C₁₋₆ alkyl)-;
each R¹ is independently halogen, —OH, —CN, C₁₋₆ alkyl, —C(O)NH₂, C₂₋₆ alkenyl, C₂₋₆ alkynyl, C₁₋₆ alkoxy, C₃₋₈ cycloalkyl, phenyl, 5- to 12-membered heteroaryl, or 4- to 10-membered heterocyclyl,
wherein each C₁₋₆ alkyl, C₂₋₆ alkenyl, C₂₋₆ alkynyl, C₁₋₆ alkoxy, C₃₋₈ cycloalkyl, phenyl, 5- to 12-membered heteroaryl, and 4- to 10-membered heterocyclyl of R¹ is optionally substituted with one to four R^{1a};
each R^{1a} is independently halogen, —OH, —CN, —C(O)NH₂, C₁₋₆ alkyl, C₁₋₆ alkoxy, C₃₋₈ cycloalkyl, phenyl, or 5- to 10-membered heteroaryl,
wherein each C₁₋₆ alkyl, C₃₋₈ cycloalkyl, 4- to 10-membered heterocyclyl, phenyl, and 5- to 10-membered heteroaryl of R^{1a} is optionally substituted with one to three R^{1b};
each R^{1b} is independently C₁₋₆ alkyl, C₁₋₆ haloalkyl, halogen, oxo, —OH, or —NH₂;
m is an integer from 0 to 3;
R² is hydrogen, C₁₋₃ alkyl, C₁₋₃ haloalkyl, cyclopropyl, C₁₋₃ alkoxy, —O(C₁₋₃ haloalkyl), —O(cyclopropyl), halogen, or —CN;
L² is a bond, C₁₋₆ alkyl, —(C₀₋₆ alkyl)-cyclopropyl-(C₀₋₆ alkyl)-, —(C₁₋₆ alkyl)O—, or —(C₁₋₆ alkyl)O(C₁₋₆ alkyl)-;
R⁵ is hydrogen, —CN, C₁₋₆ alkyl, C₃₋₈ cycloalkyl, phenyl, 4- to 10-membered heterocyclyl, or 5- to 10-membered heteroaryl,
wherein each C₁₋₆ alkyl, C₃₋₈ cycloalkyl, phenyl, 4- to 10-membered heterocyclyl, and 5- to 10-membered heteroaryl of R⁵ is optionally substituted with one or two R^{b};
each R^{5a} is independently hydrogen or C₁₋₆ alkyl; and
each R^{5b} is independently oxo, C₁₋₃ alkyl, or C₁₋₃ alkoxy.

118-120. (canceled)

121. A compound selected from 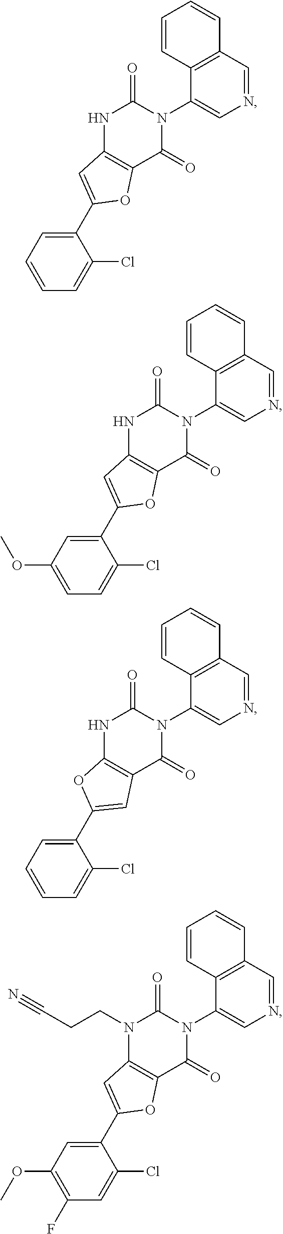 [SMILES: O=c1[nH]c2cc(-c3ccccc3Cl)oc2c(=O)n1-c1cncc2ccccc12 ; COc1ccc(Cl)c(-c2cc3[nH]c(=O)n(-c4cncc5ccccc45)c(=O)c3o2)c1 ; O=c1[nH]c2oc(-c3ccccc3Cl)cc2c(=O)n1-c1cncc2ccccc12 ; COc1cc(-c2cc3c(o2)c(=O)n(-c2cncc4ccccc24)c(=O)n3CCC#N)c(Cl)cc1F] 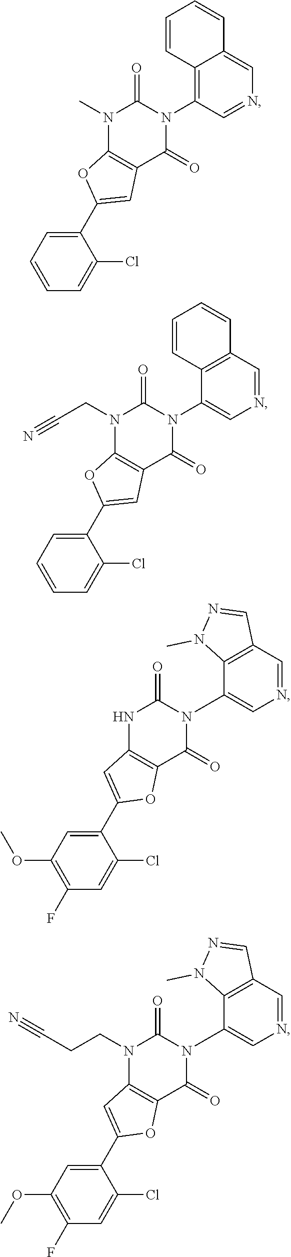 [SMILES: Cn1c(=O)n(-c2cncc3ccccc23)c(=O)c2cc(-c3ccccc3Cl)oc21 ; N#CCn1c(=O)n(-c2cncc3ccccc23)c(=O)c2cc(-c3ccccc3Cl)oc21 ; COc1cc(-c2cc3[nH]c(=O)n(-c4cncc5cnn(C)c45)c(=O)c3o2)c(Cl)cc1F ; COc1cc(-c2cc3c(o2)c(=O)n(-c2cncc4cnn(C)c24)c(=O)n3CCC#N)c(Cl)cc1F] 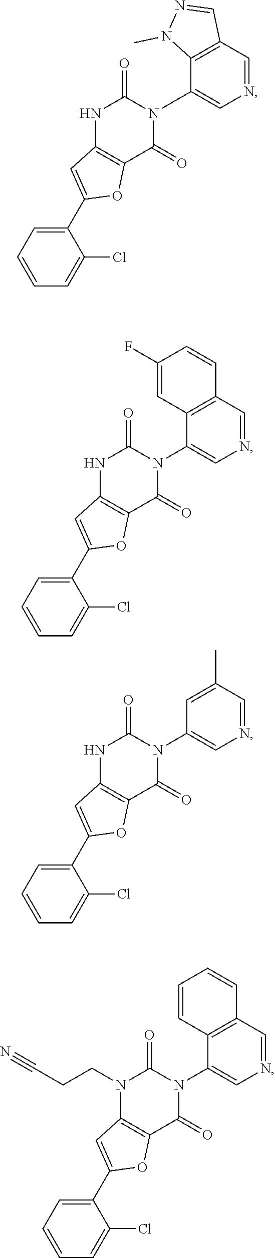 [SMILES: Cn1ncc2cncc(-n3c(=O)[nH]c4cc(-c5ccccc5Cl)oc4c3=O)c21 ; O=c1[nH]c2cc(-c3ccccc3Cl)oc2c(=O)n1-c1cncc2ccc(F)cc12 ; Cc1cncc(-n2c(=O)[nH]c3cc(-c4ccccc4Cl)oc3c2=O)c1 ; N#CCCn1c(=O)n(-c2cncc3ccccc23)c(=O)c2oc(-c3ccccc3Cl)cc21] 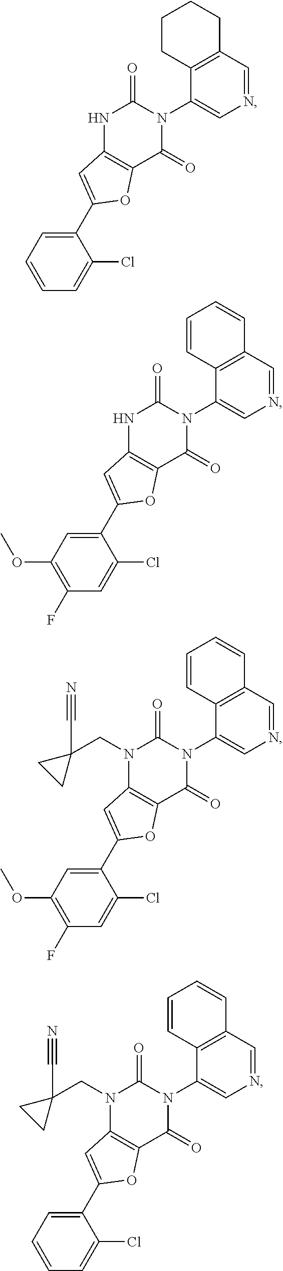 [SMILES: O=c1[nH]c2cc(-c3ccccc3Cl)oc2c(=O)n1-c1cncc2c1CCCC2 ; COc1cc(-c2cc3[nH]c(=O)n(-c4cncc5ccccc45)c(=O)c3o2)c(Cl)cc1F ; COc1cc(-c2cc3c(o2)c(=O)n(-c2cncc4ccccc24)c(=O)n3CC2(C#N)CC2)c(Cl)cc1F ; N#CC1(Cn2c(=O)n(-c3cncc4ccccc34)c(=O)c3oc(-c4ccccc4Cl)cc32)CC1] 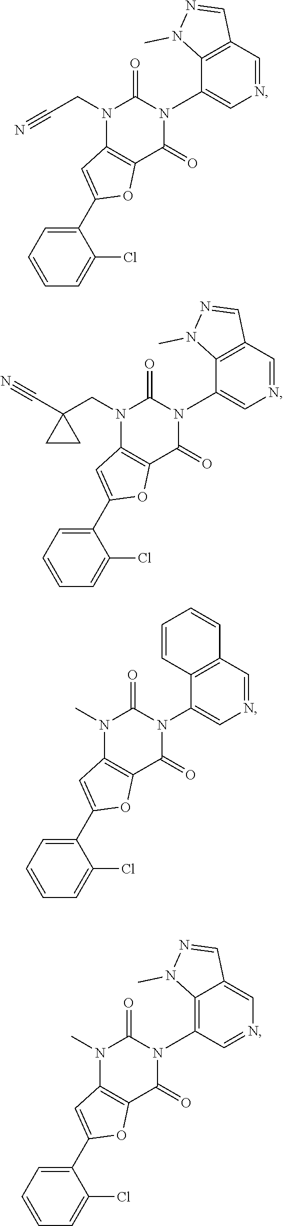 [SMILES: Cn1ncc2cncc(-n3c(=O)c4oc(-c5ccccc5Cl)cc4n(CC#N)c3=O)c21 ; Cn1ncc2cncc(-n3c(=O)c4oc(-c5ccccc5Cl)cc4n(CC4(C#N)CC4)c3=O)c21 ; Cn1c(=O)n(-c2cncc3ccccc23)c(=O)c2oc(-c3ccccc3Cl)cc21 ; Cn1ncc2cncc(-n3c(=O)c4oc(-c5ccccc5Cl)cc4n(C)c3=O)c21] 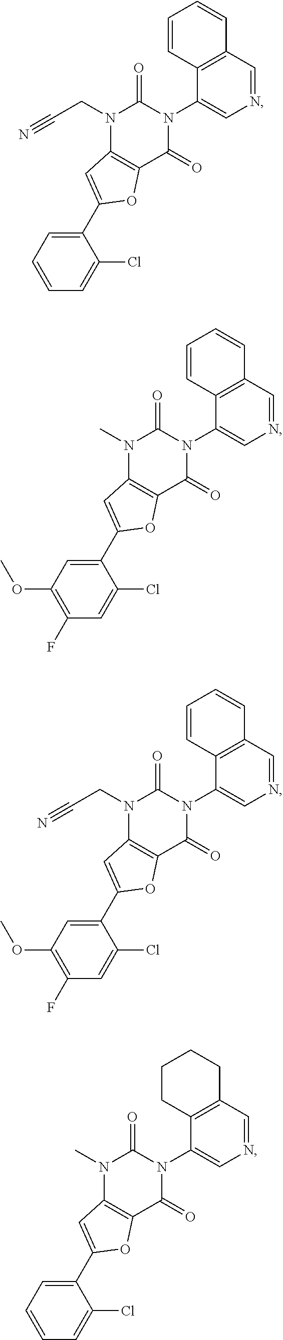 [SMILES: N#CCn1c(=O)n(-c2cncc3ccccc23)c(=O)c2oc(-c3ccccc3Cl)cc21 ; COc1cc(-c2cc3c(o2)c(=O)n(-c2cncc4ccccc24)c(=O)n3C)c(Cl)cc1F ; COc1cc(-c2cc3c(o2)c(=O)n(-c2cncc4ccccc24)c(=O)n3CC#N)c(Cl)cc1F ; Cn1c(=O)n(-c2cncc3c2CCCC3)c(=O)c2oc(-c3ccccc3Cl)cc21] 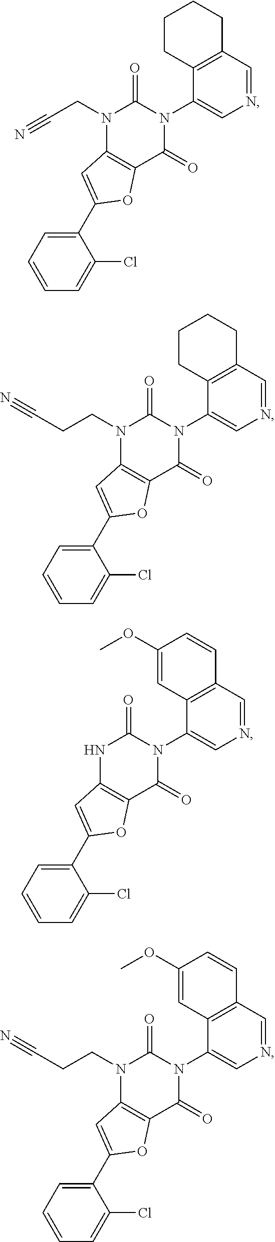 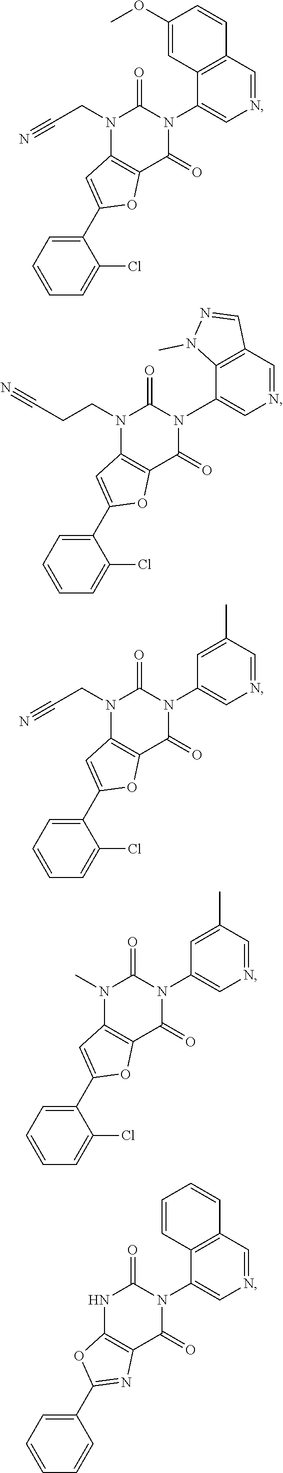 [SMILES: COc1ccc2cncc(-n3c(=O)c4oc(-c5ccccc5Cl)cc4n(CC#N)c3=O)c2c1 ; Cn1ncc2cncc(-n3c(=O)c4oc(-c5ccccc5Cl)cc4n(CCC#N)c3=O)c21 ; Cc1cncc(-n2c(=O)c3oc(-c4ccccc4Cl)cc3n(CC#N)c2=O)c1 ; Cc1cncc(-n2c(=O)c3oc(-c4ccccc4Cl)cc3n(C)c2=O)c1 ; O=c1[nH]c2oc(-c3ccccc3)nc2c(=O)n1-c1cncc2ccccc12] 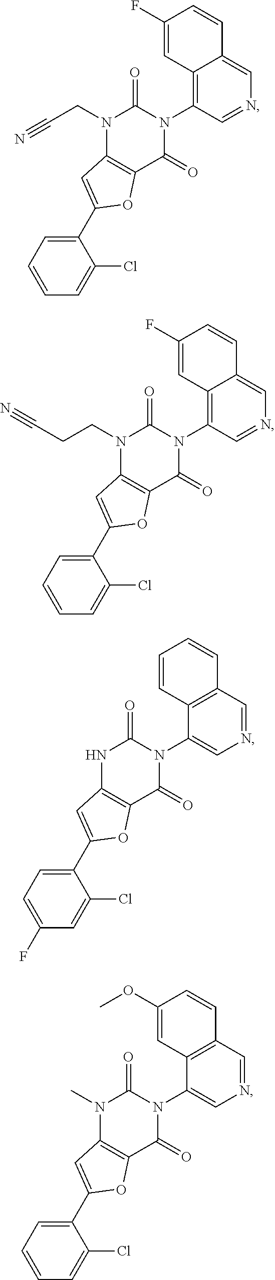 [SMILES: N#CCn1c(=O)n(-c2cncc3ccc(F)cc23)c(=O)c2oc(-c3ccccc3Cl)cc21 ; N#CCCn1c(=O)n(-c2cncc3ccc(F)cc23)c(=O)c2oc(-c3ccccc3Cl)cc21 ; O=c1[nH]c2cc(-c3ccc(F)cc3Cl)oc2c(=O)n1-c1cncc2ccccc12 ; COc1ccc2cncc(-n3c(=O)c4oc(-c5ccccc5Cl)cc4n(C)c3=O)c2c1] 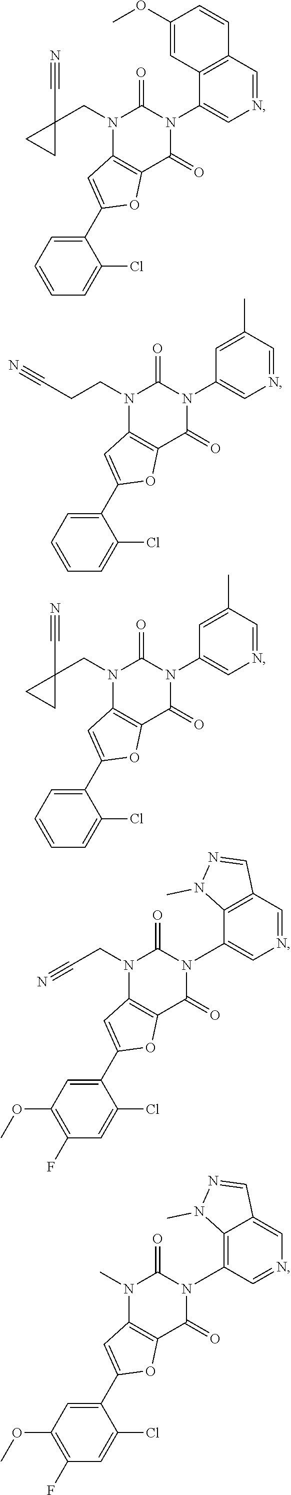 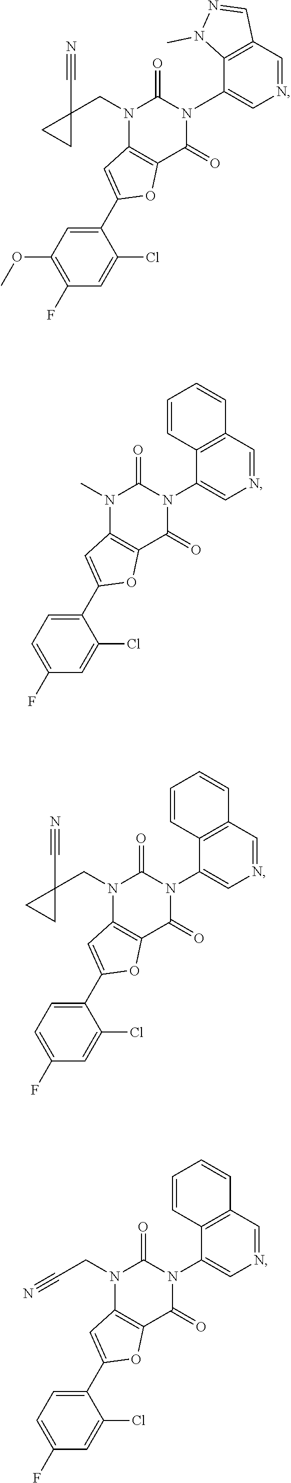 [SMILES: COc1cc(-c2cc3c(o2)c(=O)n(-c2cncc4cnn(C)c24)c(=O)n3CC2(C#N)CC2)c(Cl)cc1F ; Cn1c(=O)n(-c2cncc3ccccc23)c(=O)c2oc(-c3ccc(F)cc3Cl)cc21 ; N#CC1(Cn2c(=O)n(-c3cncc4ccccc34)c(=O)c3oc(-c4ccc(F)cc4Cl)cc32)CC1 ; N#CCn1c(=O)n(-c2cncc3ccccc23)c(=O)c2oc(-c3ccc(F)cc3Cl)cc21] 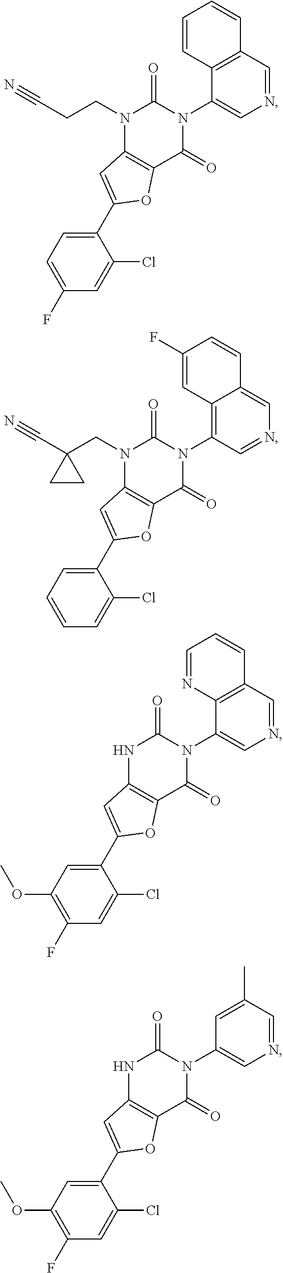 [SMILES: N#CCCn1c(=O)n(-c2cncc3ccccc23)c(=O)c2oc(-c3ccc(F)cc3Cl)cc21 ; N#CC1(Cn2c(=O)n(-c3cncc4ccc(F)cc34)c(=O)c3oc(-c4ccccc4Cl)cc32)CC1 ; COc1cc(-c2cc3[nH]c(=O)n(-c4cncc5cccnc45)c(=O)c3o2)c(Cl)cc1F ; COc1cc(-c2cc3[nH]c(=O)n(-c4cncc(C)c4)c(=O)c3o2)c(Cl)cc1F] 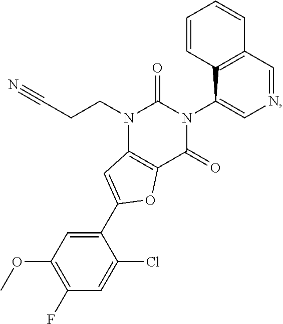 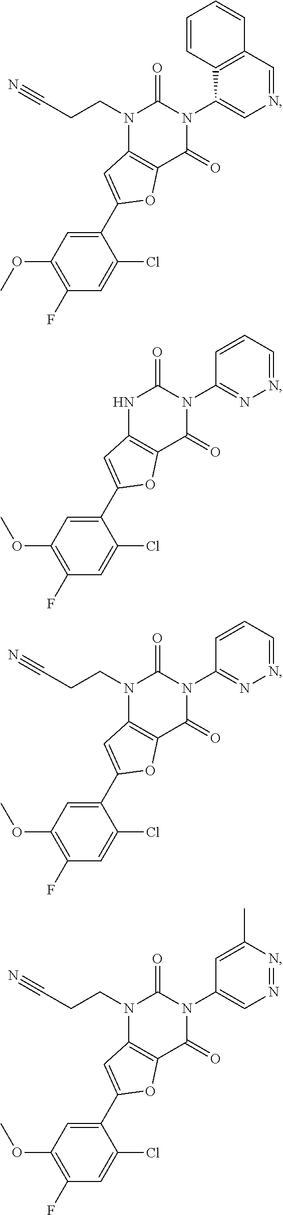 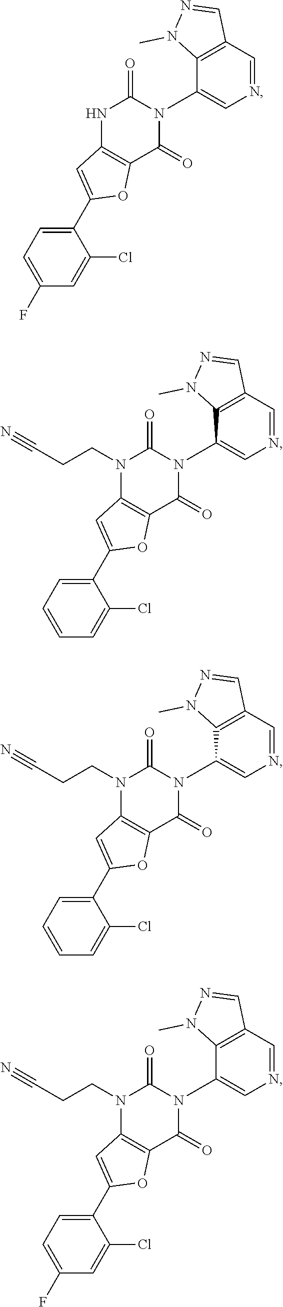 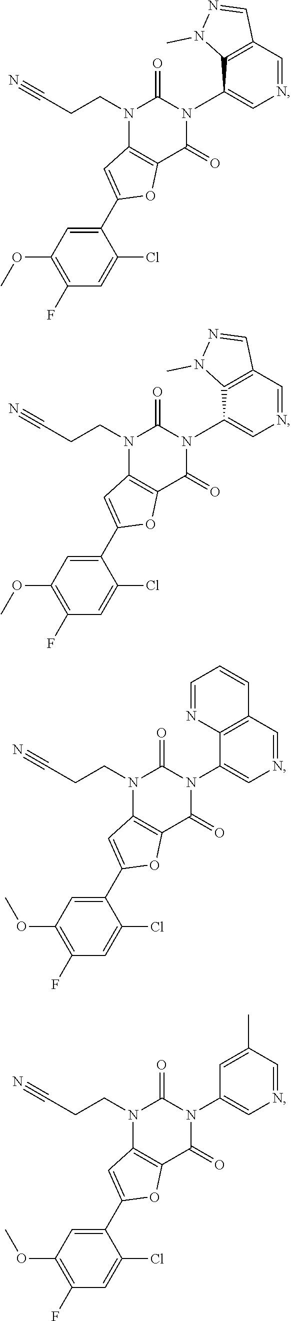 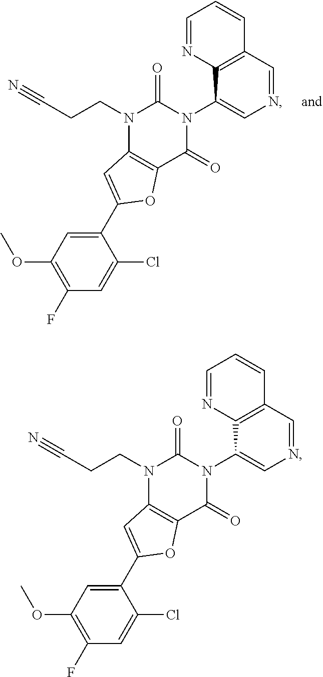
or a pharmaceutically acceptable salt thereof.

122. A pharmaceutical composition comprising the compound of claim 1, or a pharmaceutically acceptable salt thereof, and a pharmaceutically acceptable carrier or excipient.

123. A method of treating or preventing a viral infection in a human in need thereof, wherein the method comprises administering to the human the compound of claim 1, or a pharmaceutically acceptable salt thereof.

124. The method of claim 123, wherein the viral infection is a coronavirus infection.

125-132. (canceled)
